SEQUENCE LISTING (222 sequences)

SEQ ID NO: 1 - protein, 10 aa, synthetic construct
GYTFTSYNMH

SEQ ID NO: 2 - protein, 17 aa, synthetic construct
AIYPGSGDTSYNQKFKG

SEQ ID NO: 3 - protein, 13 aa, synthetic construct
VVYYSNSYWYFDV

SEQ ID NO: 4 - protein, 10 aa, synthetic construct
RASSSVSYMH

SEQ ID NO: 5 - protein, 7 aa, synthetic construct
APSNLAS

SEQ ID NO: 6 - protein, 9 aa, synthetic construct
QQWSFNPPT

SEQ ID NO: 7 - protein, 122 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSS

SEQ ID NO: 8 - protein, 106 aa, synthetic construct [sequence omitted]

SEQ ID NO: 9 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 225 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 11 - protein, 577 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - protein, 591 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGFTFDDYAIGWFRQAPGKEREGVSCIRVSDGSTYYADSVKGRFTISRDNSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 5 aa, synthetic construct
DYEMH

SEQ ID NO: 14 - protein, 17 aa, synthetic construct
ALDPKTGDTAYSQKFKG

SEQ ID NO: 15 - protein, 6 aa, synthetic construct
FYSYTY

SEQ ID NO: 16 - protein, 16 aa, synthetic construct
RSSQSLVHSNRNTYLH

SEQ ID NO: 17 - protein, 7 aa, synthetic construct
KVSNRFS

SEQ ID NO: 18 - protein, 9 aa, synthetic construct
SQNTHVPPT

SEQ ID NO: 19 - protein, 115 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 20 - protein, 112 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 21 - protein, 219 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 218 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 23 - protein, 570 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 584 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGFTFDDYAIGWFRQAPGKEREGVSCIRVSDGSTYYADSVKGRFTISRDNSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 5 aa, synthetic construct
SYWIE

SEQ ID NO: 26 - protein, 17 aa, synthetic construct
EILPGSDTTNYNEKFKD

SEQ ID NO: 27 - protein, 11 aa, synthetic construct
DRGNYRAWFGY

SEQ ID NO: 28 - protein, 11 aa, synthetic construct
RASQDISNYLN

SEQ ID NO: 29 - protein, 7 aa, synthetic construct
YTSRLHS

SEQ ID NO: 30 - protein, 9 aa, synthetic construct
QQGEALPWT

SEQ ID NO: 31 - protein, 120 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 32 - protein, 107 aa, synthetic construct
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIK

SEQ ID NO: 33 - protein, 214 aa, synthetic construct
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 223 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 35 - protein, 575 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 36 - protein, 589 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGFTFDDYAIGWFRQAPGKEREGVSCIRVSDGSTYYADSVKGRFTISRDNSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 4 aa, synthetic construct
TGGS

SEQ ID NO: 38 - protein, 5 aa, synthetic construct
GGGGS

SEQ ID NO: 39 - protein, 5 aa, synthetic construct
INFLG

SEQ ID NO: 40 - protein, 16 aa, synthetic construct
HISIGDQTDYADSAKG

SEQ ID NO: 41 - protein, 9 aa, synthetic construct
FSRIYPYDY

SEQ ID NO: 42 - protein, 117 aa, synthetic construct
EVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSS

SEQ ID NO: 43 - protein, 117 aa, synthetic construct
EVQLLESGGGLVQPGGSLRLSCAASGDVHKINFLSWYRQAPGKEREKVAHISIGDQTDYADSAKGRFTISRDESKNTLYLQMNSLRAEDTAVYYCRAFSRIYPYDYWGQGTLVTVSS

SEQ ID NO: 44 - protein, 117 aa, synthetic construct
EVQLLESGGGLVQPGGSLRLSCAASGDVHKINFLGWYRQAPGKEREKVAHISIGDQTDYADSAKGRFTISRDESKNTLYLQMNSLRAEDTAVYYCRAFSRIYPYDYWGQGTLVTVSS

SEQ ID NO: 45 - protein, 117 aa, synthetic construct
EVQLLESGGGLVQPGGSLRLSCAASGDVHKINFLSWYRQAPGKGLEKVAHISIGDQTDYADSAKGRFTISRDESKNTLYLQMNSLRAEDTAVYYCRAFSRIYPYDYWGQGTLVTVSS

SEQ ID NO: 46 - protein, 117 aa, synthetic construct
EVQLLESGGGLVQPGGSLRLSCAASGDVHKINFLGWYRQAPGKGLEKVAHISIGDQTDYADSAKGRFTISRDESKNTLYLQMNSLRAEDTAVYYCRAFSRIYPYDYWGQGTLVTVSS

SEQ ID NO: 47 - protein, 117 aa, synthetic construct
EVQLLESGGGLVQPGGSLRLSCAASGDVHKINFLGWYRQAPGKEREKVAHISIGDQTDYADSAKGRFTISRDESKNTVYLQMNSLRAEDTAVYFCRAFSRIYPYDYWGQGTLVTVSS

SEQ ID NO: 48 - protein, 117 aa, synthetic construct
EVQLLESGGGLVQPGGSLRLSCAASGDVHKINFLGWYRQAPGKGLEKVAHISIGDQTDYADSAKGRFTISRDESKNTVYLQMNSLRAEDTAVYFCRAFSRIYPYDYWGQGTLVTVSS

SEQ ID NO: 49 - protein, 5 aa, synthetic construct
DYAIG

SEQ ID NO: 50 - protein, 16 aa, synthetic construct
IRVSDGSTYYADSVKG

SEQ ID NO: 51 - protein, 22 aa, synthetic construct
GSLYTCVQSIVWPARPYYDMDY

SEQ ID NO: 52 - protein, 127 aa, synthetic construct
EVQLLESGGGLVQPGGSLRLSCAASGFTFDDYAIGWFRQAPGKEREGVSCIRVSDGSTYYADSVKGRFTISRDNSKNTLYLQMNSLRAEDTAVYYCAAGSLYTCVQSIVWPARPYYDMDYWGQGTLV

SEQ ID NO: 53 - protein, 12 aa, synthetic construct
DKTHTCPPCPAP

SEQ ID NO: 54 - skipped

SEQ ID NO: 55 - protein, 10 aa, synthetic construct
THTCPPCPAP

SEQ ID NO: 56 - protein, 214 aa, synthetic construct
PVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 57 - protein, 214 aa, synthetic construct
PVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 58 - protein, 214 aa, synthetic construct
PVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 60 - protein, 455 aa, synthetic construct; identical to SEQ ID NO: 59

SEQ ID NO: 61 - protein, 469 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGFTFDDYAIGWFRQAPGKEREGVSCIRVSDGSTYYADSVKGRFTISRDNSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 5 aa, synthetic construct
INFLS

SEQ ID NO: 63 - protein, 575 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLSWYRQAPGKEREKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 64 - protein, 575 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKEREKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 65 - protein, 575 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLSWYRQAPGKGLEKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 66 - protein, 575 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKGLEKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 67 - protein, 575 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKEREKVAHISIGDQTDYADSAKGRFTISRDESKNTVYLQMNSLRAEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 68 - protein, 575 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKGLEKVAHISIGDQTDYADSAKGRFTISRDESKNTVYLQMNSLRAEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 69 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLSWYRQAPGKEREKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 70 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKEREKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 71 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLSWYRQAPGKGLEKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 72 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKGLEKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 73 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKEREKVAHISIGDQTDYADSAKGRFTISRDESKNTVYLQMNSLRAEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 74 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKGLEKVAHISIGDQTDYADSAKGRFTISRDESKNTVYLQMNSLRAEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 75 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLSWYRQAPGKEREKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 76 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKEREKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 77 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLSWYRQAPGKGLEKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 78 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKGLEKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 79 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKEREKVAHISIGDQTDYADSAKGRFTISRDESKNTVYLQMNSLRAEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 80 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKGLEKVAHISIGDQTDYADSAKGRFTISRDESKNTVYLQMNSLRAEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 81 - protein, 131 aa, synthetic construct
EVQLLESGGGLVQPGGSLRLSCAASGFTFPDYAIGWFRQAPGKEREGVSCIRVSDGSTYYADSVKGRFTISRDNAKNTLYLQMNSLRAEDTAVYYCAAGSLYTCVNSIVWPARPYYDMDYWGQGTLVTVSS

SEQ ID NO: 82 - protein, 131 aa, synthetic construct
EVQLLESGGGLVQPGGSLRLSCAASGFTFPDYAIGWFRQAPGKEREGVSCIRVSDGSTYYADSVKGRFTISRDNAKNTLYLQMNSLRAEDTAVYYCAAGSAYTCVQSIVWPARPYYDMDYWGQGTLVTVSS

SEQ ID NO: 83 - protein, 131 aa, synthetic construct
EVQLLESGGGLVQPGGSLRLSCAASGFTFRDYAIGWFRQAPGKEREGVSCIRVSDGSTYYADSVKGRFTISRDNAKNTLYLQMNSLRAEDTAVYYCAAGSLYTCVQSIVWPARPYYDMDYWGQGTLVTVSS

SEQ ID NO: 84 - protein, 131 aa, synthetic construct
EVQLLESGGGLVQPGGSLRLSCAASGFTFPDYAIGWFRQAPGKEREGVSCIRVSDGSTYYADSVKGRFTISRDNAKNTLYLQMNSLRAEDTAVYYCAAGGLYTCVQSIVWPARPYYDMDYWGQGTLVTVSS

SEQ ID NO: 85 - protein, 131 aa, synthetic construct
EVQLLESGGGLVQPGGSLRLSCAASGFTFPDYAIGWFRQAPGKEREGVSCIRVSDGSTYYADSVKGRFTISRDNAKNTLYLQMNSLRAEDTAVYYCAAGSLITCVQSIVWPARPYYDMDYWGQGTLVTVSS

SEQ ID NO: 86 - protein, 131 aa, synthetic construct
EVQLLESGGGLVQPGGSLRLSCAASGFTFPDYAIGWFRQAPGKEREGVSCIRVSDGSTYYADSVKGRFTISRDNAKNTLYLQMNSLRAEDTAVYYCAAGPLYTCVQSIVWPARPYYDMDYWGQGTLVTVSS

SEQ ID NO: 87 - protein, 131 aa, synthetic construct
EVQLLESGGGLVQPGGSLRLSCAASGFTFDSYAIGWFRQAPGKEREGVSCIRVSDGSTYYADSVKGRFTISRDNAKNTLYLQMNSLRAEDTAVYYCAAGSLYTCVQSIVWPARPYYDMDYWGQGTLVTVSS

SEQ ID NO: 88 - protein, 131 aa, synthetic construct
EVQLLESGGGLVQPGGSLRLSCAASGFTFPDYAIGWFRQAPGKEREGVSCITVSDGSTYYADSVKGRFTISRDNAKNTLYLQMNSLRAEDTAVYYCAAGSLYTCVQSIVWPARPYYDMDYWGQGTLVTVSS

SEQ ID NO: 89 - protein, 131 aa, synthetic construct
EVQLLESGGGLVQPGGSLRLSCAASGFTFPDYAIGWFRQAPGKEREGVSCIRVSDGSTYYADSVKGRFTISRDNAKNTLYLQMNSLRAEDTAVYYCAAGSLYTCVQSIVWPARPYYDMDYWGQGTLVTVSS

SEQ ID NO: 90 - protein, 7 aa, synthetic construct
EPKSCDK

SEQ ID NO: 91 - protein, 8 aa, synthetic construct
THTCPPCP

SEQ ID NO: 92 - protein, 220 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 93 - protein, 213 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 94 - protein, 218 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 95 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 96 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 97 - protein, 103 aa, synthetic construct
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSV

SEQ ID NO: 98 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 99 - protein, 106 aa, synthetic construct
GQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 100 - protein, 107 aa, synthetic construct [sequence omitted]

SEQ ID NO: 101 - protein, 105 aa, synthetic construct
ELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTIS

SEQ ID NO: 102 - protein, 104 aa, synthetic construct
PVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTIS

SEQ ID NO: 103 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 103 aa, synthetic construct
ASTKGPSVFPLAPSSKSTSGGTASLGCLVKDYFPEPVTVSWNSGALTSGVHTFPAVLQSSGLYSLSSVVTVPSSSLGTQTYICNVNHKPSNTKVDKRVEPKSC

SEQ ID NO: 105 - protein, 106 aa, synthetic construct
GQPKAAPSVRLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 106 - protein, 103 aa, synthetic construct
ASTKGPSVCPLAPSSKSTSGGTASLGCLVKDYFPEPVTVSWNSGALTSGVHTFPAVLQSSGLYSLSSVVTVPSSSLGTQTYICNVNHKPSNTKVDKRVEPKSV

SEQ ID NO: 107 - protein, 106 aa, synthetic construct
GQPKAAPSVRLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 108 - protein, 106 aa, synthetic construct
APLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTIS

SEQ ID NO: 109 - protein, 106 aa, synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTIS

SEQ ID NO: 110 - protein, 577 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 111 - protein, 5 aa, synthetic construct
SYSMN

SEQ ID NO: 112 - protein, 17 aa, synthetic construct
SISGQSNYIYYADSVKG

SEQ ID NO: 113 - protein, 11 aa, synthetic construct
GGNYFVEYFQY

SEQ ID NO: 114 - protein, 12 aa, synthetic construct
RASQYISSNNLA

SEQ ID NO: 115 - protein, 7 aa, synthetic construct
GASNRAT

SEQ ID NO: 116 - protein, 9 aa, synthetic construct
QQYADSPIT

SEQ ID NO: 117 - protein, 120 aa, synthetic construct
EVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSS

SEQ ID NO: 118 - protein, 108 aa, synthetic construct
EIVLTQSPGTLSLSPGERATLSCRASQYISSNNLAWYQQKPGQAPRLLIYGASNRATGIPDRFSGSGSGTDFTLTISRLEPEDFAVYYCQQYADSPITFGQGTKLEIK

SEQ ID NO: 119 - protein, 215 aa, synthetic construct
EIVLTQSPGTLSLSPGERATLSCRASQYISSNNLAWYQQKPGQAPRLLIYGASNRATGIPDRFSGSGSGTDFTLTISRLEPEDFAVYYCQQYADSPITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 225 aa, synthetic construct
EVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 121 - protein, 575 aa, synthetic construct
EVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 122 - protein, 589 aa, synthetic construct
EVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGFTFDDYAIGWFRQAPGKEREGVSCIRVSDGSTYYADSVKGRFTISRDNSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 7 aa, synthetic construct
RNSAVWN

SEQ ID NO: 124 - protein, 18 aa, synthetic construct
RTYYRSKWYNDYAVSVKS

SEQ ID NO: 125 - protein, 13 aa, synthetic construct
GLRQNQFYYYMDV

SEQ ID NO: 126 - protein, 11 aa, synthetic construct
RASQSVASNLA

SEQ ID NO: 127 - protein, 7 aa, synthetic construct
GASTRAT

SEQ ID NO: 128 - protein, 9 aa, synthetic construct
QQYNNWPFT

SEQ ID NO: 129 - protein, 125 aa, synthetic construct
QVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLRQNQFYYYMDVWGKGTTVTVSS

SEQ ID NO: 130 - protein, 107 aa, synthetic construct
EIVMTQSPATLSVSPGERATLSCRASQSVASNLAWYQQKPGQAPRLLIYGASTRATGIPARFSGSGSGTEFTLTISSLQSEDFAVYYCQQYNNWPFTFGPGTKVDIK

SEQ ID NO: 131 - protein, 214 aa, synthetic construct
EIVMTQSPATLSVSPGERATLSCRASQSVASNLAWYQQKPGQAPRLLIYGASTRATGIPARFSGSGSGTEFTLTISSLQSEDFAVYYCQQYNNWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 230 aa, synthetic construct
QVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLRQNQFYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 133 - protein, 594 aa, synthetic construct
QVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLRQNQFYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGFTFDDYAIGWFRQAPGKEREGVSCIRVSDGSTYYADSVKGRFTISRDNSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 580 aa, synthetic construct
QVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLRQNQFYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 135 - protein, 5 aa, synthetic construct
SYWIE

SEQ ID NO: 136 - protein, 17 aa, synthetic construct
EILPGSDTTNYAQNFQD

SEQ ID NO: 137 - protein, 11 aa, synthetic construct
DRGNYRAWFGY

SEQ ID NO: 138 - protein, 11 aa, synthetic construct
RASQDISNYLN

SEQ ID NO: 139 - protein, 7 aa, synthetic construct
YTSRLHS

SEQ ID NO: 140 - protein, 9 aa, synthetic construct
QQGNALPWT

SEQ ID NO: 141 - protein, 120 aa, synthetic construct
EVQLVQSGAEVKKPGASVKVSCKASGYKFSSYWIEWVKQAPGQGLEWIGEILPGSDTTNYAQNFQDRVTFTSDTSISTAYMELSRLRSDDTAVYYCARDRGNYRAWFGYWGQGTLVTVSS

SEQ ID NO: 142 - protein, 107 aa, synthetic construct
DIQMTQSPSSLSASVGDRVTITCRASQDISNYLNWYQQKPGKAPKFLIYYTSRLHSGVPSRFSGSGSGTDYTLTISSLQPEDFATYYCQQGNALPWTFGGGTKVEIK

SEQ ID NO: 143 - protein, 214 aa, synthetic construct
DIQMTQSPSSLSASVGDRVTITCRASQDISNYLNWYQQKPGKAPKFLIYYTSRLHSGVPSRFSGSGSGTDYTLTISSLQPEDFATYYCQQGNALPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 225 aa, synthetic construct
EVQLVQSGAEVKKPGASVKVSCKASGYKFSSYWIEWVKQAPGQGLEWIGEILPGSDTTNYAQNFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 145 - protein, 575 aa, synthetic construct
EVQLVQSGAEVKKPGASVKVSCKASGYKFSSYWIEWVKQAPGQGLEWIGEILPGSDTTNYAQNFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 146 - protein, 589 aa, synthetic construct
EVQLVQSGAEVKKPGASVKVSCKASGYKFSSYWIEWVKQAPGQGLEWIGEILPGSDTTNYAQNFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGFTFTDYAIGWFRQAPGKEREGVSCIRVSDGSTYYADSVKGRFTISRDSSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 17 aa, synthetic construct
EILPGSDTTNYAQQFQD

SEQ ID NO: 148 - protein, 120 aa, synthetic construct
EVQLVQSGAEVKKPGASVKVSCKASGYKFSSYWIEWVKQAPGQGLEWIGEILPGSDTTNYAQQFQDRVTFTSDTSISTAYMELSRLRSDDTAVYYCARDRGNYRAWFGYWGQGTLVTVSS

SEQ ID NO: 149 - protein, 225 aa, synthetic construct
EVQLVQSGAEVKKPGASVKVSCKASGYKFSSYWIEWVKQAPGQGLEWIGEILPGSDTTNYAQQFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 150 - protein, 575 aa, synthetic construct
EVQLVQSGAEVKKPGASVKVSCKASGYKFSSYWIEWVKQAPGQGLEWIGEILPGSDTTNYAQQFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 151 - protein, 589 aa, synthetic construct
EVQLVQSGAEVKKPGASVKVSCKASGYKFSSYWIEWVKQAPGQGLEWIGEILPGSDTTNYAQQFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGFTFTDYAIGWFRQAPGKEREGVSCIRVSDGSTYYADSVKGRFTISRDSSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 17 aa, synthetic construct
EILPGSDATNYAQKFQD

SEQ ID NO: 153 - protein, 120 aa, synthetic construct
EVQLVQSGAEVKKPGASVKVSCKASGYKFSSYWIEWVKQAPGQGLEWIGEILPGSDATNYAQKFQDRVTFTSDTSISTAYMELSRLRSDDTAVYYCARDRGNYRAWFGYWGQGTLVTVSS

SEQ ID NO: 154 - protein, 225 aa, synthetic construct
EVQLVQSGAEVKKPGASVKVSCKASGYKFSSYWIEWVKQAPGQGLEWIGEILPGSDATNYAQKFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 155 - protein, 575 aa, synthetic construct
EVQLVQSGAEVKKPGASVKVSCKASGYKFSSYWIEWVKQAPGQGLEWIGEILPGSDATNYAQKFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 156 - protein, 589 aa, synthetic construct
EVQLVQSGAEVKKPGASVKVSCKASGYKFSSYWIEWVKQAPGQGLEWIGEILPGSDATNYAQKFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGFTFTDYAIGWFRQAPGKEREGVSCIRVSDGSTYYADSVKGRFTISRDSSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 17 aa, synthetic construct
EILPGSDTTNYAQKFQD

SEQ ID NO: 158 - protein, 7 aa, synthetic construct
YTSRLES

SEQ ID NO: 159 - protein, 120 aa, synthetic construct
EVQLVQSGAEVKKPGASVKVSCKASGYKFSSYWIEWVKQAPGQGLEWIGEILPGSDTTNYAQKFQDRVTFTSDTSISTAYMELSRLRSDDTAVYYCARDRGNYRAWFGYWGQGTLVTVSS

SEQ ID NO: 160 - protein, 107 aa, synthetic construct
DIQMTQSPSSLSASVGDRVTITCRASQDISNYLNWYQQKPGKAPKFLIYYTSRLESGVPSRFSGSGSGTDYTLTISSLQPEDFATYYCQQGNALPWTFGGGTKVEIK

SEQ ID NO: 161 - protein, 214 aa, synthetic construct
DIQMTQSPSSLSASVGDRVTITCRASQDISNYLNWYQQKPGKAPKFLIYYTSRLESGVPSRFSGSGSGTDYTLTISSLQPEDFATYYCQQGNALPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 225 aa, synthetic construct
EVQLVQSGAEVKKPGASVKVSCKASGYKFSSYWIEWVKQAPGQGLEWIGEILPGSDTTNYAQKFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 163 - protein, 575 aa, synthetic construct
EVQLVQSGAEVKKPGASVKVSCKASGYKFSSYWIEWVKQAPGQGLEWIGEILPGSDTTNYAQKFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 164 - protein, 589 aa, synthetic construct
EVQLVQSGAEVKKPGASVKVSCKASGYKFSSYWIEWVKQAPGQGLEWIGEILPGSDTTNYAQKFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGFTFTDYAIGWFRQAPGKEREGVSCIRVSDGSTYYADSVKGRFTISRDSSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 7 aa, synthetic construct
YTSRLNS

SEQ ID NO: 166 - protein, 107 aa, synthetic construct
DIQMTQSPSSLSASVGDRVTITCRASQDISNYLNWYQQKPGKAPKFLIYYTSRLNSGVPSRFSGSGSGTDYTLTISSLQPEDFATYYCQQGNALPWTFGGGTKVEIK

SEQ ID NO: 167 - protein, 214 aa, synthetic construct
DIQMTQSPSSLSASVGDRVTITCRASQDISNYLNWYQQKPGKAPKFLIYYTSRLNSGVPSRFSGSGSGTDYTLTISSLQPEDFATYYCQQGNALPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 131 aa, synthetic construct
EVQLLESGGGLVQPGGSLRLSCAASGFTFTDYAIGWFRQAPGKEREGVSCIRVSDGSTYYADSVKGRFTISRDSSKNTLYLQMNSLRAEDTAVYYCAAGSLYTCVQSIVWPARPYYDMDYWGQGTLVTVSS

SEQ ID NO: 169 - protein, 8 aa, synthetic construct
GFTFTDYA

SEQ ID NO: 170 - protein, 8 aa, synthetic construct
IRVSDGST

SEQ ID NO: 171 - protein, 24 aa, synthetic construct
AAGSLYTCVQSIVWPARPYYDMDY

SEQ ID NO: 172 - protein, 117 aa, synthetic construct
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSS

SEQ ID NO: 173 - protein, 5 aa, synthetic construct
INFYG

SEQ ID NO: 174 - protein, 16 aa, synthetic construct
HISIGDQTDYADSAKG

SEQ ID NO: 175 - protein, 9 aa, synthetic construct
LSRIWPYDY

SEQ ID NO: 176 - protein, 575 aa, synthetic construct
EVQLVQSGAEVKKPGASVKVSCKASGYKFSSYWIEWVKQAPGQGLEWIGEILPGSDATNYAQKFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSDVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 177 - protein, 575 aa, synthetic construct
EVQLVQSGAEVKKPGASVKVSCKASGYKFSSYWIEWVKQAPGQGLEWIGEILPGSDTTNYAQKFQDRVTFTSDTSISTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSDVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 178 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 179 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 180 - protein, 448 aa, synthetic construct
QLOLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 181 - protein, 214 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 182 - protein, 5 aa, synthetic construct
SYNMH

SEQ ID NO: 183 - protein, 17 aa, synthetic construct
CIRVSDGSTYYADSVKG

SEQ ID NO: 184 - DNA, 1710 bp, synthetic construct
  source 1..1710: mol_type = other DNA
caagtgcagctggtgcagtctggcgccgaagtgaagaaacctggcgcctctgtgaaggtgtcctgcaaggcttctggctacacctttaccgactacgagatgcactgggtccgacaggctccaggacaaggcttggaatggatgggcgccctggatcctaagaccggcgataccgcttactcccagaaattcaagggcagagtgaccctgaccgccgacaagtctacctccaccgcctacatggaactgtccagcctgacctctgaggacaccgccgtgtactactgcacccggttctactcctacacctactggggccagggcaccctggttacagtgtcctctgcttccaccaagggacccagcgttttccctctggctccatcctccaagtccacctctggtggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaataccaaggtggacaagagagtggaacccaagtcctgcgacaagacaggcggatctgaggtccagctggtcgaatctggcggaggattggttcagcctggcggctctctgagactgtcttgtgtggctagcggcgacgtgcacaagatcaactttctcggctggtacagacaggcccctggcaaagagagagagaaggtcgcccacatctccatcggcgaccagaccgattacgccgactctgctaagggcagattcaccatctctcgggacgagtccaagaacatggtgtacctgcagatgaactccctgaagccagaggataccgctgtgtatttctgccgggccttcagccggatctacccttacgattattggggacagggaacactcgtgacagtgtctagcggaggcggaggctctacccatacctgtcctccatgtcctgctcctccagtggctggcccttccgtgtttctgttccctccaaagcctaaggacaccctgatgatctctcggacccctgaagtgacctgcgtggtggtggatgtgaagcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggacagccccgggaacctcaagtctgtaccctgcctcctagccgggaagagatgaccaagaaccaggtgtccctgtcctgtgccgtgaagggcttctacccctctgatatcgccgtggaatgggagtccaatggccagcctgagaacaactacaagaccacacctcctgtgctggactccgacggctcattcttcctggtgtccaagctgacagtggacaagtccagatggcagcagggcaacgtgttctcctgctccgtgatgcacgaggccctgcacaatcactacacacagaagtccctgtctctgagccccggcaaa

SEQ ID NO: 185 - DNA, 1752 bp, synthetic construct
  source 1..1752: mol_type = other DNA
caagtgcagctggtgcagtctggcgccgaagtgaagaaacctggcgcctctgtgaaggtgtcctgcaaggcttctggctacacctttaccgactacgagatgcactgggtccgacaggctccaggacaaggcttggaatggatgggcgccctggatcctaagaccggcgataccgcttactcccagaaattcaagggcagagtgaccctgaccgccgacaagtctacctccaccgcctacatggaactgtccagcctgacctctgaggacaccgccgtgtactactgcacccggttctactcctacacctactggggccagggcaccctggttacagtgtcctctgcttccaccaagggacccagcgttttccctctggctccatcctccaagtccacctctggtggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtcgacaagagagtggaacccaagtcctgcgacaagacaggcggatctgaggtgcagctgctcgaatctggcggaggattggttcagcctggcggctctctgagactgtcttgtgctgcctccggcttcaccttcgacgattacgccatcggctggttcagacaggcccctggcaaagagagagagggcgtcagctgcatcagagtgtctgacggctctacctactacgccgactccgtgaagggcagattcaccatctctcgggacaactccaagaacaccctgtacctgcagatgaactccctgagagccgaggataccgctgtgtattattgcgccgctggctccctgtatacctgcgtgcagtctatcgtgtggcccgccagaccttactacgacatggactattggggacagggaacactcgtgacagtgtctagcggaggcggcggatctacccatacctgtcctccatgtcctgctcctccagtggctggcccttccgtgtttctgttccctccaaagcctaaggacaccctgatgatctctcggacccctgaagtgacctgcgtggtggtggatgtgaagcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggacagcccagggaaccccaggtttacaccctgcctccatgccgggaagagatgaccaagaaccaggtgtccctgtggtgcctggttaagggcttctacccctccgatatcgccgtggaatgggagtctaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgagccccggcaaa

SEQ ID NO: 186 - DNA, 657 bp, synthetic construct
  source 1..657: mol_type = other DNA
gacgtcgtgatgacccagtctcctctgtctctgcctgtgacacctggcgagcctgcctccatctcttgcagatcttctcagtccctggtgcactccaaccggaacacctacctgcactggtatctgcagaagcccggacagtctccccagctgctgatctacaaggtgtccaacagattctctggcgtgcccgacagattcagcggctctggctctggcaccgacttcaccctgaagatctctagagtggaagccgaggacgtgggcgtgtactactgctctcagaatacccacgtgccacctacctttggccagggcaccaagctggaaatcaagagaaccgtggccgctccttccgtgttcatcttcccaccatctgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggcctgtctagccctgtgaccaagtctttcaaccggggcgagtgc

SEQ ID NO: 187 - DNA, 1731 bp, synthetic construct
  source 1..1731: mol_type = other DNA
gaggtgcagctggttgaatctggcggaggattggttcagcctggcggctctctgagactgtcttgtgctgcctctggctacaccttcaccagctacaacatgcactgggtccgacaggcccctggcaaaggattggaatgggtcggagctatctaccctggctccggcgatacctcctacaaccagaagttcaagggcagattcaccatctccgtggacaagtccaagaacaccctgtacctgcagatgaactccctgagagccgaggacaccgccgtgtactattgtgccagagtggtgtactactccaactcctactggtacttcgacgtgtggggccagggaacactggtcacagtgtcctctgcttccaccaagggaccctctgtgttccctctggctccttccagcaagtctacctctggtggaaccgctgctctgggctgcctggtcaaggattactttcctgagcctgtgaccgtgtcctggaattctggtgctctgacctccggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagagagtggaacccaagtcctgcgataagaccggcggatctgaagtccagttggtggaaagcggaggtggacttgtgcagccaggtggaagcctgagactgagttgtgtggcttctggcgacgtgcacaagatcaactttctcggctggtacaggcaggctcccggcaaagaaagggaaaaagtggcccacatctccatcggcgaccagaccgattacgccgactctgccaaaggccggtttaccatctctcgggacgagagcaagaacatggtgtatctccagatgaacagtctgaagcccgaggatacagctgtgtacttctgccgggccttcagccggatctacccttacgattattggggacagggcaccctggttaccgtttctagtggcggaggcggctctacccatacctgtcctccatgtcctgctccacctgtggccggcccttccgtgttcctgtttcctccaaagcctaaggacaccctgatgatctctcggacccctgaagtgacctgcgtggtggtggatgtgaaacacgaggatcccgaagtgaagttcaattggtacgtggacggcgtggaagtgcacaacgccaagaccaagcctagagaggaacagtacaactccacctaccgcgtggtgtctgtgctgaccgttctgcaccaggactggctgaacggcaaagagtacaagtgcaaggtgtccaacaaggccctgcctgcccccatcgaaaagaccatctctaaggctaagggccagcctcgcgagcctcaagtctgtacactgcctcctagccgggaagagatgaccaagaatcaggtgtccctgtcctgcgccgtgaagggcttctacccttctgatatcgccgtggaatgggagtccaacggccagcctgagaacaactacaagaccacacctcctgtgctggactccgacggctcattcttcctggtgtccaagctgaccgtggataagagcagatggcagcagggcaacgtgttctcctgctctgtgatgcacgaggccctgcacaaccactatacccagaaatccctgtctctgagccctggcaag

SEQ ID NO: 188 - DNA, 1773 bp, synthetic construct
  source 1..1773: mol_type = other DNA
gaggtgcagctggtggaatctggcggaggattggttcagcctggcggctctctgagactgtcttgtgctgcctccggctacaccttcaccagctacaacatgcactgggtccgacaggcccctggcaaaggattggaatgggtcggagctatctaccccggctctggcgacacctcctacaaccagaagttcaagggcagattcaccatctccgtggacaagtccaagaacaccctgtacctgcagatgaactccctgagagccgaggacaccgccgtgtactactgtgccagagtggtgtactacagcaactcctactggtacttcgacgtgtggggccagggcacactggtcacagtttcttccgcctccaccaagggacccagcgttttccctctggctccatcctccaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgataagaccggcggaagcgaagtgcagctgcttgaaagcggaggtggacttgtgcagccaggcggaagcctgagattgtcctgtgccgcttctggctttaccttcgacgactacgccatcggctggttcagacaggctcccggaaaagagagagagggcgtcagctgcatcagagtgtctgacggctctacctactacgccgactccgtgaaaggccggttcaccatcagccgggacaacagcaagaatactctgtatctccaaatgaacagcctgcgcgctgaggataccgctgtgtattattgcgccgctggctccctgtatacctgcgtgcagtctatcgtgtggcccgccagaccttactacgacatggattactggggacagggaaccctggttaccgtgtctagcggcggaggcggatctacccatacctgtcctccatgtcctgctccacctgtggccggcccttccgtgttcctgtttcctccaaagcctaaggacaccctgatgatctctcggacccctgaagtgacctgcgtggtggtggatgtgaaacacgaggatcccgaagtgaagttcaattggtacgtggacggcgtggaagtgcacaacgccaagaccaagcctagagaggaacagtacaactccacctaccgcgtggtgtccgtgctgaccgttctgcatcaggattggctgaacggcaaagagtacaagtgcaaggtgtccaacaaggccctgcctgcccccatcgaaaagaccatctccaaggccaagggccagcctagggaaccccaggtttacaccctgccaccttgccgggaagagatgaccaagaaccaggtgtccctgtggtgcctcgtgaagggcttctacccttccgatatcgccgtggaatgggagagcaatggccagcctgagaacaactacaagaccacacctcctgtgctggactccgacggctcattcttcctgtactccaagctgaccgtggataagtctcggtggcagcagggcaacgtgttctcctgctctgtgatgcacgaggccctgcacaaccactatacccagaagtccctgtctctgagccccggcaag

SEQ ID NO: 189 - DNA, 639 bp, synthetic construct
  source 1..639: mol_type = other DNA
gacatccagatgacccagtctccatcctctctgtctgcctctgtgggcgacagagtgacaatcacctgtagagcctccagctccgtgtcctacatgcactggtatcagcagaagcccggcaaggcccctaagcctctgatctacgctccttctaatctggcctccggcgtgccctctagattttctggctctggatctggcaccgacttcaccctgaccatcagttctctgcagcctgaggacttcgccacctactactgccagcagtggtctttcaaccctcctacctttggccagggcaccaaggtggaaatcaagagaaccgtggccgctccttccgtgttcatcttcccaccatctgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtccttcaacagaggcgagtgt

SEQ ID NO: 190 - DNA, 1740 bp, synthetic construct
  source 1..1740: mol_type = other DNA
caagtgcagctgcagcagtcaggtccaggactggtgaagccctcgcagaccctctcactcacctgtgccatctccggggacagtgtctccagaaacagtgctgtttggaattggatcaggcagtccccatcgagaggccttgagtggctgggaaggacatactacaggtccaagtggtataatgattatgcagtatctgtgaaaagtcgaataaccatcaacccagacacatccaagaaccagttctccctgcaactgaactctgtgactcccgaggacacggctgtgtattactgtgcaagggggttacgacagaaccagttctactactacatggacgtctggggcaaagggaccacggtcaccgtctcctccgctagtaccaagggacccagcgtgttccctctggcaccttccagcaagtctacctctggcggaacagccgctctgggctgtctggtcaaggactactttcccgagcctgtgaccgtgtcctggaattctggcgctctgaccagcggagtgcatacctttccagctgtgctgcagtcctccggcctgtactctctgtcctctgtcgtgaccgtgccttccagctctctgggcacccagacctacatctgcaatgtgaaccacaagccttccaacaccaaggtggacaagagggtggaacccaagtcctgcgacaagaccggcggatctgaagtgcagctggtcgagtctggcggaggattggttcaacctggcggctccctgagactgtcttgtgtggctagcggagatgtgcataagatcaattttctcggctggtacagacaggcccctggcaaagagagagagaaggtcgcccacatctccatcggcgaccagaccgattacgccgactctgccaagggcagattcaccatctctcgggacgagtccaagaacatggtgtacctgcagatgaactccctgaagcctgaggataccgccgtgtacttctgccgggccttctctcggatctacccctacgattattggggccagggcaccctggtcacagtgtcatctggaggcggcggatctactcacacgtgcccaccgtgcccagcacctccggtggccggaccgtcagtcttcctcttccccccaaaacccaaggacaccctcatgatctcgcgaacccctgaggtcacatgcgtggtggtggacgtgaaacacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagccccaatcgagaaaaccatctccaaagccaaagggcagccccgagaaccacaggtctgcaccctgcccccatcccgggaggagatgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgttagcaagctcaccgtggacaagagccggtggcagcaggggaacgtcttctcatgctccgtgatgcatgaggctctgcacaaccgcttcacgcagaagagcctctccctgtctccgggtaaa

SEQ ID NO: 191 - DNA, 1782 bp, synthetic construct
  source 1..1782: mol_type = other DNA
caagtgcagctgcagcagtcaggtccaggactggtgaagccctcgcagaccctctcactcacctgtgccatctccggggacagtgtctccagaaacagtgctgtttggaattggatcaggcagtccccatcgagaggccttgagtggctgggaaggacatactacaggtccaagtggtataatgattatgcagtatctgtgaaaagtcgaataaccatcaacccagacacatccaagaaccagttctccctgcaactgaactctgtgactcccgaggacacggctgtgtattactgtgcaagggggttacgacagaaccagttctactactacatggacgtctggggcaaagggaccacggtcaccgtctcctccgctagcaccaagggacccagcgtgttccctctggcaccttccagcaagtctacctctggcggaacagccgctctgggctgtctggtcaaggactactttcccgagcctgtgaccgtgtcctggaattctggcgctctgaccagcggagtgcatacctttccagctgtgctgcagtcctccggcctgtactctctgtcctctgtcgtgaccgtgccttccagctctctgggcacccagacctacatctgcaatgtgaaccacaagccttccaacaccaaggtggacaagagggtggaacccaagtcctgcgacaagaccggcggatctgaggtccaattgttggaatctggaggggggctcgtgcagccggggggaagtttgcgcctttcatgcgctgcatcagggtttactttcgacgactacgctattggctggttcaggcaagcgccgggcaaggaacgcgaaggcgtgtcctgcataagagtttcagatggcagtacctattacgccgatagcgtcaagggacgattcacgatcagccgggataatagcaaaaatacactttatttgcaaatgaactctcttagggcagaagatactgcggtctattattgtgcggccgggtccctttatacctgcgtgcagtctatcgtatggcctgctaggccctattacgatatggattactggggacagggtacactcgtgacggtatccagtggaggcggcggatctactcacacgtgcccaccgtgcccagcacctccggtggccggaccgtcagtcttcctcttccccccaaaacccaaggacaccctcatgatctcccgtacccctgaggtcacatgcgtggtggtggacgtgaaacacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagccccaatcgagaaaaccatctccaaagccaaagggcagccccgagaaccacaggtctacaccctgcccccatgccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagccggtggcagcaggggaacgtcttctcatgctccgtgatgcatgaggctctgcacaaccactacacgcagaagagcttaagcctgtctccgggtaaa

SEQ ID NO: 192 - DNA, 642 bp, synthetic construct
  source 1..642: mol_type = other DNA
gaaatagtgatgacgcagtctccagccaccctgtctgtgtctcccggggaaagagccaccctctcctgccgagccagtcagagtgttgccagcaacttagcctggtaccagcagaaacctggccaggctcccaggctcctcatctatggtgcatccaccagagccactggtatcccagccaggttcagtggcagtgggtctgggacagagttcactctcaccatcagcagcctgcagtctgaagattttgcagtttattactgtcaacagtataataactggccattcactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 193 - DNA, 1767 bp, synthetic construct
  source 1..1767: mol_type = other DNA
gaggtgcagctggtggaaagtggaggtggcctggttaagcctggcgggagcctgcgcctgagctgcgctgccagcggcttcactttctcatcttacagcatgaactgggtgcgccaggcccctggcaaaggccttgagtgggttagtagcatcagcggccagtcaaactacatttattacgccgattccgtcaagggcaggtttacaatctcccgtgataatgcgaagaatagcctctatctccagatgaattccctgcgcgccgaagatacagccgtgtattactgcgcgaggggcgggaattactttgttgaatacttccagtactggggccagggtaccctcgtcaccgtttccagtgcaagcaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgaaccaaaaagctgcgacaagaccggaggatctgaggtccagctgctagagtctggcggaggattggttcagcctggcggatctctgagactgtcttgtgccgcctctggcttcaccttcgacgattacgccatcggctggttcagacaggcccctggcaaagagagagagggcgtcagctgcatcagagtgtctgacggctccacctactacgccgattctgtgaagggcagattcaccatctcccgcgacaactccaagaacaccctgtacctgcagatgaactccctgcgggctgaggacaccgccgtgtactattgtgctgccggctctctgtacacctgtgtgcagtctatcgtgtggcccgccagaccttactacgacatggactattggggccagggcaccctagtgaccgtctcaagcggtggcggaggatctacccacacctgtcctccatgccctgctccacccgtggccggcccatccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgcccccatcgaaaagaccatctccaaggccaagggccagcctagggaaccccaggtttacaccctgccaccttgccgggaagagatgaccaagaaccaggtgtccctgtggtgcctcgtgaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaa

SEQ ID NO: 194 - DNA, 1725 bp, synthetic construct
  source 1..1725: mol_type = other DNA
gaggtgcagctggtggaaagtggaggtggcctggttaagcctggcgggagcctgcgcctgagctgcgctgccagcggcttcactttctcatcttacagcatgaactgggtgcgccaggcccctggcaaaggccttgagtgggttagtagcatcagcggccagtcaaactacatttattacgccgattccgtcaagggcaggtttacaatctcccgtgataatgcgaagaatagcctctatctccagatgaattccctgcgcgccgaagatacagccgtgtattactgcgcgaggggcgggaattactttgttgaatacttccagtactggggccagggtaccctcgtcaccgtttccagtgcaagcaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgaaccaaaaagctgcgacaagaccggaggatctgaagtgcagctggtcgagtctggcggaggattggttcaacctggcggctccctgagactgtcttgtgtggctagcggagatgtgcataagatcaattttctcggctggtacagacaggcccctggcaaagagagagagaaggtcgcccacatctccatcggcgaccagaccgattacgccgactctgccaagggcagattcaccatctctcgggacgagtccaagaacatggtgtacctgcagatgaactccctgaagcctgaggataccgccgtgtacttctgccgggccttctctcggatctacccctacgattattggggccagggcaccctggtcacagtgtcatctggtggcggaggatctacccacacctgtcctccatgccctgctccacccgtggccggcccttccgtgttcctgtttcctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgcccccatcgaaaagaccatctctaaggctaagggccagcctcgcgagcctcaagtctgtacactgcctcctagccgggaagagatgaccaagaaccaggtgtccctgtcttgcgccgtgaagggcttctacccttccgatatcgccgtggaatgggagtccaatggccagcctgagaacaactacaagacaacccctcctgtgctggactccgacggctcattcttcctggtgtccaagctgacagtggacaagtccagatggcagcagggcaacgtgttctcctgctctgtgatgcacgaggccctgcacaaccggttcacccagaaatccctgtctctgtcccctggcaag

SEQ ID NO: 195 - DNA, 645 bp, synthetic construct
  source 1..645: mol_type = other DNA
gagattgtcctgacacagtcccctggcacccttagcctgtcccccggtgaaagagcgaccctgtcctgccgtgcctcacaatacatctcttccaacaatctggcgtggtaccaacagaagcccgggcaagctccccgcctcctgatttacggggcatccaatagagccaccgggatccctgatagattctccggcagcggtagcggtaccgatttcaccctgaccatctctcgcctggagcctgaagacttcgcggtgtactattgtcaacagtatgctgattccccaatcacattcggccagggtactaagcttgaaatcaagcggacagtggccgcaccgtccgtgttcatcttcccaccttccgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 196 - DNA, 1767 bp, synthetic construct
  source 1..1767: mol_type = other DNA
gaagtgcagctggttcagtctggcgccgaagtgaagaaacctggcgcctctgtgaaggtgtcctgcaaggcttccggctacaagttctccagctactggatcgagtgggtcaagcaggctcctggacagggactcgagtggatcggagagatcctgcctggctctgacaccaccaactacaacgagaagttcaaggaccgggccaccttcacctccgacacctctatcaacaccgcctacatggaactgtcccggctgagatctgacgacaccgccgtgtactactgcgccagagacagaggcaactacagagcttggtttggctactggggccagggcacactggtcacagtttcttctgcaagcaccaagggaccctctgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgataagaccggcggatctgaggtgcagctgttggaatctggcggtggattggttcagcctggcggctctctgagactgtcttgtgccgcttctggcttcaccttcgacgactacgccatcggctggttcagacaggcccctggcaaagagagagagggcgtcagctgcatcagagtgtctgacggctctacctactacgccgactccgtgaagggcagattcaccatctctcgggacaactccaagaacaccctgtacctgcagatgaactccctgagagccgaggacaccgccgtgtactattgtgctgctggctccctgtacacctgtgtgcagtctatcgtgtggcccgccagaccttactacgacatggattattggggccagggcaccctggtcacagtttctagcggaggcggaggcagcacccatacctgtcctccatgtcctgctcctccagtggctggcccttccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggacagcccagggaaccccaggtttacaccctgcctccatgccgggaagagatgaccaagaaccaggtgtccctgtggtgcctggttaagggcttctacccctccgatatcgccgtggaatgggagtctaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgagccccggcaaa

SEQ ID NO: 197 - DNA, 1725 bp, synthetic construct
  source 1..1725: mol_type = other DNA
gaagtgcagctggttcagtctggcgccgaagtgaagaaacctggcgcctctgtgaaggtgtcctgcaaggcttccggctacaagttctccagctactggatcgagtgggtcaagcaggctcctggacagggactcgagtggatcggagagatcctgcctggctctgacaccaccaactacaacgagaagttcaaggaccgggccaccttcacctccgacacctctatcaacaccgcctacatggaactgtcccggctgagatctgacgacaccgccgtgtactactgcgccagagacagaggcaactacagagcttggtttggctactggggccagggcacactggtcacagtttcttctgcaagtactaagggcccctctgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgataagaccggcggatctgaggtgcagctggtggaatctggcggaggattggttcagcctggcggctctctgagactgtcttgtgtggctagcggcgacgtgcacaagatcaactttctcggctggtacagacaggcccctggcaaagagagagagaaggtcgcccacatctccatcggcgaccagaccgattacgccgactctgccaagggcagattcaccatctctcgggacgagtccaagaacatggtgtacctgcagatgaactccctgaagcctgaggataccgccgtgtacttctgccgggccttctctcggatctacccctacgattattggggccagggcaccctggtcacagtttctagcggcggaggcggctctacccatacctgtcctccatgtcctgctcctccagtggctggcccatccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgtcctgtgccgtgaagggcttctacccttccgatatcgccgtggaatgggagagcaatggccagcctgagaacaactacaagaccacacctcctgtgctggactccgacggctcattcttcctggtgtccaagctgacagtggacaagtccagatggcagcagggcaacgtgttctcctgctccgtgatgcacgaggccctgcacaacaggttcacccagaagtccctgtctctgagccctggcaag

SEQ ID NO: 198 - DNA, 642 bp, synthetic construct
  source 1..642: mol_type = other DNA
gacatccagatgacccagtctccatcctctctgtccgcctctgtgggcgacagagtgaccatcacctgtagagccagccaggacatctccaactacctgaactggtatcagcagaaacctggcggcgctgtgaagttcctgatctactacacctctcggctgcactccggcgtgccctctagattttctggctctggatccggcaccgactataccctgacaatctccagcctgcagcctgaggacttcgctacctacttctgccagcaaggcgaggctctgccttggacatttggcggcggaacaaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 199 - DNA, 1767 bp, synthetic construct
  source 1..1767: mol_type = other DNA
gaggtgcagttggtccaatcaggagcagaagtcaaaaaacccggtgcttccgttaaagtatcttgcaaagctagcgggtacaagttctcttcctattggatcgagtgggttaagcaggcacctggacagggtcttgaatggataggcgaaatccttcccggtagcgacaccacaaactatgctcaaaagttccaagatagagttactttcacttcagatacttccatatctactgcatatatggaactgtcccgtttgaggagtgatgacacagcagtgtactactgtgcccgtgaccgtggaaactaccgtgcatggttcggctattggggtcagggtacactcgtaacagtgagctcagcaagcaccaagggaccctctgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgataagaccggcggatctgaggtacagctgctggaatcaggaggcggcttggttcagccaggaggctcacttcggctctcctgcgcagcaagtgggtttacatttactgattatgctataggctggtttcggcaagcacctggtaaggaacgcgaaggtgtgtcatgtattcgcgtaagcgacggatcaacatactatgcagacagtgtcaaaggtagatttactattagtcgtgacagctcaaagaatactctgtacttgcaaatgaattcccttcgtgccgaagatactgccgtttactattgcgccgcaggatcactctacacctgcgtgcagagcattgtttggcctgccaggccttattatgatatggactattggggccaaggcactttggtcactgttagctctggaggcggaggcagcacccatacctgtcctccatgtcctgctcctccagtggctggcccttccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggacagcccagggaaccccaggtttacaccctgcctccatgccgggaagagatgaccaagaaccaggtgtccctgtggtgcctggttaagggcttctacccctccgatatcgccgtggaatgggagtctaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgagccccggcaaa

SEQ ID NO: 200 - DNA, 1725 bp, synthetic construct
  source 1..1725: mol_type = other DNA
gaggtgcagttggtccaatcaggagcagaagtcaaaaaacccggtgcttccgttaaagtatcttgcaaagctagcgggtacaagttctcttcctattggatcgagtgggttaagcaggcacctggacagggtcttgaatggataggcgaaatccttcccggtagcgacaccacaaactatgctcaaaagttccaagatagagttactttcacttcagatacttccatatctactgcatatatggaactgtcccgtttgaggagtgatgacacagcagtgtactactgtgcccgtgaccgtggaaactaccgtgcatggttcggctattggggtcagggtacactcgtaacagtgagctcagcaagtactaagggcccctctgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgataagaccggcggatctgaggtgcagctggtggaatctggcggaggattggttcagcctggcggctctctgagactgtcttgtgtggctagcggcgacgtgcacaagatcaactttctcggctggtacagacaggcccctggcaaagagagagagaaggtcgcccacatctccatcggcgaccagaccgattacgccgactctgccaagggcagattcaccatctctcgggacgagtccaagaacatggtgtacctgcagatgaactccctgaagcctgaggataccgccgtgtacttctgccgggccttctctcggatctacccctacgattattggggccagggcaccctggtcacagtttctagcggcggaggcggctctacccatacctgtcctccatgtcctgctcctccagtggctggcccatccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgtcctgtgccgtgaagggcttctacccttccgatatcgccgtggaatgggagagcaatggccagcctgagaacaactacaagaccacacctcctgtgctggactccgacggctcattcttcctggtgtccaagctgacagtggacaagtccagatggcagcagggcaacgtgttctcctgctccgtgatgcacgaggccctgcacaacaggttcacccagaagtccctgtctctgagccctggcaag

SEQ ID NO: 201 - DNA, 642 bp, synthetic construct
  source 1..642: mol_type = other DNA
gacatccagatgacccagagtccctctagtctctcagcttccgtgggggaccgcgtcactataacttgccgtgcctcacaagacatatcaaactacctcaactggtatcaacaaaagcctggaaaagctcctaagtttttgatttattacacttccaggctggagtccggtgtcccatccaggttcagcggcagtggaagcggaaccgactatacactgactatttctagcttgcaacccgaggactttgccacctattattgccagcaaggtaacgccctgccctggaccttcggtggcggaaccaaggtggaaatcaagcggacagtggccgcaccgtccgtgttcatcttcccaccttccgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 202 - DNA, 1767 bp, synthetic construct
  source 1..1767: mol_type = other DNA
gaggttcagttggtacaatctggagccgaggtgaaaaaaccaggtgcaagcgtcaaggtttcctgcaaagctagtgggtacaaattcagttcatactggatagagtgggttaagcaagctcctggacaaggtttggagtggattggtgaaatcttgcctggcagcgatactacaaactacgctcagcagtttcaggaccgcgtaacattcacctctgatacatctatatcaactgcatatatggaacttagcaggctcaggtccgacgacactgccgtctattactgcgctcgggatagggggaattatcgtgcttggtttggttattggggtcaagggacactggttacagtttcttcagcaagcaccaagggaccctctgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgataagaccggcggatctgaggtacagctgctggaatcaggaggcggcttggttcagccaggaggctcacttcggctctcctgcgcagcaagtgggtttacatttactgattatgctataggctggtttcggcaagcacctggtaaggaacgcgaaggtgtgtcatgtattcgcgtaagcgacggatcaacatactatgcagacagtgtcaaaggtagatttactattagtcgtgacagctcaaagaatactctgtacttgcaaatgaattcccttcgtgccgaagatactgccgtttactattgcgccgcaggatcactctacacctgcgtgcagagcattgtttggcctgccaggccttattatgatatggactattggggccaaggcactttggtcactgttagctctggaggcggaggcagcacccatacctgtcctccatgtcctgctcctccagtggctggcccttccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggacagcccagggaaccccaggtttacaccctgcctccatgccgggaagagatgaccaagaaccaggtgtccctgtggtgcctggttaagggcttctacccctccgatatcgccgtggaatgggagtctaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgagccccggcaaa

SEQ ID NO: 203 - DNA, 1725 bp, synthetic construct
  source 1..1725: mol_type = other DNA
gaggttcagttggtacaatctggagccgaggtgaaaaaaccaggtgcaagcgtcaaggtttcctgcaaagctagtgggtacaaattcagttcatactggatagagtgggttaagcaagctcctggacaaggtttggagtggattggtgaaatcttgcctggcagcgatactacaaactacgctcagcagtttcaggaccgcgtaacattcacctctgatacatctatatcaactgcatatatggaacttagcaggctcaggtccgacgacactgccgtctattactgcgctcgggatagggggaattatcgtgcttggtttggttattggggtcaagggacactggttacagtttcttcagcaagtactaagggcccctctgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgataagaccggcggatctgaggtgcagctggtggaatctggcggaggattggttcagcctggcggctctctgagactgtcttgtgtggctagcggcgacgtgcacaagatcaactttctcggctggtacagacaggcccctggcaaagagagagagaaggtcgcccacatctccatcggcgaccagaccgattacgccgactctgccaagggcagattcaccatctctcgggacgagtccaagaacatggtgtacctgcagatgaactccctgaagcctgaggataccgccgtgtacttctgccgggccttctctcggatctacccctacgattattggggccagggcaccctggtcacagtttctagcggcggaggcggctctacccatacctgtcctccatgtcctgctcctccagtggctggcccatccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgtcctgtgccgtgaagggcttctacccttccgatatcgccgtggaatgggagagcaatggccagcctgagaacaactacaagaccacacctcctgtgctggactccgacggctcattcttcctggtgtccaagctgacagtggacaagtccagatggcagcagggcaacgtgttctcctgctccgtgatgcacgaggccctgcacaacaggttcacccagaagtccctgtctctgagccctggcaag

SEQ ID NO: 204 - DNA, 642 bp, synthetic construct
  source 1..642: mol_type = other DNA
gatattcagatgacacaatcccctagcagtctcagtgcctcagtaggggaccgcgtcacaataacatgtagagcctcccaggatatttctaactacttgaactggtaccagcaaaagcccggtaaagcccctaaattccttatttactacacaagccgattgcactctggtgtaccatccaggtttagtggaagcggctctggtacagattacacactgaccatttcatccctgcaaccagaggatttcgccacatattactgccaacagggaaacgccctcccttggacctttggtgggggaactaaagttgagatcaagcggacagtggccgcaccgtccgtgttcatcttcccaccttccgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 205 - DNA, 1767 bp, synthetic construct
  source 1..1767: mol_type = other DNA
gaggtgcagcttgttcaaagcggtgcagaggttaagaagcctggagctagcgtaaaagtgtcctgcaaagctagtgggtataagttctctagctactggatcgagtgggtgaaacaagccccagggcaggggttggagtggattggagaaatactccccggatcagacgccactaactatgcacagaagtttcaagacagggttaccttcacatcagacacatccatatcaaccgcatatatggagttgtccaggctgcggagcgacgacaccgctgtctattattgtgccagggaccgggggaactacagggcttggtttgggtactggggacagggtacactcgtcacagtgtcctccgcaagcaccaagggaccctctgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgataagaccggcggatctgaggtacagctgctggaatcaggaggcggcttggttcagccaggaggctcacttcggctctcctgcgcagcaagtgggtttacatttactgattatgctataggctggtttcggcaagcacctggtaaggaacgcgaaggtgtgtcatgtattcgcgtaagcgacggatcaacatactatgcagacagtgtcaaaggtagatttactattagtcgtgacagctcaaagaatactctgtacttgcaaatgaattcccttcgtgccgaagatactgccgtttactattgcgccgcaggatcactctacacctgcgtgcagagcattgtttggcctgccaggccttattatgatatggactattggggccaaggcactttggtcactgttagctctggaggcggaggcagcacccatacctgtcctccatgtcctgctcctccagtggctggcccttccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggacagcccagggaaccccaggtttacaccctgcctccatgccgggaagagatgaccaagaaccaggtgtccctgtggtgcctggttaagggcttctacccctccgatatcgccgtggaatgggagtctaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgagccccggcaaa

SEQ ID NO: 206 - DNA, 1725 bp, synthetic construct
  source 1..1725: mol_type = other DNA
gaggtgcagcttgttcaaagcggtgcagaggttaagaagcctggagctagcgtaaaagtgtcctgcaaagctagtgggtataagttctctagctactggatcgagtgggtgaaacaagccccagggcaggggttggagtggattggagaaatactccccggatcagacgccactaactatgcacagaagtttcaagacagggttaccttcacatcagacacatccatatcaaccgcatatatggagttgtccaggctgcggagcgacgacaccgctgtctattattgtgccagggaccgggggaactacagggcttggtttgggtactggggacagggtacactcgtcacagtgtcctccgcaagtactaagggcccctctgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgataagaccggcggatctgaggtgcagctggtggaatctggcggaggattggttcagcctggcggctctctgagactgtcttgtgtggctagcggcgacgtgcacaagatcaactttctcggctggtacagacaggcccctggcaaagagagagagaaggtcgcccacatctccatcggcgaccagaccgattacgccgactctgccaagggcagattcaccatctctcgggacgagtccaagaacatggtgtacctgcagatgaactccctgaagcctgaggataccgccgtgtacttctgccgggccttctctcggatctacccctacgattattggggccagggcaccctggtcacagtttctagcggcggaggcggctctacccatacctgtcctccatgtcctgctcctccagtggctggcccatccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgtcctgtgccgtgaagggcttctacccttccgatatcgccgtggaatgggagagcaatggccagcctgagaacaactacaagaccacacctcctgtgctggactccgacggctcattcttcctggtgtccaagctgacagtggacaagtccagatggcagcagggcaacgtgttctcctgctccgtgatgcacgaggccctgcacaacaggttcacccagaagtccctgtctctgagccctggcaag

SEQ ID NO: 207 - DNA, 642 bp, synthetic construct; identical to SEQ ID NO: 204
  source 1..642: mol_type = other DNA

SEQ ID NO: 208 - DNA, 1767 bp, synthetic construct; identical to SEQ ID NO: 199
  source 1..1767: mol_type = other DNA

SEQ ID NO: 209 - DNA, 1724 bp, synthetic construct
  source 1..1724: mol_type = other DNA
aggtgcagttggtccaatcaggagcagaagtcaaaaaacccggtgcttccgttaaagtatcttgcaaagctagcgggtacaagttctcttcctattggatcgagtgggttaagcaggcacctggacagggtcttgaatggataggcgaaatccttcccggtagcgacaccacaaactatgctcaaaagttccaagatagagttactttcacttcagatacttccatatctactgcatatatggaactgtcccgtttgaggagtgatgacacagcagtgtactactgtgcccgtgaccgtggaaactaccgtgcatggttcggctattggggtcagggtacactcgtaacagtgagctcagcaagtactaagggcccctctgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgataagaccggcggatctgaggtgcagctggtggaatctggcggaggattggttcagcctggcggctctctgagactgtcttgtgtggctagcggcgacgtgcacaagatcaactttctcggctggtacagacaggcccctggcaaagagagagagaaggtcgcccacatctccatcggcgaccagaccgattacgccgactctgccaagggcagattcaccatctctcgggacgagtccaagaacatggtgtacctgcagatgaactccctgaagcctgaggataccgccgtgtacttctgccgggccttctctcggatctacccctacgattattggggccagggcaccctggtcacagtttctagcggcggaggcggctctacccatacctgtcctccatgtcctgctcctccagtggctggcccatccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgtcctgtgccgtgaagggcttctacccttccgatatcgccgtggaatgggagagcaatggccagcctgagaacaactacaagaccacacctcctgtgctggactccgacggctcattcttcctggtgtccaagctgacagtggacaagtccagatggcagcagggcaacgtgttctcctgctccgtgatgcacgaggccctgcacaacaggttcacccagaagtccctgtctctgagccctggcaag

SEQ ID NO: 210 - DNA, 642 bp, synthetic construct; identical to SEQ ID NO: 201
  source 1..642: mol_type = other DNA

SEQ ID NO: 211 - DNA, 1767 bp, synthetic construct; identical to SEQ ID NO: 199
  source 1..1767: mol_type = other DNA

SEQ ID NO: 212 - DNA, 1725 bp, synthetic construct; identical to SEQ ID NO: 200
  source 1..1725: mol_type = other DNA

SEQ ID NO: 213 - DNA, 642 bp, synthetic construct
  source 1..642: mol_type = other DNA
gacatacagatgactcagtctccatcttccctttccgcttctgtgggtgatcgagtaacaattacatgccgggcatcacaggatatttctaactatcttaattggtatcaacaaaaaccaggtaaggctccaaagtttctgatctattatacctctcggctgaacagtggtgtccccagtaggttcagcggtagtggcagcggaactgactacaccctgaccatttcttctctgcaacccgaagattttgcaacctactattgtcaacaaggcaacgctcttccttggacctttggcggaggcacaaaggtcgaaataaaacggacagtggccgcaccgtccgtgttcatcttcccaccttccgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 214 - DNA, 1767 bp, synthetic construct; identical to SEQ ID NO: 205
  source 1..1767: mol_type = other DNA

SEQ ID NO: 215 - DNA, 1725 bp, synthetic construct
  source 1..1725: mol_type = other DNA
gaagtgcagttggtgcagtctggggccgaagttaagaaaccaggggcctctgttaaagtaagttgcaaggcttccggttacaagttttcaagctactggatcgagtgggtcaagcaggccccaggccaagggttggaatggataggagagatccttccagggagcgatgccacaaactacgcccaaaaattccaggacagggttacttttactagtgacacttccatatcaacagcctatatggagctgtcccgattgcgctccgatgatactgccgtgtactattgcgccagggatagaggcaattaccgcgcatggtttggatactgggggcagggaacacttgtgacagttagctccgcaagcaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgaaccaaaaagctgtgataaaaccggaggttcagacgtgcagttggtggaatctggaggcggtgttgtacagcccggcggctctttgagactctcatgtgtcgccagcggctacgtacacaagatcaatttttatggctggtataggcaagctcccggtaaagagcgcgagaaagtcgctcatattagcatcggagatcagactgactacgccgactccgctaaagggcgcttcaccatatctagggatgaatcaaagaacactgtgtatctccagatgaactctctccgcccagaggatactgccgcttactattgtcgtgctctttcccgtatatggccttatgattactggggacaggggacattggttactgtgagttctggtggcggaggatctacccacacctgtcctccatgccctgctccacccgtggccggcccttccgtgttcctgtttcctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgcccccatcgaaaagaccatctctaaggctaagggccagcctcgcgagcctcaagtctgtacactgcctcctagccgggaagagatgaccaagaaccaggtgtccctgtcttgcgccgtgaagggcttctacccttccgatatcgccgtggaatgggagtccaatggccagcctgagaacaactacaagacaacccctcctgtgctggactccgacggctcattcttcctggtgtccaagctgacagtggacaagtccagatggcagcagggcaacgtgttctcctgctctgtgatgcacgaggccctgcacaaccggttcacccagaaatccctgtctctgtcccctggcaag

SEQ ID NO: 216 - DNA, 642 bp, synthetic construct; identical to SEQ ID NO: 204
  source 1..642: mol_type = other DNA

SEQ ID NO: 217 - DNA, 1767 bp, synthetic construct; identical to SEQ ID NO: 199
  source 1..1767: mol_type = other DNA

SEQ ID NO: 218 - DNA, 1725 bp, synthetic construct
  source 1..1725: mol_type = other DNA
gaggtgcagttggtccaatcaggagcagaagtcaaaaaacccggtgcttccgttaaagtatcttgcaaagctagcgggtacaagttctcttcctattggatcgagtgggttaagcaggcacctggacagggtcttgaatggataggcgaaatccttcccggtagcgacaccacaaactatgctcaaaagttccaagatagagttactttcacttcagatacttccatatctactgcatatatggaactgtcccgtttgaggagtgatgacacagcagtgtactactgtgcccgtgaccgtggaaactaccgtgcatggttcggctattggggtcagggtacactcgtaacagtgagctcagcaagcaccaagggaccctctgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaaccaaaaagctgtgacaagactggtggctcagacgtacaattggttgaaagtggtgggggcgtagtccagcccggaggcagcctccgactttcctgcgtcgcatctggatacgtccataaaatcaacttttacggctggtacagacaagcaccagggaaagagcgggaaaaagttgcccacatatccattggcgaccagacagattatgccgacagcgccaagggtcgatttactataagtcgggacgaatcaaagaatactgtgtatctccagatgaacagtcttcgacctgaagacaccgccgcttattattgtagggctttgtccaggatctggccatacgattactgggggcaagggacactcgtaaccgttagttctggtggcggaggatctacccacacctgtcctccatgccctgctccacccgtggccggcccttccgtgttcctgtttcctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgcccccatcgaaaagaccatctctaaggctaagggccagcctcgcgagcctcaagtctgtacactgcctcctagccgggaagagatgaccaagaaccaggtgtccctgtcttgcgccgtgaagggcttctacccttccgatatcgccgtggaatgggagtccaatggccagcctgagaacaactacaagacaacccctcctgtgctggactccgacggctcattcttcctggtgtccaagctgacagtggacaagtccagatggcagcagggcaacgtgttctcctgctctgtgatgcacgaggccctgcacaaccggttcacccagaaatccctgtctctgtcccctggcaag

SEQ ID NO: 219 - DNA, 642 bp, synthetic construct; identical to SEQ ID NO: 201
  source 1..642: mol_type = other DNA

SEQ ID NO: 220 - protein, 103 aa, synthetic construct
ASTKGPSVFPLAPSSKSTSGGTADLGCLVKDYFPEPVTVSWNSGALTSGVHTFPAVLQSSGLYSLSSVVTVPSSSLGTQTYICNVNHKPSNTKVDKRVEPKSC

SEQ ID NO: 221 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 222 - protein, 109 aa, synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAK

(19) United States
(12) Patent Application Publication
(10) Pub. No.: US 2025/0115678 A1
(43) Pub. Date: Apr. 10, 2025

(54) T CELL BINDING COMPOSITIONS AND METHODS

(71) Applicant: ASTRAZENECA AB, Sodertalje (SE)

(72) Inventors: Mark COBBOLD, Wilmington, DE (US); Corinne CAYATTE, Gaithersburg, MD (US); Thomas CIUCCI, Wilmington, DE (US); Yariv MAZOR, Wilmington, DE (US); Saso CEMERSKI, Wilmington, DE (US); Simon DOVEDI, Cambridge (GB); Christopher LLOYD, Cambridge (GB); Jonathan SEAMAN, Cambridge (GB); Dominique LISIERO, Wilmington, DE (US); Hanzhi ZHANG, Wilmington, DE (US); Qun DU, Wilmington, DE (US); Tilbe CREIGH-PULATMEN, Cambridge (GB); Gilad KAPLAN, Gaithersburg, MD (US); Cristina BERGAMASCHI, Wilmington, DE (US); Xiuling LI, Wilmington, DE (US); Ying FU, Wilmington, DE (US); Amber LEE, Wilmington, DE (US); Alan SANDERCOCK, Cambridge (GB); Vijay CHENNUPATI, Cambridge (GB); Sterling R. PAYNE, Wilmington, DE (US); Abigail Bowlsbey LARA, Gaithersburg, MD (US); Laure CASTAN, Cambridge (GB); Isabelle SERMADIRAS, Cambridge (GB); Owen VICKERY, Cambridge (GB); Suzanne SITNIKOVA, Cambridge (GB); Even WALSENG, Gaithersburg, MD (US)

(21) Appl. No.: 18/910,389

(22) Filed: Oct. 9, 2024

Related U.S. Application Data
(60) Provisional application No. 63/658,583, filed on Jun. 11, 2024.
(60) Provisional application No. 63/589,297, filed on Oct. 10, 2023.

(51) Int. Cl. C07K 16/30; A61K 39/00; A61P 35/00; C07K 16/18; C07K 16/28

(52) U.S. Cl. CPC C07K 16/303; A61P 35/00; C07K 16/18; C07K 16/2809; C07K 16/2887; C07K 16/30; A61K 2039/505; C07K 2317/24; C07K 2317/55; C07K 2317/569; C07K 2317/73; C07K 2317/92

(57) ABSTRACT

The disclosure generally relates to binding proteins that comprise antigen binding sites, a T cell receptor binding site, and a T cell co-stimulatory molecule binding site. The disclosure also relates to pharmaceutical compositions comprising such binding proteins, nucleic acid molecules encoding such binding proteins, and vectors comprising such nucleic acid molecules. The disclosure further relates to methods of treating a disorder or condition using such binding proteins and pharmaceutical compositions, binding proteins and pharmaceutical compositions for use in the treatment of a disorder or condition, and the use of such binding proteins and pharmaceutical compositions for the manufacture of a medicament for treating a disorder or condition.

### CROSS-REFERENCE TO RELATED APPLICATION

This application claims priority to and the benefit of U.S. Provisional Patent Application Ser. No. 63/589,297, filed Oct. 10, 2023, and U.S. Provisional Patent Application Ser. No. 63/658,583, filed Jun. 11, 2024. The entire contents of each of these applications are hereby incorporated by reference herein.

### SEQUENCE LISTING STATEMENT

A computer readable form of the Sequence Listing is filed with this application by electronic submission and is incorporated into this application by reference in its entirety. The Sequence Listing is contained in the file created on Sep. 22, 2024 having the file name “23-1331-WO.xml” and is 276,466 bytes in size.

### FIELD OF THE DISCLOSURE

The disclosure generally relates to binding proteins that comprise antigen binding sites, a T cell receptor binding site, and a T cell co-stimulatory molecule binding site. The disclosure also relates to pharmaceutical compositions comprising such binding proteins, nucleic acid molecules encoding such binding proteins, and vectors comprising such nucleic acid molecules. The disclosure further relates to methods of treating a disorder or condition using such binding proteins and pharmaceutical compositions, binding proteins and pharmaceutical compositions for use in the treatment of a disorder or condition, and the use of such binding proteins and pharmaceutical compositions for the manufacture of a medicament for treating a disorder or condition.

### BACKGROUND OF THE DISCLOSURE

Recruitment of T cell cytotoxic activity to destroy tumor cells is a worthwhile but complicated treatment strategy for cancer. The development of CD3-based bispecific T cell engagers (TCEs) as cancer therapeutics has been ongoing for the past 30 years. TCEs simultaneously bind a tumor associated antigen (TAA) and cluster of differentiation 3 (CD3) on a T cell to form a T cell receptor (TCR)-independent artificial immune synapse, circumventing human leukocyte antigen (HLA) restriction, and inducing T cell activation and cytolysis of the tumor cell.

The first generation of TCEs were simple bispecific T cell engagers (BiTEs), composed of two tandem single-chain variable fragments (scFvs) including a strong CD3-binding arm and a TAA binding domain. To date, there is only one Food and Drug Administration-approved BiTE, blinatumomab, which targets CD3 (using the Orthoclone OKT3 antibody) and cluster of differentiation 19 (CD19). The strong in vitro cytolytic activity observed during the development of BiTEs created excitement around their potential use for treating cancer. However, the unanticipated high cytokine release syndrome (CRS) observed in the clinic somewhat tempered that excitement. Teachey et al., “Cytokine release syndrome after blinatumomab treatment related to abnormal macrophage activation and ameliorated with cytokine-directed therapy,” Blood 121: 5154-57 (2013). Another observed disadvantage of BiTE formats is that they exhibit very short half-life and have poor manufacturability. Ellerman, “Bispecific T cell engagers: Towards understanding variables influencing the in vitro potency and tumor selectivity and their modulation to enhance their efficacy and safety,” Methods 154: 102-17 (2019).

The second generation of TCEs include a fragment crystallizable (Fc) domain which can be modified to confer half-life extension and mutations to eliminate Fc receptor (FcR) binding, and present improved manufacturability. Vafa et al., “Perspective: Designing T cell Engagers With Better Therapeutic Windows,” Front. Oncol. 10: 446 (2020). Nevertheless, those molecules still include high affinity-CD3 binding domains, linking to induction of neurotoxicity and CRS in the clinic. More recent efforts have been focused on developing CD3 binding domains with reduced affinity with the hope to maintain potent T cell activation while significantly reducing associated cytokine release. Trinklein et al., “Efficient tumor killing and minimal cytokine release with novel T cell agonist bispecific antibodies,” MAbs 11: 639-52 (2019).

Importantly, both CD3-based BiTEs and novel immunoglobulin G (IgG)-format TCEs bind and activate both cluster of differentiation 4 (CD4) and cluster of differentiation 8 (CD8) T cells, potentially engaging unfavorable T cells such as regulatory T cells (Treg), which have been shown to potentially decrease the cytolytic activity of CD8 T cells. Duell et al., “Frequency of regulatory T cells determines the outcome of the T cell-engaging antibody blinatumomab in patients with B-precursor ALL,” Leukemia 31: 2181-90 (2017).

While T cell engager molecules offer promise, the therapeutic approach has faced challenges to date. There is a need in the art for improved T-cell binding proteins with increased activity and reduced off-target effects.

### SUMMARY OF THE DISCLOSURE

In a first aspect the disclosure provides a binding protein comprising four polypeptide chains that form two tumor-associated antigen (TAA) binding sites, a T cell receptor binding site, and a T cell co-stimulatory molecule binding site, wherein the first and second polypeptide chains have a structure represented by the formula: V_{L}-C_{L} and a third polypeptide chain has a structure represented by the formula: V_{H1}-C_{H1}-V_{HHa}-F_{Ca} and a fourth polypeptide chain has a structure represented by the formula: V_{H1}-C_{H1}-V_{HHb}-F_{Cb}, wherein the first polypeptide and the third polypeptide form the first of the two TAA binding sites, and the second polypeptide and the fourth polypeptide form the second of the two TAA binding sites, and wherein: V_{L} is an immunoglobulin light chain variable domain and V_{H1} is an immunoglobulin heavy chain variable domain that together form a TAA binding domain that specifically binds a tumor-associated antigen; C_{L} is an immunoglobulin light chain constant domain; C_{H1} is an immunoglobulin CH1 heavy chain constant domain; V_{HHa} is a single chain variable domain that specifically binds a T cell receptor; V_{HHb} is a single chain variable domain that specifically binds T cell co-stimulatory molecule; F_{Ca} is C_{H2a} and C_{H3a} immunoglobulin heavy chain constant domains; and F_{Cb} is C_{H2b} and C_{H3b} immunoglobulin heavy chain constant domains.

In a second aspect the disclosure provides novel binding proteins.

### BRIEF DESCRIPTION OF THE DRAWINGS

The accompanying drawings are included to provide a further understanding of the methods and compositions of the disclosure and are incorporated in and constitute a part of this disclosure. The drawings illustrate one or more aspects of the disclosure and together with the description serve to explain the principles and operation of the disclosure.

FIG. 1: FIG. 1A shows the in vitro cytolytic activity of LM1486 and CD3 engagers with monovalent or bivalent engagement to CD20. FIG. 1B shows the corresponding EC₅₀s to cytolysis in vitro assay. FIG. 1C shows corresponding CD4T cell activation profiles as % of cells expressing CD25. FIG. 1D shows corresponding CD8T cell activation profiles as % of cells expressing CD25.

FIG. 2 shows cytokine release profiles for LM1486 for IL-6 (FIG. 2A), IL-10 (FIG. 2B), TNF-α (FIG. 2C), and IL-17A (FIG. 2D).

FIG. 3 shows the in vitro cytolytic activity of LM1486 with different CD20+ B cell lines incubated with PBMCs at an E:T ratio of 5:1.

FIG. 4 illustrates the structure of LM1486 (FIG. 4A) and the structure of modified binding proteins with a null binding domain replacing the anti-CD8 domain (FIG. 4B), or a null binding domain replacing the anti-TCR binding domain (FIG. 4C). FIG. 4D shows the in vitro cytolytic activity of LM1486 and control binding proteins TENG0501 and TENG0502. FIG. 4E shows corresponding T cell activation profiles as % CD4 and CD8 T cells expressing CD25.

FIG. 5 illustrates the structure of LM1486 (FIG. 5A) and the structure of modified binding proteins with a null binding domain replacing the anti-CD20 domain on the anti-CD8 arm (FIG. 5B) or with a null binding domain replacing the anti-CD20 binding domain on the anti-TCR arm (FIG. 5C). FIG. 5D shows the in vitro cytolytic activity of LM1486 and control binding proteins TENG0499 and TENG0500. FIG. 5E shows corresponding T cell activation profiles as % CD4 and CD8 T cells expressing CD25.

FIG. 6 shows the tumor burden, total flux (p/s), which are presented for PBS treated groups (FIG. 6A), LM1486 at 0.8 mg/kg (FIG. 6B), LM1486 at 0.08 mg/kg (FIG. 6C), and LM1486 at 0.008 mg/kg (FIG. 6D).

FIG. 7 shows tumor growth inhibition (FIG. 7A), percentage survival (FIG. 7B), and percentage body weigh loss (FIG. 7C) as compared to conventional CD3 engagers in a fully humanized in vivo model with subcutaneous B cell tumor.

FIG. 8 shows the anti-tumor efficacy of LM1486 in a disseminated DLBCL in vivo model: Total Flux (FIG. 8A), TNF-α (FIG. 8B), IL-10 (FIG. 8C), IL-2 (FIG. 8D), IL-17A (FIG. 8E), and % weight loss (FIG. 8F).

FIG. 9 shows the in vitro cytolytic activity of LM1486 and LM1486-2 engagers. FIGS. 9A and 9D show cytotoxicity profiles in a range of T cell engager concentrations for 2 different PBMC donors. FIG. 9B, 9C, and FIG. 9E, 9F show associated CD4 and CD8 T cell activation profiles as % of CD25 surface expression for 2 donors, respectively.

FIG. 10 shows binding of LRC150016 to various cell lines; combined LRRC15pos cell lines (FIG. 10A), Saos-2 (FIG. 10B), and RPMI (FIG. 10C).

FIG. 11 shows LRC150016 engager mediates T-cell effector functions in an antigen (LRRC15) dependent manner. FIG. 11A shows in vitro cytolytic activity of LRC150016 against LRRC15pos (Saos-2 WT) and LRRC15neg (Saos-2 KO and A431-WT) target cell lines. FIG. 11B shows cytokine profile measured in the supernatant of T cell in vitro cytolysis experiment (as outlined in FIG. 11A) using a multiplexing cytokine assay.

FIG. 12 shows in vitro cytolysis and CD8-biased T-cell activation profiles induced by LRC150016, compared to a comparator CD3 TCE. FIG. 12A shows the in vitro cytolytic activity mediated by LRC150016 or Comparator CD3 TCE. FIG. 12B shows LRC150016 or Comparator CD3 TCE mediated CD8+ T cells activation, as measured by % of CD25 surface expression.

FIG. 12C shows LRC150016 or Comparator CD3 TCE mediated CD4+ T cells activation, as measured by % of CD25 surface expression.

FIG. 13 shows in vitro cytokine profile of LRC150016, compared to Comparator CD3 TCE: TNFα (FIG. 13A), IL-6 (FIG. 13B), IL-10 (FIG. 13C), and IL-2 (FIG. 13D).

FIG. 14 shows the LRC150016 specific in vitro cytolytic activity across an array of cell lines expressing various levels of LRRC15. FIG. 14A shows Eₘₐₓ and FIG. 14B shows EC₅₀.

FIG. 15 shows a diagram of the LRC150016 Binding Protein.

FIG. 16 shows in vitro cytolysis induced by LM1653 compared to a conventional GPC3×CD3 engager, against hepatocellular carcinoma (HCC) cell lines expressing different levels of GPC3. FIG. 16A shows expression of GPC3 across 4 HCC cell lines. FIG. 16B-E show the in vitro cytolytic activity of LM1653 and of a monovalent GPC3×CD3 engager against the HCC cell lines HepG2 (FIG. 16B), Hep3B (FIG. 16C), Huh-7 (FIG. 16D), and PLC/PRF/5 (FIG. 16E).

FIG. 17 shows CD8+ T cell (upper panels) and CD4+ T cell (bottom panels) activation profiles induced by LM1653 and by a monovalent GPC3×CD3 engager in cytotoxicity assay against HCC cell lines HepG2 (FIG. 17A), Hep3B (FIG. 17B), Huh-7 (FIG. 17C), and PLC/PRF/5 (FIG. 17D).

FIG. 18 shows in vitro cytokine release profiles of LM1653 compared to a conventional GPC3×CD3 engager: TNF-α (FIG. 18A), IL-10 (FIG. 18B), IL-2 (FIG. 18C), and IFN-7 (FIG. 18D), in an in vitro cytolysis assay, using HCC Hep3B cell line as target cells at E:T ratio of 10:1.

FIG. 19 shows non-specific activity of LM1653 and of a conventional GPC3×CD3 engager in a plate-bound assay. CD8 and CD4 T cell activation profiles (defined as % of parental cells expressing CD25) are depicted in FIG. 19A and FIG. 19B, respectively. Histograms represent mean±SEM; *, p<0.05, 2-way ANOVA. Released IL-6 (FIG. 19C), TNF-α (FIG. 19D), IFN-7 (FIG. 19E), IL-2 (FIG. 19F), IL-10 (FIG. 19G), and IL-17 (FIG. 19H) were measured in the supernatant at 48 hours, using a multiplex assay (Luminex). Results from one representative donor PBMC are shown.

FIG. 20 shows the in vitro cytolytic activity (FIG. 20A-B) and cytokine secretion (FIG. 20C-J) of LM1653 and of a monovalent GPC3×CD3 engager using purified CD4 T cells (upper panels) and purified CD8 T cells (bottom panels), in a Xcelligence assay.

FIG. 21 shows LM1653-dependent killing of GPC3⁺Hep3B cell line by patients-derived tumor infiltrating lymphocytes obtained from HCC stage II (FIG. 21A) and stage IIIB (FIG. 21B).

FIG. 22 shows the in vitro cytolytic activity of LM1653 and of a monovalent GPC3×CD3 engager against non small cell lung cancer lines NCI-H661 (FIG. 22A) and NCI-H2172 (FIG. 22B) and ovarian cancer cell lines OV-90 (FIG. 22C) and Kuramochi (FIG. 22D).

FIG. 23 shows comparable in vitro cytolysis and T cell activation profile induced by LM1653 and LM1653-2 (same sequence as LM1653, but a different lot). FIG. 23A-B Cytolysis activity was measured at day 4, using PBMC at the ratio of 10:1 in the XCELLIGENCE system (n=1 PBMC donor). The tables show the corresponding EC₅₀ and killing Eₘₐₓ for GPC3+ cell line Hep3B (FIG. 23A) and Huh-7 (FIG. 23B). T cell activation profile was determined in cytotoxicity assay, using the Hep3B (FIG. 23C-D) and Huh-7 cell line (FIG. 23E-F). The percentage of CD8 (filled symbol) and CD4 (empty symbol) T cells expressing CD25 is reported.

FIG. 24 shows that LM1653 confers anti-tumor efficacy in vivo against GPC3^{high} and GPC3^{low} HCC xenograft models. FIG. 24A shows 5e06 Hep3B cells, expressing high levels of GPC3. On day 21, animals were humanized with 10e06 panT cells expanded in vitro. Tumor burden was measured overtime and reported as mean±SEM. 1, 40% tumor-free; **, p<0.01 and ***, p<0.001, 2-way Anova. FIG. 24B shows 5e06 PLC/PRF/5 cells, expressing low levels of GPC3. Tumor burden was measured overtime and reported as mean±SEM. **, p<0.0001, 2-way Anova. Dotted lines represent TCE administrations.

FIG. 25 shows that LM1653 (GPC3 TITAN) binds human hepatoma cells Hep3B at EC₅₀: 8 nM (FIG. 25A) and HepG2 at EC₅₀: 2 nM (FIG. 25B). GPC3 TITAN binds to human PBMC at EC₅₀: 6 nM (FIG. 25C).

FIG. 26 depicts comparator CD3×CD20 T cell engagers (FIGS. 26A and 26B).

FIG. 27 depicts the superior B cell depletion (FIG. 27A and FIG. 27B), CD4 T-cell sparing activity (FIG. 27C and FIG. 27D), and selective CD8+ T-cell activation (FIG. 27E and FIG. 27F) of LM1486 (CD20 TITAN) in PBMCs derived from whole blood of patients with SLE (FIG. 27A, FIG. 27C, and FIG. 27E) and myositis (FIG. 27B, FIG. 27D, and FIG. 27F).

FIG. 28 compares the cytokine release at varying concentrations of mosunetuzumab and LM1486 (CD20 TITAN) in an assay of non-specific T-cell activation of IL-17A (FIG. 28A) and TNFα (FIG. 28B).

FIG. 29 depicts the humanized mouse model of B-cell depletion.

FIG. 30 depicts depletion of B cells in a humanized mouse model after treatment with vehicle, NIP228 (control) or varying mono doses of LM1486 (CD20 TITAN) in spleen (FIG. 30A), blood (FIG. 30B), bone marrow (FIG. 30C), kidney (FIG. 30D), thymus (FIG. 30E), and lymph nodes (FIG. 30F).

FIG. 31 shows potent in vitro cytolysis by LM1953. FIG. 31A shows the in vitro cytolytic activity of LM1953 on C4-2 cells and the corresponding EC₅₀. FIG. 31B shows the in vitro cytolytic activity of LM1953 on 22Rv1 cells and the corresponding EC₅₀. A CD8-biased T cell activation profile was induced by LM1953.

FIG. 32 shows corresponding CD4 and CD8 T cell activation profiles as % of cells expressing CD25 on the cell surface in the C4-2 cytolysis assay (FIG. 32A) and in the 22Rv1 cytolysis assay (FIG. 32B).

FIG. 33 shows in vitro cytokine release profiles of LM1953 on C4-2 cells for IL-6 (FIG. 33A) and TNF-α (FIG. 33C), and on 22Rv1 cells for IL-6 (FIG. 33B) and TNF-α (FIG. 33D).

FIG. 34 shows that LM1953 confers anti-tumor efficacy in vivo against STEAP2+ prostate cancer xenograft model 22Rv1.

FIG. 35 shows that the LRRC15 variants preferentially activate CD8+ T cells, have functional activity (FIG. 35A), are cytotoxic (FIG. 35B), and IL-6 conc (FIG. 35C).

FIG. 36 shows binding of the LRRC15 TCE variants to EphB6-expressing As293 cells.

FIG. 37 shows that plasmablasts were depleted in mice three days following a single treatment of TCP-42197 (BCMA TCE) in both the bone marrow (FIG. 37A) and in the spleen (FIG. 37B). Serum human IgG was significantly lower in the TCP-42197 treatment groups compared to the isotype control treated group (FIG. 37C).

FIG. 38 shows that TPP-42197 (BCMA TCE) highly depletes plasmablasts compared to Comparator T.

FIG. 39 shows that TPP-42197 (BCMA TCE) selectively activates CD8+ T cells (FIG. 39A) over CD4+ T cells (FIG. 39B).

FIG. 40 show that the cytokine profile of TPP-42197 (BCMA TCE) is superior to that of the Comparator T with lower cytokine secretion in general and in particular those involved in CRS events IL-17A (FIG. 40A), IL-22 (FIG. 40B), IL-10 (FIG. 40C), IL-6 (FIG. 40D), TNFα (FIG. 40E), and IFNγ (FIG. 40F).

FIG. 41 shows that LM1653-2 induced significant tumor growth inhibition in Hep3B tumor bearing mice without significant associated cytokine secretion, in comparison to a conventional GPC3×CD3 engager. FIG. 41A shows the tumor burden, which was monitored twice a week by bioluminescence imaging (BLI) until Day 24, 2 days post treatment. Serum cytokine concentrations are shown for each treatment group: IFN-7 (FIG. 41B), TNFα (FIG. 41C), IL-10 (FIG. 41D), and IL-2 (FIG. 41E).

FIG. 42 shows LM1653-3 (same sequence as LM1653 but a different lot) induced significant tumor growth inhibition in NCI-H661 lung tumor bearing mice without significant associated cytokine secretion, in comparison to vehicle-treated animals. FIG. 42A shows the tumor burden, which was monitored twice a week by bioluminescence imaging (BLI). Serum cytokine concentrations are shown for each treatment group: IFNγ (FIG. 42B), TNFα (FIG. 42C), IL-10 (FIG. 42D), and IL-2 (FIG. 42E).

FIG. 43 shows administration of LM1653 in cynomolgus monkeys results in transient CD8+ T cell margination and limited increase in circulating cytokine levels. FIG. 43A shows the absolute count of cytotoxic CD8+ T cells in blood over the course of the treatment, reported as mean±SD per group. Doses: 1 mg/kg/week IV (Δ), 6 mg/kg/week IV ( ) or 6 mg/kg/week SC (0) followed by a 2-week recovery period. Animals that received vehicle only as both IV & SC (O) were used as controls. FIGS. 43B-E shows serum IL-10 (FIG. 43B), IL-6 (FIG. 43C), MCP-1 (FIG. 43D), and MIP-1b (FIG. 43E) levels overtime, during the treatment, determined using a multiple assay (MSD) and reported as mean per treatment group. Solid and dotted lines represent values obtained for male and female, respectively.

FIG. 44 shows the schematic of the cynomolgus monkeys dosage study for LM1486 (CD20 TITAN).

FIG. 45 shows the results of B-cell depletion in blood and tissues. FIG. 45A shows the results of circulating CD19+ B cells and FIG. 45B shows tissue specific CD19+ B cells in spleen, bone marrow and lymph nodes.

FIG. 46 shows absolute circulating levels of CD8+(FIG. 46A) and CD4+(FIG. 46B) T cells post first dose, and activated CD8/CD4 (CD25+) (FIG. 46C). LM1486 and CD20 TITAN are used interchangeably.

FIG. 47 shows group mean plasma cytokine profiles for IL-6 (FIG. 47A), TNFα (FIG. 47B), and IFNγ (FIG. 47C) as a time course from Day −14 to Day 16.

FIG. 48 shows the diagrams and amino acid sequences for the various T-Cell engagers exemplified herein. LM1486-2 (CD20) (FIG. 48A), LM1653 (GPC3) (FIG. 48B), TPP-42197 (BCMA) (FIG. 48C), LM1953 (STEAP2) (FIG. 48D), TPP-46956 (LRRC15) (FIG. 48E), TPP-46957 (LRRC15) (FIG. 48F), TPP-46958 (LRRC15) (FIG. 48G), TPP-46959 (LRRC15) (FIG. 48H), TPP-46960 (LRRC15) (FIG. 48I), TPP-47826 (LRRC15) (FIG. 48J), TPP-49058 (LRRC15) (FIG. 48K) and LM1486 (FIG. 48L).

### DETAILED DESCRIPTION OF THE DISCLOSURE

The disclosure generally relates to binding proteins that comprise antigen binding sites, a T cell receptor binding site, and a T cell co-stimulatory molecule binding site. The disclosure also relates to pharmaceutical compositions comprising such binding proteins, nucleic acid molecules encoding such binding proteins, and vectors comprising such nucleic acid molecules. The disclosure further relates to methods of treating a disorder or condition using such binding proteins and pharmaceutical compositions, binding proteins and pharmaceutical compositions for use in the treatment of a disorder or condition, and the use of such binding proteins and pharmaceutical compositions for the manufacture of a medicament for treating a disorder or condition.

It is to be understood that the particular aspects of the disclosure are described herein are not limited to specific aspects presented and can vary. It also will be understood that the terminology used herein is for the purpose of describing particular aspects only and, unless specifically defined herein, is not intended to be limiting. Moreover, particular aspects disclosed herein can be combined with other aspects disclosed herein, as would be recognized by a skilled person, without limitation.

Unless otherwise indicated or otherwise evident from the context and understanding of one of ordinary skill in the art, values herein that are expressed as ranges can assume any specific value or sub-range within the stated ranges in different aspects of the disclosure, to the tenth of the unit of the lower limit of the range, unless the context clearly dictates otherwise.

Throughout this disclosure, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps.

As used herein, the singular forms “a,” “an,” and “the” include plural referents unless the context clearly indicates otherwise. Unless otherwise required by context, singular terms shall include pluralities and plural terms shall include the singular.

Percentages disclosed herein can vary in amount by +10%, 20%, or 30% from values disclosed and remain within the scope of the contemplated disclosure.

As used herein, ranges and amounts can be expressed as “about” a particular value or range. The term “about” also includes the exact amount. For example, “about 5%” means “about 5%” and also “5%.” The term “about” can also refer to +10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As utilized in accordance with the present disclosure, unless otherwise indicated, all technical and scientific terms shall be understood to have the same meaning as commonly understood by one of ordinary skill in the art.

The term “binding protein,” as used herein, refers to a non-naturally occurring (or recombinant) molecule which comprises multiple polypeptide chains that form at least one antigen binding site.

A “recombinant” molecule is one that has been prepared, expressed, created, or isolated by recombinant DNA technology means.

The term “antibody,” as used herein, refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The variable domain may be subjected to further protein engineering to humanize the framework regions if the antibody was derived from a non-human source. The carboxyl-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the CH3 domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

“Antigen-binding fragment thereof” refer to at least the minimal portion of an antibody which is capable of binding to a specified antigen which the antibody targets, e.g., at least some of the complementarity determining regions (CDRs) of the variable domain of a heavy chain (V_{H}) and the variable domain of a light chain (V_{L}) in the context of a typical antibody produced by a B cell. Antibodies or antigen-binding fragments thereof can be or be derived from polyclonal, monoclonal, human, humanized, or chimeric antibodies, single chain antibodies, epitope-binding fragments, e.g., Fab, Fab′ and F(ab′)2, Fd, Fvs, single-chain Fvs (scFvs), single-chain antibodies, disulfide-linked Fvs (sdFvs), fragments comprising either a V_{L} or V_{H} domain alone or in conjunction with a portion of the opposite domain (e.g., a whole V_{L} domain and a partial V_{H} domain with one, two, or three CDRs), and fragments produced by a Fab expression library. ScFv molecules are known in the art and are described, e.g., in U.S. Pat. No. 5,892,019.

The term “native Fc,” as used herein, refers to a molecule comprising the sequence of a non-antigen binding fragment resulting from digestion of an antibody or produced by other means, whether in monomeric or multimeric form, and can contain the hinge region. The original immunoglobulin source of the native Fc is preferably of human origin and can be any of the immunoglobulins. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3 and IgG4). One example of a native Fc is a disulfide-bonded dimer resulting from papain digestion of an IgG. The term “native Fc,” as used herein, is generic to the monomeric, dimeric, and multimeric forms.

The term “Fc variant,” as used herein, refers to a molecule or sequence that is modified from a native Fc but still comprises a binding site for the salvage receptor, FcRn (neonatal Fc receptor). Exemplary Fc variants, and their interaction with the salvage receptor, are known in the art. Thus, the term “Fc variant” can comprise a molecule or sequence that is humanized from a non-human native Fc. Furthermore, a native Fc comprises regions that can be removed or mutated to produce an Fc variant to alter certain residues that provide structural features or biological activity that are not required for the binding proteins of the disclosure. Thus, the term “Fc variant” comprises a molecule or sequence that lacks one or more native Fc sites or residues, or in which one or more Fc sites or residues has been modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

The term “Fc,” as used herein, encompasses native Fc and Fc variants as defined above. As with Fc variants and native Fc molecules, the term “Fc” includes molecules in monomeric or multimeric form, whether digested from whole antibody or produced by other means.

Binding proteins encompassed by this disclosure can be of or be derived from any type (e.g., IgG, IgE, IgM, IgD, IgA, and IgY), class (e.g., IgG1, IgG2, IgG3, IgG4, IgA1, and IgA2), or subclass of immunoglobulin molecule.

The terms “knob,” “hole,” and “knob in hole” as used herein refer to a previously developed system to direct the correct pairing of heavy chains from differing antibodies. A “knob” and a “hole” are engineered into the two heavy chains of differing antibodies in order to promote correct pairing. The “knob in hole” approach is an effective way to produce bispecific antibodies by driving heterodimerization with mutations in the CH3 domain of each half antibody.

The term “antigen” or “target antigen,” as used herein, refers to a molecule or a portion of a molecule that is capable of being recognized by and bound by the antigen binding portion of the binding proteins of the disclosure. The target antigen is capable of being used in an animal to produce antibodies capable of binding to an epitope of that antigen. A target antigen may have one or more epitopes. With respect to each target antigen recognized by the antigen binding portion of the binding protein, is capable of competing with an intact antibody that recognizes the target antigen.

The term “antigen binding site,” as used herein, refers to a site created on the surface of a binding protein of the disclosure where an antigen or an epitope on an antigen is bound.

The term “linker,” as used herein, refers to one or more amino acid residues inserted between domains of the binding protein of the disclosure. For example, a linker may be inserted between domains, at the sequence level. The precise location of a domain transition can be determined by locating peptide stretches that do not form secondary structural elements such as beta-sheets or alpha-helices as demonstrated by experimental data or as can be assumed by techniques of modeling or secondary structure prediction. Linkers may or may not be needed depending on the where the stop and start residues of protein fusions are chosen because often natural linkers are found between immunoglobulin domains.

As used herein, the term “polynucleotide” includes a singular nucleic acid as well as multiple nucleic acids, and refers to an isolated nucleic acid molecule or construct, e.g., messenger RNA (mRNA) or plasmid DNA (pDNA). The term “nucleic acid” includes any nucleic acid type, such as DNA or RNA.

As used herein, the term “vector” can refer to a nucleic acid molecule as introduced into a host cell, thereby producing a transformed host cell. A vector can include nucleic acid sequences that allows the vector to replicate in a host cell, such as an origin of replication. A vector can also include one or more selectable marker gene and other genetic elements known in the art. Specific types of vector envisioned here can be associated with or incorporated into viruses to facilitate cell transformation.

As used herein, the terms “treat,” “treatment,” or “treatment of” refer to reducing disease pathology, reducing or eliminating disease symptoms, promoting increased survival rates, and/or reducing discomfort. For example, treating can refer to the ability of a therapy to reduce disease symptoms, signs, or causes when administered to a subject. Treating also refers to mitigating or decreasing at least one clinical symptom and/or inhibition or delay in the progression of the condition and/or prevention or delay of the onset of a disease or illness.

The terms “administration” or “administering,” as used herein, refer to providing, contacting, and/or delivering a binding protein by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

As used herein, the terms “subject,” “individual,” or “patient,” refer to any subject, particularly a mammalian subject, for whom diagnosis, prognosis, or therapy is desired. Mammalian subjects include, for example, humans, non-human primates, dogs, cats, guinea pigs, rabbits, rats, mice, horses, cattle, bears, and so on.

As used herein, the term an “effective amount” or a “therapeutically effective amount” of an administered therapeutic substance, such as a binding protein, is an amount sufficient to carry out a specifically stated or intended purpose, such as treating or treatment of cancer. An “effective amount” can be determined empirically in a routine manner in relation to the stated purpose.

The term “pharmaceutical composition,” as used herein, refer to a compound or composition capable of inducing a desired therapeutic effect when properly administered to a subject. In some aspects, the disclosure provides a pharmaceutical composition comprising a pharmaceutically acceptable carrier and a therapeutically effective amount of binding proteins of the disclosure. The terms “pharmaceutically acceptable carrier” or “physiologically acceptable carrier,” as used herein, refer to one or more formulation materials suitable for accomplishing or enhancing the delivery of one or more binding proteins of the disclosure.

In some aspects, the binding proteins disclosed herein may be formulated with a pharmaceutically acceptable carrier, excipient, or stabilizer, as pharmaceutical compositions. In certain aspects, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, for example, as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

In some aspects provided herein is a binding protein comprising two tumor-associated antigen (TAA) binding sites. In some aspects, the tumor associated antigen (TAA) is cluster of differentiation 20 (CD20). CD20 is a transmembrane protein involved in Ca⁺⁺ channeling, B cell activation, and proliferation. CD20 is a membrane-embedded surface molecule which plays a role in the development and differentiation of B cells into plasma cells. In some aspects, the binding protein comprises a fragment of rituximab (see, e.g., U.S. Pat. No. 5,736,137). As used herein, CD20 is used interchangeably with LM1486.

In some aspects, the tumor associated antigen (TAA) is Glypican-3 (GPC3). GPC3 is is a Heparan Sulfate Proteoglycans (HSPG) that is highly expressed in hepatocellular carcinoma, where it can attract Wnt proteins to the cell surface and promote cell proliferation. As used herein, GPC3 is used interchangeably with LM1653.

In some aspects, the tumor associated antigen (TAA) is leucine rich repeat containing 15 (LRRC15). Leucine Rich Repeat Containing 15 (LRRC15) is a 581 amino acid type I cell membrane protein belongs to Leucine-rich repeat (LRR) super family with binding activity to extracellular matrix (ECM) components. LRRC15 is involved in cell-cell/cell-ECM interactions, cell adhesion, wound healing, and osteogenic differentiation. LRRC15 is highly expressed on cancer-associated fibroblasts (CAFs) within the stroma of various epithelial solid tumours and on mesenchymal tumours such as sarcomas, glioblastoma, and melanoma. LRRC15⁺ CAF signature is shown to be associated with PDx therapy resistance across various tumour indications like metastatic urothelial carcinoma (mUCC), renal cell cancer (RCC), head and neck squamous cell carcinoma (HNSCC), and non-small cell lung cancer (NSCLC).

In some aspects, the tumor associated antigen (TAA) is B-cell maturation antigen (BCMA), also known as tumor necrosis factor receptor superfamily member 17 (TNFRSF17). BCMA is a member of the TNF-receptor superfamily, which is preferentially expressed in mature B lymphocytes and is important for B cell development, proliferation, and survival. BCMA is associated with hematological malignancies, including, but not limited to multiple myeloma, and autoimmune/inflammatory diseases, including, but not limited to, scleroderma, systemic lupus erythematosus (SLE), myositis, rheumatoid arthritis (RA), anti-neutrophil cytoplasmic autoantibody (ANCA) vasculitis, or Sjogren's syndrome.

In some aspects, the tumor associated antigen (TAA) is six-transmembrane epithelial antigen of prostate-2 (STEAP2). STEAP2 is a membrane-embedded hemoprotein with a transmembrane domain (TMD) that chelates a heme prosthetic group, or metalloreductase. STEAP2 is associated with cancer progression by driving cellular proliferation, migration, and invasion. It also influences the transcriptional profile of genes that are part of the metastatic cascade. STEAP2 is associated with cancers, including, but not limited to prostate cancer and Ewing's sarcoma.

In some aspects provided herein is a binding protein comprising one T cell receptor (TCR) binding site. The TCR comprises a heterodimer including the highly variable alpha (a) and beta (0) chains. The multicomponent complex of the TCR comprises the CD3 co-receptor, which plays a significant role in activating T cells. In one aspect there is provided a TCR binding protein comprising a heavy chain CDR1 (HCDR1), a heavy chain CDR2 (HCDR2) and a heavy chain CDR3 (HCDR3) comprising the amino acid sequences of SEQ ID NO: 62, SEQ ID NO: 40 and SEQ ID NO: 41 respectively. In one embodiment, the TCR binding protein comprises a VHH chain according to SEQ ID NO: 43 or SEQ ID NO: 45. In another aspect, the TCR binding protein comprises a VHH chain according to SEQ ID NO: 44, SEQ ID NO: 46, SEQ ID NO: 47 or SEQ ID NO: 48.

In some aspects provided herein is a binding protein comprising one T cell costimulatory molecule binding site. A co-stimulatory molecule comprises a co-stimulatory domain capable of potentiating or modulating the response of immune effector cells. Co-stimulatory domains can include sequences, for example, from one or more of CD3zeta (or CD3z), CD28, CD137 (4-1BB), OX-40, ICOS, CD27, GITR, CD2, IL-2Rβ and MyD88/CD40. In some aspects, the T cell costimulatory molecule is CD8. In some aspects, the T cell costimulatory molecule is CD137 (4-1BB).

In one-non-limiting embodiment, the binding protein activates T cells only when bound to a tumor associated antigen at one or both of the tumor associated antigen binding sites.

Some aspects described herein provides a binding protein comprising four polypeptide chains that form two tumor-associated antigen (TAA) binding sites, a T cell receptor binding site, and a T cell co-stimulatory molecule binding site, wherein the first and second polypeptide chains have a structure represented by the formula: V_{L}-C_{L} and a third polypeptide chain has a structure represented by the formula: V_{H1}-C_{H1}-V_{HHa}-F_{Ca} and a fourth polypeptide chain has a structure represented by the formula: V_{H1}-C_{H1}-V_{HHb}-F_{Cb}, wherein the first polypeptide and the third polypeptide form the first of the two TAA binding sites, and the second polypeptide and the fourth polypeptide form the second of the two TAA binding sites, and wherein: V_{L} is an immunoglobulin light chain variable domain and V_{H1} is an immunoglobulin heavy chain variable domain that together form a TAA binding domain that specifically binds a tumor-associated antigen; C_{L} is an immunoglobulin light chain constant domain; C_{H1} is an immunoglobulin CH1 heavy chain constant domain; V_{HHa} is a single chain variable domain that specifically binds a T cell receptor; V_{HHb} is a single chain variable domain that specifically binds T cell co-stimulatory molecule; F_{Ca} is C_{H2a} and C_{H3a} immunoglobulin heavy chain constant domains; and F_{Cb} is C_{H2b} and C_{H3b} immunoglobulin heavy chain constant domains.

In some aspects, the heavy chain variable domain that specifically binds a T cell co-stimulatory molecule is an immunoglobulin heavy chain variable domain. In some aspects, the heavy chain variable domain that specifically binds a T cell co-stimulatory molecule is a single domain sequence. In particular aspects, the heavy chain variable domain that specifically binds a T cell co-stimulatory molecule is a nanobody. In particular aspects, the heavy chain variable domain that specifically binds a T cell co-stimulatory molecule is a camelid. In particular aspects, the heavy chain variable domain that specifically binds a T cell co-stimulatory molecule is a single-domain variable new antigen receptor. In various non-limiting embodiments, V_{HHb} comprises a heavy chain CDR1 (HCDR1), a heavy chain CDR2 (HCDR2), a heavy chain CDR3 (HCDR3) comprising the amino acid sequence of SEQ ID NO: 49, SEQ ID NO: 50, and SEQ ID NO: 51 respectively; or SEQ ID NO: 169, SEQ ID NO: 170 and SEQ ID NO: 171, respectively. In various other non-limiting embodiments, V_{HHb} comprises an amino acid sequence at least 90%, at least 91% at least 92%, at least 93%, at least 94%, at least 95%, at least 96%, at least 97%, at least 98%, or at least 99% identical to SEQ ID NO: 52 or 168, or 100% identical to SEQ ID NO: 52 or 168.

In some aspects, the V_{HHb} is SEQ ID NO: 52 is further modified to further enhance manufacturability. In some aspects, the V_{HHb} is SEQ ID NO: 52 but further comprises a D30R substitution. In some aspects, the V_{HHb} is SEQ ID NO: 52 but further comprises a D30P substitution. In some aspects, the V_{HHb} is SEQ ID NO: 52 but further comprises a S75A substitution. In some aspects, the V_{HHb} is SEQ ID NO: 52 but further comprises a Y102I substitution. In some aspects, the V_{HHb} is SEQ ID NO: 52 but further comprises a S100G substitution. In some aspects, the V_{HHb} is SEQ ID NO: 52 but further comprises a L101A substitution. In some aspects, the V_{HHb} is SEQ ID NO: 52 but further comprises a Q106N substitution. In some aspects, the V_{HHb} is SEQ ID NO: 52 but further comprises a D31S substitution. In some aspects, the V_{HHb} is SEQ ID NO: 52 but further comprises a S100P substitution. In some aspects, the V_{HHb} is SEQ ID NO: 52 but further comprises a R52T substitution. In some aspects, the sequence comprises one or more substitutions selected from D30R, D30P, S75A, Y102I, S100G, L101A, Q106N, D31S, S100P, ORR52T. In some aspects, the V_{HHB} is selected from SEQ ID NOs: 81-89.

In some aspects, the heavy chain variable domain that specifically binds a T cell receptor binding site is an immunoglobulin heavy chain variable domain. In some aspects, the heavy chain variable domain that specifically binds a T cell receptor binding site is a single domain sequence. In particular aspects, the heavy chain variable domain that specifically binds a T cell receptor binding site is a nanobody. In particular aspects, the heavy chain variable domain that specifically binds a T cell receptor binding site is a camelid. In particular aspects, the heavy chain variable domain that specifically binds a T cell receptor binding site is a single-domain variable new antigen receptor. In various non-limiting embodiments, V_{HHa} comprises a heavy chain CDR1 (HCDR1), a heavy chain CDR2 (HCDR2), a heavy chain CDR3 (HCDR3) comprising the amino acid sequence of SEQ ID NO: 39, SEQ ID NO: 40, and SEQ ID NO: 41 respectively; SEQ ID NO: 62, SEQ ID NO: 40 and SEQ ID NO: 41 respectively; or SEQ ID NO: 173, SEQ ID NO: 174, and SEQ ID NO: 175 respectively, respectively. In various other non-limiting embodiments, V_{HHa} comprises the amino acid sequence at least 90%, at least 91% at least 92%, at least 93%, at least 94%, at least 95%, at least 96%, at least 97%, at least 98%, or at least 99% identical to any one of SEQ ID NOs: 42-48, and 172, or any one of SEQ ID NOs: 42-48, and 172.

In some aspects, the F_{Ca} and/or F_{Cb} of the binding protein is from an IgG antibody for example IgG1, IgG2, IgG3 and IgG4. In one non-limiting embodiment F_{Ca} and/or the F_{Cb} is from an IgG1 antibody.

In some aspects, C_{H3a} and C_{H3b} are both CH3 immunoglobulin heavy chain constant domains. In some aspects, C_{H3a} and C_{H3b} in each of F_{Ca} and F_{Cb} comprise modifications to facilitate heterodimerization of F_{Ca} and F_{Cb}. In various non-limiting embodiments, the modifications are substitutions to generate a knob in one of F_{Ca} and F_{Cb} and a hole in the other of F_{Ca} and F_{Cb}. In one nonlimiting embodiment the knob is a substitution to tryptophan at position 366, and the hole is a substitution of one or more of the following: i) a substitution to valine at position 407; ii) a substitution to serine at position 366; and iii) a substitution to alanine at position 368. In another non-limiting embodiment, the F_{Ca} or the F_{Cb} containing the knob further comprises a cysteine at position 354 and/or the F_{Ca} or the F_{Cb} containing the hole comprises a cysteine at position 349 and wherein the numbering is according to the Eu index.

In some aspects, C_{H2a} and C_{H2b} are both CH2 immunoglobulin heavy chain constant domains. In some aspects, C_{H2a} and C_{H2b} immunoglobulin heavy chain constant domains each comprise the following substitutions: E233P/L234V/L235A/G236del/S267K and wherein the numbering is according to the Eu index. In other aspects, either the C_{H3a} immunoglobulin heavy chain constant domain or C_{H3b} immunoglobulin heavy chain constant domain comprises a H435R and Y436F substitution. and wherein the numbering is according to the Eu index.

In various non-limiting embodiments F_{Ca} comprises the amino acid sequence of SEQ ID NO: 56 or SEQ ID NO: 57. In various other non-limiting embodiments F_{Cb} comprises the amino acid sequence of SEQ ID NO: 58.

In some aspects, the fourth polypeptide chain comprises SEQ ID NO: 61 and the third polypeptide chain comprises either SEQ ID NO: 59 or SEQ ID NO: 60.

In some aspects, the binding protein comprises a linker. The identity and sequence of amino acid residues in the linker may vary depending on the type of secondary structural element necessary to be achieved. For example, glycine, serine, and alanine are best for linkers having maximum flexibility. Some combination of glycine, proline, threonine, and serine are useful if a more rigid and extended linker is necessary. Any amino acid residue may be considered as a linker in combination with one or more other amino acid residues, which may be the same as or different as the first amino acid residue, to construct larger peptide linkers as necessary depending on the desired properties. In some aspects described herein the binding protein can further comprise L₁, a linker positioned between C_{H1} and V_{HHa} on the third polypeptide chain and L2, a linker positioned between V_{HHa} and the F_{Ca} on the third polypeptide chain, wherein L₁ and L2 are each independently a linker or are absent. In some aspects described herein the binding protein can further comprise L3, a linker positioned between C_{H1} and V_{HHb} on the fourth polypeptide chain, and L4, a linker positioned between V_{HHb} and F_{Cb} on the fourth polypeptide chain, wherein L3 and L4 are each independently a linker or are absent. In various non-limiting embodiments L₁, L₂, L₃, and/or L₄ comprise one or more repeats of the amino acid sequence of SEQ ID NO: 37 and/or SEQ ID NO: 38.

In some aspects described herein the binding protein comprises H₁, an immunoglobulin hinge region positioned between C_{H1} and V_{HHa} on the third polypeptide chain, and H₂, an immunoglobulin hinge region positioned between V_{HHa} and the F_{Ca} on the third polypeptide chain, wherein H₁ and H₂ are each independently an immunoglobulin hinge region or are absent. In various non-limiting embodiments, H₁ comprises SEQ ID NO: 53, DK (SEQ ID NO: 54), SEQ ID NO: 90 or is absent, and wherein H₂ comprises SEQ ID NO: 53, SEQ ID NO: 55, SEQ ID NO: 90 or is absent.

In some aspects described herein the binding protein comprises H₃, an immunoglobulin hinge region positioned between C_{H1} and V_{HHb} on the fourth polypeptide chain, and H4, an immunoglobulin hinge region positioned between V_{HHb} and the F_{Cb} on the fourth polypeptide chain, wherein H₃ and H₄ are each independently an immunoglobulin hinge region or are absent. In various non-limiting embodiments, H₃ comprises SEQ ID NO: 53, DK (SEQ ID NO: 54), SEQ ID NO: 90 or is absent, and wherein H₄ comprises SEQ ID NO: 53, SEQ ID NO: 55, SEQ ID NO: 90, or is absent. In various non-limiting embodiments, H₃ comprises SEQ ID NO: 53, DK (SEQ ID NO: 54), SEQ ID NO: 90 or is absent, and wherein H₄ comprises SEQ ID NO: 53, SEQ ID NO: 55, SEQ ID NO: 91, or is absent.

Some aspects described herein provide a binding protein comprising four polypeptide chains that form two tumor-associated antigen binding sites, a T cell receptor binding site, and a T cell co-stimulatory molecule binding site, wherein the first and second polypeptide chains have a structure represented by the formula: V_{L}-C_{L}, a third polypeptide chain has a structure represented by the formula: V_{H1}-C_{H1}-H₁-L₁-V_{HHa}-H₂-L₂-F_{Ca} or V_{H1}-C_{H1}-H₁-L₁-V_{HHa}-L₂-H₂-F_{Ca}, and a fourth polypeptide chain has a structure represented by the formula: V_{H1}-C_{H1}-H₃-L₃-V_{HHb}-H₄-L₄-F_{Cb} or V_{H1}-C_{H1}-H₃-L₃-V_{HHb}-L₄-H₄-F_{Cb}, wherein L₁ is a linker positioned between C_{H1} and V_{HHa} on the third polypeptide chain, and L₂ is a linker positioned between V_{HHa} and the F_{Ca} on the third polypeptide chain, wherein L₁ and L₂ are each independently a linker or are absent, and L₃ is a linker positioned between C_{H1} and V_{HHb} on the fourth polypeptide chain, and L₄ is a linker positioned between V_{HHb} and F_{Cb} on the fourth polypeptide chain, wherein L₃ and L₄ are each independently a linker or are absent; wherein H₁ is an immunoglobulin hinge region positioned between C_{H1} and VHH on the third polypeptide chain, and H₂, an immunoglobulin hinge region positioned between V_{HHa} and the F_{Ca} on the third polypeptide chain, wherein H₁ and H₂ are each independently an immunoglobulin hinge region or are absent; wherein H₃ is an immunoglobulin hinge region positioned between C_{H1} and V_{HHb} on the fourth polypeptide chain, and H4, an immunoglobulin hinge region positioned between V_{HHb} and the F_{Cb} on the fourth polypeptide chain, wherein H₃ and H₄ are each independently an immunoglobulin hinge region or are absent.

In some aspects, the binding protein comprises a tumor-associated antigen (TAA) which is CD20, Glypican-3 (GPC3) or leucine rich repeat containing 15 (LRRC15).

In some aspects, the tumor-associated antigen (TAA) is B-cell maturation antigen (BCMA) or six-transmembrane epithelial antigen of prostate-2 (STEAP2).

In some aspects, the TAA binding domain binds to CD20 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 1, SEQ ID NO: 2, SEQ ID NO: 3, SEQ ID NO: 4, SEQ ID NO: 5, and SEQ ID NO: 6, respectively. In other aspects, the TAA binding domain binds to CD20 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 182, SEQ ID NO: 2, SEQ ID NO: 3, SEQ ID NO: 4, SEQ ID NO: 5 and SEQ ID NO: 6, respectively. In various non-limiting embodiments, the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 7 and SEQ ID NO: 8, respectively; or comprises a V_{H} according to SEQ ID NO: 7 and a V_{L} according to SEQ ID NO: 8.

In some aspects, the TAA binding domain binds to GPC3 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 13, SEQ ID NO: 14, SEQ ID NO: 15, SEQ ID NO: 16, SEQ ID NO: 17, and SEQ ID NO: 18, respectively. In various non-limiting embodiments, the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 19 and SEQ ID NO: 20, respectively; or comprises a V_{H} according to SEQ ID NO: 19 and a V_{L} according to SEQ ID NO: 20.

In some aspects, the TAA binding domain binds to LRRC15 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 25, SEQ ID NO: 26, SEQ ID NO: 27, SEQ ID NO: 28, SEQ ID NO: 29, and SEQ ID NO: 30, respectively. In various non-limiting embodiments, the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 31 and SEQ ID NO: 32, respectively; or comprises a V_{H} according to SEQ ID NO: 31 and a V_{L} according to SEQ ID NO: 32.

In some aspects, the TAA binding domain binds to LRRC15 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 136, SEQ ID NO: 137, SEQ ID NO: 138, SEQ ID NO: 139, and SEQ ID NO: 140 respectively. In various non-limiting embodiments, the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 141 and SEQ ID NO: 142, respectively; or comprises a V_{H} chain according to SEQ ID NO: 141 and a V_{L} according to SEQ ID NO: 142.

In some aspects, the TAA binding domain binds to LRRC15 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 147, SEQ ID NO: 137, SEQ ID NO: 138, SEQ ID NO: 139, and SEQ ID NO: 140 respectively. In various non-limiting embodiments, the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 148 and SEQ ID NO: 142, respectively; or comprises a V_{H} chain according to SEQ ID NO: 148 and a V_{L} according to SEQ ID NO: 142.

In some aspects, the TAA binding domain binds to LRRC15 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 152, SEQ ID NO: 137, SEQ ID NO: 138, SEQ ID NO: 139, and SEQ ID NO: 140 respectively. In various non-limiting embodiments, the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 153 and SEQ ID NO: 142, respectively; or comprises a V_{H} chain according to SEQ ID NO: 153 and a V_{L} according to SEQ ID NO: 142.

In some aspects, the TAA binding domain binds to LRRC15 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 157, SEQ ID NO: 137, SEQ ID NO: 138, SEQ ID NO: 158, and SEQ ID NO: 140 respectively. In various non-limiting embodiments, the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 159 and SEQ ID NO: 160, respectively; or comprises a V_{H} chain according to SEQ ID NO: 159 and a V_{L} according to SEQ ID NO: 160.

In some aspects, the TAA binding domain binds to LRRC15 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 157, SEQ ID NO: 137, SEQ ID NO: 138, SEQ ID NO: 165, and SEQ ID NO: 140 respectively. In various non-limiting embodiments, the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 159 and SEQ ID NO: 166, respectively; or comprises a V_{H} chain according to SEQ ID NO: 159 and a V_{L} according to SEQ ID NO: 166.

In some aspects, the TAA binding domain binds to BCMA and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 111, SEQ ID NO: 112, SEQ ID NO: 113, SEQ ID NO: 114, SEQ ID NO: 115, and SEQ ID NO: 116, respectively. In various non-limiting embodiments, the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 117 and SEQ ID NO: 118, respectively; or comprises a V_{H} according to SEQ ID NO: 117 and a V_{L} according to SEQ ID NO: 118.

In some aspects, the TAA binding domain binds to STEAP-2 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 123, SEQ ID NO: 124, SEQ ID NO: 125, SEQ ID NO: 126, SEQ ID NO: 127, and SEQ ID NO: 128, respectively. In various non-limiting embodiments, the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 129 and SEQ ID NO: 130, respectively; or comprises a V_{H} chain according to SEQ ID NO: 129 and a V_{L} according to SEQ ID NO: 130.

Some aspects described herein provide a binding protein comprising the amino acid of sequences of SEQ ID NO: 9, SEQ ID NO: 11, and SEQ ID NO: 12.

Some aspects described herein provide a binding protein comprising the amino acid of sequences of SEQ ID NO: 21, SEQ ID NO: 23, and SEQ ID NO: 24.

Some aspects described herein provide a binding protein comprising the amino acid of sequences of SEQ ID NO: 33, SEQ ID NO: 35, and SEQ ID NO: 36.

Some aspects described herein provide a binding protein comprising the amino acid of sequences of SEQ ID NO: 119, SEQ ID NO: 121, and SEQ ID NO: 122.

Some aspects described herein provide a binding protein comprising the amino acid of sequences of SEQ ID NO: 131, SEQ ID NO: 134, and SEQ ID NO: 133.

Some aspects described herein provide a binding protein comprising the amino acid of sequences of SEQ ID NO: 143, SEQ ID NO: 145, and SEQ ID NO: 146.

Some aspects described herein provide a binding protein comprising the amino acid of sequences of SEQ ID NO: 143, SEQ ID NO: 150, and SEQ ID NO: 151.

Some aspects described herein provide a binding protein comprising the amino acid of sequences of SEQ ID NO: 143, SEQ ID NO: 155, and SEQ ID NO: 156.

Some aspects described herein provide a binding protein comprising the amino acid of sequences of SEQ ID NO: 161, SEQ ID NO: 163, and SEQ ID NO: 164.

Some aspects described herein provide a binding protein comprising the amino acid of sequences of SEQ ID NO: 167, SEQ ID NO: 163, and SEQ ID NO: 164.

Some aspects described herein provide a binding protein comprising the amino acid of sequences of SEQ ID NO: 161, SEQ ID NO: 177, and SEQ ID NO: 164.

Some aspects described herein provide a binding protein comprising the amino acid of sequences of SEQ ID NO: 143, SEQ ID NO: 176, and SEQ ID NO: 156.

In some aspects the first and second polypeptide chains comprise the amino acid sequence of SEQ ID NO: 9, the third polypeptide chain comprises the amino acid sequence of SEQ ID NO: 11, and the fourth polypeptide chain comprises the amino acid sequence of SEQ ID NO: 12.

In some aspects the first and second polypeptide chains comprise the amino acid sequence of SEQ ID NO: 21, the third polypeptide chain comprises the amino acid sequence of SEQ ID NO: 23, and the fourth polypeptide chain comprises the amino acid sequence of SEQ ID NO: 24.

In some aspects the first and second polypeptide chains comprise the amino acid sequence of SEQ ID NO: 33, the third polypeptide chain comprises the amino acid sequence of SEQ ID NO: 35, and the fourth polypeptide chain comprises the amino acid sequence of SEQ ID NO: 36.

In some aspects the first and second polypeptide chains comprise the amino acid sequence of SEQ ID NO: 119, the third polypeptide chain comprises the amino acid sequence of SEQ ID NO: 121, and the fourth polypeptide chain comprises the amino acid sequence of SEQ ID NO: 122.

In some aspects the first and second polypeptide chains comprise the amino acid sequence of SEQ ID NO: 131, the third polypeptide chain comprises the amino acid sequence of SEQ ID NO: 134, and the fourth polypeptide chain comprises the amino acid sequence of SEQ ID NO: 133.

In some aspects the first and second polypeptide chains comprise the amino acid sequence of SEQ ID NO: 143, the third polypeptide chain comprises the amino acid sequence of SEQ ID NO: 145, and the fourth polypeptide chain comprises the amino acid sequence of SEQ ID NO: 146.

In some aspects the first and second polypeptide chains comprise the amino acid sequence of SEQ ID NO: 143, the third polypeptide chain comprises the amino acid sequence of SEQ ID NO: 150, and the fourth polypeptide chain comprises the amino acid sequence of SEQ ID NO: 151.

In some aspects the first and second polypeptide chains comprise the amino acid sequence of SEQ ID NO: 143, the third polypeptide chain comprises the amino acid sequence of SEQ ID NO: 155, and the fourth polypeptide chain comprises the amino acid sequence of SEQ ID NO: 156.

In some aspects the first and second polypeptide chains comprise the amino acid sequence of SEQ ID NO: 161, the third polypeptide chain comprises the amino acid sequence of SEQ ID NO: 163, and the fourth polypeptide chain comprises the amino acid sequence of SEQ ID NO: 164.

In some aspects the first and second polypeptide chains comprise the amino acid sequence of SEQ ID NO: 167, the third polypeptide chain comprises the amino acid sequence of SEQ ID NO: 163, and the fourth polypeptide chain comprises the amino acid sequence of SEQ ID NO: 164.

In some aspects the first and second polypeptide chains comprise the amino acid sequence of SEQ ID NO: 161, the third polypeptide chain comprises the amino acid sequence of SEQ ID NO: 177, and the fourth polypeptide chain comprises the amino acid sequence of SEQ ID NO: 164.

In some aspects the first and second polypeptide chains comprise the amino acid sequence of SEQ ID NO: 143, the third polypeptide chain comprises the amino acid sequence of SEQ ID NO: 176, and the fourth polypeptide chain comprises the amino acid sequence of SEQ ID NO: 156.

Some aspects described herein provide a binding protein comprising a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 25, SEQ ID NO: 26, SEQ ID NO: 27, SEQ ID NO: 28, SEQ ID NO: 29, and SEQ ID NO: 30 respectively. In various non-limiting embodiments, the binding protein comprises a V_{H} chain according to SEQ ID NO: 31 and a V_{L} according to SEQ ID NO: 32. In various other non-limiting embodiments, the binding protein comprises a LC chain according to SEQ ID NO: 33 and a V_{H}-C_{H1} to SEQ ID NO: 34. In various other non-limiting embodiments, the binding protein comprises a polypeptide chain according to SEQ ID NO: 36 and/or SEQ ID NO: 35.

Some aspects described herein provide a binding protein comprising a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 136, SEQ ID NO: 137, SEQ ID NO: 138, SEQ ID NO: 139, and SEQ ID NO: 140 respectively. In various non-limiting embodiments, the binding protein comprises a V_{H} chain according to SEQ ID NO: 141 and a V_{L} according to SEQ ID NO: 142. In various other non-limiting embodiments, the binding protein comprises a LC according to SEQ ID NO: 143 and a V_{H}-C_{H1} to SEQ ID NO: 144. In various other non-limiting embodiments, the binding protein comprises a polypeptide chain according to SEQ ID NO: 146 and/or SEQ ID NO: 145.

Some aspects described herein provide a binding protein comprising a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 147, SEQ ID NO: 137, SEQ ID NO: 138, SEQ ID NO: 139, and SEQ ID NO: 140 respectively. In various non-limiting embodiments, the binding protein comprises a V_{H} chain according to SEQ ID NO: 148 and a V_{L} according to SEQ ID NO: 142. In various other non-limiting embodiments, the binding protein comprises a LC according to SEQ ID NO: 143 and a V_{H}-C_{H1} to SEQ ID NO: 149. In various other non-limiting embodiments, the binding protein comprises a polypeptide chain according to SEQ ID NO: 151 and/or SEQ ID NO: 150.

Some aspects described herein provide a binding protein comprising a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 152, SEQ ID NO: 137, SEQ ID NO: 138, SEQ ID NO: 139, and SEQ ID NO: 140 respectively. In various non-limiting embodiments, the binding protein comprises a V_{H} chain according to SEQ ID NO: 153 and a V_{L} according to SEQ ID NO: 142. In various other non-limiting embodiments, the binding protein comprises a LC according to SEQ ID NO: 143 and a V_{H}-C_{H1} to SEQ ID NO: 154. In various other non-limiting embodiments, the binding protein comprises a polypeptide chain according to SEQ ID NO: 156 and/or SEQ ID NO: 155. In various other non-limiting embodiments, the binding protein comprises a polypeptide chain according to SEQ ID NO: 156 and/or SEQ ID NO: 176.

Some aspects described herein provide a binding protein comprising a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 157, SEQ ID NO: 137, SEQ ID NO: 138, SEQ ID NO: 158, and SEQ ID NO: 140 respectively. In various non-limiting embodiments, the binding protein comprises a V_{H} chain according to SEQ ID NO: 159 and a V_{L} according to SEQ ID NO: 160. In various other non-limiting embodiments, the binding protein comprises a LC according to SEQ ID NO: 161 and a V_{H}-C_{H1} to SEQ ID NO: 162. In various other non-limiting embodiments, the binding protein comprises a polypeptide chain according to SEQ ID NO: 164 and/or SEQ ID NO: 163.

Some aspects described herein provide a binding protein comprising a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 157, SEQ ID NO: 137, SEQ ID NO: 138, SEQ ID NO: 165, and SEQ ID NO: 140 respectively. In various non-limiting embodiments, the binding protein comprises a V_{H} chain according to SEQ ID NO: 159 and a V_{L} according to SEQ ID NO: 166. In various other non-limiting embodiments, the binding protein comprises a LC according to SEQ ID NO: 167 and a V_{H}-C_{H1} to SEQ ID NO: 162. In various other non-limiting embodiments, the binding protein comprises a polypeptide chain according to SEQ ID NO: 164 and/or SEQ ID NO: 163.

In particular aspects provided herein are amino acid sequences with a conservative substitution in which there are up to 10, up to 8, up to 5, or up to 3 amino acids substituted by amino acids having analogical or similar properties, compared to the amino acid sequence of the sequences disclosed herein.

In some aspects, the light chain (V_{L}-C_{L}) associates with the V_{H} and C_{H1} of a heavy chain to form an “antigen binding arm” and the variable domains in the antigen binding arm interact to form the “antigen binding site”. As described herein, “antigen binding site” and “antigen binding domain” are used interchangeably.

### Lambda Charge Pairs

The terms “charge pair(s)” and “charge mutation(s)” are used interchangeably throughout this specification and refer to a positively charged amino acid residue and a negatively charged amino acid residue, one of which is located in the a light chain region (e.g. constant light chain region) and the other in a heavy chain region (e.g. constant heavy chain region 1 (C_{H1})) of an antigen binding arm, located at positions intended to promote association of the light and heavy chains. By “lambda charge pair”, it is meant a charge pair where a positively or negatively charged amino acid residue is located in a lambda light chain. By “kappa charge pair”, it is meant a charge pair where a positively or negatively charged amino acid residue in the light chain is located in a kappa light chain.

Without wishing to be bound by theory, it is believed that the oppositely charged amino acid residues in the charge pair increase the attraction of the heavy chain to the light chain in an antigen binding arm, thereby promoting formation of the antigen binding arm with the correct heavy and light chain.

At least one of the amino acid residues of the charge pair have been engineered into the antigen binding arm (i.e. at least one amino acid residue in the pair is not a wild-type amino acid residue). In some aspects, both amino acid residues in the charge pair are engineered into the antigen binding arm (i.e. both amino acid residues in the pair are not wild-type amino acid residues).

The amino acid residues of the charge pair are typically naturally occurring. Naturally occurring positively charged amino acid residues according to the present disclosure include arginine, lysine and histidine. Naturally occurring negatively charged amino acid residues according to the present disclosure include glutamic acid, serine, threonine and aspartic acid. Although serine and threonine are often described in the art as ‘uncharged’, they have an isoelectric point below 6 and therefore are partially negatively charged at neutral pH. For the purposes of the charge pairs disclosed herein, serine and threonine are examples of negatively charged amino acid residues (together with glutamic acid and aspartic acid).

Hence, a charge pair may comprise a positively charged amino acid residue selected from arginine, lysine or histidine located at one of the positions in the charge pair and a negatively charged amino acid residue selected from aspartic acid, glutamic acid, serine or threonine located at the other position in the charge pair. For example, the charge pair may comprise any one of the following pairs of amino acid residues: arginine and aspartic acid; arginine and glutamic acid; arginine and serine; arginine and threonine; lysine and aspartic acid; lysine and glutamic acid; lysine and serine; lysine and threonine; histidine and aspartic acid; histidine and glutamic acid; histidine and serine; and histidine and threonine.

In some aspects, the positively charged amino acid residue in the charge pair is located on the light chain and the negatively charged amino acid residue in the charge pair is located on the heavy chain. In other aspects, the negatively charged amino acid residue is located on the light chain and the positively charged amino acid residue in the charge pair is located on the heavy chain.

The binding proteins described herein may comprise a lambda charge pair in one of the antigen binding arms. As exemplified herein, lambda charge pairs can be introduced at several positions to improve pairing of the correct light and heavy chains in the antigen binding arm.

In some aspects, the lambda charge pair comprises a positively or negatively charged amino acid residue at position 117, 119, 134, 136 or 178 of the constant light chain lambda region (CLλ). In some aspects, the lambda charge pair comprises a positively or negatively charged amino acid residue at position 141, 185, 128, 145, 183, 185, 173, or 187 of the C_{H1}. As noted elsewhere, the numbering is according to Eu numbering. Positions 117, 119, 134, 136, and 178 of the CLλ according to Eu numbering corresponds to amino acid positions 10, 12, 27, 29, and 71 of SEQ ID NOs: 98 and 99. Positions 141, 185, 128, 145, 183, 185, 173, and 187 of the C_{H1} according to Eu numbering corresponds to amino acid positions 24, 68, 11, 28, 66, 68, 56, and 70 of SEQ ID NOs: 95-99.

In some aspects, lambda charge pair located at one or more of the following pairs of positions: (i) position 117 in the CLλ and position 141 in the C_{H1}; (ii) position 117 in the CLλ and position 185 in the CH1; (iii) position 119 in the CLλ and position 128 in the CH1; (iv) position 134 in the CLλ and position 128 in the C_{H1}; (v) position 134 in the CLλ and position 145 in the C_{H1}; (vi) position 134 in the CLλ and position 183 in the CH1; (vii) position 136 in the CLλ and position 185 in the C_{H1}; (viii) position 178 in the CLλ and position 173 in the CH1; and (ix) position 117 in the CLλ and position 187 in the CH1.

In some aspects, the lambda charge pair is located at charge pair is located at position 117 in the CLλ and position 141 in the CH1. For example, the lambda charge pair can be selected from the following list: arginine at position 117 of the CLλ and aspartic acid at position 141 of the CH1; arginine at position 117 of the CLλ and glutamic acid at position 141 of the CH1; arginine at position 117 of the CLλ and serine at position 141 of the C_{H1}; arginine at position 117 of the CLλ and threonine at position 141 of the CH1; lysine at position 117 of the CLλ and aspartic acid at position 141 of the CH1; lysine at position 117 of the CLλ and glutamic acid at position 141 of the CH1; lysine at position 117 of the CLλ and serine at position 141 of the CH1; and lysine at position 117 of the CLλ and threonine at position 141 of the CH1.

In some aspects, the lambda charge pair is located at charge pair is located at position 117 in the CLλ and position 185 in the CH1. For example, the lambda charge pair can be selected from the following list: arginine at position 117 of the CLλ and aspartic acid at position 185 of the CH1; arginine at position 117 of the CLλ and glutamic acid at position 185 of the CH1; arginine at position 117 of the CLλ and serine at position 185 of the CH1; arginine at position 117 of the CLλ and threonine at position 185 of the CH1; lysine at position 117 of the CLλ and aspartic acid at position 185 of the CH1; lysine at position 117 of the CLλ and glutamic acid at position 185 of the CH1; lysine at position 117 of the CLλ and serine at position 185 of the CH1; and lysine at position 117 of the CLλ and threonine at position 185 of the CH1.

In some aspects, the lambda charge pair is located at charge pair is located at position 119 in the CLλ and position 128 in the CH1. For example, the lambda charge pair can be selected from the following list: arginine at position 119 of the CLλ and aspartic acid at position 128 of the CH1; arginine at position 119 of the CLλ and glutamic acid at position 128 of the CH1; arginine at position 119 of the CLλ and serine at position 128 of the CH1; arginine at position 119 of the CLλ and threonine at position 128 of the CH1; lysine at position 119 of the CLλ and aspartic acid at position 128 of the CH1; lysine at position 119 of the CLλ and glutamic acid at position 128 of the CH1; lysine at position 119 of the CLλ and serine at position 128 of the CH1; and lysine at position 119 of the CLλ and threonine at position 128 of the CH1.

In some aspects, the lambda charge pair is located at charge pair is located at position 134 in the CLλ and position 128 in the CH1. For example, the lambda charge pair can be selected from the following list: arginine at position 134 of the CLλ and aspartic acid at position 128 of the CH1; arginine at position 134 of the CLλ and glutamic acid at position 128 of the CH1; arginine at position 134 of the CLλ and serine at position 128 of the CH1; arginine at position 134 of the CLλ and threonine at position 128 of the CH1; lysine at position 134 of the CLλ and aspartic acid at position 128 of the CH1; lysine at position 134 of the CLλ and glutamic acid at position 128 of the CH1; lysine at position 134 of the CLλ and serine at position 128 of the CH1; and lysine at position 134 of the CLλ and threonine at position 128 of the CH1.

In some aspects, the lambda charge pair is located at charge pair is located at position 134 in the CLλ and position 145 in the CH1. For example, the lambda charge pair can be selected from the following list: arginine at position 134 of the CLλ and aspartic acid at position 145 of the CH1; arginine at position 134 of the CLλ and glutamic acid at position 145 of the CH1; arginine at position 134 of the CLλ and serine at position 145 of the CH1; arginine at position 134 of the CLλ and threonine at position 145 of the CH1; lysine at position 134 of the CLλ and aspartic acid at position 145 of the CH1; lysine at position 134 of the CLλ and glutamic acid at position 145 of the CH1; lysine at position 134 of the CLλ and serine at position 145 of the CH1; and lysine at position 134 of the CLλ and threonine at position 145 of the CH1.

In some aspects, the lambda charge pair is located at charge pair is located at position 134 in the CLλ and position 183 in the CH1. For example, the lambda charge pair can be selected from the following list: arginine at position 134 of the CLλ and aspartic acid at position 183 of the CH1; arginine at position 134 of the CLλ and glutamic acid at position 183 of the CH1; arginine at position 134 of the CLλ and serine at position 183 of the CH1; arginine at position 134 of the CLλ and threonine at position 183 of the CH1; lysine at position 134 of the CLλ and aspartic acid at position 183 of the CH1; lysine at position 134 of the CLλ and glutamic acid at position 183 of the CH1; lysine at position 134 of the CLλ and serine at position 183 of the CH1; and lysine at position 134 of the CLλ and threonine at position 183 of the CH1.

In some aspects, the lambda charge pair is a lysine at position 134 of the CLX, and an aspartic acid or a serine at position 183 of the CH1. In the CH1 sequences provided as SEQ ID NO: 97 or SEQ ID NO: 98, EU position 183 is a serine and therefore it is not necessary to introduce a modification in the CH1 of SEQ ID NO: 100 in order to produce a charge pair with a positively charged amino acid at position 134 of the CLX.

In some aspects, the lambda charge pair is located at charge pair is located at position 136 in the CLλ and position 185 in the CH1. For example, the lambda charge pair can be selected from the following list: arginine at position 136 of the CLλ and aspartic acid at position 185 of the CH1; arginine at position 136 of the CLλ and glutamic acid at position 185 of the CH1; arginine at position 136 of the CLλ and serine at position 185 of the CH1; arginine at position 136 of the CLλ and threonine at position 185 of the CH1; lysine at position 136 of the CLλ and aspartic acid at position 185 of the CH1; lysine at position 136 of the CLλ and glutamic acid at position 185 of the CH1; lysine at position 136 of the CLλ and serine at position 185 of the CH1; and lysine at position 136 of the CLλ and threonine at position 185 of the CH1.

In some aspects, the lambda charge pair is located at charge pair is located at position 178 in the CLλ and position 173 in the CH1. For example, the lambda charge pair can be selected from the following list: arginine at position 178 of the CLλ and aspartic acid at position 173 of the CH1; arginine at position 178 of the CLλ and glutamic acid at position 173 of the CH1; arginine at position 178 of the CLλ and serine at position 173 of the CH1; arginine at position 178 of the C_{L}, and threonine at position 173 of the CH1; lysine at position 178 of the C_{L}, and aspartic acid at position 173 of the CH1; lysine at position 178 of the C_{L}, and glutamic acid at position 173 of the CH1; lysine at position 178 of the C_{L}, and serine at position 173 of the CH1; and lysine at position 178 of the CLλ and threonine at position 173 of the CH1.

In some aspects, the first antigen binding arm comprises more than one lambda charge pair. For example, the first antigen binding arm may comprise two, three, four, five, six, seven, eight or nine lambda charge pairs at positions (i) to (ix) described above.

In various aspects, the CLλ of the first polypeptide chain and/or CLλ of the second polypeptide chain each comprises an amino acid sequence having at least 90%, 91%, 92%, 93%, 94%, 95%, 96%, 97%, 98%, or 99% identity to SEQ ID NOs: 98-99.

In various other aspects, CLλ comprises an amino acid sequence according to SEQ ID NO: 105, and the corresponding C_{H1} comprises an amino acid sequence according to SEQ ID NO: 104. In various other aspects, C_{L}, comprises an amino acid sequence according to SEQ ID NO: 105, and the corresponding C_{H1} comprises an amino acid sequence according to SEQ ID NO: 220.

In various other aspects, CLλ comprises an amino acid sequence according to SEQ ID NO: 107, and the corresponding C_{H1} comprises an amino acid sequence according to SEQ ID NO: 106.

### Engineered Disulfides

In some aspects, the binding proteins contain engineered disulfides in addition to or instead of the lambda charge pairs. By “engineered disulfides” it is meant that a native interchain disulfide bond at the C_{H1}-C_{L} interface (e.g. at 220 of the C_{H1} and 212 of the C_{L}) of one of the antigen binding arms has been replaced by an engineered (non-native) interchain disulfide, while the other antigen binding arm contains the native interchain disulfide bond at the C_{H1}-C_{L} interface. An engineered disulfide is typically formed by engineering cysteines into the C_{L} of a light chain and the C_{H1} of the corresponding heavy chain and replacing the cysteines that normally form the interchain disulfide. Disclosure related to the introduction of engineered disulfide into binding proteins for the purpose of promoting heterodimerization can found e.g., in U.S. Pat. No. 9,527,927 and Mazor, 2015, which are herein incorporated by reference in their entirety.

The formation of disulfide bonds between cysteine residues occurs during the folding of many proteins that enter the secretory pathway. As the polypeptide chain collapses, cysteines brought into proximity can form covalent linkages during a process catalyzed by members of the protein disulfide isomerase family. The term “disulfide link” or “disulfide linked” as used herein, refers to the single covalent bond formed from the coupling of thiol groups, especially of cysteine residues. In some aspects, the covalent linkage between two cysteines is between the two sulfur atoms of each residue. However, depending on the environment, not all protein species may have a disulfide present at all times, for example, in the event of disulfide reduction. Thus, the term “disulfide link” or “disulfide linked” (whether native or engineered), in some aspects, also refers to the presence of two cysteine residues that are capable of forming a disulfide link, irrespective of whether or not they are actually linked at that individual point in time.

In some aspects herein, “V12 DS” or “V12” as used herein refers to removal of the native interchain disulfide bond in the C_{H1}/CLλ interface and the subsequent introduction of the alternative disulfide bond comprising four mutations: F126C/C220V in the C_{H1} domain, and mutations S122C/C212V in the lambda constant domain.

Thus, in some aspects: (i) the C_{L} of the first polypeptide chain and the CH1 of the third polypeptide chain, wherein the C_{L} of the first polypeptide chain is a CLλ and wherein the pair of engineered cysteines are located at position 122 of the CLλ of the first polypeptide chain and position 126 of the CH1 of the third polypeptide chain, and wherein the CLλ of the first polypeptide chain comprises a non-cysteine residue at position 212 and the CH1 of the third polypeptide chain comprises a non-cysteine residue at position 220, optionally wherein the non-cysteine residues are valines; or (ii) the C_{L} of the second polypeptide chain and the CH1 of the fourth polypeptide chain, wherein the C_{L} of the first polypeptide chain is a CLλ and wherein the pair of engineered cysteines are located at position 122 of the CLλ of the second polypeptide chain and position 126 of the CH1 of the fourth polypeptide chain, and wherein the CLλ of the second polypeptide chain comprises a non-cysteine residue at position 212 and the CH1 of the fourth polypeptide chain comprises a non-cysteine residue at position 220, optionally wherein the non-cysteine residues are valines.

In some aspects, the pair of cysteines are engineered into either: (i) the C_{L} of the first polypeptide chain and the CH1 of the third polypeptide chain, wherein the C_{L} of the first polypeptide chain is a CLκ and wherein the pair of engineered cysteines are located at position 121 of the CLκ of the first polypeptide chain and position 126 of the CH1 of the third polypeptide chain, and wherein the CLκ of the first polypeptide chain comprises a non-cysteine residue at position 214 and the CH1 of the third polypeptide chain comprises a non-cysteine residue at position 220, optionally wherein the non-cysteine residues are valines; or (ii) the C_{L} of the second polypeptide chain and the CH1 of the fourth polypeptide chain, wherein the C_{L} of the first polypeptide chain is a CLκ and wherein the pair of engineered cysteines are located at position 121 of the CLκ of the second polypeptide chain and position 126 of the CH1 of the fourth polypeptide chain, and wherein the CLκ of the second polypeptide chain comprises a non-cysteine residue at position 214 and the CH1 of the fourth polypeptide chain comprises a non-cysteine residue at position 220, optionally wherein the non-cysteine residues are valines.

An exemplary amino acid sequence of a CLλ comprising an engineered cysteine is provided as SEQ ID NO: 97 and an exemplary amino acid sequence of the CH1 comprising the corresponding engineered cysteine to form the engineered disulfide is provided as SEQ ID NO: 99.

In the binding proteins antibodies exemplified herein, the engineered disulfide may be present on the “first” antigen binding arm containing the lambda charge pairs and the native disulfide is present on the “second” antigen binding arm that does not contain the lambda charge pairs. However, the opposite arrangement is also specifically contemplated, i.e. where the native disulfide is present on the first antigen binding arm and the engineered disulfide is present on the second antigen binding arm.

In some aspects, the pair of cysteines are engineered into a constant light chain kappa region (CLκ) and CH1 and are located at position 121 of the CLκ and position 126 of the CH1, and wherein the same CLκ comprises a non-cysteine residue at position 214 and the same CH1 comprises a non-cysteine residue at position 220. In some aspects, the non-cysteine residues are valines.

### Kappa Charge Pairs

In some aspects, the antigen binding arm comprises a constant light chain kappa region (CLκ). As described herein, techniques such as light chain affinity chromatography that utilizes affinity resins specific for either CLκ or CLλ can be used to selectively purify antibodies based on their light chain. Examples of such affinity resins include the LambdaFabSelect and KappaSelect resins available from GE Healthcare. Such methods can be used to selectively purify binding proteins containing both CLκ and CLλ and can therefore be used to improve production of bispecific antibodies in this format.

An example of an CLκ amino acid sequence is provided as SEQ ID NO: 100.

In some aspects, one of the binding arms comprises a kappa charge pair. As described above, kappa charge pairs refer to a positively charged amino acid residue and a negatively charged amino acid residue, one of which is located in the kappa light chain (e.g. CLκ) and the other in the heavy chain (e.g. CH1) of an antigen binding arm, located at positions intended to promote association of the light chain and CH1 of the second antigen binding arm.

In some aspects, the second antigen binding arm comprises a kappa charge pair located at position 133 in the CLκ and position 183 in the CH1. In some aspects, the negatively charged amino acid residue in the kappa charge pair is at position 133 of the CLκ and the positively charged amino acid residue in the kappa charge pair 183 of the CH1. In other aspects, the positively charged amino acid residue in the kappa charge pair is at position 133 of the CLκ and the negatively charged amino acid residue in the kappa charge pair 183 of the CH1. In some aspects, the negatively charged amino acid residue (e.g. at position 133 of the CLκ) is a glutamic acid, and wherein the positively charged amino acid residue (e.g. at position 183 of the CH1) is a lysine. As noted elsewhere, this numbering is according to Eu numbering.

Position 133 of the CLκ according to Eu numbering corresponds to amino acid position 26 of SEQ ID NO: 100. Position 183 of the CH1 according to Eu numbering corresponds to amino acid position 66 of SEQ ID NOs: 95 and 97.

In certain aspects, the binding protein comprises a first antigen binding arm with a lambda charge pair as described above and a second antigen binding arm with a kappa charge pair as described above, wherein the binding protein comprises engineered disulfides. For example, in one aspect, the first antigen binding arm comprises a lambda charge pair (e.g. at position 117 in the CLλ and position 141 in the CH1) and the disulfide link between the light chain and CH1 is formed between a pair of cysteines engineered into the CLλ and CH1; and the second antigen binding arm comprises a kappa charge pair (e.g. at position 133 in the CLκ and position 183 in the CH1) and the disulfide link between the light chain and CH1 is formed between a pair of native cysteines in the CLκ of the second light chain and CH1.

In another aspect, the first antigen binding arm comprises a lambda charge pair (e.g. at position 117 in the CLλ and position 141 in the CH1) and the disulfide link between the first light chain and CH1 is formed between a pair of native cysteines in CLλ of the light chain and CH1; and the second antigen binding arm comprises a kappa charge pair (e.g. at position 133 in the CLκ and position 183 in the CH1) and the disulfide link between the light chain and CH1 of the second antigen binding arm is formed between a pair of cysteines engineered in the CLκ of the light chain and CH1.

In some aspects, the CLκ of the first polypeptide chain or the second polypeptide chain comprises an amino acid sequence having at least 90%, 91%, 92%, 93%, 94%, 95%, 96%, 97%, 98%, or 99% identity to SEQ ID NO: 100.

In various aspects, the corresponding C_{H1} in each of the one or more lambda charge pairs comprises an amino acid sequence having at least 90%, 91%, 92%, 93%, 94%, 95%, 96%, 97%, 98%, or 99% to SEQ ID NO: 95-97, and/or the corresponding C_{H1} in the kappa charge pair comprises an amino acid sequence having at least 90%, 91%, 92%, 93%, 94%, 95%, 96%, 97%, 98%, or 99% to SEQ ID NO: 100.

### Compositions and Methods of Treatment

The binding proteins of the disclosure may be prepared using domains or sequences obtained or derived from any human or non-human antibody, including, for example, human, murine, or humanized antibodies. Some aspects of the present disclosure relate to an isolated nucleic acid molecule encoding a binding protein as described herein. In various aspects the isolated nucleic acid molecules comprise a nucleotide sequence according to any one of SEQ ID NOs: 184-219. In one aspect, the isolated nucleic acid molecule comprises the nucleotide sequence according to any one of SEQ ID NOs: 184-186. In one aspect, the isolated nucleic acid molecule comprises the nucleotide sequence according to any one of SEQ ID NOs: 187-189. In one aspect, the isolated nucleic acid molecule comprises the nucleotide sequence according to any one of SEQ ID NOs: 190-192. In one aspect, the isolated nucleic acid molecule comprises the nucleotide sequence according to any one of SEQ ID NOs: 193-196. In one aspect, the isolated nucleic acid molecule comprises the nucleotide sequence according to any one of SEQ ID NOs: 196-198.

In one aspect, the isolated nucleic acid molecule comprises the nucleotide sequence according to any one of SEQ ID NOs: 199-201. In one aspect, the isolated nucleic acid molecule comprises the nucleotide sequence according to any one of SEQ ID NOs: 202-204. In one aspect, the isolated nucleic acid molecule comprises the nucleotide sequence according to any one of SEQ ID NOs: 205-207. In one aspect, the isolated nucleic acid molecule comprises the nucleotide sequence according to any one of SEQ ID NOs: 208-210. In one aspect, the isolated nucleic acid molecule comprises the nucleotide sequence according to any one of SEQ ID NOs: 211-213. In one aspect, the isolated nucleic acid molecule comprises the nucleotide sequence according to any one of SEQ ID NOs: 214-216. In one aspect, the isolated nucleic acid molecule comprises the nucleotide sequence according to any one of SEQ ID NOs: 217-219.

The isolated nucleic acid molecule may be included in a vector. In one aspect, the vector comprises the isolated nucleic acid molecule of any one of SEQ ID NOs: 184-219.

In some aspects, the methods disclosed herein relate to treating a subject for cancer by administering an effective amount of a binding protein. The disclosure also provides a therapeutically effective amount of a binding protein for use in treating cancer in a subject. The disclosure also provides use of a binding protein and/or pharmaceutical composition for the manufacture of a medicament. The disclosure also provides use of a binding protein and/or pharmaceutical composition for the manufacture of a medicament for treating cancer.

In the method of treatment of the present disclosure, the binding proteins can preferentially activate a subset of T cells in the subject. The subset of T cells can be CD8+ T cells. The CD8+ T cells can be preferentially activated as compared to CD4 T cells. The preferential activation of CD8+ T cells reduces engagement with tumor promoting T cells and CD4+ T cells which produce the majority of cytokines that cause release syndrome (CRS). Additionally, the preferential engagement of the CD8+ T cells induce pyroptosis.

The activation of T cells through methods of the present disclosure can be determined by measuring the percentage of surface interleukin-2 receptor alpha chain-positive (CD25+) T cells. The percentage of surface CD25+ T cells that are CD8 T cells can be higher than the percentage of surface CD25+ T cells that are CD4 T cells. In particular aspects, activation of T cells can be determined by the percentage of CD69+/CD25+ T cells. In particular aspects, activation of T cells can be determined by measuring levels of cytokines released by activated T cells.

The method of treatment of the present disclosure can result in reduced engagement of regulatory T cells (Tregs), increased cytolytic activity, and/or reduced incidence of cytokine release syndrome (CRS) relative to that resulting from bispecific T-cell engager (BiTEs) previously known in the art. The disclosure also provides a therapeutically effective amount of a binding protein for use in reducing engagement of regulatory T cells (Tregs), increasing cytolytic activity, and/or reducing incidence of cytokine release syndrome (CRS) relative to that resulting from bispecific T-cell engager (BiTEs) previously known in the art.

In some aspects, the cancer is a B-cell malignancy, liver cancer, hepatocellular carcinoma (HCC), lung cancer, non-small cell lung cancer (NSCLC), squamous non-small cell lung cancer (sqNSCLC), ovarian cancer, clear cell ovarian cancer, carcinoma, Merkel cell carcinoma, gastric cancer, hepatoblastoma, nephroblastoma, melanoma, sarcoma, renal cell carcinoma, head and neck squamous cell carcinoma, urothelial cell carcinoma, osteosarcoma, glioblastoma, thyroid cancer, multiple myeloma, prostate cancer or Ewing's sarcoma.

In some aspects, the cancer includes B cell malignancies, liver cancer, hepatocellular carcinoma (HCC), melanoma, sarcoma, renal cell carcinoma, head and neck squamous cell carcinoma, urothelial cell carcinoma, osteosarcoma, glioblastoma, lung cancer, non-small cell lung cancer, and/or thyroid cancer. In some aspects, the cancer includes B cell malignancies, liver cancer, hepatocellular carcinoma (HCC), melanoma, sarcoma, renal cell carcinoma, head and neck squamous cell carcinoma, urothelial cell carcinoma, osteosarcoma, glioblastoma, lung cancer, non-small cell lung cancer, multiple myeloma, prostate cancer, Ewing's sarcoma, ovarian cancer and/or thyroid cancer.

In some aspects, the B cell malignancy, including chronic malignancy, is non-Hodgkin's lymphoma (NHL), chronic lymphocytic leukemia (CLL), mantle cell lymphoma (MCL), diffuse large B-cell lymphoma (DLBCL), follicular lymphoma (FL), Burkett lymphoma, or primary mediastinal large B cell lymphoma (PMBCL).

In some aspects, the B cell malignancy, including chronic malignancy is non-Hodgkin's lymphoma (NHL), chronic lymphocytic leukemia (CLL), mantle cell lymphoma (MCL), diffuse large B-cell lymphoma (DLBCL), follicular lymphoma (FL), Burkett lymphoma, primary mediastinal large B cell lymphoma (PMBCL) or small lymphocytic leukemia (SLL).

In various non-limiting embodiments, the CLL, MCL, FL, and/or DLBCL is positive for AA amyloidosis. In various other non-limiting embodiments, the CLL, MCL, FL, and/or DLBCL is fibrin-associated (FA).

In some embodiments the cancer is a B cell malignancy. In another embodiment, the cancer is hepatocellular carcinoma. In another embodiment, the cancer is multiple myeloma. In a further embodiment, the cancer is osteosarcoma.

In some aspects the tumor-associated antigen is CD20 and the cancer is a B-cell malignancy. In various non-limited embodiments the B-cell malignancy is non-Hodgkin's lymphoma (NHL), chronic lymphocytic leukemia (CLL), mantle cell lymphoma (MCL), diffuse large B-cell lymphoma (DLBCL), follicular lymphoma (FL), Burkett lymphoma, or primary mediastinal large B cell lymphoma (PMBCL). In another embodiment the B-cell malignancy is small lymphocytic leukemia (SLL).

In some aspects, the tumor-associated antigen is Glypican-3 (GPC3) and the cancer is liver cancer or hepatocellular carcinoma (HCC), non-small cell lung cancer (NSCLC), squamous non-small cell lung cancer (sqNSCLC), ovarian cancer, clear cell ovarian cancer, carcinoma, Merkel cell carcinoma, gastric cancer, hepatoblastoma, or nephroblastoma.

In some aspects, the tumor-associated antigen is leucine rich repeat containing 15 (LRRC15) and the cancer is melanoma, sarcoma, renal cell carcinoma, head and neck squamous cell carcinoma, urothelial cell carcinoma, osteosarcoma, glioblastoma, lung cancer, non-small cell lung cancer, or thyroid cancer.

In some aspects, the tumor-associated antigen is B cell maturation antigen (BCMA) and the cancer is multiple myeloma.

In some aspects, the tumor-associated antigen is STEAP2 and the cancer is prostate cancer or Ewing's sarcoma.

In some aspects, the methods disclosed herein relate to treating an inflammatory disease in a subject in need thereof by administering an effective amount of a binding protein. The disclosure also provides a therapeutically effective amount of a binding protein for use in treating an inflammatory disease in a subject. The disclosure also provides use of a binding protein and/or pharmaceutical composition for the manufacture of a medicament. The disclosure also provides use of a binding protein and/or pharmaceutical composition for the manufacture of a medicament for treating an inflammatory disease.

In some aspects, the methods disclosed herein relate to treating an inflammatory disease and/or autoimmune disorder in a subject in need thereof by administering an effective amount of a binding protein. The disclosure also provides a therapeutically effective amount of a binding protein for use in treating an inflammatory disease and/or autoimmune disorder in a subject. The disclosure also provides use of a binding protein and/or pharmaceutical composition for the manufacture of a medicament. The disclosure also provides use of a binding protein and/or pharmaceutical composition for the manufacture of a medicament for treating an inflammatory disease and/or autoimmune disorder.

In some aspects, the inflammatory disease and/or autoimmune disorder is, but is not limited to, systemic lupus erythematosus (SLE), myositis, rheumatoid arthritis (RA), Sjogren's syndrome, or anti-neutrophil cytoplasmic autoantibody (ANCA) vasculitis. In one embodiment the inflammatory disease and/or autoimmune disorder is systemic lupus erythematosus (SLE). In another embodiment the inflammatory disease and/or autoimmune disorder is myositis. In another embodiment the inflammatory disease and/or autoimmune disorder is rheumatoid arthritis (RA). In another embodiment the inflammatory disease and/or autoimmune disorder is Sjogren's syndrome. In a further embodiment the inflammatory disease and/or autoimmune disorder is anti-neutrophil cytoplasmic autoantibody (ANCA) vasculitis.

In some aspects, the methods disclosed herein relate to treating a subject for an inflammatory disease and/or autoimmune disorder where B cells are involved by administering an effective amount of a binding protein. The disclosure also provides a therapeutically effective amount of a binding protein for use in treating an inflammatory disease where B cells are involved in a subject.

In some aspects, the tumor-associated antigen is CD20 and the inflammatory disease or autoimmune disorder involves B cells.

In some aspects, the tumor-associated antigen is B cell maturation antigen (BCMA) and the autoimmune disorder is scleroderma, systemic lupus erythematosus (SLE), myositis, rheumatoid arthritis (RA) or Sjogren's syndrome.

In various aspects the binding proteins, compositions, pharmaceutical compositions, and/or medicaments can be administered using any suitable route of administration, including but not limited to oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes aspects in which exactly one member of the group is present in, employed in, or otherwise relevant to a given product or process. The disclosure includes aspects in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any elements can be removed from the group.

It should it be understood that, in general, where the disclosure, or aspects of the disclosure, is/are referred to as comprising particular elements and/or features, certain aspects of the disclosure or aspects of the disclosure consist, or consist essentially of, such elements and/or features. For purposes of simplicity, those aspects have not been specifically set forth in haec verba herein.

In view of the present disclosure, the binding proteins, pharmaceutical compositions, nucleic acid molecules, vectors, methods, compositions for use, and medicaments described herein can be configured by the person of ordinary skill in the art to meet the desired need.

Without limiting the disclosure, a number of embodiments of the disclosure are described herein for purpose of illustration.

Embodiment 1. A binding protein comprising four polypeptide chains that form two tumor-associated antigen (TAA) binding sites, a T cell receptor binding site, and a T cell co-stimulatory molecule binding site, wherein the first and second polypeptide chains have a structure represented by the formula: V_{L}-C_{L} a third polypeptide chain has a structure represented by the formula: V_{H1}-C_{H1}-V_{HHa}-F_{Ca} and a fourth polypeptide chain has a structure represented by the formula: V_{H1}-C_{H1}-V_{HHb}-F_{Cb} wherein the first polypeptide and the third polypeptide form the first of the two TAA binding sites, and the second polypeptide and the fourth polypeptide form the second of the two TAA binding sites, and wherein: V_{L} is an immunoglobulin light chain variable domain and V_{H1} is an immunoglobulin heavy chain variable domain that together form a TAA binding domain that specifically binds a tumor-associated antigen; C_{L} is an immunoglobulin light chain constant domain; C_{H1} is an immunoglobulin CH1 heavy chain constant domain; V_{HHa} is a single chain variable domain that specifically binds a T cell receptor; V_{HHb} is a single chain variable domain that specifically binds T cell co-stimulatory molecule; F_{Ca} is C_{H2a} and C_{H3a} immunoglobulin heavy chain constant domains; and F_{Cb} is C_{H2b} and C_{H3b} immunoglobulin heavy chain constant domains.

Embodiment 2. The binding protein of embodiment 1, further comprising L₁, a linker positioned between C_{H1} and V_{HHa} on the third polypeptide chain, and L₂, a linker positioned between V_{HHa} and the F_{Ca} on the third polypeptide chain, wherein L₁ and L₂ are each independently a linker or are absent.

Embodiment 3. The binding protein of either embodiment 1 or embodiment 2, further comprising L₃, a linker positioned between C_{H1} and V_{HHb} on the fourth polypeptide chain, and L₄, a linker positioned between V_{HHb} and F_{Cb} on the fourth polypeptide chain, wherein L₃ and L₄ are each independently a linker or are absent.

Embodiment 4. The binding protein of any one of embodiments 1-3 further comprising H1, an immunoglobulin hinge region positioned between C_{H1} and V_{HHa} on the third polypeptide chain, and H₂, an immunoglobulin hinge region positioned between V_{HHa} and the F_{Ca} on the third polypeptide chain, wherein H₁ and H₂ are each independently an immunoglobulin hinge region or are absent.

Embodiment 5. The binding protein of embodiment 4, wherein H₁ comprises SEQ ID NO: 53 or DK (SEQ ID NO: 54) or SEQ ID NO: 90 or is absent, and wherein H₂ comprises SEQ ID NO: 53 or SEQ ID NO: 55 or SEQ ID NO: 90 or is absent.

Embodiment 6. The binding protein of any one of embodiments 1-5, further comprising H₃, an immunoglobulin hinge region positioned between C_{H1} and V_{HHb} on the fourth polypeptide chain, and H4, an immunoglobulin hinge region positioned between V_{HHb} and the F_{Cb} on the fourth polypeptide chain, wherein H₃ and H₄ are each independently an immunoglobulin hinge region or are absent.

Embodiment 7. The binding protein of embodiment 6, wherein H₃ comprises SEQ ID NO: 53 or DK (SEQ ID NO: 54) or SEQ ID NO: 90 or is absent, and wherein H₄ comprises SEQ ID NO: 53 or SEQ ID NO: 55 or SEQ ID NO: 90 or is absent.

Embodiment 8. The binding protein of any one of embodiments 1-7, wherein the first and second polypeptide chains have a structure represented by the formula: V_{L}-C_{L} a third polypeptide chain has a structure represented by the formula: V_{H1}-C_{H1}-H₁-L₁-V_{HHa}-H₂-L₂-F_{Ca} or V_{H1}-C_{H1}-H₁-L₁-V_{HHa}-L₂-H₂-F_{Ca} and a fourth polypeptide chain has a structure represented by the formula: V_{H1}-C_{H1}-H₃-L₃-V_{HHb}-H₄-L₄-F_{Cb} or V_{H1}-C_{H1}-H₃-L₃-V_{HHb}-L₄-H₄-F_{Cb}.

Embodiment 9. The binding protein of any one of embodiments 1-8, wherein F_{Ca} and/or F_{Cb} is from an IgG antibody.

Embodiment 10. The binding protein of embodiment 9, wherein the F_{Ca} and/or the F_{Cb} is from an IgG1 antibody.

Embodiment 11. The binding protein of any one of embodiments 1-10, wherein the binding protein activates T cells only when bound to a tumor associated antigen at one or both of the antigen binding sites.

Embodiment 12. The binding protein of any one of embodiments 2-11, wherein L₁, L₂, L₃, and/or L₄ comprise one or more repeats of the amino acid sequence of SEQ ID NO: 37, SEQ ID NO: 38 and/or SEQ ID NO: 111.

Embodiment 13. The binding protein of any one of embodiments 1-12, wherein the V_{HHa} comprises a heavy chain CDR1 (HCDR1), a heavy chain CDR2 (HCDR2) and a heavy chain CDR3 (HCDR3), comprising the amino acid sequences of SEQ ID NO: 39, SEQ ID NO: 40 and SEQ ID NO: 41, respectively.

Embodiment 14. The binding protein of any one of embodiments 1-12, wherein the V_{HHa} comprises a heavy chain CDR1 (HCDR1), a heavy chain CDR2 (HCDR2) and a heavy chain CDR3 (HCDR3) comprising the amino acid sequence of SEQ ID NO: 62, SEQ ID NO: 40 and SEQ ID NO: 41, respectively.

Embodiment 15. The binding protein of any one of embodiments 1-14, wherein the V_{HHa} comprises the amino acid sequence at least 90%, at least 91% at least 92%, at least 93%, at least 94%, at least 95%, at least 96%, at least 97%, at least 98%, or at least 99% identical to any one of SEQ ID NOs: 42-48, or any one of SEQ ID NOs: 42-48.

Embodiment 16. The binding protein of any one of embodiments 1-15, wherein the T cell costimulatory molecule is CD8.

Embodiment 17. The binding protein of any one of embodiments 1-16, wherein the V_{HHb} comprises a heavy chain CDR1 (HCDR1), a heavy chain CDR2 (HCDR2) and a heavy chain CDR3 (HCDR3) comprising the amino acid sequences of SEQ ID NO: 49, SEQ ID NO: 50 and SEQ ID NO: 51, respectively.

Embodiment 18. The binding protein of any one of embodiments 1-17, wherein the V_{HHb} comprises an amino acid sequence at least 90%, at least 91% at least 92%, at least 93%, at least 94%, at least 95%, at least 96%, at least 97%, at least 98%, or at least 99% identical to SEQ ID NO: 52, or SEQ ID NO: 52.

Embodiment 19. The binding protein of any one of embodiments 1-18, wherein C_{H3a} and C_{H3b} in each of F_{Ca} and F_{Cb} comprise modifications to facilitate heterodimerization of F_{Ca} and F_{Cb}.

Embodiment 20. The binding protein of embodiment 19, wherein the modifications are substitutions to generate a knob in one of F_{Ca} and F_{Cb} and a hole in the other of F_{Ca} and F_{Cb}, wherein the knob is a substitution to tryptophan at position 366, and wherein the hole is a substitution of one or more of the following: (i) a substitution to valine at position 407; (ii) a substitution to serine at position 366; and (iii) a substitution to alanine at position 368.

Embodiment 21. The binding protein of embodiment 20, wherein the F_{Ca} or the F_{Cb} containing the knob further comprises a cysteine at position 354 and/or the F_{Ca} or the F_{Cb} containing the hole comprises a cysteine at position 349.

Embodiment 22. The binding protein of any one of embodiments 1-21 wherein the C_{H2a} and C_{H2b} immunoglobulin heavy chain constant domains each comprise the following substitutions: E233P/L234V/L235A/G236del/S267K.

Embodiment 23. The binding protein of any one of embodiments 1-22, wherein either the C_{H3a} immunoglobulin heavy chain constant domain or C_{H3b} immunoglobulin heavy chain constant domain comprises a H435R and Y436F substitution.

Embodiment 24. The binding protein of any one of embodiments 1-23, wherein F_{Ca} comprises the amino acid sequence of SEQ ID NO: 56 or SEQ ID NO: 57.

Embodiment 25. The binding protein of any one of embodiments 1-24, wherein F_{Cb} comprises the amino acid sequence of SEQ ID NO: 58.

Embodiment 26. The binding protein of any one of embodiments 1-25, wherein the third polypeptide chain comprises either SEQ ID NO: 59 or SEQ ID NO: 60 and the fourth polypeptide chain comprises SEQ ID NO: 61.

Embodiment 27. The binding protein of any one of embodiments 1-25, wherein the C_{L} of the first polypeptide chain and/or the C_{L} of the second polypeptide chain comprises a constant light chain lambda region (CLλ); and the binding protein comprises at least one lambda charge pair wherein the lambda charge pair is either between the CLλ of the first polypeptide chain and a corresponding C_{H1} that is the C_{H1} of the third polypeptide chain, or the lambda charge pair is between the CLλ of the second polypeptide and a corresponding C_{H1} that is the C_{H1} of the fourth polypeptide chain, and where the lambda charge pair is located at one or more of the following pairs of positions: (i) position 117 in the CLλ and position 141 in the corresponding C_{H1}; (ii) position 117 in the CLλ and position 185 in the corresponding C_{H1}; (iii) position 119 in the CLλ and position 128 in the corresponding C_{H1}; (iv) position 134 in the CLλ and position 128 in the corresponding C_{H1}; (v) position 134 in the CLλ and position 145 in the corresponding C_{H1}; (vi) position 134 in the CLλ and position 183 in the corresponding C_{H1}; (vii) position 136 in the CLλ and position 185 in the corresponding C_{H1}; (viii) position 178 in the CLλ and position 173 in the corresponding C_{H1}; and (ix) position 117 in the CLλ and position 187 in the corresponding C_{H1}; wherein the lambda charge pair comprises a positively charged amino acid residue selected from arginine, lysine and histidine located at one of the positions in the lambda charge pair and a negatively charged amino acid residue selected from aspartic acid, glutamic acid, serine and threonine located at the other position in the lambda charge pair; and wherein the numbering is according to the EU index.

Embodiment 28. The binding protein of embodiment 27, wherein the lambda charge pair is selected from the following list: (a) arginine at position 117 of the CLλ and aspartic acid at position 141 in the corresponding C_{H1}; (b) arginine at position 117 of the CLλ and glutamic acid at position 141 in the corresponding C_{H1}; (c) arginine at position 117 of the CLλ and serine at position 141 in the corresponding C_{H1}; (d) arginine at position 117 of the CLλ and threonine at position 141 in the corresponding C_{H1}; (e) lysine at position 117 of the CLλ and aspartic acid at position 141 in the corresponding C_{H1}; (f) lysine at position 117 of the CLλ and glutamic acid at position 141 in the corresponding C_{H1}; (g) lysine at position 117 of the CLλ and serine at position 141 in the corresponding C_{H1}; and (h) lysine at position 117 of the CLλ and threonine at position 141 in the corresponding C_{H1}.

Embodiment 29. The binding protein of either embodiment 27 or embodiment 28, wherein the CLλ of the first polypeptide chain and/or CLλ of the second polypeptide chain each comprises an amino acid sequence having at least 90%, 91%, 92%, 93%, 94%, 95%, 96%, 97%, 98%, or 99% identity to SEQ ID NOs: 98-99.

Embodiment 30. The binding protein of any one of embodiments 1-29, wherein the C_{L} of the first polypeptide chain and/or the C_{L} of the second polypeptide chain comprises a constant light chain kappa region (CLκ); and a kappa charge pair wherein the kappa charge pair is either between the CLκ of the first polypeptide chain and a corresponding C_{H1} that is the C_{H1} of the third polypeptide chain, or the kappa charge pair is between the CLκ of the second polypeptide and a corresponding C_{H1} that is the C_{H1} of the fourth polypeptide chain; wherein the kappa charge pair is located at position 117 in the CLκ and position 141 in the corresponding C_{H1} and comprises a positively charged amino acid residue selected from arginine, lysine, and histidine located at one of the positions in the kappa charge pair, and a negatively charged amino acid residue selected from aspartic acid, glutamic acid, serine and threonine located at the other position in the kappa charge pair.

Embodiment 31. The binding protein of embodiment 30, wherein the negatively charged amino acid residue in the kappa charge pair is located at position 133 of the CLκ, and the positively charged amino acid residue is located at position 183 of the corresponding C_{H1}, optionally wherein the negatively charged amino acid residue at position 133 of the CLκ is a glutamic acid, and optionally wherein the positively charged amino acid residue at position 183 of the corresponding C_{H1} is a lysine.

Embodiment 32. The binding protein of either embodiment 30 or embodiment 31, wherein the CLκ of the first polypeptide chain or the second polypeptide chain comprises an amino acid sequence having at least 90%, 91%, 92%, 93%, 94%, 95%, 96%, 97%, 98%, or 99% identity to SEQ ID NO: 100.

Embodiment 33. The binding protein of any one of embodiments 30-32 wherein the corresponding CH1 in each of the one or more lambda charge pairs comprises an amino acid sequence having at least 90%, 91%, 92%, 93%, 94%, 95%, 96%, 97%, 98%, or 99% to SEQ ID NO: 95-97, and/or the corresponding C_{H1} in the kappa charge pair comprises an amino acid sequence having at least 90%, 91%, 92%, 93%, 94%, 95%, 96%, 97%, 98%, or 99% to SEQ ID NO: 100.

Embodiment 34. The binding protein of any one of embodiments 27-33, wherein the C_{L}, comprises an amino acid sequence according to SEQ ID NO: 105, and the corresponding C_{H1} comprises an amino acid sequence according to SEQ ID NO: 104.

Embodiment 35. The binding protein of any one of embodiments 1-34, wherein either: (i) a disulfide link between the C_{L} of the first polypeptide chain and the C_{H1} of the third polypeptide chain is formed between a pair of cysteines engineered into the C_{L} of the first polypeptide chain and the C_{H1} of the third polypeptide chain, and a disulfide link between the C_{L} of the second polypeptide chain and the C_{H1} of the fourth polypeptide chain is formed between a pair of native cysteines; or (ii) a disulfide link between the C_{L} of the second polypeptide chain and the C_{H1} of the fourth polypeptide chain is formed between a pair of cysteines engineered into the C_{L} of the second polypeptide chain and the C_{H1} of the fourth polypeptide chain, and a disulfide link between the C_{L} of the first polypeptide chain and the C_{H1} of the third polypeptide chain is formed between a pair of native cysteines.

Embodiment 36. The binding protein of embodiment 35, wherein the pair of cysteines are engineered into either: (i) the C_{L} of the first polypeptide chain and the C_{H1} of the third polypeptide chain, wherein the C_{L} of the first polypeptide chain is a CLλ and wherein the pair of engineered cysteines are located at position 122 of the CLλ of the first polypeptide chain and position 126 of the C_{H1} of the third polypeptide chain, and wherein the C_{L}, of the first polypeptide chain comprises a non-cysteine residue at position 212 and the C_{H1} of the third polypeptide chain comprises a non-cysteine residue at position 220, optionally wherein the non-cysteine residues are valines; or (ii) the C_{L} of the second polypeptide chain and the C_{H1} of the fourth polypeptide chain, wherein the C_{L} of the first polypeptide chain is a CLλ and wherein the pair of engineered cysteines are located at position 122 of the CLλ of the second polypeptide chain and position 126 of the C_{H1} of the fourth polypeptide chain, and wherein the CLλ of the second polypeptide chain comprises a non-cysteine residue at position 212 and the C_{H1} of the fourth polypeptide chain comprises a non-cysteine residue at position 220, optionally wherein the non-cysteine residues are valines.

Embodiment 37. The binding protein of embodiment 35, wherein the pair of cysteines are engineered into either: (i) the C_{L} of the first polypeptide chain and the C_{H1} of the third polypeptide chain, wherein the C_{L} of the first polypeptide chain is a CLκ and wherein the pair of engineered cysteines are located at position 121 of the CLκ of the first polypeptide chain and position 126 of the C_{H1} of the third polypeptide chain, and wherein the CLκ of the first polypeptide chain comprises a non-cysteine residue at position 214 and the C_{H1} of the third polypeptide chain comprises a non-cysteine residue at position 220, optionally wherein the non-cysteine residues are valines; or (ii) the C_{L} of the second polypeptide chain and the C_{H1} of the fourth polypeptide chain, wherein the C_{L} of the first polypeptide chain is a CLκ and wherein the pair of engineered cysteines are located at position 121 of the CLκ of the second polypeptide chain and position 126 of the C_{H1} of the fourth polypeptide chain, and wherein the CLκ of the second polypeptide chain comprises a non-cysteine residue at position 214 and the C_{H1} of the fourth polypeptide chain comprises a non-cysteine residue at position 220, optionally wherein the non-cysteine residues are valines.

Embodiment 38. The binding protein of any one of embodiments 35-37, wherein the C_{L}, comprises an amino acid sequence according to SEQ ID NO: 107, and the corresponding C_{H1} comprises an amino acid sequence according to SEQ ID NO: 106.

Embodiment 39. The binding protein of any one of embodiments 1-38, wherein the tumor-associated antigen (TAA) is CD20, Glypican-3 (GPC3), or leucine rich repeat containing 15 (LRRC15).

Embodiment 40. The binding protein of any one of embodiments 1-39, wherein the TAA binding domain binds to CD20 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2) and a light chain CDR3 (LCDR3), comprising the amino acid sequences of SEQ ID NO: 1, SEQ ID NO: 2, SEQ ID NO: 3, SEQ ID NO: 4, SEQ ID NO: 5 and SEQ ID NO: 6, respectively.

Embodiment 41. The binding protein of embodiment 40, wherein the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 7 and SEQ ID NO: 8, respectively; or comprises a V_{H} chain according to SEQ ID NO: 7 and a light chain according to SEQ ID NO: 8.

Embodiment 42. The binding protein of any one of embodiments 1-35, wherein the TAA binding domain binds to GPC3 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 13, SEQ ID NO: 14, SEQ ID NO: 15, SEQ ID NO: 16, SEQ ID NO: 17, and SEQ ID NO: 18, respectively.

Embodiment 43. The binding protein of embodiment 36, wherein the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 19 and SEQ ID NO: 20, respectively; or comprises a V_{H} chain according to SEQ ID NO: 19 and a light chain according to SEQ ID NO: 20.

Embodiment 44. The binding protein of any one of embodiments 1-39, wherein the TAA binding domain binds to LRRC15 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 25, SEQ ID NO: 26, SEQ ID NO: 27, SEQ ID NO: 28, SEQ ID NO: 29, and SEQ ID NO: 30 respectively.

Embodiment 45. The binding protein of embodiment 44, wherein the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 31 and SEQ ID NO: 32, respectively; or comprises a V_{H} chain according to SEQ ID NO: 31 and a light chain according to SEQ ID NO: 32.

Embodiment 46. The binding protein of any one of embodiments 1-45 comprising: (a) the amino acid of sequences of SEQ ID NO: 9, SEQ ID NO: 11, and SEQ ID NO: 12; (b) the amino acid of sequences of SEQ ID NO: 21, SEQ ID NO: 23, and SEQ ID NO: 24; or (c) the amino acid of sequences of SEQ ID NO: 33, SEQ ID NO: 35, and SEQ ID NO: 36.

Embodiment 47. The binding protein of embodiment 46, wherein the first and second polypeptide chains comprise the amino acid sequence of SEQ ID NO: 9, the third polypeptide chain comprises the amino acid sequence of SEQ ID NO: 11, and the fourth polypeptide chain comprises the amino acid sequence of SEQ ID NO: 12.

Embodiment 48. The binding protein of embodiment 46, wherein the first and second polypeptide chains comprise the amino acid sequence of SEQ ID NO: 21, the third polypeptide chain comprises the amino acid sequence of SEQ ID NO: 23, and the fourth polypeptide chain comprises the amino acid sequence of SEQ ID NO: 24.

Embodiment 49. The binding protein of embodiment 46, wherein the first and second polypeptide chains comprise the amino acid sequence of SEQ ID NO: 33, the third polypeptide chain comprises the amino acid sequence of SEQ ID NO: 35, and the fourth polypeptide chain comprises the amino acid sequence of SEQ ID NO: 36.

Embodiment 50. A pharmaceutical composition comprising the binding protein of any one of embodiments 1-49 and a pharmaceutically acceptable carrier.

Embodiment 51. An isolated nucleic acid molecule encoding the binding protein of any one of embodiments 1-49.

Embodiment 52. A vector comprising the isolated nucleic acid molecule of embodiment 51.

Embodiment 53. A method of treating cancer in a subject in need thereof, comprising administering a therapeutically effective amount of the binding protein of any one of embodiments 1-49 or the pharmaceutical composition of embodiment 50 to the subject.

Embodiment 54. A method of treating an inflammatory disease and/or autoimmune disorder in a subject in need thereof, comprising administering a therapeutically effective amount of the binding protein of any one of embodiments 1-49 or the pharmaceutical composition of embodiment 50 to the subject.

Embodiment 55. The method of either embodiment 53, or embodiment 54, wherein the binding protein preferentially activates a subset of T cells in the subject.

Embodiment 56. The method of embodiment 55, wherein the subset of T cells are CD8+ T cells.

Embodiment 57. The method of embodiment 56, wherein the CD8+ T cells are preferentially activated as compared to CD4+ T cells.

Embodiment 58. The method of any one of embodiments 53-57, wherein the activation of T cells is determined by measuring the percentage of surface CD25+ T cells.

Embodiment 59. The method of embodiment 58, wherein the percentage of surface CD25+ T cells that are CD8+ T cells is higher than the percentage of surface CD25+ T cells that are CD4+ T cells.

Embodiment 60. A method of treating an inflammatory disease and/or autoimmune disorder in a subject in need thereof, comprising administering a therapeutically effective amount of the binding protein of any one of embodiments 1-49 or the pharmaceutical composition of embodiment 50 to the subject.

Embodiment 61. The method of embodiment 53, wherein the tumor-associated antigen of the binding protein is CD20 and the cancer is a B-cell malignancy.

Embodiment 62. The method of embodiment 61, wherein the B-cell malignancy is non-Hodgkin's lymphoma (NHL), chronic lymphocytic leukemia (CLL), mantle cell lymphoma (MCL), diffuse large B-cell lymphoma (DLBCL), follicular lymphoma (FL), Burkett lymphoma, or primary mediastinal large B cell lymphoma (PMBCL).

Embodiment 63. The method of embodiment 53, wherein the tumor-associated antigen of the binding protein is Glypican-3 (GPC3) and the cancer is liver cancer, hepatocellular carcinoma (HCC), non-small cell lung cancer (NSCLC), squamous non-small cell lung cancer (sqNSCLC), ovarian cancer, clear cell ovarian cancer, carcinoma, Merkel cell carcinoma, gastric cancer, hepatoblastoma, or nephroblastoma.

Embodiment 64. The method of embodiment 53, wherein the tumor-associated antigen of the binding protein is leucine rich repeat containing 15 (LRRC15) and the cancer is melanoma, sarcoma, renal cell carcinoma, head and neck squamous cell carcinoma, urothelial cell carcinoma, osteosarcoma, glioblastoma, lung cancer, non-small cell lung cancer, or thyroid cancer.

Embodiment 65. The method of embodiment 54, wherein the inflammatory disease and/or autoimmune disorder is systemic lupus erythematosus (SLE), myositis, rheumatoid arthritis (RA), Sjogren's syndrome, or anti-neutrophil cytoplasmic autoantibody (ANCA) vasculitis.

Embodiment 66. A method of treating systemic lupus erythematosus (SLE) in a subject in need thereof, comprising administering a therapeutically effective amount of the binding protein of any one of embodiments 1-49 or the pharmaceutical composition of embodiment 50 to the subject.

Embodiment 67. A method of treating myositis in a subject in need thereof, comprising administering a therapeutically effective amount of the binding protein of any one of embodiments 1-49 or the pharmaceutical composition of embodiment 50 to the subject.

Embodiment 68. A method of treating rheumatoid arthritis (RA) in a subject in need thereof, comprising administering a therapeutically effective amount of the binding protein of any one of embodiments 1-49 or the pharmaceutical composition of embodiment 50 to the subject.

Embodiment 69. A method of treating Sjogren's syndrome in a subject in need thereof, comprising administering a therapeutically effective amount of the binding protein of any one of embodiments 1-49 or the pharmaceutical composition of embodiment 50 to the subject.

Embodiment 70. The method of any one of embodiments 53-69, wherein the binding protein is administered by subcutaneous administration.

Embodiment 71. A binding protein of any one of embodiments 1-49, or the pharmaceutical composition of embodiment 50, for use as a medicament.

Embodiment 72. A binding protein of any one of embodiments 1-49, or the pharmaceutical composition of embodiment 50, for use in the treatment of cancer.

Embodiment 73. A binding protein of any one of embodiments 1-49, or the pharmaceutical composition of embodiment 50, for use in the treatment of an inflammatory disease and/or autoimmune disorder.

Embodiment 74. The binding protein or pharmaceutical composition for use of embodiment 72, wherein the tumor-associated antigen is CD20 and the cancer is a B-cell malignancy.

Embodiment 75. The binding protein or pharmaceutical composition for use of embodiment 74, wherein the B-cell malignancy is non-Hodgkin's lymphoma (NHL), chronic lymphocytic leukemia (CLL), mantle cell lymphoma (MCL), diffuse large B-cell lymphoma (DLBCL), follicular lymphoma (FL), Burkett lymphoma, or primary mediastinal large B cell lymphoma (PMBCL).

Embodiment 76. The binding protein or pharmaceutical composition for use of embodiment 72, wherein the tumor-associated antigen is Glypican-3 (GPC3) and the cancer is liver cancer or hepatocellular carcinoma (HCC), non-small cell lung cancer (NSCLC), squamous non-small cell lung cancer (sqNSCLC), ovarian cancer, clear cell ovarian cancer, carcinoma, Merkel cell carcinoma, gastric cancer, hepatoblastoma, or nephroblastoma.

Embodiment 77. The binding protein or pharmaceutical composition for use of embodiment 72, wherein the tumor-associated antigen is leucine rich repeat containing 15 (LRRC15) and the cancer is melanoma, sarcoma, renal cell carcinoma, head and neck squamous cell carcinoma, urothelial cell carcinoma, osteosarcoma, glioblastoma, lung cancer, non-small cell lung cancer, or thyroid cancer.

Embodiment 78. The binding protein or pharmaceutical composition for use of embodiment 73, wherein the inflammatory disease and/or autoimmune disorder is systemic lupus erythematosus (SLE), myositis, rheumatoid arthritis (RA), Sjogren's syndrome, or ANCA vasculitis.

Embodiment 79. A binding protein of any one of embodiments 1-49, or the pharmaceutical composition of embodiment 50, for use in the treatment of systemic lupus erythematosus (SLE).

Embodiment 80. A binding protein of any one of embodiments 1-49, or the pharmaceutical composition of embodiment 50, for use in the treatment of myositis.

Embodiment 81. A binding protein of any one of embodiments 1-49, or the pharmaceutical composition of embodiment 50, for use in the treatment of rheumatoid arthritis (RA).

Embodiment 82. A binding protein of any one of embodiments 1-49, or the pharmaceutical composition of embodiment 50, for use in the treatment of Sjogren's syndrome.

Embodiment 83. Use of a binding protein of any one of embodiments 1-49, or the pharmaceutical composition of embodiment 50, for the manufacture of a medicament for treating cancer.

Embodiment 84. Use of a binding protein of any one of embodiments 1-49, or the pharmaceutical composition of embodiment 50, for the manufacture of a medicament for treating an inflammatory and/or autoimmune disorder.

Embodiment 85. The use of embodiment 83, wherein the cancer is a B-cell malignancy, liver cancer, or HCC.

Embodiment 86. The use of embodiment 85, wherein the B-cell malignancy is non-Hodgkin's lymphoma (NHL), chronic lymphocytic leukemia (CLL), mantle cell lymphoma (MCL), diffuse large B-cell lymphoma (DLBCL), follicular lymphoma (FL), Burkett lymphoma, or primary mediastinal large B cell lymphoma (PMBCL).

Embodiment 87. The use of embodiment 83, wherein the cancer is melanoma, sarcoma, renal cell carcinoma, head and neck squamous cell carcinoma, urothelial cell carcinoma, osteosarcoma, glioblastoma, lung cancer, non-small cell lung cancer, or thyroid cancer.

Embodiment 88. The use of embodiment 84, wherein the inflammatory and/or autoimmune disorder is systemic lupus erythematosus (SLE), myositis, rheumatoid arthritis (RA), Sjogren's syndrome, or ANCA vasculitis.

Embodiment 89. Use of a binding protein of any one of embodiments 1-49, or the pharmaceutical composition of embodiment 50, for the manufacture of a medicament for the treatment of systemic lupus erythematosus (SLE).

Embodiment 90. Use of a binding protein of any one of embodiments 1-49, or the pharmaceutical composition of embodiment 50, for the manufacture of a medicament for the treatment of myositis.

Embodiment 91. Use of a binding protein of any one of embodiments 1-49, or the pharmaceutical composition of embodiment 50, for the manufacture of a medicament for the treatment of rheumatoid arthritis (RA).

Embodiment 92. Use of a binding protein of any one of embodiments 1-49, or the pharmaceutical composition of embodiment 50, for the manufacture of a medicament for the treatment of Sjogren's syndrome.

### EXAMPLES

The Examples that follow are illustrative of specific aspects of the disclosure, and various uses thereof. They are set forth for explanatory purposes only and should not be construed as limiting the scope of the disclosure in any way.

### Example 1: Binding Protein Structures and Sequences

A novel class of binding proteins engineered to improve safety and increase efficacy is described herein. A multitude of binding protein formats were tested to determine the placement of the TCR binding domain, the placement of the T-cell co-stimulatory domain, the linker size and cleavability and the Fc portion to be used.

TABLE 1
Amino acid and Nucleic acid sequences for LM1486 (CD20), LM1653 (GPC3),
LRC150016 (LRRC15), TPP-46956-TPP-46960 (LRRC15), TPP-42197 (BCMA) and
LM1953 (STEAP-2)

| SEQ ID | | |
|---|---|---|
| NO: | Description | Sequence |
| 1 | LM1486/ | GYTFTSYNMH |
| | LM1486-2 CD20 | |
| | VH CDR1 | |
| 2 | LM1486/ | AIYPGSGDTSYNQKFKG |
| | LM1486-2 | |
| | CD20 VH | |
| | CDR2 | |
| 3 | LM1486/ | VVYYSNSYWYFDV |
| | LM1486-2 | |
| | CD20 VH | |
| | CDR3 | |
| 4 | LM1486/ | RASSSVSYMH |
| | LM1486-2 | |
| | CD20 VL | |
| | CDR1 | |
| 5 | LM1486/ | APSNLAS |
| | LM1486-2 | |
| | CD20 VL | |
| | CDR2 | |
| 6 | LM1486/ | QQWSFNPPT |
| | LM1486-2 | |
| | CD20 VL | |
| | CDR3 | |
| 7 | LM1486/ | EVQLVESGGGLVQPGGSLRLSCAASGYTFTSYNMHWVRQA |
| | LM1486-2 | PGKGLEWVGAIYPGSGDTSYNQKFKGRFTISVDKSKNTLYL |
| | CD20 VH | QMNSLRAEDTAVYYCARVVYYSNSYWYFDVWGQGTLVTV |
| | | SS |
| 8 | LM1486/ | DIQMTQSPSSLSASVGDRVTITCRASSSVSYMHWYQQKPGK |
| | LM1486-2 | APKPLIYAPSNLASGVPSRFSGSGSGTDFTLTISSLQPEDFATY |
| | CD20 VL | YCQQWSFNPPTFGQGTKVEIK |
| 9 | LM1486/ | DIQMTQSPSSLSASVGDRVTITCRASSSVSYMHWYQQKPGK |
| | LM1486-2 | APKPLIYAPSNLASGVPSRFSGSGSGTDFTLTISSLQPEDFATY |
| | CD20 Light | YCQQWSFNPPTFGQGTKVEIKRTVAAPSVFIFPPSDEQLKSGT |
| | Chain | ASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| 10 | LM1486/ | EVQLVESGGGLVQPGGSLRLSCAASGYTFTSYNMHWVRQA |
| | LM1486-2 | PGKGLEWVGAIYPGSGDTSYNQKFKGRFTISVDKSKNTLYL |
| | CD20 VH- | QMNSLRAEDTAVYYCARVVYYSNSYWYFDVWGQGTLVTV |
| | CH1 | SSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKRVEPKSC |
| 11 | LM1486-2- | EVQLVESGGGLVQPGGSLRLSCAASGYTFTSYNMHWVRQA |
| | hole (TCR) | PGKGLEWVGAIYPGSGDTSYNQKFKGRFTISVDKSKNTLYL |
| | CD20 HC | QMNSLRAEDTAVYYCARVVYYSNSYWYFDVWGQGTLVTV |
| | TCR (FC | SSASTKGPSVFPLAPSSKSTSGGTAALGCLVKDYFPEPVTVS |
| | hole) | WNSGALTSGVHTFPAVLQSSGLYSLSSVVTVPSSSLGTQTYIC |
| | | NVNHKPSNTKVDKRVEPKSCDKTGGSEVQLVESGGGLVQPG |
| | | GSLRLSCVASGDVHKINFLGWYRQAPGKEREKVAHISIGDQT |
| | | DYADSAKGRFTISRDESKNMVYLQMNSLKPEDTAVYFCRAF |
| | | SRIYPYDYWGQGTLVTVSSGGGGSTHTCPPCPAPPVAGPSVF |
| | | LFPPKPKDTLMISRTPEVTCVVVDVKHEDPEVKFNWYVDGV |
| | | EVHNAKTKPREEQYNSTYRVVSVLTVLHQDWLNGKEYKCK |
| | | VSNKALPAPIEKTISKAKGQPREPQVCTLPPSREEMTKNQVSL |
| | | SCAVKGFYPSDIAVEWESNGQPENNYKTTPPVLDSDGSFFLV |
| | | SKLTVDKSRWQQGNVFSCSVMHEALHNHYTQKSLSLSPGK |
| 12 | LM1486/ | EVQLVESGGGLVQPGGSLRLSCAASGYTFTSYNMHWVRQA |
| | LM1486-2- | PGKGLEWVGAIYPGSGDTSYNQKFKGRFTISVDKSKNTLYL |
| | knob (CD8) | QMNSLRAEDTAVYYCARVVYYSNSYWYFDVWGQGTLVTV |
| | CD20 HC | SSASTKGPSVFPLAPSSKSTSGGTAALGCLVKDYFPEPVTVS |
| | CD8 (FC | WNSGALTSGVHTFPAVLQSSGLYSLSSVVTVPSSSLGTQTYIC |
| | knob) | NVNHKPSNTKVDKRVEPKSCDKTGGSEVQLLESGGGLVQPG |
| | | GSLRLSCAASGFTFDDYAIGWFRQAPGKEREGVSCIRVSDGS |
| | | TYYADSVKGRFTISRDNSKNTLYLQMNSLRAEDTAVYYCAA |
| | | GSLYTCVQSIVWPARPYYDMDYWGQGTLVTVSSGGGGSTH |
| | | TCPPCPAPPVAGPSVFLFPPKPKDTLMISRTPEVTCVVVDVKH |
| | | EDPEVKFNWYVDGVEVHNAKTKPREEQYNSTYRVVSVLTV |
| | | LHQDWLNGKEYKCKVSNKALPAPIEKTISKAKGQPREPQVY |
| | | TLPPCREEMTKNQVSLWCLVKGFYPSDIAVEWESNGQPENN |
| | | YKTTPPVLDSDGSFFLYSKLTVDKSRWQQGNVFSCSVMHEA |
| | | LHNHYTQKSLSLSPGK |
| 13 | LM1653 | DYEMH |
| | GPC3 VH | |
| | CDR1 | |
| 14 | LM1653 | ALDPKTGDTAYSQKFKG |
| | GPC3 VH | |
| | CDR2 | |
| 15 | LM1653 | FYSYTY |
| | GPC3 VH | |
| | CDR3 | |
| 16 | LM1653 | RSSQSLVHSNRNTYLH |
| | GPC3 VL | |
| | CDR1 | |
| 17 | LM1653 | KVSNRFS |
| | GPC3 VL | |
| | CDR2 | |
| 18 | LM1653 | SQNTHVPPT |
| | GPC3 VL | |
| | CDR3 | |
| 19 | LM1653 | QVQLVQSGAEVKKPGASVKVSCKASGYTFTDYEMHWVRQ |
| | GPC3 VH | APGQGLEWMGALDPKTGDTAYSQKFKGRVTLTADKSTSTA |
| | | YMELSSLTSEDTAVYYCTRFYSYTYWGQGTLVTVSS |
| 20 | LM1653 | DVVMTQSPLSLPVTPGEPASISCRSSQSLVHSNRNTYLHWYL |
| | GPC3 VL | QKPGQSPQLLIYKVSNRFSGVPDRFSGSGSGTDFTLKISRVEA |
| | | EDVGVYYCSQNTHVPPTFGQGTKLEIK |
| 21 | LM1653 | DVVMTQSPLSLPVTPGEPASISCRSSQSLVHSNRNTYLHWYL |
| | GPC3 Light | QKPGQSPQLLIYKVSNRFSGVPDRFSGSGSGTDFTLKISRVEA |
| | Chain | EDVGVYYCSQNTHVPPTFGQGTKLEIKRTVAAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSPV |
| | | TKSFNRGEC |
| 22 | LM1653 | QVQLVQSGAEVKKPGASVKVSCKASGYTFTDYEMHWVRQ |
| | GPC3 VH- | APGQGLEWMGALDPKTGDTAYSQKFKGRVTLTADKSTSTA |
| | CH1 | YMELSSLTSEDTAVYYCTRFYSYTYWGQGTLVTVSSASTKG |
| | | PSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSC |
| 23 | LM1653 | QVQLVQSGAEVKKPGASVKVSCKASGYTFTDYEMHWVRQ |
| | GPC3-hole | APGQGLEWMGALDPKTGDTAYSQKFKGRVTLTADKSTSTA |
| | (TCR) | YMELSSLTSEDTAVYYCTRFYSYTYWGQGTLVTVSSASTKG |
| | HC TCR (FC | PSVFPLAPSSKSTSGGTAALGCLVKDYFPEPVTVSWNSGALT |
| | hole) | SGVHTFPAVLQSSGLYSLSSVVTVPSSSLGTQTYICNVNHKPS |
| | | NTKVDKRVEPKSCDKTGGSEVQLVESGGGLVQPGGSLRLSC |
| | | VASGDVHKINFLGWYRQAPGKEREKVAHISIGDQTDYADSA |
| | | KGRFTISRDESKNMVYLQMNSLKPEDTAVYFCRAFSRIYPYD |
| | | YWGQGTLVTVSSGGGGSTHTCPPCPAPPVAGPSVFLFPPKPK |
| | | DTLMISRTPEVTCVVVDVKHEDPEVKFNWYVDGVEVHNAK |
| | | TKPREEQYNSTYRVVSVLTVLHQDWLNGKEYKCKVSNKAL |
| | | PAPIEKTISKAKGQPREPQVCTLPPSREEMTKNQVSLSCAVKG |
| | | FYPSDIAVEWESNGQPENNYKTTPPVLDSDGSFFLVSKLTVD |
| | | KSRWQQGNVFSCSVMHEALHNHYTQKSLSLSPGK |
| 24 | LM1653 | QVQLVQSGAEVKKPGASVKVSCKASGYTFTDYEMHWVRQ |
| | GPC3-knob | APGQGLEWMGALDPKTGDTAYSQKFKGRVTLTADKSTSTA |
| | (CD8) | YMELSSLTSEDTAVYYCTRFYSYTYWGQGTLVTVSSASTKG |
| | HC CD8 (FC | PSVFPLAPSSKSTSGGTAALGCLVKDYFPEPVTVSWNSGALT |
| | knob) | SGVHTFPAVLQSSGLYSLSSVVTVPSSSLGTQTYICNVNHKPS |
| | | NTKVDKRVEPKSCDKTGGSEVQLLESGGGLVQPGGSLRLSC |
| | | AASGFTFDDYAIGWFRQAPGKEREGVSCIRVSDGSTYYADS |
| | | VKGRFTISRDNSKNTLYLQMNSLRAEDTAVYYCAAGSLYTC |
| | | VQSIVWPARPYYDMDYWGQGTLVTVSSGGGGSTHTCPPCP |
| | | APPVAGPSVFLFPPKPKDTLMISRTPEVTCVVVDVKHEDPEV |
| | | KFNWYVDGVEVHNAKTKPREEQYNSTYRVVSVLTVLHQD |
| | | WLNGKEYKCKVSNKALPAPIEKTISKAKGQPREPQVYTLPPC |
| | | REEMTKNQVSLWCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| 25 | LRC150016 | SYWIE |
| | VH CDR1 | |
| 26 | LRC150016 | EILPGSDTTNYNEKFKD |
| | VH CDR2 | |
| 27 | LRC150016 | DRGNYRAWFGY |
| | VH CDR3 | |
| 28 | LRC150016 | RASQDISNYLN |
| | VL CDR1 | |
| 29 | LRC150016 | YTSRLHS |
| | VL CDR2 | |
| 30 | LRC150016 | QQGEALPWT |
| | VL CDR3 | |
| 31 | LRC150016 | EVQLVQSGAEVKKPGASVKVSCKASGYKFSSYWIEWVKQA |
| | VH | PGQGLEWIGEILPGSDTTNYNEKFKDRATFTSDTSINTAYME |
| | | LSRLRSDDTAVYYCARDRGNYRAWFGYWGQGTLVTVSS |
| 32 | LRC150016 | DIQMTQSPSSLSASVGDRVTITCRASQDISNYLNWYQQKPGG |
| | VL | AVKFLIYYTSRLHSGVPSRFSGSGSGTDYTLTISSLQPEDFAT |
| | | YFCQQGEALPWTFGGGTKVEIK |
| 33 | LRC150016 | DIQMTQSPSSLSASVGDRVTITCRASQDISNYLNWYQQKPGG |
| | Light Chain | AVKFLIYYTSRLHSGVPSRFSGSGSGTDYTLTISSLQPEDFAT |
| | | YFCQQGEALPWTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 34 | LRC150016 | EVQLVQSGAEVKKPGASVKVSCKASGYKFSSYWIEWVKQA |
| | VH-CHI | PGQGLEWIGEILPGSDTTNYNEKFKDRATFTSDTSINTAYME |
| | | LSRLRSDDTAVYYCARDRGNYRAWFGYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSC |
| 35 | LRC150016- | EVQLVQSGAEVKKPGASVKVSCKASGYKFSSYWIEWVKQA |
| | hole (TCR) | PGQGLEWIGEILPGSDTTNYNEKFKDRATFTSDTSINTAYME |
| | HC (FC hole) | LSRLRSDDTAVYYCARDRGNYRAWFGYWGQGTLVTVSSAS |
| | | TKGPSVFPLAPSSKSTSGGTAALGCLVKDYFPEPVTVSWNSG |
| 36 | LRC150016- | ALTSGVHTFPAVLQSSGLYSLSSVVTVPSSSLGTQTYICNVNH |
| | knob (CD8) | KPSNTKVDKRVEPKSCDKTGGSEVQLVESGGGLVQPGGSLR |
| | HC CD8 (FC | LSCVASGDVHKINFLGWYRQAPGKEREKVAHISIGDQTDYA |
| | knob) | DSAKGRFTISRDESKNMVYLQMNSLKPEDTAVYFCRAFSRIY |
| | | PYDYWGQGTLVTVSSGGGGSTHTCPPCPAPPVAGPSVFLFPP |
| | | KPKDTLMISRTPEVTCVVVDVKHEDPEVKFNWYVDGVEVH |
| | | NAKTKPREEQYNSTYRVVSVLTVLHQDWLNGKEYKCKVSN |
| | | KALPAPIEKTISKAKGQPREPQVCTLPPSREEMTKNQVSLSCA |
| | | VKGFYPSDIAVEWESNGQPENNYKTTPPVLDSDGSFFLVSKL |
| | | TVDKSRWQQGNVFSCSVMHEALHNRFTQKSLSLSPGK |
| | | EVQLVQSGAEVKKPGASVKVSCKASGYKFSSYWIEWVKQA |
| | | PGQGLEWIGEILPGSDTTNYNEKFKDRATFTSDTSINTAYME |
| | | LSRLRSDDTAVYYCARDRGNYRAWEGYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDKTGGSEVQLLESGGGLVQPGGSLR |
| | | LSCAASGFTFDDYAIGWFRQAPGKEREGVSCIRVSDGSTYYA |
| | | DSVKGRFTISRDNSKNTLYLQMNSLRAEDTAVYYCAAGSLY |
| | | TCVQSIVWPARPYYDMDYWGQGTLVTVSSGGGGSTHTCPP |
| | | CPAPPVAGPSVFLFPPKPKDTLMISRTPEVTCVVVDVKHEDP |
| | | EVKFNWYVDGVEVHNAKTKPREEQYNSTYRVVSVLTVLHQ |
| | | DWLNGKEYKCKVSNKALPAPIEKTISKAKGQPREPQVYTLPP |
| | | CREEMTKNQVSLWCLVKGFYPSDIAVEWESNGQPENNYKTT |
| | | PPVLDSDGSFFLYSKLTVDKSRWQQGNVFSCSVMHEALHNH |
| | | YTQKSLSLSPGK |
| 37 | linker | TGGS |
| 38 | linker | GGGGS |
| 39 | TCR V_{HHa} | INFLG |
| | CDR1 | |
| 40 | TCR V_{HHa} | HISIGDQTDYADSAKG |
| | CDR2 | |
| 41 | TCR V_{HHa} | FSRIYPYDY |
| | CDR3 | |
| 42 | TCR V_{HHa} | EVQLVESGGGLVQPGGSLRLSCVASGDVHKINFLGWYRQAP |
| | | GKEREKVAHISIGDQTDYADSAKGRFTISRDESKNMVYLQM |
| | | NSLKPEDTAVYFCRAFSRIYPYDYWGQGTLVTVSS |
| 43 | TCR hu2.1 | EVQLLESGGGLVQPGGSLRLSCAASGDVHKINFLSWYRQAP |
| | V_{HHa} | GKEREKVAHISIGDQTDYADSAKGRFTISRDESKNTLYLQMN |
| | | SLRAEDTAVYYCRAFSRIYPYDYWGQGTLVTVSS |
| 44 | TCR hu2.2 | EVQLLESGGGLVQPGGSLRLSCAASGDVHKINFLGWYRQAP |
| | V_{HHa} | GKEREKVAHISIGDQTDYADSAKGRFTISRDESKNTLYLQMN |
| | | SLRAEDTAVYYCRAFSRIYPYDYWGQGTLVTVSS |
| 45 | TCR hu2.3 | EVQLLESGGGLVQPGGSLRLSCAASGDVHKINFLSWYRQAP |
| | V_{HHa} | GKGLEKVAHISIGDQTDYADSAKGRFTISRDESKNTLYLQMN |
| | | SLRAEDTAVYYCRAFSRIYPYDYWGQGTLVTVSS |
| 46 | TCR hu2.4 | EVQLLESGGGLVQPGGSLRLSCAASGDVHKINFLGWYRQAP |
| | V_{HHa} | GKGLEKVAHISIGDQTDYADSAKGRFTISRDESKNTLYLQMN |
| | | SLRAEDTAVYYCRAFSRIYPYDYWGQGTLVTVSS |
| 47 | TCR hu2.5 | EVQLLESGGGLVQPGGSLRLSCAASGDVHKINFLGWYRQAP |
| | V_{HHa} | GKEREKVAHISIGDQTDYADSAKGRFTISRDESKNTVYLQMN |
| | | SLRAEDTAVYFCRAFSRIYPYDYWGQGTLVTVSS |
| 48 | TCR hu2.6 | EVQLLESGGGLVQPGGSLRLSCAASGDVHKINFLGWYRQAP |
| | V_{HHa} | GKGLEKVAHISIGDQTDYADSAKGRFTISRDESKNTVYLQM |
| | | NSLRAEDTAVYFCRAFSRIYPYDYWGQGTLVTVSS |
| 49 | CD8 V_{HHb} | DYAIG |
| | CDR1 | |
| 50 | CD8 V_{HHb} | IRVSDGSTYYADSVKG |
| | CDR2 | |
| 51 | CD8 V_{HHb} | GSLYTCVQSIVWPARPYYDMDY |
| | CDR3 | |
| 52 | CD8 V_{HHb} | EVQLLESGGGLVQPGGSLRLSCAASGFTFDDYAIGWFRQAP |
| | LM1486 (CD20 | GKEREGVSCIRVSDGSTYYADSVKGRFTISRDNSKNTLYLQM |
| | CD8) | NSLRAEDTAVYYCAAGSLYTCVQSIVWPARPYYDMDYWGQ |
| | BCMA CD8 | GTLVTVSS |
| | LM1953 | |
| | (STEAP2 | |
| | CD8) | |
| | LRC150016 | |
| | (LRRC15) | |
| 53 | IgG1 hinge | DKTHTCPPCPAP |
| | sequence | |
| 54 | IgG1 Split | DK |
| | Hinge 1 | |
| 55 | IgG1 Split | THTCPPCPAP |
| | Hinge 2 | |
| 56 | Fcₐ (hole) | PVAGPSVFLFPPKPKDTLMISRTPEVTCVVVDVKHEDPEVKF |
| | | NWYVDGVEVHNAKTKPREEQYNSTYRVVSVLTVLHQDWL |
| | | NGKEYKCKVSNKALPAPIEKTISKAKGQPREPQVCTLPPSREE |
| | | MTKNQVSLSCAVKGFYPSDIAVEWESNGQPENNYKTTPPVL |
| | | DSDGSFFLVSKLTVDKSRWQQGNVFSCSVMHEALHNHYTQ |
| | | KSLSLSPGK |
| 57 | Fcₐ (hole) | PVAGPSVFLFPPKPKDTLMISRTPEVTCVVVDVKHEDPEVKF |
| | (RF) | NWYVDGVEVHNAKTKPREEQYNSTYRVVSVLTVLHQDWL |
| | | NGKEYKCKVSNKALPAPIEKTISKAKGQPREPQVCTLPPSREE |
| | | MTKNQVSLSCAVKGFYPSDIAVEWESNGQPENNYKTTPPVL |
| | | DSDGSFFLVSKLTVDKSRWQQGNVFSCSVMHEALHNRFTQK |
| | | SLSLSPGK |
| 58 | Fc_{b} (knob) | PVAGPSVFLFPPKPKDTLMISRTPEVTCVVVDVKHEDPEVKF |
| | | NWYVDGVEVHNAKTKPREEQYNSTYRVVSVLTVLHQDWL |
| | | NGKEYKCKVSNKALPAPIEKTISKAKGQPREPQVYTLPPCRE |
| | | EMTKNQVSLWCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| 59 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | | NHKPSNTKVDKRVEPKSCDKTGGSEVQLVESGGGLVQPGGS |
| | | LRLSCVASGDVHKINFLGWYRQAPGKEREKVAHISIGDQTD |
| | | YADSAKGRFTISRDESKNMVYLQMNSLKPEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 60 | Backbone | ASTKGPSVFPLAPSSKSTSGGTAALGCLVKDYFPEPVTVSWN |
| | (TCR/hole/RF) | SGALTSGVHTFPAVLQSSGLYSLSSVVTVPSSSLGTQTYICNV |
| | | NHKPSNTKVDKRVEPKSCDKTGGSEVQLVESGGGLVQPGGS |
| | | LRLSCVASGDVHKINFLGWYRQAPGKEREKVAHISIGDQTD |
| | | YADSAKGRFTISRDESKNMVYLQMNSLKPEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNRFTQKSLSLSPGK |
| 61 | Backbone | ASTKGPSVFPLAPSSKSTSGGTAALGCLVKDYFPEPVTVSWN |
| | (CD8/knob) | SGALTSGVHTFPAVLQSSGLYSLSSVVTVPSSSLGTQTYICNV |
| | | NHKPSNTKVDKRVEPKSCDKTGGSEVQLLESGGGLVQPGGS |
| | | LRLSCAASGFTEDDYAIGWFRQAPGKEREGVSCIRVSDGSTY |
| | | YADSVKGRFTISRDNSKNTLYLQMNSLRAEDTAVYYCAAGS |
| | | LYTCVQSIVWPARPYYDMDYWGQGTLVTVSSGGGGSTHTC |
| | | PPCPAPPVAGPSVFLFPPKPKDTLMISRTPEVTCVVVDVKHED |
| | | PEVKFNWYVDGVEVHNAKTKPREEQYNSTYRVVSVLTVLH |
| | | QDWLNGKEYKCKVSNKALPAPIEKTISKAKGQPREPQVYTL |
| | | PPCREEMTKNQVSLWCLVKGFYPSDIAVEWESNGQPENNYK |
| | | TTPPVLDSDGSFFLYSKLTVDKSRWQQGNVFSCSVMHEALH |
| | | NHYTQKSLSLSPGK |
| 62 | TCR V_{HHa} hu | INFLS |
| | 2.1, 2.3 | |
| | CDR1 | |
| 63 | LRC150016- | EVQLVQSGAEVKKPGASVKVSCKASGYKFSSYWIEWVKQA |
| | hole (TCR) | PGQGLEWIGEILPGSDTTNYNEKFKDRATFTSDTSINTAYME |
| | HC TCR (FC | LSRLRSDDTAVYYCARDRGNYRAWFGYWGQGTLVTVSSAS |
| | hole) hu2.1 | TKGPSVFPLAPSSKSTSGGTAALGCLVKDYFPEPVTVSWNSG |
| | V_{HHa} | ALTSGVHTFPAVLQSSGLYSLSSVVTVPSSSLGTQTYICNVNH |
| | | KPSNTKVDKRVEPKSCDKTGGSEVQLLESGGGLVQPGGSLR |
| | | LSCAASGDVHKINFLSWYRQAPGKEREKVAHISIGDQTDYA |
| | | DSAKGRFTISRDESKNTLYLQMNSLRAEDTAVYYCRAFSRIY |
| | | PYDYWGQGTLVTVSSGGGGSTHTCPPCPAPPVAGPSVFLFPP |
| | | KPKDTLMISRTPEVTCVVVDVKHEDPEVKFNWYVDGVEVH |
| | | NAKTKPREEQYNSTYRVVSVLTVLHQDWLNGKEYKCKVSN |
| | | KALPAPIEKTISKAKGQPREPQVCTLPPSREEMTKNQVSLSCA |
| | | VKGFYPSDIAVEWESNGQPENNYKTTPPVLDSDGSFFLVSKL |
| | | TVDKSRWQQGNVFSCSVMHEALHNRFTQKSLSLSPGK |
| 64 | LRC150016- | EVQLVQSGAEVKKPGASVKVSCKASGYKFSSYWIEWVKQA |
| | hole (TCR) | PGQGLEWIGEILPGSDTTNYNEKFKDRATFTSDTSINTAYME |
| | HC TCR (FC | LSRLRSDDTAVYYCARDRGNYRAWFGYWGQGTLVTVSSAS |
| | hole) hu2.2 | TKGPSVFPLAPSSKSTSGGTAALGCLVKDYFPEPVTVSWNSG |
| | V_{HHa} | ALTSGVHTFPAVLQSSGLYSLSSVVTVPSSSLGTQTYICNVNH |
| | | KPSNTKVDKRVEPKSCDKTGGSEVQLLESGGGLVQPGGSLR |
| | | LSCAASGDVHKINFLGWYRQAPGKEREKVAHISIGDQTDYA |
| | | DSAKGRFTISRDESKNTLYLQMNSLRAEDTAVYYCRAFSRIY |
| | | PYDYWGQGTLVTVSSGGGGSTHTCPPCPAPPVAGPSVFLFPP |
| | | KPKDTLMISRTPEVTCVVVDVKHEDPEVKFNWYVDGVEVH |
| | | NAKTKPREEQYNSTYRVVSVLTVLHQDWLNGKEYKCKVSN |
| | | KALPAPIEKTISKAKGQPREPQVCTLPPSREEMTKNQVSLSCA |
| | | VKGFYPSDIAVEWESNGQPENNYKTTPPVLDSDGSFFLVSKL |
| | | TVDKSRWQQGNVFSCSVMHEALHNRFTQKSLSLSPGK |
| 65 | LRC150016- | EVQLVQSGAEVKKPGASVKVSCKASGYKFSSYWIEWVKQA |
| | hole (TCR) | PGQGLEWIGEILPGSDTTNYNEKFKDRATFTSDTSINTAYME |
| | HC TCR (FC | LSRLRSDDTAVYYCARDRGNYRAWFGYWGQGTLVTVSSAS |
| | hole) hu2.3 | TKGPSVFPLAPSSKSTSGGTAALGCLVKDYFPEPVTVSWNSG |
| | V_{HHa} | ALTSGVHTFPAVLQSSGLYSLSSVVTVPSSSLGTQTYICNVNH |
| | | KPSNTKVDKRVEPKSCDKTGGSEVQLLESGGGLVQPGGSLR |
| | | LSCAASGDVHKINFLSWYRQAPGKGLEKVAHISIGDQTDYA |
| | | DSAKGRFTISRDESKNTLYLQMNSLRAEDTAVYYCRAFSRIY |
| | | PYDYWGQGTLVTVSSGGGGSTHTCPPCPAPPVAGPSVFLFPP |
| | | KPKDTLMISRTPEVTCVVVDVKHEDPEVKFNWYVDGVEVH |
| | | NAKTKPREEQYNSTYRVVSVLTVLHQDWLNGKEYKCKVSN |
| | | KALPAPIEKTISKAKGQPREPQVCTLPPSREEMTKNQVSLSCA |
| | | VKGFYPSDIAVEWESNGQPENNYKTTPPVLDSDGSFFLVSKL |
| | | TVDKSRWQQGNVFSCSVMHEALHNRFTQKSLSLSPGK |
| 66 | LRC150016- | EVQLVQSGAEVKKPGASVKVSCKASGYKFSSYWIEWVKQA |
| | hole (TCR) | PGQGLEWIGEILPGSDTTNYNEKFKDRATFTSDTSINTAYME |
| | HC TCR (FC | LSRLRSDDTAVYYCARDRGNYRAWFGYWGQGTLVTVSSAS |
| | hole) hu2.4 | TKGPSVFPLAPSSKSTSGGTAALGCLVKDYFPEPVTVSWNSG |
| | V_{HHa} | ALTSGVHTFPAVLQSSGLYSLSSVVTVPSSSLGTQTYICNVNH |
| | | KPSNTKVDKRVEPKSCDKTGGSEVQLLESGGGLVQPGGSLR |
| | | LSCAASGDVHKINFLGWYRQAPGKGLEKVAHISIGDQTDYA |
| | | DSAKGRFTISRDESKNTLYLQMNSLRAEDTAVYYCRAFSRIY |
| | | PYDYWGQGTLVTVSSGGGGSTHTCPPCPAPPVAGPSVFLFPP |
| | | KPKDTLMISRTPEVTCVVVDVKHEDPEVKFNWYVDGVEVH |
| | | NAKTKPREEQYNSTYRVVSVLTVLHQDWLNGKEYKCKVSN |
| | | KALPAPIEKTISKAKGQPREPQVCTLPPSREEMTKNQVSLSCA |
| | | VKGFYPSDIAVEWESNGQPENNYKTTPPVLDSDGSFFLVSKL |
| | | TVDKSRWQQGNVFSCSVMHEALHNRFTQKSLSLSPGK |
| 67 | LRC150016- | EVQLVQSGAEVKKPGASVKVSCKASGYKFSSYWIEWVKQA |
| | hole (TCR) | PGQGLEWIGEILPGSDTTNYNEKFKDRATFTSDTSINTAYME |
| | HC TCR (FC | LSRLRSDDTAVYYCARDRGNYRAWEGYWGQGTLVTVSSAS |
| | hole) hu2.5 | TKGPSVFPLAPSSKSTSGGTAALGCLVKDYFPEPVTVSWNSG |
| | V_{HHa} | ALTSGVHTFPAVLQSSGLYSLSSVVTVPSSSLGTQTYICNVNH |
| | | KPSNTKVDKRVEPKSCDKTGGSEVQLLESGGGLVQPGGSLR |
| | | LSCAASGDVHKINFLGWYRQAPGKEREKVAHISIGDQTDYA |
| | | DSAKGRFTISRDESKNTVYLQMNSLRAEDTAVYFCRAFSRIY |
| | | PYDYWGQGTLVTVSSGGGGSTHTCPPCPAPPVAGPSVFLFPP |
| | | KPKDTLMISRTPEVTCVVVDVKHEDPEVKFNWYVDGVEVH |
| | | NAKTKPREEQYNSTYRVVSVLTVLHQDWLNGKEYKCKVSN |
| | | KALPAPIEKTISKAKGQPREPQVCTLPPSREEMTKNQVSLSCA |
| | | VKGFYPSDIAVEWESNGQPENNYKTTPPVLDSDGSFFLVSKL |
| | | TVDKSRWQQGNVFSCSVMHEALHNRFTQKSLSLSPGK |
| 68 | LRC150016- | EVQLVQSGAEVKKPGASVKVSCKASGYKFSSYWIEWVKQA |
| | hole (TCR) | PGQGLEWIGEILPGSDTTNYNEKFKDRATFTSDTSINTAYME |
| | HC TCR (FC | LSRLRSDDTAVYYCARDRGNYRAWFGYWGQGTLVTVSSAS |
| | hole) hu2.6 | TKGPSVFPLAPSSKSTSGGTAALGCLVKDYFPEPVTVSWNSG |
| | V_{HHa} | ALTSGVHTFPAVLQSSGLYSLSSVVTVPSSSLGTQTYICNVNH |
| | | KPSNTKVDKRVEPKSCDKTGGSEVQLLESGGGLVQPGGSLR |
| | | LSCAASGDVHKINFLGWYRQAPGKGLEKVAHISIGDQTDYA |
| | | DSAKGRFTISRDESKNTVYLQMNSLRAEDTAVYFCRAFSRIY |
| | | PYDYWGQGTLVTVSSGGGGSTHTCPPCPAPPVAGPSVFLFPP |
| | | KPKDTLMISRTPEVTCVVVDVKHEDPEVKFNWYVDGVEVH |
| | | NAKTKPREEQYNSTYRVVSVLTVLHQDWLNGKEYKCKVSN |
| | | KALPAPIEKTISKAKGQPREPQVCTLPPSREEMTKNQVSLSCA |
| | | VKGFYPSDIAVEWESNGQPENNYKTTPPVLDSDGSFFLVSKL |
| | | TVDKSRWQQGNVFSCSVMHEALHNRFTQKSLSLSPGK |
| 69 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | TCR hu2.1 | NHKPSNTKVDKRVEPKSCDKTGGSEVQLLESGGGLVQPGGS |
| | V_{HHa} | LRLSCAASGDVHKINFLSWYRQAPGKEREKVAHISIGDQTDY |
| | | ADSAKGRFTISRDESKNTLYLQMNSLRAEDTAVYYCRAFSRI |
| | | YPYDYWGQGTLVTVSSGGGGSTHTCPPCPAPPVAGPSVFLFP |
| | | PKPKDTLMISRTPEVTCVVVDVKHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSREEMTKNQVSLSC |
| | | AVKGFYPSDIAVEWESNGQPENNYKTTPPVLDSDGSFFLVSK |
| | | LTVDKSRWQQGNVFSCSVMHEALHNHYTQKSLSLSPGK |
| 70 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | TCR hu2.2 | NHKPSNTKVDKRVEPKSCDKTGGSEVQLLESGGGLVQPGGS |
| | V_{HHa} | LRLSCAASGDVHKINFLGWYRQAPGKEREKVAHISIGDQTD |
| | | YADSAKGRFTISRDESKNTLYLQMNSLRAEDTAVYYCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 71 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | TCR hu2.3 | NHKPSNTKVDKRVEPKSCDKTGGSEVQLLESGGGLVQPGGS |
| | V_{HHa} | LRLSCAASGDVHKINFLSWYRQAPGKGLEKVAHISIGDQTDY |
| | | ADSAKGRFTISRDESKNTLYLQMNSLRAEDTAVYYCRAFSRI |
| | | YPYDYWGQGTLVTVSSGGGGSTHTCPPCPAPPVAGPSVFLFP |
| | | PKPKDTLMISRTPEVTCVVVDVKHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSREEMTKNQVSLSC |
| | | AVKGFYPSDIAVEWESNGQPENNYKTTPPVLDSDGSFFLVSK |
| | | LTVDKSRWQQGNVFSCSVMHEALHNHYTQKSLSLSPGK |
| 72 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | TCR hu2.4 | NHKPSNTKVDKRVEPKSCDKTGGSEVQLLESGGGLVQPGGS |
| | V_{HHa} | LRLSCAASGDVHKINFLGWYRQAPGKGLEKVAHISIGDQTD |
| | | YADSAKGRFTISRDESKNTLYLQMNSLRAEDTAVYYCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 73 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | TCR hu2.5 | NHKPSNTKVDKRVEPKSCDKTGGSEVQLLESGGGLVQPGGS |
| | V_{HHa} | LRLSCAASGDVHKINFLGWYRQAPGKEREKVAHISIGDQTD |
| | | YADSAKGRFTISRDESKNTVYLQMNSLRAEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 74 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | TCR hu2.6 | NHKPSNTKVDKRVEPKSCDKTGGSEVQLLESGGGLVQPGGS |
| | V_{HHa} | LRLSCAASGDVHKINFLGWYRQAPGKGLEKVAHISIGDQTD |
| | | YADSAKGRFTISRDESKNTVYLQMNSLRAEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 75 | Backbone | ASTKGPSVFPLAPSSKSTSGGTAALGCLVKDYFPEPVTVSWN |
| | (TCR/hole/RF) | SGALTSGVHTFPAVLQSSGLYSLSSVVTVPSSSLGTQTYICNV |
| | TCR hu2.1 | NHKPSNTKVDKRVEPKSCDKTGGSEVQLLESGGGLVQPGGS |
| | V_{HHa} | LRLSCAASGDVHKINFLSWYRQAPGKEREKVAHISIGDQTDY |
| | | ADSAKGRFTISRDESKNTLYLQMNSLRAEDTAVYYCRAFSRI |
| | | YPYDYWGQGTLVTVSSGGGGSTHTCPPCPAPPVAGPSVFLFP |
| | | PKPKDTLMISRTPEVTCVVVDVKHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSREEMTKNQVSLSC |
| | | AVKGFYPSDIAVEWESNGQPENNYKTTPPVLDSDGSFFLVSK |
| | | LTVDKSRWQQGNVFSCSVMHEALHNRFTQKSLSLSPGK |
| 76 | Backbone | ASTKGPSVFPLAPSSKSTSGGTAALGCLVKDYFPEPVTVSWN |
| | (TCR/hole/RF) | SGALTSGVHTFPAVLQSSGLYSLSSVVTVPSSSLGTQTYICNV |
| | TCR hu2.2 | NHKPSNTKVDKRVEPKSCDKTGGSEVQLLESGGGLVQPGGS |
| | V_{HHa} | LRLSCAASGDVHKINFLGWYRQAPGKEREKVAHISIGDQTD |
| | | YADSAKGRFTISRDESKNTLYLQMNSLRAEDTAVYYCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNRFTQKSLSLSPGK |
| 77 | Backbone | ASTKGPSVFPLAPSSKSTSGGTAALGCLVKDYFPEPVTVSWN |
| | (TCR/hole/RF) | SGALTSGVHTFPAVLQSSGLYSLSSVVTVPSSSLGTQTYICNV |
| | TCR hu2.3 | NHKPSNTKVDKRVEPKSCDKTGGSEVQLLESGGGLVQPGGS |
| | V_{HHa} | LRLSCAASGDVHKINFLSWYRQAPGKGLEKVAHISIGDQTDY |
| | | ADSAKGRFTISRDESKNTLYLQMNSLRAEDTAVYYCRAFSRI |
| | | YPYDYWGQGTLVTVSSGGGGSTHTCPPCPAPPVAGPSVFLFP |
| | | PKPKDTLMISRTPEVTCVVVDVKHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSREEMTKNQVSLSC |
| | | AVKGFYPSDIAVEWESNGQPENNYKTTPPVLDSDGSFFLVSK |
| | | LTVDKSRWQQGNVFSCSVMHEALHNRFTQKSLSLSPGK |
| 78 | Backbone | ASTKGPSVFPLAPSSKSTSGGTAALGCLVKDYFPEPVTVSWN |
| | (TCR/hole/RF) | SGALTSGVHTFPAVLQSSGLYSLSSVVTVPSSSLGTQTYICNV |
| | TCR hu2.4 | NHKPSNTKVDKRVEPKSCDKTGGSEVQLLESGGGLVQPGGS |
| | V_{HHa} | LRLSCAASGDVHKINFLGWYRQAPGKGLEKVAHISIGDQTD |
| | | YADSAKGRFTISRDESKNTLYLQMNSLRAEDTAVYYCRAFS |
| | | RIYPYDYWGQGTLVTVSSGGGGSTHTCPPCPAPPVAGPSVEL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNRFTQKSLSLSPGK |
| 79 | Backbone | ASTKGPSVFPLAPSSKSTSGGTAALGCLVKDYFPEPVTVSWN |
| | (TCR/hole/RF) | SGALTSGVHTFPAVLQSSGLYSLSSVVTVPSSSLGTQTYICNV |
| | TCR hu2.5 | NHKPSNTKVDKRVEPKSCDKTGGSEVQLLESGGGLVQPGGS |
| | V_{HHa} | LRLSCAASGDVHKINFLGWYRQAPGKEREKVAHISIGDQTD |
| | | YADSAKGRFTISRDESKNTVYLQMNSLRAEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNRFTQKSLSLSPGK |
| 80 | Backbone | ASTKGPSVFPLAPSSKSTSGGTAALGCLVKDYFPEPVTVSWN |
| | (TCR/hole/RF) | SGALTSGVHTFPAVLQSSGLYSLSSVVTVPSSSLGTQTYICNV |
| | TCR hu2.6 | NHKPSNTKVDKRVEPKSCDKTGGSEVQLLESGGGLVQPGGS |
| | V_{HHa} | LRLSCAASGDVHKINFLGWYRQAPGKGLEKVAHISIGDQTD |
| | | YADSAKGRFTISRDESKNTVYLQMNSLRAEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNRFTQKSLSLSPGK |
| 81 | CD8 V_{HHb} | EVQLLESGGGLVQPGGSLRLSCAASGFTFPDYAIGWFRQAPG |
| | D30P/S75A/ | KEREGVSCIRVSDGSTYYADSVKGRFTISRDNAKNTLYLQM |
| | Q106N | NSLRAEDTAVYYCAAGSLYTCVNSIVWPARPYYDMDYWGQ |
| | | GTLVTVSS |
| 82 | CD8 V_{HHb} | EVQLLESGGGLVQPGGSLRLSCAASGFTFPDYAIGWFRQAPG |
| | D30P/S75A/ | KEREGVSCIRVSDGSTYYADSVKGRFTISRDNAKNTLYLQM |
| | L101A | NSLRAEDTAVYYCAAGSAYTCVQSIVWPARPYYDMDYWG |
| | | QGTLVTVSS |
| 83 | CD8 V_{HHb} | EVQLLESGGGLVQPGGSLRLSCAASGFTFRDYAIGWFRQAPG |
| | S75A/D30R | KEREGVSCIRVSDGSTYYADSVKGRFTISRDNAKNTLYLQM |
| | | NSLRAEDTAVYYCAAGSLYTCVQSIVWPARPYYDMDYWGQ |
| | | GTLVTVSS |
| 84 | CD8 V_{HHb} | EVQLLESGGGLVQPGGSLRLSCAASGFTFPDYAIGWFRQAPG |
| | D30P/S75A/ | KEREGVSCIRVSDGSTYYADSVKGRFTISRDNAKNTLYLQM |
| | S100G | NSLRAEDTAVYYCAAGGLYTCVQSIVWPARPYYDMDYWG |
| | | QGTLVTVSS |
| 85 | CD8 V_{HHb} | EVQLLESGGGLVQPGGSLRLSCAASGFTFPDYAIGWFRQAPG |
| | D30P/S75A/ | KEREGVSCIRVSDGSTYYADSVKGRFTISRDNAKNTLYLQM |
| | Y102I | NSLRAEDTAVYYCAAGSLITCVQSIVWPARPYYDMDYWGQ |
| | | GTLVTVSS |
| 86 | CD8 V_{HHb} | EVQLLESGGGLVQPGGSLRLSCAASGFTFPDYAIGWFRQAPG |
| | D30P/S75A/ | KEREGVSCIRVSDGSTYYADSVKGRFTISRDNAKNTLYLQM |
| | S100P | NSLRAEDTAVYYCAAGPLYTCVQSIVWPARPYYDMDYWGQ |
| | | GTLVTVSS |
| 87 | CD8 V_{HHb} | EVQLLESGGGLVQPGGSLRLSCAASGFTFDSYAIGWFRQAPG |
| | S75A/D31S | KEREGVSCIRVSDGSTYYADSVKGRFTISRDNAKNTLYLQM |
| | | NSLRAEDTAVYYCAAGSLYTCVQSIVWPARPYYDMDYWGQ |
| | | GTLVTVSS |
| 88 | CD8 V_{HHb} | EVQLLESGGGLVQPGGSLRLSCAASGFTFPDYAIGWFRQAPG |
| | D30P/S75A/ | KEREGVSCITVSDGSTYYADSVKGRFTISRDNAKNTLYLQM |
| | R52T | NSLRAEDTAVYYCAAGSLYTCVQSIVWPARPYYDMDYWGQ |
| | | GTLVTVSS |
| 89 | CD8 V_{HHb} | EVQLLESGGGLVQPGGSLRLSCAASGFTFPDYAIGWFRQAPG |
| | D30P/S75A | KEREGVSCIRVSDGSTYYADSVKGRFTISRDNAKNTLYLQM |
| | | NSLRAEDTAVYYCAAGSLYTCVQSIVWPARPYYDMDYWGQ |
| | | GTLVTVSS |
| 90 | IgG1 IMGT | EPKSCDK |
| | Upper Hinge | |
| | Region | |
| 91 | IMGT Lower | THTCPPCP |
| | Hinge Region | |
| 92 | LM1486 | EVQLVESGGGLVQPGGSLRLSCAASGYTFTSYNMHWVRQA |
| | CD20 VH- | PGKGLEWVGAIYPGSGDTSYNQKFKGRFTISVDKSKNTLYL |
| | CH1 (IMGT) | QMNSLRAEDTAVYYCARVVYYSNSYWYFDVWGQGTLVTV |
| | | SSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKRV |
| 93 | LM1653 | QVQLVQSGAEVKKPGASVKVSCKASGYTFTDYEMHWVRQ |
| | GPC3 VH- | APGQGLEWMGALDPKTGDTAYSQKFKGRVTLTADKSTSTA |
| | CH1 (IMGT) | YMELSSLTSEDTAVYYCTRFYSYTYWGQGTLVTVSSASTKG |
| | | PSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRV |
| 94 | LRC150016 | EVQLVQSGAEVKKPGASVKVSCKASGYKFSSYWIEWVKQA |
| | VH-CH1 | PGQGLEWIGEILPGSDTTNYNEKFKDRATFTSDTSINTAYME |
| | (IMGT) | LSRLRSDDTAVYYCARDRGNYRAWFGYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRV |
| 95 | WT C_{H1} | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKRVEPKSC |
| 96 | WT C_{H1} | ASTKGPSVFPLAPSSKSTSGGTAALGCLVKDYFPEPVTVSW |
| | (IMGT) | NSGALTSGVHTFPAVLQSSGLYSLSSVVTVPSSSLGTQTYIC |
| | | NVNHKPSNTKVDKRV |
| 97 | V12 C_{H1} | ASTKGPSVCPLAPSSKSTSGGTAALGCLVKDYFPEPVTVSW |
| | (F126C/C220V) | NSGALTSGVHTFPAVLQSSGLYSLSSVVTVPSSSLGTQTYIC |
| | | NVNHKPSNTKVDKRVEPKSV |
| 98 | WT C_{Lλ} | GQPKAAPSVTLFPPSSEELQANKATLVCLISDFYPGAVTVAW |
| | | KADSSPVKAGVETTTPSKQSNNKYAASSYLSLTPEQWKSHR |
| | | SYSCQVTHEGSTVEKTVAPTECS |
| 99 | V12 LC | GQPKAAPSVTLFPPCSEELQANKATLVCLISDFYPGAVTVAW |
| | lambda | KADSSPVKAGVETTTPSKQSNNKYAASSYLSLTPEQWKSHR |
| | constant | SYSCQVTHEGSTVEKTVAPTEVS |
| | (CLλ) | |
| 100 | WT LC kappa | RTVAAPSVFIFPPSDEQLKSGTASVVCLLNNFYPREAKVQWK |
| | constant (Cκ) | VDNALQSGNSQESVTEQDSKDSTYSLSSTLTLSKADYEKHK |
| | region | VYACEVTHQGLSSPVTKSFNRGEC |
| 101 | IgG1 CH2 | ELLGGPSVFLFPPKPKDTLMISRTPEVTCVVVDVSHEDPEVKF |
| | Wild-type | NWYVDGVEVHNAKTKPREEQYNSTYRVVSVLTVLHQDWL |
| | | NGKEYKCKVSNKALPAPIEKTIS |
| 102 | IgG1 CH2 Fc- | PVAGPSVFLFPPKPKDTLMISRTPEVTCVVVDVKHEDPEVKF |
| | null | NWYVDGVEVHNAKTKPREEQYNSTYRVVSVLTVLHQDWL |
| | | NGKEYKCKVSNKALPAPIEKTIS |
| 103 | IgG1 CH3 | GQPREPQVYTLPPSREEMTKNQVSLTCLVKGFYPSDIAVEWE |
| | Wild-type | SNGQPENNYKTTPPVLDSDGSFFLYSKLTVDKSRWQQGNVF |
| | | SCSVMHEALHNHYTQKSLSLSPGK |
| 104 | C_{H1} with | ASTKGPSVFPLAPSSKSTSGGTASLGCLVKDYFPEPVTVSW |
| | A141S | NSGALTSGVHTFPAVLQSSGLYSLSSVVTVPSSSLGTQTYIC |
| | | NVNHKPSNTKVDKRVEPKSC |
| 105 | CLλ with | GQPKAAPSVRLFPPSSEELQANKATLVCLISDFYPGAVTVAW |
| | T117R | KADSSPVKAGVETTTPSKQSNNKYAASSYLSLTPEQWKSHR |
| | | SYSCQVTHEGSTVEKTVAPTECS |
| 106 | V12 C_{H1} with | ASTKGPSVCPLAPSSKSTSGGTASLGCLVKDYFPEPVTVSW |
| | A141S | NSGALTSGVHTFPAVLQSSGLYSLSSVVTVPSSSLGTQTYIC |
| | | NVNHKPSNTKVDKRVEPKSV |
| 107 | V12 CLλ with | GQPKAAPSVRLFPPCSEELQANKATLVCLISDFYPGAVTVAW |
| | T117R | KADSSPVKAGVETTTPSKQSNNKYAASSYLSLTPEQWKSHR |
| | | SYSCQVTHEGSTVEKTVAPTEVS |
| 108 | IgG1 CH2 | APELLGGPSVFLFPPKPKDTLMISRTPEVTCVVVDVSHEDPEV |
| | WT (IMGT) | KFNWYVDGVEVHNAKTKPREEQYNSTYRVVSVLTVLHQD |
| | | WLNGKEYKCKVSNKALPAPIEKTIS |
| 109 | IgG1 CH2 Fc- | APPVAGPSVFLFPPKPKDTLMISRTPEVTCVVVDVKHEDPEV |
| | null (IMGT) | KFNWYVDGVEVHNAKTKPREEQYNSTYRVVSVLTVLHQD |
| | | WLNGKEYKCKVSNKALPAPIEKTIS |
| 110 | LM1486 | EVQLVESGGGLVQPGGSLRLSCAASGYTFTSYNMHWVRQA |
| | CD20 VH- | PGKGLEWVGAIYPGSGDTSYNQKFKGRFTISVDKSKNTLYL |
| | CH-TCR- | QMNSLRAEDTAVYYCARVVYYSNSYWYFDVWGQGTLVTV |
| | CH2CH3 | SSASTKGPSVFPLAPSSKSTSGGTAALGCLVKDYFPEPVTVS |
| | (hole/RF) | WNSGALTSGVHTFPAVLQSSGLYSLSSVVTVPSSSLGTQTYIC |
| | | NVNHKPSNTKVDKRVEPKSCDKTGGSEVQLVESGGGLVQPG |
| | | GSLRLSCVASGDVHKINFLGWYRQAPGKEREKVAHISIGDQT |
| | | DYADSAKGRFTISRDESKNMVYLQMNSLKPEDTAVYFCRAF |
| | | SRIYPYDYWGQGTLVTVSSGGGGSTHTCPPCPAPPVAGPSVF |
| | | LFPPKPKDTLMISRTPEVTCVVVDVKHEDPEVKFNWYVDGV |
| | | EVHNAKTKPREEQYNSTYRVVSVLTVLHQDWLNGKEYKCK |
| | | VSNKALPAPIEKTISKAKGQPREPQVCTLPPSREEMTKNQVSL |
| | | SCAVKGFYPSDIAVEWESNGQPENNYKTTPPVLDSDGSFFLV |
| | | SKLTVDKSRWQQGNVFSCSVMHEALHNRFTQKSLSLSPGK |
| 111 | TPP-42197 | SYSMN |
| | BCMA | |
| | VH CDR1 | |
| 112 | TPP-42197 | SISGQSNYIYYADSVKG |
| | BCMA | |
| | VH CDR2 | |
| 113 | TPP-42197 | GGNYFVEYFQY |
| | BCMA | |
| | VH CDR3 | |
| 114 | TPP-42197 | RASQYISSNNLA |
| | BCMA VL | |
| | CDR1 | |
| 115 | TPP-42197 | GASNRAT |
| | BCMA VL | |
| | CDR2 | |
| 116 | TPP-42197 | QQYADSPIT |
| | BCMA VL | |
| | CDR3 | |
| 117 | TPP-42197 | EVQLVESGGGLVKPGGSLRLSCAASGFTFSSYSMNWVRQAP |
| | BCMA VH | GKGLEWVSSISGQSNYIYYADSVKGRFTISRDNAKNSLYLQM |
| | | NSLRAEDTAVYYCARGGNYFVEYFQYWGQGTLVTVSS |
| 118 | TPP-42197 | EIVLTQSPGTLSLSPGERATLSCRASQYISSNNLAWYQQKPGQ |
| | BCMA VL | APRLLIYGASNRATGIPDRFSGSGSGTDFTLTISRLEPEDFAVY |
| | | YCQQYADSPITFGQGTKLEIK |
| 119 | TPP-42197 | EIVLTQSPGTLSLSPGERATLSCRASQYISSNNLAWYQQKPGQ |
| | BCMA light | APRLLIYGASNRATGIPDRFSGSGSGTDFTLTISRLEPEDFAVY |
| | chain | YCQQYADSPITFGQGTKLEIKRTVAAPSVFIFPPSDEQLKSGT |
| | | ASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| 120 | TPP-42197 | EVQLVESGGGLVKPGGSLRLSCAASGFTFSSYSMNWVRQAP |
| | BCMA VH- | GKGLEWVSSISGQSNYIYYADSVKGRFTISRDNAKNSLYLQM |
| | CH1 | NSLRAEDTAVYYCARGGNYFVEYFQYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDK |
| 121 | TPP-42197 | EVQLVESGGGLVKPGGSLRLSCAASGFTFSSYSMNWVRQAP |
| | hole (TCR) | GKGLEWVSSISGQSNYIYYADSVKGRFTISRDNAKNSLYLQM |
| | BCMA HC | NSLRAEDTAVYYCARGGNYFVEYFQYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDKTGGSEVQLVESGGGLVQPGGSLRL |
| | | SCVASGDVHKINFLGWYRQAPGKEREKVAHISIGDQTDYAD |
| | | SAKGRFTISRDESKNMVYLQMNSLKPEDTAVYFCRAFSRIYP |
| | | YDYWGQGTLVTVSSGGGGSTHTCPPCPAPPVAGPSVFLFPPK |
| | | PKDTLMISRTPEVTCVVVDVKHEDPEVKFNWYVDGVEVHN |
| | | AKTKPREEQYNSTYRVVSVLTVLHQDWLNGKEYKCKVSNK |
| | | ALPAPIEKTISKAKGQPREPQVCTLPPSREEMTKNQVSLSCAV |
| | | KGFYPSDIAVEWESNGQPENNYKTTPPVLDSDGSFFLVSKLT |
| | | VDKSRWQQGNVFSCSVMHEALHNRFTQKSLSLSPGK |
| 122 | TPP-42197- | EVQLVESGGGLVKPGGSLRLSCAASGFTFSSYSMNWVRQAP |
| | knob (CD8) | GKGLEWVSSISGQSNYIYYADSVKGRFTISRDNAKNSLYLQM |
| | BCMA HC | NSLRAEDTAVYYCARGGNYFVEYFQYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDKTGGSEVQLLESGGGLVQPGGSLRLS |
| | | CAASGFTFDDYAIGWFRQAPGKEREGVSCIRVSDGSTYYADS |
| | | VKGRFTISRDNSKNTLYLQMNSLRAEDTAVYYCAAGSLYTC |
| | | VQSIVWPARPYYDMDYWGQGTLVTVSSGGGGSTHTCPPCP |
| | | APPVAGPSVFLFPPKPKDTLMISRTPEVTCVVVDVKHEDPEV |
| | | KFNWYVDGVEVHNAKTKPREEQYNSTYRVVSVLTVLHQD |
| | | WLNGKEYKCKVSNKALPAPIEKTISKAKGQPREPQVYTLPPC |
| | | REEMTKNQVSLWCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| 123 | LM1953 | RNSAVWN |
| | STEAP2 HC | |
| | CDR1 | |
| 124 | LM1953 | RTYYRSKWYNDYAVSVKS |
| | STEAP2 HC | |
| | CDR2 | |
| 125 | LM1953 | GLRQNQFYYYMDV |
| | STEAP2 HC | |
| | CDR3 | |
| 126 | LM1953 | RASQSVASNLA |
| | STEAP2 LC | |
| | CDR1 | |
| 127 | LM1953 | GASTRAT |
| | STEAP2 LC | |
| | CDR2 | |
| 128 | LM1953 | QQYNNWPFT |
| | STEAP2 LC | |
| | CDR3 | |
| 129 | LM1953 | QVQLQQSGPGLVKPSQTLSLTCAISGDSVSRNSAVWNWIRQS |
| | STEAP2 VH | PSRGLEWLGRTYYRSKWYNDYAVSVKSRITINPDTSKNQFSL |
| | | QLNSVTPEDTAVYYCARGLRQNQFYYYMDVWGKGTTVTVS |
| | | S |
| 130 | LM1953 | EIVMTQSPATLSVSPGERATLSCRASQSVASNLAWYQQKPG |
| | STEAP2 VL | QAPRLLIYGASTRATGIPARFSGSGSGTEFTLTISSLQSEDFAV |
| | | YYCQQYNNWPFTFGPGTKVDIK |
| 131 | LM1953 | EIVMTQSPATLSVSPGERATLSCRASQSVASNLAWYQQKPG |
| | STEAP2 LC | QAPRLLIYGASTRATGIPARFSGSGSGTEFTLTISSLQSEDFAV |
| | | YYCQQYNNWPFTFGPGTKVDIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 132 | LM1953 | QVQLQQSGPGLVKPSQTLSLTCAISGDSVSRNSAVWNWIRQS |
| | STEAP2 VH- | PSRGLEWLGRTYYRSKWYNDYAVSVKSRITINPDTSKNQFSL |
| | CH1 | QLNSVTPEDTAVYYCARGLRQNQFYYYMDVWGKGTTVTVS |
| | | SASTKGPSVFPLAPSSKSTSGGTAALGCLVKDYFPEPVTVSW |
| | | NSGALTSGVHTFPAVLQSSGLYSLSSVVTVPSSSLGTQTYICN |
| | | VNHKPSNTKVDKRVEPKSCDK |
| 133 | LM1953 | QVQLQQSGPGLVKPSQTLSLTCAISGDSVSRNSAVWNWIRQS |
| | STEAP2 knob | PSRGLEWLGRTYYRSKWYNDYAVSVKSRITINPDTSKNQFSL |
| | (CD8) | QLNSVTPEDTAVYYCARGLRQNQFYYYMDVWGKGTTVTVS |
| | | SASTKGPSVFPLAPSSKSTSGGTAALGCLVKDYFPEPVTVSW |
| | | NSGALTSGVHTFPAVLQSSGLYSLSSVVTVPSSSLGTQTYICN |
| | | VNHKPSNTKVDKRVEPKSCDKTGGSEVQLLESGGGLVQPGG |
| | | SLRLSCAASGFTFDDYAIGWFRQAPGKEREGVSCIRVSDGST |
| | | YYADSVKGRFTISRDNSKNTLYLQMNSLRAEDTAVYYCAAG |
| | | SLYTCVQSIVWPARPYYDMDYWGQGTLVTVSSGGGGSTHT |
| | | CPPCPAPPVAGPSVFLFPPKPKDTLMISRTPEVTCVVVDVKHE |
| | | DPEVKFNWYVDGVEVHNAKTKPREEQYNSTYRVVSVLTVL |
| | | HQDWLNGKEYKCKVSNKALPAPIEKTISKAKGQPREPQVYT |
| | | LPPCREEMTKNQVSLWCLVKGFYPSDIAVEWESNGQPENNY |
| | | KTTPPVLDSDGSFFLYSKLTVDKSRWQQGNVFSCSVMHEAL |
| | | HNHYTQKSLSLSPGK |
| 134 | LM1953 | QVQLQQSGPGLVKPSQTLSLTCAISGDSVSRNSAVWNWIRQS |
| | STEAP2 hole | PSRGLEWLGRTYYRSKWYNDYAVSVKSRITINPDTSKNQFSL |
| | (TCR) | QLNSVTPEDTAVYYCARGLRQNQFYYYMDVWGKGTTVTVS |
| | | SASTKGPSVFPLAPSSKSTSGGTAALGCLVKDYFPEPVTVSW |
| | | NSGALTSGVHTFPAVLQSSGLYSLSSVVTVPSSSLGTQTYICN |
| | | VNHKPSNTKVDKRVEPKSCDKTGGSEVQLVESGGGLVQPGG |
| | | SLRLSCVASGDVHKINFLGWYRQAPGKEREKVAHISIGDQTD |
| | | YADSAKGRFTISRDESKNMVYLQMNSLKPEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNRFTQKSLSLSPGK |
| 135 | TPP-46956 | SYWIE |
| | TPP-46957 | |
| | TPP-46958 | |
| | TPP-46959 | |
| | TPP-46960 | |
| | TPP-47826 | |
| | TPP-49058 | |
| | LRRC15 VH | |
| | CDR1 | |
| 136 | TPP-46956 | EILPGSDTTNYAQNFQD |
| | LRRC15 VH | |
| | CDR2 | |
| 137 | TPP-46956 | DRGNYRAWFGY |
| | TPP-46957 | |
| | TPP-46958 | |
| | TPP-46959 | |
| | TPP-46960 | |
| | TPP-47826 | |
| | TPP-49058 | |
| | LRRC15 VH | |
| | CDR3 | |
| 138 | TPP-46956 | RASQDISNYLN |
| | TPP-46957 | |
| | TPP-46958 | |
| | TPP-46959 | |
| | TPP-46960 | |
| | TPP-47826 | |
| | TPP-49058 | |
| | LRRC15 VL | |
| | CDR1 | |
| 139 | TPP-46956 | YTSRLHS |
| | TPP-46957 | |
| | TPP-46958 | |
| | TPP-47826 | |
| | LRRC15 VL | |
| | CDR2 | |
| 140 | TPP-46956 | QQGNALPWT |
| | TPP-46957 | |
| | TPP-46958 | |
| | TPP-46959 | |
| | TPP-46960 | |
| | TPP-47826 | |
| | TPP-49058 | |
| | LRRC15 VL | |
| | CDR3 | |
| 141 | TPP-46956 | EVQLVQSGAEVKKPGASVKVSCKASGYKFSSYWIEWVKQA |
| | LRRC15 VH | PGQGLEWIGEILPGSDTTNYAQNFQDRVTFTSDTSISTAYMEL |
| | | SRLRSDDTAVYYCARDRGNYRAWFGYWGQGTLVTVSS |
| 142 | TPP-46956 | DIQMTQSPSSLSASVGDRVTITCRASQDISNYLNWYQQKPGK |
| | TPP-46957 | APKFLIYYTSRLHSGVPSRFSGSGSGTDYTLTISSLQPEDFATY |
| | TPP-46958 | YCQQGNALPWTFGGGTKVEIK |
| | TPP-47826 | |
| | LRRC15 VL | |
| 143 | TPP-46956 | DIQMTQSPSSLSASVGDRVTITCRASQDISNYLNWYQQKPGK |
| | TPP-46957 | APKFLIYYTSRLHSGVPSRFSGSGSGTDYTLTISSLQPEDFATY |
| | TPP-46958 | YCQQGNALPWTFGGGTKVEIKRTVAAPSVFIFPPSDEQLKSG |
| | TPP-47826 | TASVVCLLNNFYPREAKVQWKVDNALQSGNSQESVTEQDS |
| | LRRC15 | KDSTYSLSSTLTLSKADYEKHKVYACEVTHQGLSSPVTKSFN |
| | Light chain | RGEC |
| 144 | TPP-46956 | EVQLVQSGAEVKKPGASVKVSCKASGYKFSSYWIEWVKQA |
| | LRRC15 VH- | PGQGLEWIGEILPGSDTTNYAQNFQDRVTFTSDTSISTAYMEL |
| | CH1 | SRLRSDDTAVYYCARDRGNYRAWFGYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDK |
| 145 | TPP-46956 | EVQLVQSGAEVKKPGASVKVSCKASGYKFSSYWIEWVKQA |
| | LRRC15 hole | PGQGLEWIGEILPGSDTTNYAQNFQDRVTFTSDTSISTAYMEL |
| | (TCR) | SRLRSDDTAVYYCARDRGNYRAWFGYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDKTGGSEVQLVESGGGLVQPGGSLRL |
| | | SCVASGDVHKINFLGWYRQAPGKEREKVAHISIGDQTDYAD |
| | | SAKGRFTISRDESKNMVYLQMNSLKPEDTAVYFCRAFSRIYP |
| | | YDYWGQGTLVTVSSGGGGSTHTCPPCPAPPVAGPSVFLFPPK |
| | | PKDTLMISRTPEVTCVVVDVKHEDPEVKFNWYVDGVEVHN |
| | | AKTKPREEQYNSTYRVVSVLTVLHQDWLNGKEYKCKVSNK |
| | | ALPAPIEKTISKAKGQPREPQVCTLPPSREEMTKNQVSLSCAV |
| | | KGFYPSDIAVEWESNGQPENNYKTTPPVLDSDGSFFLVSKLT |
| | | VDKSRWQQGNVFSCSVMHEALHNRFTQKSLSLSPGK |
| 146 | TPP-46956 | EVQLVQSGAEVKKPGASVKVSCKASGYKFSSYWIEWVKQA |
| | LRRC15 | PGQGLEWIGEILPGSDTTNYAQNFQDRVTFTSDTSISTAYMEL |
| | knob (CD8) | SRLRSDDTAVYYCARDRGNYRAWFGYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDKTGGSEVQLLESGGGLVQPGGSLRLS |
| | | CAASGFTFTDYAIGWFRQAPGKEREGVSCIRVSDGSTYYADS |
| | | VKGRFTISRDSSKNTLYLQMNSLRAEDTAVYYCAAGSLYTC |
| | | VQSIVWPARPYYDMDYWGQGTLVTVSSGGGGSTHTCPPCP |
| | | APPVAGPSVFLFPPKPKDTLMISRTPEVTCVVVDVKHEDPEV |
| | | KFNWYVDGVEVHNAKTKPREEQYNSTYRVVSVLTVLHQD |
| | | WLNGKEYKCKVSNKALPAPIEKTISKAKGQPREPQVYTLPPC |
| | | REEMTKNQVSLWCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| 147 | TPP-46957 | EILPGSDTTNYAQQFQD |
| | LRRC15 VH | |
| | CDR2 | |
| 148 | TPP-46957 | EVQLVQSGAEVKKPGASVKVSCKASGYKFSSYWIEWVKQA |
| | LRRC15 VH | PGQGLEWIGEILPGSDTTNYAQQFQDRVTFTSDTSISTAYMEL |
| | | SRLRSDDTAVYYCARDRGNYRAWFGYWGQGTLVTVSS |
| 149 | TPP-46957 | EVQLVQSGAEVKKPGASVKVSCKASGYKFSSYWIEWVKQA |
| | LRRC15 VH- | PGQGLEWIGEILPGSDTTNYAQQFQDRVTFTSDTSISTAYMEL |
| | CH1 | SRLRSDDTAVYYCARDRGNYRAWFGYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDK |
| 150 | TPP-46957 | EVQLVQSGAEVKKPGASVKVSCKASGYKFSSYWIEWVKQA |
| | LRRC15 hole | PGQGLEWIGEILPGSDTTNYAQQFQDRVTFTSDTSISTAYMEL |
| | (TCR) | SRLRSDDTAVYYCARDRGNYRAWFGYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDKTGGSEVQLVESGGGLVQPGGSLRL |
| | | SCVASGDVHKINFLGWYRQAPGKEREKVAHISIGDQTDYAD |
| | | SAKGRFTISRDESKNMVYLQMNSLKPEDTAVYFCRAFSRIYP |
| | | YDYWGQGTLVTVSSGGGGSTHTCPPCPAPPVAGPSVFLFPPK |
| | | PKDTLMISRTPEVTCVVVDVKHEDPEVKFNWYVDGVEVHN |
| | | AKTKPREEQYNSTYRVVSVLTVLHQDWLNGKEYKCKVSNK |
| | | ALPAPIEKTISKAKGQPREPQVCTLPPSREEMTKNQVSLSCAV |
| | | KGFYPSDIAVEWESNGQPENNYKTTPPVLDSDGSFFLVSKLT |
| | | VDKSRWQQGNVFSCSVMHEALHNRFTQKSLSLSPGK |
| 151 | TPP-46957 | EVQLVQSGAEVKKPGASVKVSCKASGYKFSSYWIEWVKQA |
| | LRRC15 | PGQGLEWIGEILPGSDTTNYAQQFQDRVTFTSDTSISTAYMEL |
| | knob (CD8) | SRLRSDDTAVYYCARDRGNYRAWFGYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDKTGGSEVQLLESGGGLVQPGGSLRLS |
| | | CAASGFTFTDYAIGWFRQAPGKEREGVSCIRVSDGSTYYADS |
| | | VKGRFTISRDSSKNTLYLQMNSLRAEDTAVYYCAAGSLYTC |
| | | VQSIVWPARPYYDMDYWGQGTLVTVSSGGGGSTHTCPPCP |
| | | APPVAGPSVFLFPPKPKDTLMISRTPEVTCVVVDVKHEDPEV |
| | | KFNWYVDGVEVHNAKTKPREEQYNSTYRVVSVLTVLHQD |
| | | WLNGKEYKCKVSNKALPAPIEKTISKAKGQPREPQVYTLPPC |
| | | REEMTKNQVSLWCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| 152 | TPP-46958 | EILPGSDATNYAQKFQD |
| | TPP-47826 | |
| | LRRC15 VH | |
| | CDR2 | |
| 153 | TPP-46958 | EVQLVQSGAEVKKPGASVKVSCKASGYKFSSYWIEWVKQA |
| | TPP-47826 | PGQGLEWIGEILPGSDATNYAQKFQDRVTFTSDTSISTAYME |
| | LRRC15 VH | LSRLRSDDTAVYYCARDRGNYRAWFGYWGQGTLVTVSS |
| 154 | TPP-46958 | EVQLVQSGAEVKKPGASVKVSCKASGYKFSSYWIEWVKQA |
| | TPP-47826 | PGQGLEWIGEILPGSDATNYAQKFQDRVTFTSDTSISTAYME |
| | LRRC15 VH- | LSRLRSDDTAVYYCARDRGNYRAWFGYWGQGTLVTVSSAS |
| | CH1 | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDK |
| 155 | TPP-46958 | EVQLVQSGAEVKKPGASVKVSCKASGYKFSSYWIEWVKQA |
| | LRRC15 hole | PGQGLEWIGEILPGSDATNYAQKFQDRVTFTSDTSISTAYME |
| | (TCR) | LSRLRSDDTAVYYCARDRGNYRAWFGYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDKTGGSEVQLVESGGGLVQPGGSLR |
| | | LSCVASGDVHKINFLGWYRQAPGKEREKVAHISIGDQTDYA |
| | | DSAKGRFTISRDESKNMVYLQMNSLKPEDTAVYFCRAFSRIY |
| | | PYDYWGQGTLVTVSSGGGGSTHTCPPCPAPPVAGPSVFLFPP |
| | | KPKDTLMISRTPEVTCVVVDVKHEDPEVKFNWYVDGVEVH |
| | | NAKTKPREEQYNSTYRVVSVLTVLHQDWLNGKEYKCKVSN |
| | | KALPAPIEKTISKAKGQPREPQVCTLPPSREEMTKNQVSLSCA |
| | | VKGFYPSDIAVEWESNGQPENNYKTTPPVLDSDGSFFLVSKL |
| | | TVDKSRWQQGNVFSCSVMHEALHNRFTQKSLSLSPGK |
| 156 | TPP-46958 | EVQLVQSGAEVKKPGASVKVSCKASGYKFSSYWIEWVKQA |
| | TPP-47826 | PGQGLEWIGEILPGSDATNYAQKFQDRVTFTSDTSISTAYME |
| | LRRC15 | LSRLRSDDTAVYYCARDRGNYRAWFGYWGQGTLVTVSSAS |
| | knob (CD8) | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDKTGGSEVQLLESGGGLVQPGGSLR |
| | | LSCAASGFTFTDYAIGWFRQAPGKEREGVSCIRVSDGSTYYA |
| | | DSVKGRFTISRDSSKNTLYLQMNSLRAEDTAVYYCAAGSLY |
| | | TCVQSIVWPARPYYDMDYWGQGTLVTVSSGGGGSTHTCPP |
| | | CPAPPVAGPSVFLFPPKPKDTLMISRTPEVTCVVVDVKHEDP |
| | | EVKFNWYVDGVEVHNAKTKPREEQYNSTYRVVSVLTVLHQ |
| | | DWLNGKEYKCKVSNKALPAPIEKTISKAKGQPREPQVYTLPP |
| | | CREEMTKNQVSLWCLVKGFYPSDIAVEWESNGQPENNYKTT |
| | | PPVLDSDGSFFLYSKLTVDKSRWQQGNVFSCSVMHEALHNH |
| | | YTQKSLSLSPGK |
| 157 | TPP-46959 | EILPGSDTTNYAQKFQD |
| | TPP-46960 | |
| | TPP-49058 | |
| | LRRC15 VH | |
| | CDR2 | |
| 158 | TPP-46959 | YTSRLES |
| | TPP-49058 | |
| | LRRC15 VL | |
| | CDR2 | |
| 159 | TPP-46959 | EVQLVQSGAEVKKPGASVKVSCKASGYKFSSYWIEWVKQA |
| | TPP-46960 | PGQGLEWIGEILPGSDTTNYAQKFQDRVTFTSDTSISTAYMEL |
| | TPP-49058 | SRLRSDDTAVYYCARDRGNYRAWFGYWGQGTLVTVSS |
| | LRRC15 VH | |
| 160 | TPP-46959 | DIQMTQSPSSLSASVGDRVTITCRASQDISNYLNWYQQKPGK |
| | TPP-49058 | APKFLIYYTSRLESGVPSRFSGSGSGTDYTLTISSLQPEDFATY |
| | LRRC15 VL | YCQQGNALPWTFGGGTKVEIK |
| 161 | TPP-46959 | DIQMTQSPSSLSASVGDRVTITCRASQDISNYLNWYQQKPGK |
| | TPP-49058 | APKFLIYYTSRLESGVPSRFSGSGSGTDYTLTISSLQPEDFATY |
| | LRRC15 | YCQQGNALPWTFGGGTKVEIKRTVAAPSVFIFPPSDEQLKSG |
| | Light chain | TASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| 162 | TPP-46959 | EVQLVQSGAEVKKPGASVKVSCKASGYKFSSYWIEWVKQA |
| | TPP-46960 | PGQGLEWIGEILPGSDTTNYAQKFQDRVTFTSDTSISTAYMEL |
| | TPP-49058 | SRLRSDDTAVYYCARDRGNYRAWFGYWGQGTLVTVSSAST |
| | LRRC15 VH- | KGPSVFPLAPSSKSTSGGTAALGCLVKDYFPEPVTVSWNSGA |
| | CH1 | LTSGVHTFPAVLQSSGLYSLSSVVTVPSSSLGTQTYICNVNHK |
| | | PSNTKVDKRVEPKSCDK |
| 163 | TPP-46959 | EVQLVQSGAEVKKPGASVKVSCKASGYKFSSYWIEWVKQA |
| | TPP-46960 | PGQGLEWIGEILPGSDTTNYAQKFQDRVTFTSDTSISTAYMEL |
| | LRRC15 hole | SRLRSDDTAVYYCARDRGNYRAWFGYWGQGTLVTVSSAST |
| | (TCR) | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDKTGGSEVQLVESGGGLVQPGGSLRL |
| | | SCVASGDVHKINFLGWYRQAPGKEREKVAHISIGDQTDYAD |
| | | SAKGRFTISRDESKNMVYLQMNSLKPEDTAVYFCRAFSRTYP |
| | | YDYWGQGTLVTVSSGGGGSTHTCPPCPAPPVAGPSVFLFPPK |
| | | PKDTLMISRTPEVTCVVVDVKHEDPEVKFNWYVDGVEVHN |
| | | AKTKPREEQYNSTYRVVSVLTVLHQDWLNGKEYKCKVSNK |
| | | ALPAPIEKTISKAKGQPREPQVCTLPPSREEMTKNQVSLSCAV |
| | | KGFYPSDIAVEWESNGQPENNYKTTPPVLDSDGSFFLVSKLT |
| | | VDKSRWQQGNVFSCSVMHEALHNRFTQKSLSLSPGK |
| 164 | TPP-46959 | EVQLVQSGAEVKKPGASVKVSCKASGYKFSSYWIEWVKQA |
| | TPP-46960 | PGQGLEWIGEILPGSDTTNYAQKFQDRVTFTSDTSISTAYMEL |
| | TPP-49058 | SRLRSDDTAVYYCARDRGNYRAWFGYWGQGTLVTVSSAST |
| | LRRC15 | KGPSVFPLAPSSKSTSGGTAALGCLVKDYFPEPVTVSWNSGA |
| | knob (CD8) | LTSGVHTFPAVLQSSGLYSLSSVVTVPSSSLGTQTYICNVNHK |
| | | PSNTKVDKRVEPKSCDKTGGSEVQLLESGGGLVQPGGSLRLS |
| | | CAASGFTFTDYAIGWFRQAPGKEREGVSCIRVSDGSTYYADS |
| | | VKGRFTISRDSSKNTLYLQMNSLRAEDTAVYYCAAGSLYTC |
| | | VQSIVWPARPYYDMDYWGQGTLVTVSSGGGGSTHTCPPCP |
| | | APPVAGPSVFLFPPKPKDTLMISRTPEVTCVVVDVKHEDPEV |
| | | KFNWYVDGVEVHNAKTKPREEQYNSTYRVVSVLTVLHQD |
| | | WLNGKEYKCKVSNKALPAPIEKTISKAKGQPREPQVYTLPPC |
| | | REEMTKNQVSLWCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| 165 | TPP-46960 | YTSRLNS |
| | LRRC15 VL | |
| | CDR2 | |
| 166 | TPP-46960 | DIQMTQSPSSLSASVGDRVTITCRASQDISNYLNWYQQKPGK |
| | LRRC15 VL | APKFLIYYTSRLNSGVPSRFSGSGSGTDYTLTISSLQPEDFATY |
| | | YCQQGNALPWTFGGGTKVEIK |
| 167 | TPP-46960 | DIQMTQSPSSLSASVGDRVTITCRASQDISNYLNWYQQKPGK |
| | LRRC15 | APKFLIYYTSRLNSGVPSRFSGSGSGTDYTLTISSLQPEDFATY |
| | Light chain | YCQQGNALPWTFGGGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| 168 | CD8 V_{HHb} | EVQLLESGGGLVQPGGSLRLSCAASGFTFTDYAIGWFRQAPG |
| | D30T/N74S | KEREGVSCIRVSDGSTYYADSVKGRFTISRDSSKNTLYLQMN |
| | | SLRAEDTAVYYCAAGSLYTCVQSIVWPARPYYDMDYWGQG |
| | | TLVTVSS |
| 169 | CD8 _{VHHb} | GFTFTDYA |
| | D30T/N74S | |
| | CDR1 | |
| | IMGT | |
| 170 | CD8 V_{HHb} | IRVSDGST |
| | D30T/N74S | |
| | CDR2 | |
| | IMGT | |
| 171 | CD8 V_{HHb} | AAGSLYTCVQSIVWPARPYYDMDY |
| | D30T/N74S | |
| | CDR3 | |
| | IMGT | |
| 172 | TCR V_{HHa} | DVQLVESGGGVVQPGGSLRLSCVASGYVHKINFYGWYRQA |
| | alternative | PGKEREKVAHISIGDQTDYADSAKGRFTISRDESKNTVYLQM |
| | | NSLRPEDTAAYYCRALSRIWPYDYWGQGTLVTVSS |
| 173 | TCR V_{HHa} | INFYG |
| | CDR1 | |
| | alternative | |
| 174 | TCR V_{HHa} | HISIGDQTDYADSAKG |
| | CDR2 | |
| | alternative | |
| 175 | TCR V_{HHa} | LSRIWPYDY |
| | CDR3 | |
| | alternative | |
| 176 | TPP-47826 | EVQLVQSGAEVKKPGASVKVSCKASGYKFSSYWIEWVKQA |
| | LRRC15 hole | PGQGLEWIGEILPGSDATNYAQKFQDRVTFTSDTSISTAYME |
| | (TCR) | LSRLRSDDTAVYYCARDRGNYRAWFGYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDKTGGSDVQLVESGGGVVQPGGSLR |
| | | LSCVASGYVHKINFYGWYRQAPGKEREKVAHISIGDQTDYA |
| | | DSAKGRFTISRDESKNTVYLQMNSLRPEDTAAYYCRALSRIW |
| | | PYDYWGQGTLVTVSSGGGGSTHTCPPCPAPPVAGPSVFLFPP |
| | | KPKDTLMISRTPEVTCVVVDVKHEDPEVKFNWYVDGVEVH |
| | | NAKTKPREEQYNSTYRVVSVLTVLHQDWLNGKEYKCKVSN |
| | | KALPAPIEKTISKAKGQPREPQVCTLPPSREEMTKNQVSLSCA |
| | | VKGFYPSDIAVEWESNGQPENNYKTTPPVLDSDGSFFLVSKL |
| | | TVDKSRWQQGNVFSCSVMHEALHNRFTQKSLSLSPGK |
| 177 | TPP-49058 | EVQLVQSGAEVKKPGASVKVSCKASGYKFSSYWIEWVKQA |
| | LRRC15 hole | PGQGLEWIGEILPGSDTTNYAQKFQDRVTFTSDTSISTAYMEL |
| | (TCR) | SRLRSDDTAVYYCARDRGNYRAWFGYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDKTGGSDVQLVESGGGVVQPGGSLRL |
| | | SCVASGYVHKINFYGWYRQAPGKEREKVAHISIGDQTDYAD |
| | | SAKGRFTISRDESKNTVYLQMNSLRPEDTAAYYCRALSRIWP |
| | | YDYWGQGTLVTVSSGGGGSTHTCPPCPAPPVAGPSVFLFPPK |
| | | PKDTLMISRTPEVTCVVVDVKHEDPEVKFNWYVDGVEVHN |
| | | AKTKPREEQYNSTYRVVSVLTVLHQDWLNGKEYKCKVSNK |
| | | ALPAPIEKTISKAKGQPREPQVCTLPPSREEMTKNQVSLSCAV |
| | | KGFYPSDIAVEWESNGQPENNYKTTPPVLDSDGSFFLVSKLT |
| | | VDKSRWQQGNVFSCSVMHEALHNRFTQKSLSLSPGK |
| 178 | Comparator T | EVQLVESGGGLVQPGGSLRLSCAASGFTFNTYAMNWVRQA |
| | HC (CD3) | PGKGLEWVARIRSKYNNYATYYAASVKGRFTISRDDSKNSL |
| | | YLQMNSLKTEDTAVYYCARHGNFGNSYVSWFAYWGQGTL |
| | | VTVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEAAGGPSVFL |
| | | FPPKPKDTLMISRTPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFLLYS |
| | | KLTVDKSRWQEGNVFSCSVMHEALHNHYTQKSLSLSLGK |
| 179 | Comparator T | QTVVTQEPSLTVSPGGTVTLTCRSSTGAVTTSNYANWVQQK |
| | LC (CD3) | PGQAPRGLIGGTNKRAPGTPARFSGSLLGGKAALTLSGVQPE |
| | | DEAEYYCALWYSNLWVFGGGTKLTVLGQPKAAPSVTLFPPS |
| | | SEELQANKATLVCLISDFYPGAVTVAWKADSSPVKAGVETT |
| | | TPSKQSNNKYAASSYLSLTPEQWKSHRSYSCQVTHEGSTVE |
| | | KTVAPTECS |
| 180 | Comparator T | QLQLQESGPGLVKPSETLSLTCTVSGGSISSGSYFWGWIRQPP |
| | HC (BCMA) | GKGLEWIGSIYYSGITYYNPSLKSRVTISVDTSKNQFSLKLSS |
| | | VTAADTAVYYCARHDGAVAGLFDYWGQGTLVTVSSASTKG |
| | | PSVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEAAGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEK |
| | | TISKAKGQPREPQVYTLPPSQEEMTKNQVSLTCLVKGFYPSDI |
| | | AVEWESNGQPENNYKTTPPVLDSDGSFFLYSRLTVDKSRWQ |
| | | EGNVFSCSVMHEALHNHYTQKSLSLSLGK |
| 181 | Comparator T | SYVLTQPPSVSVAPGQTARITCGGNNIGSKSVHWYQQPPGQA |
| | LC (BCMA) | PVVVVYDDSDRPSGIPERFSGSNSGNTATLTISRVEAGDEAV |
| | | YYCQVWDSSSDHVVFGGGTKLTVLGQPKAAPSVTLFPPSSE |
| | | ELQANKATLVCLISDFYPGAVTVAWKGDSSPVKAGVETTTP |
| | | SKQSNNKYAASSYLSLTPEQWKSHRSYSCQVTHEGSTVEKT |
| | | VAPTECS |
| 182 | LM1486/LM1486- | SYNMH |
| | 2 CD20 | |
| | VH CDR1 | |
| | Kabat | |
| 183 | CD8 V_{HHb} | CIRVSDGSTYYADSVKG |
| | CDR2 | |
| | (alternate) | |
| | LM1653 | |
| 184 | GPC3 | CAAGTGCAGCTGGTGCAGTCTGGCGCCGAAGTGAAGAAA |
| | Heavy Chain | CCTGGCGCCTCTGTGAAGGTGTCCTGCAAGGCTTCTGGCT |
| | Hole (TCR) | ACACCTTTACCGACTACGAGATGCACTGGGTCCGACAGGC |
| | | TCCAGGACAAGGCTTGGAATGGATGGGCGCCCTGGATCCT |
| | | AAGACCGGCGATACCGCTTACTCCCAGAAATTCAAGGGCA |
| | | GAGTGACCCTGACCGCCGACAAGTCTACCTCCACCGCCTA |
| | | CATGGAACTGTCCAGCCTGACCTCTGAGGACACCGCCGTG |
| | | TACTACTGCACCCGGTTCTACTCCTACACCTACTGGGGCC |
| | | AGGGCACCCTGGTTACAGTGTCCTCTGCTTCCACCAAGGG |
| | | ACCCAGCGTTTTCCCTCTGGCTCCATCCTCCAAGTCCACCT |
| | | CTGGTGGAACAGCTGCTCTGGGCTGCCTGGTCAAGGACTA |
| | | CTTTCCTGAGCCTGTGACCGTGTCCTGGAACTCTGGCGCTC |
| | | TGACATCTGGCGTGCACACCTTTCCAGCTGTGCTGCAGTC |
| | | CTCCGGCCTGTACTCTCTGTCCTCTGTCGTGACCGTGCCTT |
| | | CCAGCTCTCTGGGAACCCAGACCTACATCTGCAATGTGAA |
| | | CCACAAGCCTTCCAATACCAAGGTGGACAAGAGAGTGGA |
| | | ACCCAAGTCCTGCGACAAGACAGGCGGATCTGAGGTCCA |
| | | GCTGGTCGAATCTGGCGGAGGATTGGTTCAGCCTGGCGGC |
| | | TCTCTGAGACTGTCTTGTGTGGCTAGCGGCGACGTGCACA |
| | | AGATCAACTTTCTCGGCTGGTACAGACAGGCCCCTGGCAA |
| | | AGAGAGAGAGAAGGTCGCCCACATCTCCATCGGCGACCA |
| | | GACCGATTACGCCGACTCTGCTAAGGGCAGATTCACCATC |
| | | TCTCGGGACGAGTCCAAGAACATGGTGTACCTGCAGATGA |
| | | ACTCCCTGAAGCCAGAGGATACCGCTGTGTATTTCTGCCG |
| | | GGCCTTCAGCCGGATCTACCCTTACGATTATTGGGGACAG |
| | | GGAACACTCGTGACAGTGTCTAGCGGAGGCGGAGGCTCT |
| | | ACCCATACCTGTCCTCCATGTCCTGCTCCTCCAGTGGCTGG |
| | | CCCTTCCGTGTTTCTGTTCCCTCCAAAGCCTAAGGACACCC |
| | | TGATGATCTCTCGGACCCCTGAAGTGACCTGCGTGGTGGT |
| | | GGATGTGAAGCACGAGGACCCAGAAGTGAAGTTCAATTG |
| | | GTACGTGGACGGCGTGGAAGTGCACAACGCCAAGACCAA |
| | | GCCTAGAGAGGAACAGTACAACTCCACCTACAGAGTGGT |
| | | GTCCGTGCTGACCGTGCTGCACCAGGATTGGCTGAACGGC |
| | | AAAGAGTACAAGTGCAAGGTGTCCAACAAGGCCCTGCCT |
| | | GCTCCTATCGAAAAGACCATCTCCAAGGCCAAGGGACAG |
| | | CCCCGGGAACCTCAAGTCTGTACCCTGCCTCCTAGCCGGG |
| | | AAGAGATGACCAAGAACCAGGTGTCCCTGTCCTGTGCCGT |
| | | GAAGGGCTTCTACCCCTCTGATATCGCCGTGGAATGGGAG |
| | | TCCAATGGCCAGCCTGAGAACAACTACAAGACCACACCTC |
| | | CTGTGCTGGACTCCGACGGCTCATTCTTCCTGGTGTCCAAG |
| | | CTGACAGTGGACAAGTCCAGATGGCAGCAGGGCAACGTG |
| | | TTCTCCTGCTCCGTGATGCACGAGGCCCTGCACAATCACT |
| | | ACACACAGAAGTCCCTGTCTCTGAGCCCCGGCAAA |
| 185 | LM1653 | CAAGTGCAGCTGGTGCAGTCTGGCGCCGAAGTGAAGAAA |
| | GPC3 | CCTGGCGCCTCTGTGAAGGTGTCCTGCAAGGCTTCTGGCT |
| | Heavy Chain | ACACCTTTACCGACTACGAGATGCACTGGGTCCGACAGGC |
| | Knob (CD8) | TCCAGGACAAGGCTTGGAATGGATGGGCGCCCTGGATCCT |
| | | AAGACCGGCGATACCGCTTACTCCCAGAAATTCAAGGGCA |
| | | GAGTGACCCTGACCGCCGACAAGTCTACCTCCACCGCCTA |
| | | CATGGAACTGTCCAGCCTGACCTCTGAGGACACCGCCGTG |
| | | TACTACTGCACCCGGTTCTACTCCTACACCTACTGGGGCC |
| | | AGGGCACCCTGGTTACAGTGTCCTCTGCTTCCACCAAGGG |
| | | ACCCAGCGTTTTCCCTCTGGCTCCATCCTCCAAGTCCACCT |
| | | CTGGTGGAACAGCTGCTCTGGGCTGCCTGGTCAAGGACTA |
| | | CTTTCCTGAGCCTGTGACCGTGTCCTGGAACTCTGGCGCTC |
| | | TGACATCTGGCGTGCACACCTTTCCAGCTGTGCTGCAGTC |
| | | CTCCGGCCTGTACTCTCTGTCCTCTGTCGTGACCGTGCCTT |
| | | CCAGCTCTCTGGGAACCCAGACCTACATCTGCAATGTGAA |
| | | CCACAAGCCTTCCAACACCAAGGTCGACAAGAGAGTGGA |
| | | ACCCAAGTCCTGCGACAAGACAGGCGGATCTGAGGTGCA |
| | | GCTGCTCGAATCTGGCGGAGGATTGGTTCAGCCTGGCGGC |
| | | TCTCTGAGACTGTCTTGTGCTGCCTCCGGCTTCACCTTCGA |
| | | CGATTACGCCATCGGCTGGTTCAGACAGGCCCCTGGCAAA |
| | | GAGAGAGAGGGCGTCAGCTGCATCAGAGTGTCTGACGGC |
| | | TCTACCTACTACGCCGACTCCGTGAAGGGCAGATTCACCA |
| | | TCTCTCGGGACAACTCCAAGAACACCCTGTACCTGCAGAT |
| | | GAACTCCCTGAGAGCCGAGGATACCGCTGTGTATTATTGC |
| | | GCCGCTGGCTCCCTGTATACCTGCGTGCAGTCTATCGTGTG |
| | | GCCCGCCAGACCTTACTACGACATGGACTATTGGGGACAG |
| | | GGAACACTCGTGACAGTGTCTAGCGGAGGCGGCGGATCT |
| | | ACCCATACCTGTCCTCCATGTCCTGCTCCTCCAGTGGCTGG |
| | | CCCTTCCGTGTTTCTGTTCCCTCCAAAGCCTAAGGACACCC |
| | | TGATGATCTCTCGGACCCCTGAAGTGACCTGCGTGGTGGT |
| | | GGATGTGAAGCACGAGGACCCAGAAGTGAAGTTCAATTG |
| | | GTACGTGGACGGCGTGGAAGTGCACAACGCCAAGACCAA |
| | | GCCTAGAGAGGAACAGTACAACTCCACCTACAGAGTGGT |
| | | GTCCGTGCTGACCGTGCTGCACCAGGATTGGCTGAACGGC |
| | | AAAGAGTACAAGTGCAAGGTGTCCAACAAGGCCCTGCCT |
| | | GCTCCTATCGAAAAGACCATCTCCAAGGCCAAGGGACAG |
| | | CCCAGGGAACCCCAGGTTTACACCCTGCCTCCATGCCGGG |
| | | AAGAGATGACCAAGAACCAGGTGTCCCTGTGGTGCCTGGT |
| | | TAAGGGCTTCTACCCCTCCGATATCGCCGTGGAATGGGAG |
| | | TCTAATGGCCAGCCTGAGAACAACTACAAGACCACACCTC |
| | | CTGTGCTGGACTCCGACGGCTCATTCTTCCTGTACTCCAAG |
| | | CTGACAGTGGACAAGTCCAGATGGCAGCAGGGCAACGTG |
| | | TTCTCCTGCTCCGTGATGCACGAGGCCCTGCACAATCACT |
| | | ACACACAGAAGTCCCTGTCTCTGAGCCCCGGCAAA |
| 186 | LM1653 | GACGTCGTGATGACCCAGTCTCCTCTGTCTCTGCCTGTGAC |
| | GPC3 | ACCTGGCGAGCCTGCCTCCATCTCTTGCAGATCTTCTCAGT |
| | Light Chain | CCCTGGTGCACTCCAACCGGAACACCTACCTGCACTGGTA |
| | | TCTGCAGAAGCCCGGACAGTCTCCCCAGCTGCTGATCTAC |
| | | AAGGTGTCCAACAGATTCTCTGGCGTGCCCGACAGATTCA |
| | | GCGGCTCTGGCTCTGGCACCGACTTCACCCTGAAGATCTC |
| | | TAGAGTGGAAGCCGAGGACGTGGGCGTGTACTACTGCTCT |
| | | CAGAATACCCACGTGCCACCTACCTTTGGCCAGGGCACCA |
| | | AGCTGGAAATCAAGAGAACCGTGGCCGCTCCTTCCGTGTT |
| | | CATCTTCCCACCATCTGACGAGCAGCTGAAGTCCGGCACA |
| | | GCTTCTGTCGTGTGCCTGCTGAACAACTTCTACCCTCGGGA |
| | | AGCCAAGGTGCAGTGGAAGGTGGACAATGCCCTGCAGTC |
| | | CGGCAACTCCCAAGAGTCTGTGACCGAGCAGGACTCCAA |
| | | GGACTCTACCTACAGCCTGTCCTCCACACTGACCCTGTCTA |
| | | AGGCCGACTACGAGAAGCACAAGGTGTACGCCTGTGAAG |
| | | TGACCCACCAGGGCCTGTCTAGCCCTGTGACCAAGTCTTT |
| | | CAACCGGGGCGAGTGC |
| 187 | LM1486-2 | GAGGTGCAGCTGGTTGAATCTGGCGGAGGATTGGTTCAGC |
| | CD20 | CTGGCGGCTCTCTGAGACTGTCTTGTGCTGCCTCTGGCTAC |
| | Heavy Chain | ACCTTCACCAGCTACAACATGCACTGGGTCCGACAGGCCC |
| | Hole (TCR) | CTGGCAAAGGATTGGAATGGGTCGGAGCTATCTACCCTGG |
| | | CTCCGGCGATACCTCCTACAACCAGAAGTTCAAGGGCAGA |
| | | TTCACCATCTCCGTGGACAAGTCCAAGAACACCCTGTACC |
| | | TGCAGATGAACTCCCTGAGAGCCGAGGACACCGCCGTGTA |
| | | CTATTGTGCCAGAGTGGTGTACTACTCCAACTCCTACTGGT |
| | | ACTTCGACGTGTGGGGCCAGGGAACACTGGTCACAGTGTC |
| | | CTCTGCTTCCACCAAGGGACCCTCTGTGTTCCCTCTGGCTC |
| | | CTTCCAGCAAGTCTACCTCTGGTGGAACCGCTGCTCTGGG |
| | | CTGCCTGGTCAAGGATTACTTTCCTGAGCCTGTGACCGTGT |
| | | CCTGGAATTCTGGTGCTCTGACCTCCGGCGTGCACACCTTT |
| | | CCAGCTGTGCTGCAGTCCTCTGGCCTGTACTCTCTGTCCTC |
| | | TGTCGTGACCGTGCCTTCTAGCTCTCTGGGCACCCAGACCT |
| | | ACATCTGCAACGTGAACCACAAGCCTTCCAACACCAAGGT |
| | | GGACAAGAGAGTGGAACCCAAGTCCTGCGATAAGACCGG |
| | | CGGATCTGAAGTCCAGTTGGTGGAAAGCGGAGGTGGACTT |
| | | GTGCAGCCAGGTGGAAGCCTGAGACTGAGTTGTGTGGCTT |
| | | CTGGCGACGTGCACAAGATCAACTTTCTCGGCTGGTACAG |
| | | GCAGGCTCCCGGCAAAGAAAGGGAAAAAGTGGCCCACAT |
| | | CTCCATCGGCGACCAGACCGATTACGCCGACTCTGCCAAA |
| | | GGCCGGTTTACCATCTCTCGGGACGAGAGCAAGAACATGG |
| | | TGTATCTCCAGATGAACAGTCTGAAGCCCGAGGATACAGC |
| | | TGTGTACTTCTGCCGGGCCTTCAGCCGGATCTACCCTTACG |
| | | ATTATTGGGGACAGGGCACCCTGGTTACCGTTTCTAGTGG |
| | | CGGAGGCGGCTCTACCCATACCTGTCCTCCATGTCCTGCTC |
| | | CACCTGTGGCCGGCCCTTCCGTGTTCCTGTTTCCTCCAAAG |
| | | CCTAAGGACACCCTGATGATCTCTCGGACCCCTGAAGTGA |
| | | CCTGCGTGGTGGTGGATGTGAAACACGAGGATCCCGAAGT |
| | | GAAGTTCAATTGGTACGTGGACGGCGTGGAAGTGCACAA |
| | | CGCCAAGACCAAGCCTAGAGAGGAACAGTACAACTCCAC |
| | | CTACCGCGTGGTGTCTGTGCTGACCGTTCTGCACCAGGAC |
| | | TGGCTGAACGGCAAAGAGTACAAGTGCAAGGTGTCCAAC |
| | | AAGGCCCTGCCTGCCCCCATCGAAAAGACCATCTCTAAGG |
| | | CTAAGGGCCAGCCTCGCGAGCCTCAAGTCTGTACACTGCC |
| | | TCCTAGCCGGGAAGAGATGACCAAGAATCAGGTGTCCCTG |
| | | TCCTGCGCCGTGAAGGGCTTCTACCCTTCTGATATCGCCGT |
| | | GGAATGGGAGTCCAACGGCCAGCCTGAGAACAACTACAA |
| | | GACCACACCTCCTGTGCTGGACTCCGACGGCTCATTCTTCC |
| | | TGGTGTCCAAGCTGACCGTGGATAAGAGCAGATGGCAGC |
| | | AGGGCAACGTGTTCTCCTGCTCTGTGATGCACGAGGCCCT |
| | | GCACAACCACTATACCCAGAAATCCCTGTCTCTGAGCCCT |
| | | GGCAAG |
| 188 | LM1486/ | GAGGTGCAGCTGGTGGAATCTGGCGGAGGATTGGTTCAGC |
| | LM1486-2 | CTGGCGGCTCTCTGAGACTGTCTTGTGCTGCCTCCGGCTAC |
| | CD20 | ACCTTCACCAGCTACAACATGCACTGGGTCCGACAGGCCC |
| | Heavy Chain | CTGGCAAAGGATTGGAATGGGTCGGAGCTATCTACCCCGG |
| | Knob (CD8) | CTCTGGCGACACCTCCTACAACCAGAAGTTCAAGGGCAGA |
| | | TTCACCATCTCCGTGGACAAGTCCAAGAACACCCTGTACC |
| | | TGCAGATGAACTCCCTGAGAGCCGAGGACACCGCCGTGTA |
| | | CTACTGTGCCAGAGTGGTGTACTACAGCAACTCCTACTGG |
| | | TACTTCGACGTGTGGGGCCAGGGCACACTGGTCACAGTTT |
| | | CTTCCGCCTCCACCAAGGGACCCAGCGTTTTCCCTCTGGCT |
| | | CCATCCTCCAAGTCTACCTCTGGCGGAACAGCTGCTCTGG |
| | | GCTGCCTGGTCAAGGACTACTTTCCTGAGCCTGTGACCGT |
| | | GTCCTGGAACTCTGGCGCTCTGACATCTGGCGTGCACACC |
| | | TTTCCAGCTGTGCTGCAGTCCTCCGGCCTGTACTCTCTGTC |
| | | CTCTGTCGTGACCGTGCCTTCCAGCTCTCTGGGAACCCAG |
| | | ACCTACATCTGCAATGTGAACCACAAGCCTTCCAACACCA |
| | | AGGTGGACAAGAGAGTGGAACCCAAGTCCTGCGATAAGA |
| | | CCGGCGGAAGCGAAGTGCAGCTGCTTGAAAGCGGAGGTG |
| | | GACTTGTGCAGCCAGGCGGAAGCCTGAGATTGTCCTGTGC |
| | | CGCTTCTGGCTTTACCTTCGACGACTACGCCATCGGCTGGT |
| | | TCAGACAGGCTCCCGGAAAAGAGAGAGAGGGCGTCAGCT |
| | | GCATCAGAGTGTCTGACGGCTCTACCTACTACGCCGACTC |
| | | CGTGAAAGGCCGGTTCACCATCAGCCGGGACAACAGCAA |
| | | GAATACTCTGTATCTCCAAATGAACAGCCTGCGCGCTGAG |
| | | GATACCGCTGTGTATTATTGCGCCGCTGGCTCCCTGTATAC |
| | | CTGCGTGCAGTCTATCGTGTGGCCCGCCAGACCTTACTAC |
| | | GACATGGATTACTGGGGACAGGGAACCCTGGTTACCGTGT |
| | | CTAGCGGCGGAGGCGGATCTACCCATACCTGTCCTCCATG |
| | | TCCTGCTCCACCTGTGGCCGGCCCTTCCGTGTTCCTGTTTC |
| | | CTCCAAAGCCTAAGGACACCCTGATGATCTCTCGGACCCC |
| | | TGAAGTGACCTGCGTGGTGGTGGATGTGAAACACGAGGA |
| | | TCCCGAAGTGAAGTTCAATTGGTACGTGGACGGCGTGGAA |
| | | GTGCACAACGCCAAGACCAAGCCTAGAGAGGAACAGTAC |
| | | AACTCCACCTACCGCGTGGTGTCCGTGCTGACCGTTCTGC |
| | | ATCAGGATTGGCTGAACGGCAAAGAGTACAAGTGCAAGG |
| | | TGTCCAACAAGGCCCTGCCTGCCCCCATCGAAAAGACCAT |
| | | CTCCAAGGCCAAGGGCCAGCCTAGGGAACCCCAGGTTTAC |
| | | ACCCTGCCACCTTGCCGGGAAGAGATGACCAAGAACCAG |
| | | GTGTCCCTGTGGTGCCTCGTGAAGGGCTTCTACCCTTCCGA |
| | | TATCGCCGTGGAATGGGAGAGCAATGGCCAGCCTGAGAA |
| | | CAACTACAAGACCACACCTCCTGTGCTGGACTCCGACGGC |
| | | TCATTCTTCCTGTACTCCAAGCTGACCGTGGATAAGTCTCG |
| | | GTGGCAGCAGGGCAACGTGTTCTCCTGCTCTGTGATGCAC |
| | | GAGGCCCTGCACAACCACTATACCCAGAAGTCCCTGTCTC |
| | | TGAGCCCCGGCAAG |
| 189 | LM1486/ | GACATCCAGATGACCCAGTCTCCATCCTCTCTGTCTGCCTC |
| | LM1486-2 | TGTGGGCGACAGAGTGACAATCACCTGTAGAGCCTCCAGC |
| | CD20 | TCCGTGTCCTACATGCACTGGTATCAGCAGAAGCCCGGCA |
| | Light Chain | AGGCCCCTAAGCCTCTGATCTACGCTCCTTCTAATCTGGCC |
| | | TCCGGCGTGCCCTCTAGATTTTCTGGCTCTGGATCTGGCAC |
| | | CGACTTCACCCTGACCATCAGTTCTCTGCAGCCTGAGGAC |
| | | TTCGCCACCTACTACTGCCAGCAGTGGTCTTTCAACCCTCC |
| | | TACCTTTGGCCAGGGCACCAAGGTGGAAATCAAGAGAAC |
| | | CGTGGCCGCTCCTTCCGTGTTCATCTTCCCACCATCTGACG |
| | | AGCAGCTGAAGTCCGGCACAGCTTCTGTCGTGTGCCTGCT |
| | | GAACAACTTCTACCCTCGGGAAGCCAAGGTGCAGTGGAA |
| | | GGTGGACAATGCCCTGCAGTCCGGCAACTCCCAAGAGTCT |
| | | GTGACCGAGCAGGACTCCAAGGACTCTACCTACAGCCTGT |
| | | CCTCCACACTGACCCTGTCTAAGGCCGACTACGAGAAGCA |
| | | CAAGGTGTACGCCTGTGAAGTGACCCACCAGGGACTGTCT |
| | | AGCCCCGTGACCAAGTCCTTCAACAGAGGCGAGTGT |
| 190 | LM1953 | CAAGTGCAGCTGCAGCAGTCAGGTCCAGGACTGGTGAAG |
| | STEAP2 | CCCTCGCAGACCCTCTCACTCACCTGTGCCATCTCCGGGG |
| | Heavy Chain | ACAGTGTCTCCAGAAACAGTGCTGTTTGGAATTGGATCAG |
| | Hole (TCR) | GCAGTCCCCATCGAGAGGCCTTGAGTGGCTGGGAAGGAC |
| | | ATACTACAGGTCCAAGTGGTATAATGATTATGCAGTATCT |
| | | GTGAAAAGTCGAATAACCATCAACCCAGACACATCCAAG |
| | | AACCAGTTCTCCCTGCAACTGAACTCTGTGACTCCCGAGG |
| | | ACACGGCTGTGTATTACTGTGCAAGGGGGTTACGACAGAA |
| | | CCAGTTCTACTACTACATGGACGTCTGGGGCAAAGGGACC |
| | | ACGGTCACCGTCTCCTCCGCTAGTACCAAGGGACCCAGCG |
| | | TGTTCCCTCTGGCACCTTCCAGCAAGTCTACCTCTGGCGGA |
| | | ACAGCCGCTCTGGGCTGTCTGGTCAAGGACTACTTTCCCG |
| | | AGCCTGTGACCGTGTCCTGGAATTCTGGCGCTCTGACCAG |
| | | CGGAGTGCATACCTTTCCAGCTGTGCTGCAGTCCTCCGGC |
| | | CTGTACTCTCTGTCCTCTGTCGTGACCGTGCCTTCCAGCTC |
| | | TCTGGGCACCCAGACCTACATCTGCAATGTGAACCACAAG |
| | | CCTTCCAACACCAAGGTGGACAAGAGGGTGGAACCCAAG |
| | | TCCTGCGACAAGACCGGCGGATCTGAAGTGCAGCTGGTCG |
| | | AGTCTGGCGGAGGATTGGTTCAACCTGGCGGCTCCCTGAG |
| | | ACTGTCTTGTGTGGCTAGCGGAGATGTGCATAAGATCAAT |
| | | TTTCTCGGCTGGTACAGACAGGCCCCTGGCAAAGAGAGAG |
| | | AGAAGGTCGCCCACATCTCCATCGGCGACCAGACCGATTA |
| | | CGCCGACTCTGCCAAGGGCAGATTCACCATCTCTCGGGAC |
| | | GAGTCCAAGAACATGGTGTACCTGCAGATGAACTCCCTGA |
| | | AGCCTGAGGATACCGCCGTGTACTTCTGCCGGGCCTTCTC |
| | | TCGGATCTACCCCTACGATTATTGGGGCCAGGGCACCCTG |
| | | GTCACAGTGTCATCTGGAGGCGGCGGATCTACTCACACGT |
| | | GCCCACCGTGCCCAGCACCTCCGGTGGCCGGACCGTCAGT |
| | | CTTCCTCTTCCCCCCAAAACCCAAGGACACCCTCATGATCT |
| | | CGCGAACCCCTGAGGTCACATGCGTGGTGGTGGACGTGAA |
| | | ACACGAAGACCCTGAGGTCAAGTTCAACTGGTACGTGGAC |
| | | GGCGTGGAGGTGCATAATGCCAAGACAAAGCCGCGGGAG |
| | | GAGCAGTACAACAGCACGTACCGTGTGGTCAGCGTCCTCA |
| | | CCGTCCTGCACCAGGACTGGCTGAATGGCAAGGAGTACA |
| | | AGTGCAAGGTCTCCAACAAAGCCCTCCCAGCCCCAATCGA |
| | | GAAAACCATCTCCAAAGCCAAAGGGCAGCCCCGAGAACC |
| | | ACAGGTCTGCACCCTGCCCCCATCCCGGGAGGAGATGACC |
| | | AAGAACCAGGTCAGCCTGAGCTGCGCGGTCAAAGGCTTCT |
| | | ATCCCAGCGACATCGCCGTGGAGTGGGAGAGCAATGGGC |
| | | AGCCGGAGAACAACTACAAGACCACGCCTCCCGTGCTGG |
| | | ACTCCGACGGCTCCTTCTTCCTCGTTAGCAAGCTCACCGTG |
| | | GACAAGAGCCGGTGGCAGCAGGGGAACGTCTTCTCATGCT |
| | | CCGTGATGCATGAGGCTCTGCACAACCGCTTCACGCAGAA |
| | | GAGCCTCTCCCTGTCTCCGGGTAAA |
| 191 | LM1953 | CAAGTGCAGCTGCAGCAGTCAGGTCCAGGACTGGTGAAG |
| | STEAP2 | CCCTCGCAGACCCTCTCACTCACCTGTGCCATCTCCGGGG |
| | Heavy Chain | ACAGTGTCTCCAGAAACAGTGCTGTTTGGAATTGGATCAG |
| | Knob (CD8) | GCAGTCCCCATCGAGAGGCCTTGAGTGGCTGGGAAGGAC |
| | | ATACTACAGGTCCAAGTGGTATAATGATTATGCAGTATCT |
| | | GTGAAAAGTCGAATAACCATCAACCCAGACACATCCAAG |
| | | AACCAGTTCTCCCTGCAACTGAACTCTGTGACTCCCGAGG |
| | | ACACGGCTGTGTATTACTGTGCAAGGGGGTTACGACAGAA |
| | | CCAGTTCTACTACTACATGGACGTCTGGGGCAAAGGGACC |
| | | ACGGTCACCGTCTCCTCCGCTAGCACCAAGGGACCCAGCG |
| | | TGTTCCCTCTGGCACCTTCCAGCAAGTCTACCTCTGGCGGA |
| | | ACAGCCGCTCTGGGCTGTCTGGTCAAGGACTACTTTCCCG |
| | | AGCCTGTGACCGTGTCCTGGAATTCTGGCGCTCTGACCAG |
| | | CGGAGTGCATACCTTTCCAGCTGTGCTGCAGTCCTCCGGC |
| | | CTGTACTCTCTGTCCTCTGTCGTGACCGTGCCTTCCAGCTC |
| | | TCTGGGCACCCAGACCTACATCTGCAATGTGAACCACAAG |
| | | CCTTCCAACACCAAGGTGGACAAGAGGGTGGAACCCAAG |
| | | TCCTGCGACAAGACCGGCGGATCTGAGGTCCAATTGTTGG |
| | | AATCTGGAGGGGGGCTCGTGCAGCCGGGGGGAAGTTTGC |
| | | GCCTTTCATGCGCTGCATCAGGGTTTACTTTCGACGACTAC |
| | | GCTATTGGCTGGTTCAGGCAAGCGCCGGGCAAGGAACGC |
| | | GAAGGCGTGTCCTGCATAAGAGTTTCAGATGGCAGTACCT |
| | | ATTACGCCGATAGCGTCAAGGGACGATTCACGATCAGCCG |
| | | GGATAATAGCAAAAATACACTTTATTTGCAAATGAACTCT |
| | | CTTAGGGCAGAAGATACTGCGGTCTATTATTGTGCGGCCG |
| | | GGTCCCTTTATACCTGCGTGCAGTCTATCGTATGGCCTGCT |
| | | AGGCCCTATTACGATATGGATTACTGGGGACAGGGTACAC |
| | | TCGTGACGGTATCCAGTGGAGGCGGCGGATCTACTCACAC |
| | | GTGCCCACCGTGCCCAGCACCTCCGGTGGCCGGACCGTCA |
| | | GTCTTCCTCTTCCCCCCAAAACCCAAGGACACCCTCATGA |
| | | TCTCCCGTACCCCTGAGGTCACATGCGTGGTGGTGGACGT |
| | | GAAACACGAAGACCCTGAGGTCAAGTTCAACTGGTACGT |
| | | GGACGGCGTGGAGGTGCATAATGCCAAGACAAAGCCGCG |
| | | GGAGGAGCAGTACAACAGCACGTACCGTGTGGTCAGCGT |
| | | CCTCACCGTCCTGCACCAGGACTGGCTGAATGGCAAGGAG |
| | | TACAAGTGCAAGGTCTCCAACAAAGCCCTCCCAGCCCCAA |
| | | TCGAGAAAACCATCTCCAAAGCCAAAGGGCAGCCCCGAG |
| | | AACCACAGGTCTACACCCTGCCCCCATGCCGGGAGGAGAT |
| | | GACCAAGAACCAGGTCAGCCTGTGGTGCCTGGTCAAAGG |
| | | CTTCTATCCCAGCGACATCGCCGTGGAGTGGGAGAGCAAT |
| | | GGGCAGCCGGAGAACAACTACAAGACCACGCCTCCCGTG |
| | | CTGGACTCCGACGGCTCCTTCTTCCTCTATAGCAAGCTCAC |
| | | CGTGGACAAGAGCCGGTGGCAGCAGGGGAACGTCTTCTC |
| | | ATGCTCCGTGATGCATGAGGCTCTGCACAACCACTACACG |
| | | CAGAAGAGCTTAAGCCTGTCTCCGGGTAAA |
| 192 | LM1953 | GAAATAGTGATGACGCAGTCTCCAGCCACCCTGTCTGTGT |
| | STEAP2 | CTCCCGGGGAAAGAGCCACCCTCTCCTGCCGAGCCAGTCA |
| | Light Chain | GAGTGTTGCCAGCAACTTAGCCTGGTACCAGCAGAAACCT |
| | | GGCCAGGCTCCCAGGCTCCTCATCTATGGTGCATCCACCA |
| | | GAGCCACTGGTATCCCAGCCAGGTTCAGTGGCAGTGGGTC |
| | | TGGGACAGAGTTCACTCTCACCATCAGCAGCCTGCAGTCT |
| | | GAAGATTTTGCAGTTTATTACTGTCAACAGTATAATAACT |
| | | GGCCATTCACTTTCGGCCCTGGGACCAAAGTGGATATCAA |
| | | ACGTACGGTGGCTGCACCATCTGTCTTCATCTTCCCGCCAT |
| | | CTGATGAGCAGTTGAAATCTGGAACTGCCTCTGTTGTGTG |
| | | CCTGCTGAATAACTTCTATCCCAGAGAGGCCAAAGTACAG |
| | | TGGAAGGTGGATAACGCCCTCCAATCGGGTAACTCCCAGG |
| | | AGAGTGTCACAGAGCAGGACAGCAAGGACAGCACCTACA |
| | | GCCTCAGCAGCACCCTGACGCTGAGCAAAGCAGACTACG |
| | | AGAAACACAAAGTCTACGCCTGCGAAGTCACCCATCAGG |
| | | GCCTGAGCTCGCCCGTCACAAAGAGCTTCAACAGGGGAG |
| | | AGTGT |
| 193 | TPP-42197- | GAGGTGCAGCTGGTGGAAAGTGGAGGTGGCCTGGTTAAG |
| | BCMA | CCTGGCGGGAGCCTGCGCCTGAGCTGCGCTGCCAGCGGCT |
| | Heavy Chain | TCACTTTCTCATCTTACAGCATGAACTGGGTGCGCCAGGC |
| | Knob (CD8) | CCCTGGCAAAGGCCTTGAGTGGGTTAGTAGCATCAGCGGC |
| | | CAGTCAAACTACATTTATTACGCCGATTCCGTCAAGGGCA |
| | | GGTTTACAATCTCCCGTGATAATGCGAAGAATAGCCTCTA |
| | | TCTCCAGATGAATTCCCTGCGCGCCGAAGATACAGCCGTG |
| | | TATTACTGCGCGAGGGGCGGGAATTACTTTGTTGAATACT |
| | | TCCAGTACTGGGGCCAGGGTACCCTCGTCACCGTTTCCAG |
| | | TGCAAGCACCAAGGGCCCATCCGTCTTCCCCCTGGCACCC |
| | | TCCTCCAAGAGCACCTCTGGGGGCACAGCGGCCCTGGGCT |
| | | GCCTGGTCAAGGACTACTTCCCCGAACCGGTGACGGTGTC |
| | | GTGGAACTCAGGCGCCCTGACCAGCGGCGTGCACACCTTC |
| | | CCGGCTGTCCTACAGTCCTCAGGACTCTACTCCCTCAGCA |
| | | GCGTGGTGACCGTGCCCTCCAGCAGCTTGGGCACCCAGAC |
| | | CTACATCTGCAACGTGAATCACAAGCCCAGCAACACCAAG |
| | | GTGGACAAGAGAGTTGAACCAAAAAGCTGCGACAAGACC |
| | | GGAGGATCTGAGGTCCAGCTGCTAGAGTCTGGCGGAGGA |
| | | TTGGTTCAGCCTGGCGGATCTCTGAGACTGTCTTGTGCCGC |
| | | CTCTGGCTTCACCTTCGACGATTACGCCATCGGCTGGTTCA |
| | | GACAGGCCCCTGGCAAAGAGAGAGAGGGCGTCAGCTGCA |
| | | TCAGAGTGTCTGACGGCTCCACCTACTACGCCGATTCTGT |
| | | GAAGGGCAGATTCACCATCTCCCGCGACAACTCCAAGAAC |
| | | ACCCTGTACCTGCAGATGAACTCCCTGCGGGCTGAGGACA |
| | | CCGCCGTGTACTATTGTGCTGCCGGCTCTCTGTACACCTGT |
| | | GTGCAGTCTATCGTGTGGCCCGCCAGACCTTACTACGACA |
| | | TGGACTATTGGGGCCAGGGCACCCTAGTGACCGTCTCAAG |
| | | CGGTGGCGGAGGATCTACCCACACCTGTCCTCCATGCCCT |
| | | GCTCCACCCGTGGCCGGCCCATCCGTGTTTCTGTTCCCTCC |
| | | AAAGCCTAAGGACACCCTGATGATCTCTCGGACCCCTGAA |
| | | GTGACCTGCGTGGTGGTGGATGTGAAGCACGAGGATCCCG |
| | | AAGTGAAGTTCAATTGGTACGTGGACGGCGTGGAAGTGC |
| | | ACAACGCCAAGACCAAGCCTAGAGAGGAACAGTACAACT |
| | | CCACCTACAGAGTGGTGTCCGTGCTGACCGTGCTGCACCA |
| | | GGATTGGCTGAACGGCAAAGAGTACAAGTGCAAGGTGTC |
| | | CAACAAGGCCCTGCCTGCCCCCATCGAAAAGACCATCTCC |
| | | AAGGCCAAGGGCCAGCCTAGGGAACCCCAGGTTTACACC |
| | | CTGCCACCTTGCCGGGAAGAGATGACCAAGAACCAGGTG |
| | | TCCCTGTGGTGCCTCGTGAAGGGCTTCTACCCTTCCGATAT |
| | | CGCCGTGGAATGGGAGAGCAATGGCCAGCCTGAGAACAA |
| | | CTACAAGACAACCCCTCCTGTGCTGGACTCCGACGGCTCA |
| | | TTCTTCCTGTACTCCAAGCTGACAGTGGACAAGTCCAGAT |
| | | GGCAGCAGGGCAACGTGTTCTCCTGCTCCGTGATGCACGA |
| | | GGCCCTGCACAATCACTACACCCAGAAGTCCCTGTCTCTG |
| | | TCCCCTGGAAAA |
| 194 | TPP-42197- | GAGGTGCAGCTGGTGGAAAGTGGAGGTGGCCTGGTTAAG |
| | BCMA | CCTGGCGGGAGCCTGCGCCTGAGCTGCGCTGCCAGCGGCT |
| | Heavy Chain | TCACTTTCTCATCTTACAGCATGAACTGGGTGCGCCAGGC |
| | Hole (TCR) | CCCTGGCAAAGGCCTTGAGTGGGTTAGTAGCATCAGCGGC |
| | | CAGTCAAACTACATTTATTACGCCGATTCCGTCAAGGGCA |
| | | GGTTTACAATCTCCCGTGATAATGCGAAGAATAGCCTCTA |
| | | TCTCCAGATGAATTCCCTGCGCGCCGAAGATACAGCCGTG |
| | | TATTACTGCGCGAGGGGCGGGAATTACTTTGTTGAATACT |
| | | TCCAGTACTGGGGCCAGGGTACCCTCGTCACCGTTTCCAG |
| | | TGCAAGCACCAAGGGCCCATCCGTCTTCCCCCTGGCACCC |
| | | TCCTCCAAGAGCACCTCTGGGGGCACAGCGGCCCTGGGCT |
| | | GCCTGGTCAAGGACTACTTCCCCGAACCGGTGACGGTGTC |
| | | GTGGAACTCAGGCGCCCTGACCAGCGGCGTGCACACCTTC |
| | | CCGGCTGTCCTACAGTCCTCAGGACTCTACTCCCTCAGCA |
| | | GCGTGGTGACCGTGCCCTCCAGCAGCTTGGGCACCCAGAC |
| | | CTACATCTGCAACGTGAATCACAAGCCCAGCAACACCAAG |
| | | GTGGACAAGAGAGTTGAACCAAAAAGCTGCGACAAGACC |
| | | GGAGGATCTGAAGTGCAGCTGGTCGAGTCTGGCGGAGGA |
| | | TTGGTTCAACCTGGCGGCTCCCTGAGACTGTCTTGTGTGGC |
| | | TAGCGGAGATGTGCATAAGATCAATTTTCTCGGCTGGTAC |
| | | AGACAGGCCCCTGGCAAAGAGAGAGAGAAGGTCGCCCAC |
| | | ATCTCCATCGGCGACCAGACCGATTACGCCGACTCTGCCA |
| | | AGGGCAGATTCACCATCTCTCGGGACGAGTCCAAGAACAT |
| | | GGTGTACCTGCAGATGAACTCCCTGAAGCCTGAGGATACC |
| | | GCCGTGTACTTCTGCCGGGCCTTCTCTCGGATCTACCCCTA |
| | | CGATTATTGGGGCCAGGGCACCCTGGTCACAGTGTCATCT |
| | | GGTGGCGGAGGATCTACCCACACCTGTCCTCCATGCCCTG |
| | | CTCCACCCGTGGCCGGCCCTTCCGTGTTCCTGTTTCCTCCA |
| | | AAGCCTAAGGACACCCTGATGATCTCTCGGACCCCTGAAG |
| | | TGACCTGCGTGGTGGTGGATGTGAAGCACGAGGATCCCGA |
| | | AGTGAAGTTCAATTGGTACGTGGACGGCGTGGAAGTGCAC |
| | | AACGCCAAGACCAAGCCTAGAGAGGAACAGTACAACTCC |
| | | ACCTACAGAGTGGTGTCCGTGCTGACCGTGCTGCACCAGG |
| | | ATTGGCTGAACGGCAAAGAGTACAAGTGCAAGGTGTCCA |
| | | ACAAGGCCCTGCCTGCCCCCATCGAAAAGACCATCTCTAA |
| | | GGCTAAGGGCCAGCCTCGCGAGCCTCAAGTCTGTACACTG |
| | | CCTCCTAGCCGGGAAGAGATGACCAAGAACCAGGTGTCC |
| | | CTGTCTTGCGCCGTGAAGGGCTTCTACCCTTCCGATATCGC |
| | | CGTGGAATGGGAGTCCAATGGCCAGCCTGAGAACAACTA |
| | | CAAGACAACCCCTCCTGTGCTGGACTCCGACGGCTCATTC |
| | | TTCCTGGTGTCCAAGCTGACAGTGGACAAGTCCAGATGGC |
| | | AGCAGGGCAACGTGTTCTCCTGCTCTGTGATGCACGAGGC |
| | | CCTGCACAACCGGTTCACCCAGAAATCCCTGTCTCTGTCC |
| | | CCTGGCAAG |
| 195 | TPP-42197- | GAGATTGTCCTGACACAGTCCCCTGGCACCCTTAGCCTGT |
| | BCMA | CCCCCGGTGAAAGAGCGACCCTGTCCTGCCGTGCCTCACA |
| | Light Chain | ATACATCTCTTCCAACAATCTGGCGTGGTACCAACAGAAG |
| | | CCCGGGCAAGCTCCCCGCCTCCTGATTTACGGGGCATCCA |
| | | ATAGAGCCACCGGGATCCCTGATAGATTCTCCGGCAGCGG |
| | | TAGCGGTACCGATTTCACCCTGACCATCTCTCGCCTGGAG |
| | | CCTGAAGACTTCGCGGTGTACTATTGTCAACAGTATGCTG |
| | | ATTCCCCAATCACATTCGGCCAGGGTACTAAGCTTGAAAT |
| | | CAAGCGGACAGTGGCCGCACCGTCCGTGTTCATCTTCCCA |
| | | CCTTCCGACGAGCAGCTGAAGTCTGGCACAGCCTCTGTCG |
| | | TGTGCCTGCTGAACAACTTCTACCCTCGGGAAGCCAAGGT |
| | | GCAGTGGAAGGTGGACAATGCCCTGCAGTCCGGCAACTCC |
| | | CAAGAGTCTGTGACCGAGCAGGACTCCAAGGACAGCACC |
| | | TACAGCCTGTCCTCCACACTGACCCTGTCCAAGGCCGACT |
| | | ACGAGAAGCACAAGGTGTACGCCTGCGAAGTGACCCATC |
| | | AGGGCCTGTCTAGCCCTGTGACCAAGTCTTTCAACCGGGG |
| | | CGAGTGC |
| 196 | LRC150016 | GAAGTGCAGCTGGTTCAGTCTGGCGCCGAAGTGAAGAAA |
| | LRRC15 | CCTGGCGCCTCTGTGAAGGTGTCCTGCAAGGCTTCCGGCT |
| | Heavy Chain | ACAAGTTCTCCAGCTACTGGATCGAGTGGGTCAAGCAGGC |
| | Knob (CD8) | TCCTGGACAGGGACTCGAGTGGATCGGAGAGATCCTGCCT |
| | | GGCTCTGACACCACCAACTACAACGAGAAGTTCAAGGAC |
| | | CGGGCCACCTTCACCTCCGACACCTCTATCAACACCGCCT |
| | | ACATGGAACTGTCCCGGCTGAGATCTGACGACACCGCCGT |
| | | GTACTACTGCGCCAGAGACAGAGGCAACTACAGAGCTTG |
| | | GTTTGGCTACTGGGGCCAGGGCACACTGGTCACAGTTTCT |
| | | TCTGCAAGCACCAAGGGACCCTCTGTGTTCCCTCTGGCTC |
| | | CTTCCAGCAAGTCTACCTCTGGCGGAACAGCTGCTCTGGG |
| | | CTGCCTGGTCAAGGACTACTTTCCTGAGCCTGTGACCGTG |
| | | TCCTGGAACTCTGGCGCTCTGACATCTGGCGTGCACACCT |
| | | TTCCAGCTGTGCTGCAGTCCTCCGGCCTGTACTCTCTGTCC |
| | | TCTGTCGTGACCGTGCCTTCCAGCTCTCTGGGAACCCAGA |
| | | CCTACATCTGCAATGTGAACCACAAGCCTTCCAACACCAA |
| | | GGTGGACAAGAGAGTGGAACCCAAGTCCTGCGATAAGAC |
| | | CGGCGGATCTGAGGTGCAGCTGTTGGAATCTGGCGGTGGA |
| | | TTGGTTCAGCCTGGCGGCTCTCTGAGACTGTCTTGTGCCGC |
| | | TTCTGGCTTCACCTTCGACGACTACGCCATCGGCTGGTTCA |
| | | GACAGGCCCCTGGCAAAGAGAGAGAGGGCGTCAGCTGCA |
| | | TCAGAGTGTCTGACGGCTCTACCTACTACGCCGACTCCGT |
| | | GAAGGGCAGATTCACCATCTCTCGGGACAACTCCAAGAAC |
| | | ACCCTGTACCTGCAGATGAACTCCCTGAGAGCCGAGGACA |
| | | CCGCCGTGTACTATTGTGCTGCTGGCTCCCTGTACACCTGT |
| | | GTGCAGTCTATCGTGTGGCCCGCCAGACCTTACTACGACA |
| | | TGGATTATTGGGGCCAGGGCACCCTGGTCACAGTTTCTAG |
| | | CGGAGGCGGAGGCAGCACCCATACCTGTCCTCCATGTCCT |
| | | GCTCCTCCAGTGGCTGGCCCTTCCGTGTTTCTGTTCCCTCC |
| | | AAAGCCTAAGGACACCCTGATGATCTCTCGGACCCCTGAA |
| | | GTGACCTGCGTGGTGGTGGATGTGAAGCACGAGGATCCCG |
| | | AAGTGAAGTTCAATTGGTACGTGGACGGCGTGGAAGTGC |
| | | ACAACGCCAAGACCAAGCCTAGAGAGGAACAGTACAACT |
| | | CCACCTACAGAGTGGTGTCCGTGCTGACCGTGCTGCACCA |
| | | GGATTGGCTGAACGGCAAAGAGTACAAGTGCAAGGTGTC |
| | | CAACAAGGCCCTGCCTGCTCCTATCGAAAAGACCATCTCC |
| | | AAGGCCAAGGGACAGCCCAGGGAACCCCAGGTTTACACC |
| | | CTGCCTCCATGCCGGGAAGAGATGACCAAGAACCAGGTG |
| | | TCCCTGTGGTGCCTGGTTAAGGGCTTCTACCCCTCCGATAT |
| | | CGCCGTGGAATGGGAGTCTAATGGCCAGCCTGAGAACAA |
| | | CTACAAGACCACACCTCCTGTGCTGGACTCCGACGGCTCA |
| | | TTCTTCCTGTACTCCAAGCTGACAGTGGACAAGTCCAGAT |
| | | GGCAGCAGGGCAACGTGTTCTCCTGCTCCGTGATGCACGA |
| | | GGCCCTGCACAATCACTACACCCAGAAGTCCCTGTCTCTG |
| | | AGCCCCGGCAAA |
| 197 | LRC150016 | GAAGTGCAGCTGGTTCAGTCTGGCGCCGAAGTGAAGAAA |
| | LRRC15 | CCTGGCGCCTCTGTGAAGGTGTCCTGCAAGGCTTCCGGCT |
| | Heavy Chain | ACAAGTTCTCCAGCTACTGGATCGAGTGGGTCAAGCAGGC |
| | Hole (TCR) | TCCTGGACAGGGACTCGAGTGGATCGGAGAGATCCTGCCT |
| | | GGCTCTGACACCACCAACTACAACGAGAAGTTCAAGGAC |
| | | CGGGCCACCTTCACCTCCGACACCTCTATCAACACCGCCT |
| | | ACATGGAACTGTCCCGGCTGAGATCTGACGACACCGCCGT |
| | | GTACTACTGCGCCAGAGACAGAGGCAACTACAGAGCTTG |
| | | GTTTGGCTACTGGGGCCAGGGCACACTGGTCACAGTTTCT |
| | | TCTGCAAGTACTAAGGGCCCCTCTGTGTTCCCTCTGGCTCC |
| | | TTCCAGCAAGTCTACCTCTGGCGGAACAGCTGCTCTGGGC |
| | | TGCCTGGTCAAGGACTACTTTCCTGAGCCTGTGACCGTGT |
| | | CCTGGAACTCTGGCGCTCTGACATCTGGCGTGCACACCTT |
| | | TCCAGCTGTGCTGCAGTCCTCCGGCCTGTACTCTCTGTCCT |
| | | CTGTCGTGACCGTGCCTTCCAGCTCTCTGGGAACCCAGAC |
| | | CTACATCTGCAATGTGAACCACAAGCCTTCCAACACCAAG |
| | | GTGGACAAGAGAGTGGAACCCAAGTCCTGCGATAAGACC |
| | | GGCGGATCTGAGGTGCAGCTGGTGGAATCTGGCGGAGGA |
| | | TTGGTTCAGCCTGGCGGCTCTCTGAGACTGTCTTGTGTGGC |
| | | TAGCGGCGACGTGCACAAGATCAACTTTCTCGGCTGGTAC |
| | | AGACAGGCCCCTGGCAAAGAGAGAGAGAAGGTCGCCCAC |
| | | ATCTCCATCGGCGACCAGACCGATTACGCCGACTCTGCCA |
| | | AGGGCAGATTCACCATCTCTCGGGACGAGTCCAAGAACAT |
| | | GGTGTACCTGCAGATGAACTCCCTGAAGCCTGAGGATACC |
| | | GCCGTGTACTTCTGCCGGGCCTTCTCTCGGATCTACCCCTA |
| | | CGATTATTGGGGCCAGGGCACCCTGGTCACAGTTTCTAGC |
| | | GGCGGAGGCGGCTCTACCCATACCTGTCCTCCATGTCCTG |
| | | CTCCTCCAGTGGCTGGCCCATCCGTGTTTCTGTTCCCTCCA |
| | | AAGCCTAAGGACACCCTGATGATCTCTCGGACCCCTGAAG |
| | | TGACCTGCGTGGTGGTGGATGTGAAGCACGAGGATCCCGA |
| | | AGTGAAGTTCAATTGGTACGTGGACGGCGTGGAAGTGCAC |
| | | AACGCCAAGACCAAGCCTAGAGAGGAACAGTACAACTCC |
| | | ACCTACAGAGTGGTGTCCGTGCTGACCGTGCTGCACCAGG |
| | | ATTGGCTGAACGGCAAAGAGTACAAGTGCAAGGTGTCCA |
| | | ACAAGGCCCTGCCTGCTCCTATCGAAAAGACCATCTCCAA |
| | | GGCCAAGGGCCAGCCTCGGGAACCTCAAGTCTGTACCCTG |
| | | CCTCCTAGCCGGGAAGAGATGACCAAGAACCAGGTGTCC |
| | | CTGTCCTGTGCCGTGAAGGGCTTCTACCCTTCCGATATCGC |
| | | CGTGGAATGGGAGAGCAATGGCCAGCCTGAGAACAACTA |
| | | CAAGACCACACCTCCTGTGCTGGACTCCGACGGCTCATTC |
| | | TTCCTGGTGTCCAAGCTGACAGTGGACAAGTCCAGATGGC |
| | | AGCAGGGCAACGTGTTCTCCTGCTCCGTGATGCACGAGGC |
| | | CCTGCACAACAGGTTCACCCAGAAGTCCCTGTCTCTGAGC |
| | | CCTGGCAAG |
| 198 | LRC150016 | GACATCCAGATGACCCAGTCTCCATCCTCTCTGTCCGCCTC |
| | LRRC15 | TGTGGGCGACAGAGTGACCATCACCTGTAGAGCCAGCCA |
| | Light Chain | GGACATCTCCAACTACCTGAACTGGTATCAGCAGAAACCT |
| | | GGCGGCGCTGTGAAGTTCCTGATCTACTACACCTCTCGGC |
| | | TGCACTCCGGCGTGCCCTCTAGATTTTCTGGCTCTGGATCC |
| | | GGCACCGACTATACCCTGACAATCTCCAGCCTGCAGCCTG |
| | | AGGACTTCGCTACCTACTTCTGCCAGCAAGGCGAGGCTCT |
| | | GCCTTGGACATTTGGCGGCGGAACAAAGGTGGAAATCAA |
| | | ACGAACTGTGGCTGCACCATCTGTCTTCATCTTCCCGCCAT |
| | | CTGATGAGCAGTTGAAATCTGGAACTGCCTCTGTTGTGTG |
| | | CCTGCTGAATAACTTCTATCCCAGAGAGGCCAAAGTACAG |
| | | TGGAAGGTGGATAACGCCCTCCAATCGGGTAACTCCCAGG |
| | | AGAGTGTCACAGAGCAGGACAGCAAGGACAGCACCTACA |
| | | GCCTCAGCAGCACCCTGACGCTGAGCAAAGCAGACTACG |
| | | AGAAACACAAAGTCTACGCCTGCGAAGTCACCCATCAGG |
| | | GCCTGAGCTCGCCCGTCACAAAGAGCTTCAACAGGGGAG |
| | | AGTGT |
| 199 | TPP-46956 | GAGGTGCAGTTGGTCCAATCAGGAGCAGAAGTCAAAAAA |
| | LRRC15 | CCCGGTGCTTCCGTTAAAGTATCTTGCAAAGCTAGCGGGT |
| | Heavy Chain | ACAAGTTCTCTTCCTATTGGATCGAGTGGGTTAAGCAGGC |
| | Knob (CD8) | ACCTGGACAGGGTCTTGAATGGATAGGCGAAATCCTTCCC |
| | | GGTAGCGACACCACAAACTATGCTCAAAAGTTCCAAGATA |
| | | GAGTTACTTTCACTTCAGATACTTCCATATCTACTGCATAT |
| | | ATGGAACTGTCCCGTTTGAGGAGTGATGACACAGCAGTGT |
| | | ACTACTGTGCCCGTGACCGTGGAAACTACCGTGCATGGTT |
| | | CGGCTATTGGGGTCAGGGTACACTCGTAACAGTGAGCTCA |
| | | GCAAGCACCAAGGGACCCTCTGTGTTCCCTCTGGCTCCTT |
| | | CCAGCAAGTCTACCTCTGGCGGAACAGCTGCTCTGGGCTG |
| | | CCTGGTCAAGGACTACTTTCCTGAGCCTGTGACCGTGTCCT |
| | | GGAACTCTGGCGCTCTGACATCTGGCGTGCACACCTTTCC |
| | | AGCTGTGCTGCAGTCCTCCGGCCTGTACTCTCTGTCCTCTG |
| | | TCGTGACCGTGCCTTCCAGCTCTCTGGGAACCCAGACCTA |
| | | CATCTGCAATGTGAACCACAAGCCTTCCAACACCAAGGTG |
| | | GACAAGAGAGTGGAACCCAAGTCCTGCGATAAGACCGGC |
| | | GGATCTGAGGTACAGCTGCTGGAATCAGGAGGCGGCTTG |
| | | GTTCAGCCAGGAGGCTCACTTCGGCTCTCCTGCGCAGCAA |
| | | GTGGGTTTACATTTACTGATTATGCTATAGGCTGGTTTCGG |
| | | CAAGCACCTGGTAAGGAACGCGAAGGTGTGTCATGTATTC |
| | | GCGTAAGCGACGGATCAACATACTATGCAGACAGTGTCA |
| | | AAGGTAGATTTACTATTAGTCGTGACAGCTCAAAGAATAC |
| | | TCTGTACTTGCAAATGAATTCCCTTCGTGCCGAAGATACT |
| | | GCCGTTTACTATTGCGCCGCAGGATCACTCTACACCTGCG |
| | | TGCAGAGCATTGTTTGGCCTGCCAGGCCTTATTATGATAT |
| | | GGACTATTGGGGCCAAGGCACTTTGGTCACTGTTAGCTCT |
| | | GGAGGCGGAGGCAGCACCCATACCTGTCCTCCATGTCCTG |
| | | CTCCTCCAGTGGCTGGCCCTTCCGTGTTTCTGTTCCCTCCA |
| | | AAGCCTAAGGACACCCTGATGATCTCTCGGACCCCTGAAG |
| | | TGACCTGCGTGGTGGTGGATGTGAAGCACGAGGATCCCGA |
| | | AGTGAAGTTCAATTGGTACGTGGACGGCGTGGAAGTGCAC |
| | | AACGCCAAGACCAAGCCTAGAGAGGAACAGTACAACTCC |
| | | ACCTACAGAGTGGTGTCCGTGCTGACCGTGCTGCACCAGG |
| | | ATTGGCTGAACGGCAAAGAGTACAAGTGCAAGGTGTCCA |
| | | ACAAGGCCCTGCCTGCTCCTATCGAAAAGACCATCTCCAA |
| | | GGCCAAGGGACAGCCCAGGGAACCCCAGGTTTACACCCT |
| | | GCCTCCATGCCGGGAAGAGATGACCAAGAACCAGGTGTC |
| | | CCTGTGGTGCCTGGTTAAGGGCTTCTACCCCTCCGATATCG |
| | | CCGTGGAATGGGAGTCTAATGGCCAGCCTGAGAACAACT |
| | | ACAAGACCACACCTCCTGTGCTGGACTCCGACGGCTCATT |
| | | CTTCCTGTACTCCAAGCTGACAGTGGACAAGTCCAGATGG |
| | | CAGCAGGGCAACGTGTTCTCCTGCTCCGTGATGCACGAGG |
| | | CCCTGCACAATCACTACACCCAGAAGTCCCTGTCTCTGAG |
| | | CCCCGGCAAA |
| 200 | TPP-46956 | GAGGTGCAGTTGGTCCAATCAGGAGCAGAAGTCAAAAAA |
| | LRRC15 | CCCGGTGCTTCCGTTAAAGTATCTTGCAAAGCTAGCGGGT |
| | Heavy Chain | ACAAGTTCTCTTCCTATTGGATCGAGTGGGTTAAGCAGGC |
| | Hole (TCR) | ACCTGGACAGGGTCTTGAATGGATAGGCGAAATCCTTCCC |
| | | GGTAGCGACACCACAAACTATGCTCAAAAGTTCCAAGATA |
| | | GAGTTACTTTCACTTCAGATACTTCCATATCTACTGCATAT |
| | | ATGGAACTGTCCCGTTTGAGGAGTGATGACACAGCAGTGT |
| | | ACTACTGTGCCCGTGACCGTGGAAACTACCGTGCATGGTT |
| | | CGGCTATTGGGGTCAGGGTACACTCGTAACAGTGAGCTCA |
| | | GCAAGTACTAAGGGCCCCTCTGTGTTCCCTCTGGCTCCTTC |
| | | CAGCAAGTCTACCTCTGGCGGAACAGCTGCTCTGGGCTGC |
| | | CTGGTCAAGGACTACTTTCCTGAGCCTGTGACCGTGTCCT |
| | | GGAACTCTGGCGCTCTGACATCTGGCGTGCACACCTTTCC |
| | | AGCTGTGCTGCAGTCCTCCGGCCTGTACTCTCTGTCCTCTG |
| | | TCGTGACCGTGCCTTCCAGCTCTCTGGGAACCCAGACCTA |
| | | CATCTGCAATGTGAACCACAAGCCTTCCAACACCAAGGTG |
| | | GACAAGAGAGTGGAACCCAAGTCCTGCGATAAGACCGGC |
| | | GGATCTGAGGTGCAGCTGGTGGAATCTGGCGGAGGATTG |
| | | GTTCAGCCTGGCGGCTCTCTGAGACTGTCTTGTGTGGCTA |
| | | GCGGCGACGTGCACAAGATCAACTTTCTCGGCTGGTACAG |
| | | ACAGGCCCCTGGCAAAGAGAGAGAGAAGGTCGCCCACAT |
| | | CTCCATCGGCGACCAGACCGATTACGCCGACTCTGCCAAG |
| | | GGCAGATTCACCATCTCTCGGGACGAGTCCAAGAACATGG |
| | | TGTACCTGCAGATGAACTCCCTGAAGCCTGAGGATACCGC |
| | | CGTGTACTTCTGCCGGGCCTTCTCTCGGATCTACCCCTACG |
| | | ATTATTGGGGCCAGGGCACCCTGGTCACAGTTTCTAGCGG |
| | | CGGAGGCGGCTCTACCCATACCTGTCCTCCATGTCCTGCTC |
| | | CTCCAGTGGCTGGCCCATCCGTGTTTCTGTTCCCTCCAAAG |
| | | CCTAAGGACACCCTGATGATCTCTCGGACCCCTGAAGTGA |
| | | CCTGCGTGGTGGTGGATGTGAAGCACGAGGATCCCGAAGT |
| | | GAAGTTCAATTGGTACGTGGACGGCGTGGAAGTGCACAA |
| | | CGCCAAGACCAAGCCTAGAGAGGAACAGTACAACTCCAC |
| | | CTACAGAGTGGTGTCCGTGCTGACCGTGCTGCACCAGGAT |
| | | TGGCTGAACGGCAAAGAGTACAAGTGCAAGGTGTCCAAC |
| | | AAGGCCCTGCCTGCTCCTATCGAAAAGACCATCTCCAAGG |
| | | CCAAGGGCCAGCCTCGGGAACCTCAAGTCTGTACCCTGCC |
| | | TCCTAGCCGGGAAGAGATGACCAAGAACCAGGTGTCCCT |
| | | GTCCTGTGCCGTGAAGGGCTTCTACCCTTCCGATATCGCC |
| | | GTGGAATGGGAGAGCAATGGCCAGCCTGAGAACAACTAC |
| | | AAGACCACACCTCCTGTGCTGGACTCCGACGGCTCATTCT |
| | | TCCTGGTGTCCAAGCTGACAGTGGACAAGTCCAGATGGCA |
| | | GCAGGGCAACGTGTTCTCCTGCTCCGTGATGCACGAGGCC |
| | | CTGCACAACAGGTTCACCCAGAAGTCCCTGTCTCTGAGCC |
| | | CTGGCAAG |
| 201 | TPP-46956 | GACATCCAGATGACCCAGAGTCCCTCTAGTCTCTCAGCTT |
| | LRRC15 | CCGTGGGGGACCGCGTCACTATAACTTGCCGTGCCTCACA |
| | Light Chain | AGACATATCAAACTACCTCAACTGGTATCAACAAAAGCCT |
| | | GGAAAAGCTCCTAAGTTTTTGATTTATTACACTTCCAGGCT |
| | | GGAGTCCGGTGTCCCATCCAGGTTCAGCGGCAGTGGAAGC |
| | | GGAACCGACTATACACTGACTATTTCTAGCTTGCAACCCG |
| | | AGGACTTTGCCACCTATTATTGCCAGCAAGGTAACGCCCT |
| | | GCCCTGGACCTTCGGTGGCGGAACCAAGGTGGAAATCAA |
| | | GCGGACAGTGGCCGCACCGTCCGTGTTCATCTTCCCACCT |
| | | TCCGACGAGCAGCTGAAGTCTGGCACAGCCTCTGTCGTGT |
| | | GCCTGCTGAACAACTTCTACCCTCGGGAAGCCAAGGTGCA |
| | | GTGGAAGGTGGACAATGCCCTGCAGTCCGGCAACTCCCAA |
| | | GAGTCTGTGACCGAGCAGGACTCCAAGGACAGCACCTAC |
| | | AGCCTGTCCTCCACACTGACCCTGTCCAAGGCCGACTACG |
| | | AGAAGCACAAGGTGTACGCCTGCGAAGTGACCCATCAGG |
| | | GCCTGTCTAGCCCTGTGACCAAGTCTTTCAACCGGGGCGA |
| | | GTGC |
| 202 | TPP-46957 | GAGGTTCAGTTGGTACAATCTGGAGCCGAGGTGAAAAAA |
| | LRRC15 | CCAGGTGCAAGCGTCAAGGTTTCCTGCAAAGCTAGTGGGT |
| | Heavy Chain | ACAAATTCAGTTCATACTGGATAGAGTGGGTTAAGCAAGC |
| | Knob (CD8) | TCCTGGACAAGGTTTGGAGTGGATTGGTGAAATCTTGCCT |
| | | GGCAGCGATACTACAAACTACGCTCAGCAGTTTCAGGACC |
| | | GCGTAACATTCACCTCTGATACATCTATATCAACTGCATAT |
| | | ATGGAACTTAGCAGGCTCAGGTCCGACGACACTGCCGTCT |
| | | ATTACTGCGCTCGGGATAGGGGGAATTATCGTGCTTGGTT |
| | | TGGTTATTGGGGTCAAGGGACACTGGTTACAGTTTCTTCA |
| | | GCAAGCACCAAGGGACCCTCTGTGTTCCCTCTGGCTCCTT |
| | | CCAGCAAGTCTACCTCTGGCGGAACAGCTGCTCTGGGCTG |
| | | CCTGGTCAAGGACTACTTTCCTGAGCCTGTGACCGTGTCCT |
| | | GGAACTCTGGCGCTCTGACATCTGGCGTGCACACCTTTCC |
| | | AGCTGTGCTGCAGTCCTCCGGCCTGTACTCTCTGTCCTCTG |
| | | TCGTGACCGTGCCTTCCAGCTCTCTGGGAACCCAGACCTA |
| | | CATCTGCAATGTGAACCACAAGCCTTCCAACACCAAGGTG |
| | | GACAAGAGAGTGGAACCCAAGTCCTGCGATAAGACCGGC |
| | | GGATCTGAGGTACAGCTGCTGGAATCAGGAGGCGGCTTG |
| | | GTTCAGCCAGGAGGCTCACTTCGGCTCTCCTGCGCAGCAA |
| | | GTGGGTTTACATTTACTGATTATGCTATAGGCTGGTTTCGG |
| | | CAAGCACCTGGTAAGGAACGCGAAGGTGTGTCATGTATTC |
| | | GCGTAAGCGACGGATCAACATACTATGCAGACAGTGTCA |
| | | AAGGTAGATTTACTATTAGTCGTGACAGCTCAAAGAATAC |
| | | TCTGTACTTGCAAATGAATTCCCTTCGTGCCGAAGATACT |
| | | GCCGTTTACTATTGCGCCGCAGGATCACTCTACACCTGCG |
| | | TGCAGAGCATTGTTTGGCCTGCCAGGCCTTATTATGATAT |
| | | GGACTATTGGGGCCAAGGCACTTTGGTCACTGTTAGCTCT |
| | | GGAGGCGGAGGCAGCACCCATACCTGTCCTCCATGTCCTG |
| | | CTCCTCCAGTGGCTGGCCCTTCCGTGTTTCTGTTCCCTCCA |
| | | AAGCCTAAGGACACCCTGATGATCTCTCGGACCCCTGAAG |
| | | TGACCTGCGTGGTGGTGGATGTGAAGCACGAGGATCCCGA |
| | | AGTGAAGTTCAATTGGTACGTGGACGGCGTGGAAGTGCAC |
| | | AACGCCAAGACCAAGCCTAGAGAGGAACAGTACAACTCC |
| | | ACCTACAGAGTGGTGTCCGTGCTGACCGTGCTGCACCAGG |
| | | ATTGGCTGAACGGCAAAGAGTACAAGTGCAAGGTGTCCA |
| | | ACAAGGCCCTGCCTGCTCCTATCGAAAAGACCATCTCCAA |
| | | GGCCAAGGGACAGCCCAGGGAACCCCAGGTTTACACCCT |
| | | GCCTCCATGCCGGGAAGAGATGACCAAGAACCAGGTGTC |
| | | CCTGTGGTGCCTGGTTAAGGGCTTCTACCCCTCCGATATCG |
| | | CCGTGGAATGGGAGTCTAATGGCCAGCCTGAGAACAACT |
| | | ACAAGACCACACCTCCTGTGCTGGACTCCGACGGCTCATT |
| | | CTTCCTGTACTCCAAGCTGACAGTGGACAAGTCCAGATGG |
| | | CAGCAGGGCAACGTGTTCTCCTGCTCCGTGATGCACGAGG |
| | | CCCTGCACAATCACTACACCCAGAAGTCCCTGTCTCTGAG |
| | | CCCCGGCAAA |
| 203 | TPP-46957 | GAGGTTCAGTTGGTACAATCTGGAGCCGAGGTGAAAAAA |
| | LRRC15 | CCAGGTGCAAGCGTCAAGGTTTCCTGCAAAGCTAGTGGGT |
| | Heavy Chain | ACAAATTCAGTTCATACTGGATAGAGTGGGTTAAGCAAGC |
| | Hole (TCR) | TCCTGGACAAGGTTTGGAGTGGATTGGTGAAATCTTGCCT |
| | | GGCAGCGATACTACAAACTACGCTCAGCAGTTTCAGGACC |
| | | GCGTAACATTCACCTCTGATACATCTATATCAACTGCATAT |
| | | ATGGAACTTAGCAGGCTCAGGTCCGACGACACTGCCGTCT |
| | | ATTACTGCGCTCGGGATAGGGGGAATTATCGTGCTTGGTT |
| | | TGGTTATTGGGGTCAAGGGACACTGGTTACAGTTTCTTCA |
| | | GCAAGTACTAAGGGCCCCTCTGTGTTCCCTCTGGCTCCTTC |
| | | CAGCAAGTCTACCTCTGGCGGAACAGCTGCTCTGGGCTGC |
| | | CTGGTCAAGGACTACTTTCCTGAGCCTGTGACCGTGTCCT |
| | | GGAACTCTGGCGCTCTGACATCTGGCGTGCACACCTTTCC |
| | | AGCTGTGCTGCAGTCCTCCGGCCTGTACTCTCTGTCCTCTG |
| | | TCGTGACCGTGCCTTCCAGCTCTCTGGGAACCCAGACCTA |
| | | CATCTGCAATGTGAACCACAAGCCTTCCAACACCAAGGTG |
| | | GACAAGAGAGTGGAACCCAAGTCCTGCGATAAGACCGGC |
| | | GGATCTGAGGTGCAGCTGGTGGAATCTGGCGGAGGATTG |
| | | GTTCAGCCTGGCGGCTCTCTGAGACTGTCTTGTGTGGCTA |
| | | GCGGCGACGTGCACAAGATCAACTTTCTCGGCTGGTACAG |
| | | ACAGGCCCCTGGCAAAGAGAGAGAGAAGGTCGCCCACAT |
| | | CTCCATCGGCGACCAGACCGATTACGCCGACTCTGCCAAG |
| | | GGCAGATTCACCATCTCTCGGGACGAGTCCAAGAACATGG |
| | | TGTACCTGCAGATGAACTCCCTGAAGCCTGAGGATACCGC |
| | | CGTGTACTTCTGCCGGGCCTTCTCTCGGATCTACCCCTACG |
| | | ATTATTGGGGCCAGGGCACCCTGGTCACAGTTTCTAGCGG |
| | | CGGAGGCGGCTCTACCCATACCTGTCCTCCATGTCCTGCTC |
| | | CTCCAGTGGCTGGCCCATCCGTGTTTCTGTTCCCTCCAAAG |
| | | CCTAAGGACACCCTGATGATCTCTCGGACCCCTGAAGTGA |
| | | CCTGCGTGGTGGTGGATGTGAAGCACGAGGATCCCGAAGT |
| | | GAAGTTCAATTGGTACGTGGACGGCGTGGAAGTGCACAA |
| | | CGCCAAGACCAAGCCTAGAGAGGAACAGTACAACTCCAC |
| | | CTACAGAGTGGTGTCCGTGCTGACCGTGCTGCACCAGGAT |
| | | TGGCTGAACGGCAAAGAGTACAAGTGCAAGGTGTCCAAC |
| | | AAGGCCCTGCCTGCTCCTATCGAAAAGACCATCTCCAAGG |
| | | CCAAGGGCCAGCCTCGGGAACCTCAAGTCTGTACCCTGCC |
| | | TCCTAGCCGGGAAGAGATGACCAAGAACCAGGTGTCCCT |
| | | GTCCTGTGCCGTGAAGGGCTTCTACCCTTCCGATATCGCC |
| | | GTGGAATGGGAGAGCAATGGCCAGCCTGAGAACAACTAC |
| | | AAGACCACACCTCCTGTGCTGGACTCCGACGGCTCATTCT |
| | | TCCTGGTGTCCAAGCTGACAGTGGACAAGTCCAGATGGCA |
| | | GCAGGGCAACGTGTTCTCCTGCTCCGTGATGCACGAGGCC |
| | | CTGCACAACAGGTTCACCCAGAAGTCCCTGTCTCTGAGCC |
| | | CTGGCAAG |
| 204 | TPP-46957 | GATATTCAGATGACACAATCCCCTAGCAGTCTCAGTGCCT |
| | LRRC15 | CAGTAGGGGACCGCGTCACAATAACATGTAGAGCCTCCCA |
| | Light Chain | GGATATTTCTAACTACTTGAACTGGTACCAGCAAAAGCCC |
| | | GGTAAAGCCCCTAAATTCCTTATTTACTACACAAGCCGAT |
| | | TGCACTCTGGTGTACCATCCAGGTTTAGTGGAAGCGGCTC |
| | | TGGTACAGATTACACACTGACCATTTCATCCCTGCAACCA |
| | | GAGGATTTCGCCACATATTACTGCCAACAGGGAAACGCCC |
| | | TCCCTTGGACCTTTGGTGGGGGAACTAAAGTTGAGATCAA |
| | | GCGGACAGTGGCCGCACCGTCCGTGTTCATCTTCCCACCT |
| | | TCCGACGAGCAGCTGAAGTCTGGCACAGCCTCTGTCGTGT |
| | | GCCTGCTGAACAACTTCTACCCTCGGGAAGCCAAGGTGCA |
| | | GTGGAAGGTGGACAATGCCCTGCAGTCCGGCAACTCCCAA |
| | | GAGTCTGTGACCGAGCAGGACTCCAAGGACAGCACCTAC |
| | | AGCCTGTCCTCCACACTGACCCTGTCCAAGGCCGACTACG |
| | | AGAAGCACAAGGTGTACGCCTGCGAAGTGACCCATCAGG |
| | | GCCTGTCTAGCCCTGTGACCAAGTCTTTCAACCGGGGCGA |
| | | GTGC |
| 205 | TPP-46958 | GAGGTGCAGCTTGTTCAAAGCGGTGCAGAGGTTAAGAAG |
| | LRRC15 | CCTGGAGCTAGCGTAAAAGTGTCCTGCAAAGCTAGTGGGT |
| | Heavy Chain | ATAAGTTCTCTAGCTACTGGATCGAGTGGGTGAAACAAGC |
| | Knob (CD8) | CCCAGGGCAGGGGTTGGAGTGGATTGGAGAAATACTCCC |
| | | CGGATCAGACGCCACTAACTATGCACAGAAGTTTCAAGAC |
| | | AGGGTTACCTTCACATCAGACACATCCATATCAACCGCAT |
| | | ATATGGAGTTGTCCAGGCTGCGGAGCGACGACACCGCTGT |
| | | CTATTATTGTGCCAGGGACCGGGGGAACTACAGGGCTTGG |
| | | TTTGGGTACTGGGGACAGGGTACACTCGTCACAGTGTCCT |
| | | CCGCAAGCACCAAGGGACCCTCTGTGTTCCCTCTGGCTCC |
| | | TTCCAGCAAGTCTACCTCTGGCGGAACAGCTGCTCTGGGC |
| | | TGCCTGGTCAAGGACTACTTTCCTGAGCCTGTGACCGTGT |
| | | CCTGGAACTCTGGCGCTCTGACATCTGGCGTGCACACCTT |
| | | TCCAGCTGTGCTGCAGTCCTCCGGCCTGTACTCTCTGTCCT |
| | | CTGTCGTGACCGTGCCTTCCAGCTCTCTGGGAACCCAGAC |
| | | CTACATCTGCAATGTGAACCACAAGCCTTCCAACACCAAG |
| | | GTGGACAAGAGAGTGGAACCCAAGTCCTGCGATAAGACC |
| | | GGCGGATCTGAGGTACAGCTGCTGGAATCAGGAGGCGGC |
| | | TTGGTTCAGCCAGGAGGCTCACTTCGGCTCTCCTGCGCAG |
| | | CAAGTGGGTTTACATTTACTGATTATGCTATAGGCTGGTTT |
| | | CGGCAAGCACCTGGTAAGGAACGCGAAGGTGTGTCATGT |
| | | ATTCGCGTAAGCGACGGATCAACATACTATGCAGACAGTG |
| | | TCAAAGGTAGATTTACTATTAGTCGTGACAGCTCAAAGAA |
| | | TACTCTGTACTTGCAAATGAATTCCCTTCGTGCCGAAGAT |
| | | ACTGCCGTTTACTATTGCGCCGCAGGATCACTCTACACCT |
| | | GCGTGCAGAGCATTGTTTGGCCTGCCAGGCCTTATTATGA |
| | | TATGGACTATTGGGGCCAAGGCACTTTGGTCACTGTTAGC |
| | | TCTGGAGGCGGAGGCAGCACCCATACCTGTCCTCCATGTC |
| | | CTGCTCCTCCAGTGGCTGGCCCTTCCGTGTTTCTGTTCCCT |
| | | CCAAAGCCTAAGGACACCCTGATGATCTCTCGGACCCCTG |
| | | AAGTGACCTGCGTGGTGGTGGATGTGAAGCACGAGGATC |
| | | CCGAAGTGAAGTTCAATTGGTACGTGGACGGCGTGGAAGT |
| | | GCACAACGCCAAGACCAAGCCTAGAGAGGAACAGTACAA |
| | | CTCCACCTACAGAGTGGTGTCCGTGCTGACCGTGCTGCAC |
| | | CAGGATTGGCTGAACGGCAAAGAGTACAAGTGCAAGGTG |
| | | TCCAACAAGGCCCTGCCTGCTCCTATCGAAAAGACCATCT |
| | | CCAAGGCCAAGGGACAGCCCAGGGAACCCCAGGTTTACA |
| | | CCCTGCCTCCATGCCGGGAAGAGATGACCAAGAACCAGG |
| | | TGTCCCTGTGGTGCCTGGTTAAGGGCTTCTACCCCTCCGAT |
| | | ATCGCCGTGGAATGGGAGTCTAATGGCCAGCCTGAGAAC |
| | | AACTACAAGACCACACCTCCTGTGCTGGACTCCGACGGCT |
| | | CATTCTTCCTGTACTCCAAGCTGACAGTGGACAAGTCCAG |
| | | ATGGCAGCAGGGCAACGTGTTCTCCTGCTCCGTGATGCAC |
| | | GAGGCCCTGCACAATCACTACACCCAGAAGTCCCTGTCTC |
| | | TGAGCCCCGGCAAA |
| 206 | TPP-46958 | GAGGTGCAGCTTGTTCAAAGCGGTGCAGAGGTTAAGAAG |
| | LRRC15 | CCTGGAGCTAGCGTAAAAGTGTCCTGCAAAGCTAGTGGGT |
| | Heavy Chain | ATAAGTTCTCTAGCTACTGGATCGAGTGGGTGAAACAAGC |
| | Hole (TCR) | CCCAGGGCAGGGGTTGGAGTGGATTGGAGAAATACTCCC |
| | | CGGATCAGACGCCACTAACTATGCACAGAAGTTTCAAGAC |
| | | AGGGTTACCTTCACATCAGACACATCCATATCAACCGCAT |
| | | ATATGGAGTTGTCCAGGCTGCGGAGCGACGACACCGCTGT |
| | | CTATTATTGTGCCAGGGACCGGGGGAACTACAGGGCTTGG |
| | | TTTGGGTACTGGGGACAGGGTACACTCGTCACAGTGTCCT |
| | | CCGCAAGTACTAAGGGCCCCTCTGTGTTCCCTCTGGCTCCT |
| | | TCCAGCAAGTCTACCTCTGGCGGAACAGCTGCTCTGGGCT |
| | | GCCTGGTCAAGGACTACTTTCCTGAGCCTGTGACCGTGTC |
| | | CTGGAACTCTGGCGCTCTGACATCTGGCGTGCACACCTTT |
| | | CCAGCTGTGCTGCAGTCCTCCGGCCTGTACTCTCTGTCCTC |
| | | TGTCGTGACCGTGCCTTCCAGCTCTCTGGGAACCCAGACC |
| | | TACATCTGCAATGTGAACCACAAGCCTTCCAACACCAAGG |
| | | TGGACAAGAGAGTGGAACCCAAGTCCTGCGATAAGACCG |
| | | GCGGATCTGAGGTGCAGCTGGTGGAATCTGGCGGAGGATT |
| | | GGTTCAGCCTGGCGGCTCTCTGAGACTGTCTTGTGTGGCT |
| | | AGCGGCGACGTGCACAAGATCAACTTTCTCGGCTGGTACA |
| | | GACAGGCCCCTGGCAAAGAGAGAGAGAAGGTCGCCCACA |
| | | TCTCCATCGGCGACCAGACCGATTACGCCGACTCTGCCAA |
| | | GGGCAGATTCACCATCTCTCGGGACGAGTCCAAGAACATG |
| | | GTGTACCTGCAGATGAACTCCCTGAAGCCTGAGGATACCG |
| | | CCGTGTACTTCTGCCGGGCCTTCTCTCGGATCTACCCCTAC |
| | | GATTATTGGGGCCAGGGCACCCTGGTCACAGTTTCTAGCG |
| | | GCGGAGGCGGCTCTACCCATACCTGTCCTCCATGTCCTGC |
| | | TCCTCCAGTGGCTGGCCCATCCGTGTTTCTGTTCCCTCCAA |
| | | AGCCTAAGGACACCCTGATGATCTCTCGGACCCCTGAAGT |
| | | GACCTGCGTGGTGGTGGATGTGAAGCACGAGGATCCCGA |
| | | AGTGAAGTTCAATTGGTACGTGGACGGCGTGGAAGTGCAC |
| | | AACGCCAAGACCAAGCCTAGAGAGGAACAGTACAACTCC |
| | | ACCTACAGAGTGGTGTCCGTGCTGACCGTGCTGCACCAGG |
| | | ATTGGCTGAACGGCAAAGAGTACAAGTGCAAGGTGTCCA |
| | | ACAAGGCCCTGCCTGCTCCTATCGAAAAGACCATCTCCAA |
| | | GGCCAAGGGCCAGCCTCGGGAACCTCAAGTCTGTACCCTG |
| | | CCTCCTAGCCGGGAAGAGATGACCAAGAACCAGGTGTCC |
| | | CTGTCCTGTGCCGTGAAGGGCTTCTACCCTTCCGATATCGC |
| | | CGTGGAATGGGAGAGCAATGGCCAGCCTGAGAACAACTA |
| | | CAAGACCACACCTCCTGTGCTGGACTCCGACGGCTCATTC |
| | | TTCCTGGTGTCCAAGCTGACAGTGGACAAGTCCAGATGGC |
| | | AGCAGGGCAACGTGTTCTCCTGCTCCGTGATGCACGAGGC |
| | | CCTGCACAACAGGTTCACCCAGAAGTCCCTGTCTCTGAGC |
| | | CCTGGCAAG |
| 207 | TPP-46958 | GATATTCAGATGACACAATCCCCTAGCAGTCTCAGTGCCT |
| | LRRC15 | CAGTAGGGGACCGCGTCACAATAACATGTAGAGCCTCCCA |
| | Light Chain | GGATATTTCTAACTACTTGAACTGGTACCAGCAAAAGCCC |
| | | GGTAAAGCCCCTAAATTCCTTATTTACTACACAAGCCGAT |
| | | TGCACTCTGGTGTACCATCCAGGTTTAGTGGAAGCGGCTC |
| | | TGGTACAGATTACACACTGACCATTTCATCCCTGCAACCA |
| | | GAGGATTTCGCCACATATTACTGCCAACAGGGAAACGCCC |
| | | TCCCTTGGACCTTTGGTGGGGGAACTAAAGTTGAGATCAA |
| | | GCGGACAGTGGCCGCACCGTCCGTGTTCATCTTCCCACCT |
| | | TCCGACGAGCAGCTGAAGTCTGGCACAGCCTCTGTCGTGT |
| | | GCCTGCTGAACAACTTCTACCCTCGGGAAGCCAAGGTGCA |
| | | GTGGAAGGTGGACAATGCCCTGCAGTCCGGCAACTCCCAA |
| | | GAGTCTGTGACCGAGCAGGACTCCAAGGACAGCACCTAC |
| | | AGCCTGTCCTCCACACTGACCCTGTCCAAGGCCGACTACG |
| | | AGAAGCACAAGGTGTACGCCTGCGAAGTGACCCATCAGG |
| | | GCCTGTCTAGCCCTGTGACCAAGTCTTTCAACCGGGGCGA |
| | | GTGC |
| 208 | TPP-46959 | GAGGTGCAGTTGGTCCAATCAGGAGCAGAAGTCAAAAAA |
| | LRRC15 | CCCGGTGCTTCCGTTAAAGTATCTTGCAAAGCTAGCGGGT |
| | Heavy Chain | ACAAGTTCTCTTCCTATTGGATCGAGTGGGTTAAGCAGGC |
| | Knob (CD8) | ACCTGGACAGGGTCTTGAATGGATAGGCGAAATCCTTCCC |
| | | GGTAGCGACACCACAAACTATGCTCAAAAGTTCCAAGATA |
| | | GAGTTACTTTCACTTCAGATACTTCCATATCTACTGCATAT |
| | | ATGGAACTGTCCCGTTTGAGGAGTGATGACACAGCAGTGT |
| | | ACTACTGTGCCCGTGACCGTGGAAACTACCGTGCATGGTT |
| | | CGGCTATTGGGGTCAGGGTACACTCGTAACAGTGAGCTCA |
| | | GCAAGCACCAAGGGACCCTCTGTGTTCCCTCTGGCTCCTT |
| | | CCAGCAAGTCTACCTCTGGCGGAACAGCTGCTCTGGGCTG |
| | | CCTGGTCAAGGACTACTTTCCTGAGCCTGTGACCGTGTCCT |
| | | GGAACTCTGGCGCTCTGACATCTGGCGTGCACACCTTTCC |
| | | AGCTGTGCTGCAGTCCTCCGGCCTGTACTCTCTGTCCTCTG |
| | | TCGTGACCGTGCCTTCCAGCTCTCTGGGAACCCAGACCTA |
| | | CATCTGCAATGTGAACCACAAGCCTTCCAACACCAAGGTG |
| | | GACAAGAGAGTGGAACCCAAGTCCTGCGATAAGACCGGC |
| | | GGATCTGAGGTACAGCTGCTGGAATCAGGAGGCGGCTTG |
| | | GTTCAGCCAGGAGGCTCACTTCGGCTCTCCTGCGCAGCAA |
| | | GTGGGTTTACATTTACTGATTATGCTATAGGCTGGTTTCGG |
| | | CAAGCACCTGGTAAGGAACGCGAAGGTGTGTCATGTATTC |
| | | GCGTAAGCGACGGATCAACATACTATGCAGACAGTGTCA |
| | | AAGGTAGATTTACTATTAGTCGTGACAGCTCAAAGAATAC |
| | | TCTGTACTTGCAAATGAATTCCCTTCGTGCCGAAGATACT |
| | | GCCGTTTACTATTGCGCCGCAGGATCACTCTACACCTGCG |
| | | TGCAGAGCATTGTTTGGCCTGCCAGGCCTTATTATGATAT |
| | | GGACTATTGGGGCCAAGGCACTTTGGTCACTGTTAGCTCT |
| | | GGAGGCGGAGGCAGCACCCATACCTGTCCTCCATGTCCTG |
| | | CTCCTCCAGTGGCTGGCCCTTCCGTGTTTCTGTTCCCTCCA |
| | | AAGCCTAAGGACACCCTGATGATCTCTCGGACCCCTGAAG |
| | | TGACCTGCGTGGTGGTGGATGTGAAGCACGAGGATCCCGA |
| | | AGTGAAGTTCAATTGGTACGTGGACGGCGTGGAAGTGCAC |
| | | AACGCCAAGACCAAGCCTAGAGAGGAACAGTACAACTCC |
| | | ACCTACAGAGTGGTGTCCGTGCTGACCGTGCTGCACCAGG |
| | | ATTGGCTGAACGGCAAAGAGTACAAGTGCAAGGTGTCCA |
| | | ACAAGGCCCTGCCTGCTCCTATCGAAAAGACCATCTCCAA |
| | | GGCCAAGGGACAGCCCAGGGAACCCCAGGTTTACACCCT |
| | | GCCTCCATGCCGGGAAGAGATGACCAAGAACCAGGTGTC |
| | | CCTGTGGTGCCTGGTTAAGGGCTTCTACCCCTCCGATATCG |
| | | CCGTGGAATGGGAGTCTAATGGCCAGCCTGAGAACAACT |
| | | ACAAGACCACACCTCCTGTGCTGGACTCCGACGGCTCATT |
| | | CTTCCTGTACTCCAAGCTGACAGTGGACAAGTCCAGATGG |
| | | CAGCAGGGCAACGTGTTCTCCTGCTCCGTGATGCACGAGG |
| | | CCCTGCACAATCACTACACCCAGAAGTCCCTGTCTCTGAG |
| | | CCCCGGCAAA |
| 209 | TPP-46959 | AGGTGCAGTTGGTCCAATCAGGAGCAGAAGTCAAAAAAC |
| | LRRC15 | CCGGTGCTTCCGTTAAAGTATCTTGCAAAGCTAGCGGGTA |
| | Heavy Chain | CAAGTTCTCTTCCTATTGGATCGAGTGGGTTAAGCAGGCA |
| | Hole (TCR) | CCTGGACAGGGTCTTGAATGGATAGGCGAAATCCTTCCCG |
| | | GTAGCGACACCACAAACTATGCTCAAAAGTTCCAAGATAG |
| | | AGTTACTTTCACTTCAGATACTTCCATATCTACTGCATATA |
| | | TGGAACTGTCCCGTTTGAGGAGTGATGACACAGCAGTGTA |
| | | CTACTGTGCCCGTGACCGTGGAAACTACCGTGCATGGTTC |
| | | GGCTATTGGGGTCAGGGTACACTCGTAACAGTGAGCTCAG |
| | | CAAGTACTAAGGGCCCCTCTGTGTTCCCTCTGGCTCCTTCC |
| | | AGCAAGTCTACCTCTGGCGGAACAGCTGCTCTGGGCTGCC |
| | | TGGTCAAGGACTACTTTCCTGAGCCTGTGACCGTGTCCTG |
| | | GAACTCTGGCGCTCTGACATCTGGCGTGCACACCTTTCCA |
| | | GCTGTGCTGCAGTCCTCCGGCCTGTACTCTCTGTCCTCTGT |
| | | CGTGACCGTGCCTTCCAGCTCTCTGGGAACCCAGACCTAC |
| | | ATCTGCAATGTGAACCACAAGCCTTCCAACACCAAGGTGG |
| | | ACAAGAGAGTGGAACCCAAGTCCTGCGATAAGACCGGCG |
| | | GATCTGAGGTGCAGCTGGTGGAATCTGGCGGAGGATTGGT |
| | | TCAGCCTGGCGGCTCTCTGAGACTGTCTTGTGTGGCTAGC |
| | | GGCGACGTGCACAAGATCAACTTTCTCGGCTGGTACAGAC |
| | | AGGCCCCTGGCAAAGAGAGAGAGAAGGTCGCCCACATCT |
| | | CCATCGGCGACCAGACCGATTACGCCGACTCTGCCAAGGG |
| | | CAGATTCACCATCTCTCGGGACGAGTCCAAGAACATGGTG |
| | | TACCTGCAGATGAACTCCCTGAAGCCTGAGGATACCGCCG |
| | | TGTACTTCTGCCGGGCCTTCTCTCGGATCTACCCCTACGAT |
| | | TATTGGGGCCAGGGCACCCTGGTCACAGTTTCTAGCGGCG |
| | | GAGGCGGCTCTACCCATACCTGTCCTCCATGTCCTGCTCCT |
| | | CCAGTGGCTGGCCCATCCGTGTTTCTGTTCCCTCCAAAGCC |
| | | TAAGGACACCCTGATGATCTCTCGGACCCCTGAAGTGACC |
| | | TGCGTGGTGGTGGATGTGAAGCACGAGGATCCCGAAGTG |
| | | AAGTTCAATTGGTACGTGGACGGCGTGGAAGTGCACAAC |
| | | GCCAAGACCAAGCCTAGAGAGGAACAGTACAACTCCACC |
| | | TACAGAGTGGTGTCCGTGCTGACCGTGCTGCACCAGGATT |
| | | GGCTGAACGGCAAAGAGTACAAGTGCAAGGTGTCCAACA |
| | | AGGCCCTGCCTGCTCCTATCGAAAAGACCATCTCCAAGGC |
| | | CAAGGGCCAGCCTCGGGAACCTCAAGTCTGTACCCTGCCT |
| | | CCTAGCCGGGAAGAGATGACCAAGAACCAGGTGTCCCTG |
| | | TCCTGTGCCGTGAAGGGCTTCTACCCTTCCGATATCGCCGT |
| | | GGAATGGGAGAGCAATGGCCAGCCTGAGAACAACTACAA |
| | | GACCACACCTCCTGTGCTGGACTCCGACGGCTCATTCTTCC |
| | | TGGTGTCCAAGCTGACAGTGGACAAGTCCAGATGGCAGC |
| | | AGGGCAACGTGTTCTCCTGCTCCGTGATGCACGAGGCCCT |
| | | GCACAACAGGTTCACCCAGAAGTCCCTGTCTCTGAGCCCT |
| | | GGCAAG |
| 210 | TPP-46959 | GACATCCAGATGACCCAGAGTCCCTCTAGTCTCTCAGCTT |
| | LRRC15 | CCGTGGGGGACCGCGTCACTATAACTTGCCGTGCCTCACA |
| | Light Chain | AGACATATCAAACTACCTCAACTGGTATCAACAAAAGCCT |
| | | GGAAAAGCTCCTAAGTTTTTGATTTATTACACTTCCAGGCT |
| | | GGAGTCCGGTGTCCCATCCAGGTTCAGCGGCAGTGGAAGC |
| | | GGAACCGACTATACACTGACTATTTCTAGCTTGCAACCCG |
| | | AGGACTTTGCCACCTATTATTGCCAGCAAGGTAACGCCCT |
| | | GCCCTGGACCTTCGGTGGCGGAACCAAGGTGGAAATCAA |
| | | GCGGACAGTGGCCGCACCGTCCGTGTTCATCTTCCCACCT |
| | | TCCGACGAGCAGCTGAAGTCTGGCACAGCCTCTGTCGTGT |
| | | GCCTGCTGAACAACTTCTACCCTCGGGAAGCCAAGGTGCA |
| | | GTGGAAGGTGGACAATGCCCTGCAGTCCGGCAACTCCCAA |
| | | GAGTCTGTGACCGAGCAGGACTCCAAGGACAGCACCTAC |
| | | AGCCTGTCCTCCACACTGACCCTGTCCAAGGCCGACTACG |
| | | AGAAGCACAAGGTGTACGCCTGCGAAGTGACCCATCAGG |
| | | GCCTGTCTAGCCCTGTGACCAAGTCTTTCAACCGGGGCGA |
| | | GTGC |
| 211 | TPP-46960 | GAGGTGCAGTTGGTCCAATCAGGAGCAGAAGTCAAAAAA |
| | LRRC15 | CCCGGTGCTTCCGTTAAAGTATCTTGCAAAGCTAGCGGGT |
| | Heavy Chain | ACAAGTTCTCTTCCTATTGGATCGAGTGGGTTAAGCAGGC |
| | Knob (CD8) | ACCTGGACAGGGTCTTGAATGGATAGGCGAAATCCTTCCC |
| | | GGTAGCGACACCACAAACTATGCTCAAAAGTTCCAAGATA |
| | | GAGTTACTTTCACTTCAGATACTTCCATATCTACTGCATAT |
| | | ATGGAACTGTCCCGTTTGAGGAGTGATGACACAGCAGTGT |
| | | ACTACTGTGCCCGTGACCGTGGAAACTACCGTGCATGGTT |
| | | CGGCTATTGGGGTCAGGGTACACTCGTAACAGTGAGCTCA |
| | | GCAAGCACCAAGGGACCCTCTGTGTTCCCTCTGGCTCCTT |
| | | CCAGCAAGTCTACCTCTGGCGGAACAGCTGCTCTGGGCTG |
| | | CCTGGTCAAGGACTACTTTCCTGAGCCTGTGACCGTGTCCT |
| | | GGAACTCTGGCGCTCTGACATCTGGCGTGCACACCTTTCC |
| | | AGCTGTGCTGCAGTCCTCCGGCCTGTACTCTCTGTCCTCTG |
| | | TCGTGACCGTGCCTTCCAGCTCTCTGGGAACCCAGACCTA |
| | | CATCTGCAATGTGAACCACAAGCCTTCCAACACCAAGGTG |
| | | GACAAGAGAGTGGAACCCAAGTCCTGCGATAAGACCGGC |
| | | GGATCTGAGGTACAGCTGCTGGAATCAGGAGGCGGCTTG |
| | | GTTCAGCCAGGAGGCTCACTTCGGCTCTCCTGCGCAGCAA |
| | | GTGGGTTTACATTTACTGATTATGCTATAGGCTGGTTTCGG |
| | | CAAGCACCTGGTAAGGAACGCGAAGGTGTGTCATGTATTC |
| | | GCGTAAGCGACGGATCAACATACTATGCAGACAGTGTCA |
| | | AAGGTAGATTTACTATTAGTCGTGACAGCTCAAAGAATAC |
| | | TCTGTACTTGCAAATGAATTCCCTTCGTGCCGAAGATACT |
| | | GCCGTTTACTATTGCGCCGCAGGATCACTCTACACCTGCG |
| | | TGCAGAGCATTGTTTGGCCTGCCAGGCCTTATTATGATAT |
| | | GGACTATTGGGGCCAAGGCACTTTGGTCACTGTTAGCTCT |
| | | GGAGGCGGAGGCAGCACCCATACCTGTCCTCCATGTCCTG |
| | | CTCCTCCAGTGGCTGGCCCTTCCGTGTTTCTGTTCCCTCCA |
| | | AAGCCTAAGGACACCCTGATGATCTCTCGGACCCCTGAAG |
| | | TGACCTGCGTGGTGGTGGATGTGAAGCACGAGGATCCCGA |
| | | AGTGAAGTTCAATTGGTACGTGGACGGCGTGGAAGTGCAC |
| | | AACGCCAAGACCAAGCCTAGAGAGGAACAGTACAACTCC |
| | | ACCTACAGAGTGGTGTCCGTGCTGACCGTGCTGCACCAGG |
| | | ATTGGCTGAACGGCAAAGAGTACAAGTGCAAGGTGTCCA |
| | | ACAAGGCCCTGCCTGCTCCTATCGAAAAGACCATCTCCAA |
| | | GGCCAAGGGACAGCCCAGGGAACCCCAGGTTTACACCCT |
| | | GCCTCCATGCCGGGAAGAGATGACCAAGAACCAGGTGTC |
| | | CCTGTGGTGCCTGGTTAAGGGCTTCTACCCCTCCGATATCG |
| | | CCGTGGAATGGGAGTCTAATGGCCAGCCTGAGAACAACT |
| | | ACAAGACCACACCTCCTGTGCTGGACTCCGACGGCTCATT |
| | | CTTCCTGTACTCCAAGCTGACAGTGGACAAGTCCAGATGG |
| | | CAGCAGGGCAACGTGTTCTCCTGCTCCGTGATGCACGAGG |
| | | CCCTGCACAATCACTACACCCAGAAGTCCCTGTCTCTGAG |
| | | CCCCGGCAAA |
| 212 | TPP-46960 | GAGGTGCAGTTGGTCCAATCAGGAGCAGAAGTCAAAAAA |
| | LRRC15 | CCCGGTGCTTCCGTTAAAGTATCTTGCAAAGCTAGCGGGT |
| | Heavy Chain | ACAAGTTCTCTTCCTATTGGATCGAGTGGGTTAAGCAGGC |
| | Hole (TCR) | ACCTGGACAGGGTCTTGAATGGATAGGCGAAATCCTTCCC |
| | | GGTAGCGACACCACAAACTATGCTCAAAAGTTCCAAGATA |
| | | GAGTTACTTTCACTTCAGATACTTCCATATCTACTGCATAT |
| | | ATGGAACTGTCCCGTTTGAGGAGTGATGACACAGCAGTGT |
| | | ACTACTGTGCCCGTGACCGTGGAAACTACCGTGCATGGTT |
| | | CGGCTATTGGGGTCAGGGTACACTCGTAACAGTGAGCTCA |
| | | GCAAGTACTAAGGGCCCCTCTGTGTTCCCTCTGGCTCCTTC |
| | | CAGCAAGTCTACCTCTGGCGGAACAGCTGCTCTGGGCTGC |
| | | CTGGTCAAGGACTACTTTCCTGAGCCTGTGACCGTGTCCT |
| | | GGAACTCTGGCGCTCTGACATCTGGCGTGCACACCTTTCC |
| | | AGCTGTGCTGCAGTCCTCCGGCCTGTACTCTCTGTCCTCTG |
| | | TCGTGACCGTGCCTTCCAGCTCTCTGGGAACCCAGACCTA |
| | | CATCTGCAATGTGAACCACAAGCCTTCCAACACCAAGGTG |
| | | GACAAGAGAGTGGAACCCAAGTCCTGCGATAAGACCGGC |
| | | GGATCTGAGGTGCAGCTGGTGGAATCTGGCGGAGGATTG |
| | | GTTCAGCCTGGCGGCTCTCTGAGACTGTCTTGTGTGGCTA |
| | | GCGGCGACGTGCACAAGATCAACTTTCTCGGCTGGTACAG |
| | | ACAGGCCCCTGGCAAAGAGAGAGAGAAGGTCGCCCACAT |
| | | CTCCATCGGCGACCAGACCGATTACGCCGACTCTGCCAAG |
| | | GGCAGATTCACCATCTCTCGGGACGAGTCCAAGAACATGG |
| | | TGTACCTGCAGATGAACTCCCTGAAGCCTGAGGATACCGC |
| | | CGTGTACTTCTGCCGGGCCTTCTCTCGGATCTACCCCTACG |
| | | ATTATTGGGGCCAGGGCACCCTGGTCACAGTTTCTAGCGG |
| | | CGGAGGCGGCTCTACCCATACCTGTCCTCCATGTCCTGCTC |
| | | CTCCAGTGGCTGGCCCATCCGTGTTTCTGTTCCCTCCAAAG |
| | | CCTAAGGACACCCTGATGATCTCTCGGACCCCTGAAGTGA |
| | | CCTGCGTGGTGGTGGATGTGAAGCACGAGGATCCCGAAGT |
| | | GAAGTTCAATTGGTACGTGGACGGCGTGGAAGTGCACAA |
| | | CGCCAAGACCAAGCCTAGAGAGGAACAGTACAACTCCAC |
| | | CTACAGAGTGGTGTCCGTGCTGACCGTGCTGCACCAGGAT |
| | | TGGCTGAACGGCAAAGAGTACAAGTGCAAGGTGTCCAAC |
| | | AAGGCCCTGCCTGCTCCTATCGAAAAGACCATCTCCAAGG |
| | | CCAAGGGCCAGCCTCGGGAACCTCAAGTCTGTACCCTGCC |
| | | TCCTAGCCGGGAAGAGATGACCAAGAACCAGGTGTCCCT |
| | | GTCCTGTGCCGTGAAGGGCTTCTACCCTTCCGATATCGCC |
| | | GTGGAATGGGAGAGCAATGGCCAGCCTGAGAACAACTAC |
| | | AAGACCACACCTCCTGTGCTGGACTCCGACGGCTCATTCT |
| | | TCCTGGTGTCCAAGCTGACAGTGGACAAGTCCAGATGGCA |
| | | GCAGGGCAACGTGTTCTCCTGCTCCGTGATGCACGAGGCC |
| | | CTGCACAACAGGTTCACCCAGAAGTCCCTGTCTCTGAGCC |
| | | CTGGCAAG |
| 213 | TPP-46960 | GACATACAGATGACTCAGTCTCCATCTTCCCTTTCCGCTTC |
| | LRRC15 | TGTGGGTGATCGAGTAACAATTACATGCCGGGCATCACAG |
| | Light Chain | GATATTTCTAACTATCTTAATTGGTATCAACAAAAACCAG |
| | | GTAAGGCTCCAAAGTTTCTGATCTATTATACCTCTCGGCTG |
| | | AACAGTGGTGTCCCCAGTAGGTTCAGCGGTAGTGGCAGCG |
| | | GAACTGACTACACCCTGACCATTTCTTCTCTGCAACCCGA |
| | | AGATTTTGCAACCTACTATTGTCAACAAGGCAACGCTCTT |
| | | CCTTGGACCTTTGGCGGAGGCACAAAGGTCGAAATAAAA |
| | | CGGACAGTGGCCGCACCGTCCGTGTTCATCTTCCCACCTTC |
| | | CGACGAGCAGCTGAAGTCTGGCACAGCCTCTGTCGTGTGC |
| | | CTGCTGAACAACTTCTACCCTCGGGAAGCCAAGGTGCAGT |
| | | GGAAGGTGGACAATGCCCTGCAGTCCGGCAACTCCCAAG |
| | | AGTCTGTGACCGAGCAGGACTCCAAGGACAGCACCTACA |
| | | GCCTGTCCTCCACACTGACCCTGTCCAAGGCCGACTACGA |
| | | GAAGCACAAGGTGTACGCCTGCGAAGTGACCCATCAGGG |
| | | CCTGTCTAGCCCTGTGACCAAGTCTTTCAACCGGGGCGAG |
| | | TGC |
| 214 | TPP-47826 | GAGGTGCAGCTTGTTCAAAGCGGTGCAGAGGTTAAGAAG |
| | LRRC15 | CCTGGAGCTAGCGTAAAAGTGTCCTGCAAAGCTAGTGGGT |
| | Heavy Chain | ATAAGTTCTCTAGCTACTGGATCGAGTGGGTGAAACAAGC |
| | Knob (CD8) | CCCAGGGCAGGGGTTGGAGTGGATTGGAGAAATACTCCC |
| | | CGGATCAGACGCCACTAACTATGCACAGAAGTTTCAAGAC |
| | | AGGGTTACCTTCACATCAGACACATCCATATCAACCGCAT |
| | | ATATGGAGTTGTCCAGGCTGCGGAGCGACGACACCGCTGT |
| | | CTATTATTGTGCCAGGGACCGGGGGAACTACAGGGCTTGG |
| | | TTTGGGTACTGGGGACAGGGTACACTCGTCACAGTGTCCT |
| | | CCGCAAGCACCAAGGGACCCTCTGTGTTCCCTCTGGCTCC |
| | | TTCCAGCAAGTCTACCTCTGGCGGAACAGCTGCTCTGGGC |
| | | TGCCTGGTCAAGGACTACTTTCCTGAGCCTGTGACCGTGT |
| | | CCTGGAACTCTGGCGCTCTGACATCTGGCGTGCACACCTT |
| | | TCCAGCTGTGCTGCAGTCCTCCGGCCTGTACTCTCTGTCCT |
| | | CTGTCGTGACCGTGCCTTCCAGCTCTCTGGGAACCCAGAC |
| | | CTACATCTGCAATGTGAACCACAAGCCTTCCAACACCAAG |
| | | GTGGACAAGAGAGTGGAACCCAAGTCCTGCGATAAGACC |
| | | GGCGGATCTGAGGTACAGCTGCTGGAATCAGGAGGCGGC |
| | | TTGGTTCAGCCAGGAGGCTCACTTCGGCTCTCCTGCGCAG |
| | | CAAGTGGGTTTACATTTACTGATTATGCTATAGGCTGGTTT |
| | | CGGCAAGCACCTGGTAAGGAACGCGAAGGTGTGTCATGT |
| | | ATTCGCGTAAGCGACGGATCAACATACTATGCAGACAGTG |
| | | TCAAAGGTAGATTTACTATTAGTCGTGACAGCTCAAAGAA |
| | | TACTCTGTACTTGCAAATGAATTCCCTTCGTGCCGAAGAT |
| | | ACTGCCGTTTACTATTGCGCCGCAGGATCACTCTACACCT |
| | | GCGTGCAGAGCATTGTTTGGCCTGCCAGGCCTTATTATGA |
| | | TATGGACTATTGGGGCCAAGGCACTTTGGTCACTGTTAGC |
| | | TCTGGAGGCGGAGGCAGCACCCATACCTGTCCTCCATGTC |
| | | CTGCTCCTCCAGTGGCTGGCCCTTCCGTGTTTCTGTTCCCT |
| | | CCAAAGCCTAAGGACACCCTGATGATCTCTCGGACCCCTG |
| | | AAGTGACCTGCGTGGTGGTGGATGTGAAGCACGAGGATC |
| | | CCGAAGTGAAGTTCAATTGGTACGTGGACGGCGTGGAAGT |
| | | GCACAACGCCAAGACCAAGCCTAGAGAGGAACAGTACAA |
| | | CTCCACCTACAGAGTGGTGTCCGTGCTGACCGTGCTGCAC |
| | | CAGGATTGGCTGAACGGCAAAGAGTACAAGTGCAAGGTG |
| | | TCCAACAAGGCCCTGCCTGCTCCTATCGAAAAGACCATCT |
| | | CCAAGGCCAAGGGACAGCCCAGGGAACCCCAGGTTTACA |
| | | CCCTGCCTCCATGCCGGGAAGAGATGACCAAGAACCAGG |
| | | TGTCCCTGTGGTGCCTGGTTAAGGGCTTCTACCCCTCCGAT |
| | | ATCGCCGTGGAATGGGAGTCTAATGGCCAGCCTGAGAAC |
| | | AACTACAAGACCACACCTCCTGTGCTGGACTCCGACGGCT |
| | | CATTCTTCCTGTACTCCAAGCTGACAGTGGACAAGTCCAG |
| | | ATGGCAGCAGGGCAACGTGTTCTCCTGCTCCGTGATGCAC |
| | | GAGGCCCTGCACAATCACTACACCCAGAAGTCCCTGTCTC |
| | | TGAGCCCCGGCAAA |
| 215 | TPP-47826 | GAAGTGCAGTTGGTGCAGTCTGGGGCCGAAGTTAAGAAA |
| | LRRC15 | CCAGGGGCCTCTGTTAAAGTAAGTTGCAAGGCTTCCGGTT |
| | Heavy Chain | ACAAGTTTTCAAGCTACTGGATCGAGTGGGTCAAGCAGGC |
| | Hole (TCR) | CCCAGGCCAAGGGTTGGAATGGATAGGAGAGATCCTTCC |
| | | AGGGAGCGATGCCACAAACTACGCCCAAAAATTCCAGGA |
| | | CAGGGTTACTTTTACTAGTGACACTTCCATATCAACAGCCT |
| | | ATATGGAGCTGTCCCGATTGCGCTCCGATGATACTGCCGT |
| | | GTACTATTGCGCCAGGGATAGAGGCAATTACCGCGCATGG |
| | | TTTGGATACTGGGGGCAGGGAACACTTGTGACAGTTAGCT |
| | | CCGCAAGCACCAAGGGCCCATCCGTCTTCCCCCTGGCACC |
| | | CTCCTCCAAGAGCACCTCTGGGGGCACAGCGGCCCTGGGC |
| | | TGCCTGGTCAAGGACTACTTCCCCGAACCGGTGACGGTGT |
| | | CGTGGAACTCAGGCGCCCTGACCAGCGGCGTGCACACCTT |
| | | CCCGGCTGTCCTACAGTCCTCAGGACTCTACTCCCTCAGC |
| | | AGCGTGGTGACCGTGCCCTCCAGCAGCTTGGGCACCCAGA |
| | | CCTACATCTGCAACGTGAATCACAAGCCCAGCAACACCAA |
| | | GGTGGACAAGAGAGTTGAACCAAAAAGCTGTGATAAAAC |
| | | CGGAGGTTCAGACGTGCAGTTGGTGGAATCTGGAGGCGGT |
| | | GTTGTACAGCCCGGCGGCTCTTTGAGACTCTCATGTGTCG |
| | | CCAGCGGCTACGTACACAAGATCAATTTTTATGGCTGGTA |
| | | TAGGCAAGCTCCCGGTAAAGAGCGCGAGAAAGTCGCTCA |
| | | TATTAGCATCGGAGATCAGACTGACTACGCCGACTCCGCT |
| | | AAAGGGCGCTTCACCATATCTAGGGATGAATCAAAGAAC |
| | | ACTGTGTATCTCCAGATGAACTCTCTCCGCCCAGAGGATA |
| | | CTGCCGCTTACTATTGTCGTGCTCTTTCCCGTATATGGCCT |
| | | TATGATTACTGGGGACAGGGGACATTGGTTACTGTGAGTT |
| | | CTGGTGGCGGAGGATCTACCCACACCTGTCCTCCATGCCC |
| | | TGCTCCACCCGTGGCCGGCCCTTCCGTGTTCCTGTTTCCTC |
| | | CAAAGCCTAAGGACACCCTGATGATCTCTCGGACCCCTGA |
| | | AGTGACCTGCGTGGTGGTGGATGTGAAGCACGAGGATCCC |
| | | GAAGTGAAGTTCAATTGGTACGTGGACGGCGTGGAAGTG |
| | | CACAACGCCAAGACCAAGCCTAGAGAGGAACAGTACAAC |
| | | TCCACCTACAGAGTGGTGTCCGTGCTGACCGTGCTGCACC |
| | | AGGATTGGCTGAACGGCAAAGAGTACAAGTGCAAGGTGT |
| | | CCAACAAGGCCCTGCCTGCCCCCATCGAAAAGACCATCTC |
| | | TAAGGCTAAGGGCCAGCCTCGCGAGCCTCAAGTCTGTACA |
| | | CTGCCTCCTAGCCGGGAAGAGATGACCAAGAACCAGGTG |
| | | TCCCTGTCTTGCGCCGTGAAGGGCTTCTACCCTTCCGATAT |
| | | CGCCGTGGAATGGGAGTCCAATGGCCAGCCTGAGAACAA |
| | | CTACAAGACAACCCCTCCTGTGCTGGACTCCGACGGCTCA |
| | | TTCTTCCTGGTGTCCAAGCTGACAGTGGACAAGTCCAGAT |
| | | GGCAGCAGGGCAACGTGTTCTCCTGCTCTGTGATGCACGA |
| | | GGCCCTGCACAACCGGTTCACCCAGAAATCCCTGTCTCTG |
| | | TCCCCTGGCAAG |
| 216 | TPP-47826 | GATATTCAGATGACACAATCCCCTAGCAGTCTCAGTGCCT |
| | LRRC15 | CAGTAGGGGACCGCGTCACAATAACATGTAGAGCCTCCCA |
| | Light Chain | GGATATTTCTAACTACTTGAACTGGTACCAGCAAAAGCCC |
| | | GGTAAAGCCCCTAAATTCCTTATTTACTACACAAGCCGAT |
| | | TGCACTCTGGTGTACCATCCAGGTTTAGTGGAAGCGGCTC |
| | | TGGTACAGATTACACACTGACCATTTCATCCCTGCAACCA |
| | | GAGGATTTCGCCACATATTACTGCCAACAGGGAAACGCCC |
| | | TCCCTTGGACCTTTGGTGGGGGAACTAAAGTTGAGATCAA |
| | | GCGGACAGTGGCCGCACCGTCCGTGTTCATCTTCCCACCT |
| | | TCCGACGAGCAGCTGAAGTCTGGCACAGCCTCTGTCGTGT |
| | | GCCTGCTGAACAACTTCTACCCTCGGGAAGCCAAGGTGCA |
| | | GTGGAAGGTGGACAATGCCCTGCAGTCCGGCAACTCCCAA |
| | | GAGTCTGTGACCGAGCAGGACTCCAAGGACAGCACCTAC |
| | | AGCCTGTCCTCCACACTGACCCTGTCCAAGGCCGACTACG |
| | | AGAAGCACAAGGTGTACGCCTGCGAAGTGACCCATCAGG |
| | | GCCTGTCTAGCCCTGTGACCAAGTCTTTCAACCGGGGCGA |
| | | GTGC |
| 217 | TPP-49058 | GAGGTGCAGTTGGTCCAATCAGGAGCAGAAGTCAAAAAA |
| | LRRC15 | CCCGGTGCTTCCGTTAAAGTATCTTGCAAAGCTAGCGGGT |
| | Heavy Chain | ACAAGTTCTCTTCCTATTGGATCGAGTGGGTTAAGCAGGC |
| | Knob (CD8) | ACCTGGACAGGGTCTTGAATGGATAGGCGAAATCCTTCCC |
| | | GGTAGCGACACCACAAACTATGCTCAAAAGTTCCAAGATA |
| | | GAGTTACTTTCACTTCAGATACTTCCATATCTACTGCATAT |
| | | ATGGAACTGTCCCGTTTGAGGAGTGATGACACAGCAGTGT |
| | | ACTACTGTGCCCGTGACCGTGGAAACTACCGTGCATGGTT |
| | | CGGCTATTGGGGTCAGGGTACACTCGTAACAGTGAGCTCA |
| | | GCAAGCACCAAGGGACCCTCTGTGTTCCCTCTGGCTCCTT |
| | | CCAGCAAGTCTACCTCTGGCGGAACAGCTGCTCTGGGCTG |
| | | CCTGGTCAAGGACTACTTTCCTGAGCCTGTGACCGTGTCCT |
| | | GGAACTCTGGCGCTCTGACATCTGGCGTGCACACCTTTCC |
| | | AGCTGTGCTGCAGTCCTCCGGCCTGTACTCTCTGTCCTCTG |
| | | TCGTGACCGTGCCTTCCAGCTCTCTGGGAACCCAGACCTA |
| | | CATCTGCAATGTGAACCACAAGCCTTCCAACACCAAGGTG |
| | | GACAAGAGAGTGGAACCCAAGTCCTGCGATAAGACCGGC |
| | | GGATCTGAGGTACAGCTGCTGGAATCAGGAGGCGGCTTG |
| | | GTTCAGCCAGGAGGCTCACTTCGGCTCTCCTGCGCAGCAA |
| | | GTGGGTTTACATTTACTGATTATGCTATAGGCTGGTTTCGG |
| | | CAAGCACCTGGTAAGGAACGCGAAGGTGTGTCATGTATTC |
| | | GCGTAAGCGACGGATCAACATACTATGCAGACAGTGTCA |
| | | AAGGTAGATTTACTATTAGTCGTGACAGCTCAAAGAATAC |
| | | TCTGTACTTGCAAATGAATTCCCTTCGTGCCGAAGATACT |
| | | GCCGTTTACTATTGCGCCGCAGGATCACTCTACACCTGCG |
| | | TGCAGAGCATTGTTTGGCCTGCCAGGCCTTATTATGATAT |
| | | GGACTATTGGGGCCAAGGCACTTTGGTCACTGTTAGCTCT |
| | | GGAGGCGGAGGCAGCACCCATACCTGTCCTCCATGTCCTG |
| | | CTCCTCCAGTGGCTGGCCCTTCCGTGTTTCTGTTCCCTCCA |
| | | AAGCCTAAGGACACCCTGATGATCTCTCGGACCCCTGAAG |
| | | TGACCTGCGTGGTGGTGGATGTGAAGCACGAGGATCCCGA |
| | | AGTGAAGTTCAATTGGTACGTGGACGGCGTGGAAGTGCAC |
| | | AACGCCAAGACCAAGCCTAGAGAGGAACAGTACAACTCC |
| | | ACCTACAGAGTGGTGTCCGTGCTGACCGTGCTGCACCAGG |
| | | ATTGGCTGAACGGCAAAGAGTACAAGTGCAAGGTGTCCA |
| | | ACAAGGCCCTGCCTGCTCCTATCGAAAAGACCATCTCCAA |
| | | GGCCAAGGGACAGCCCAGGGAACCCCAGGTTTACACCCT |
| | | GCCTCCATGCCGGGAAGAGATGACCAAGAACCAGGTGTC |
| | | CCTGTGGTGCCTGGTTAAGGGCTTCTACCCCTCCGATATCG |
| | | CCGTGGAATGGGAGTCTAATGGCCAGCCTGAGAACAACT |
| | | ACAAGACCACACCTCCTGTGCTGGACTCCGACGGCTCATT |
| | | CTTCCTGTACTCCAAGCTGACAGTGGACAAGTCCAGATGG |
| | | CAGCAGGGCAACGTGTTCTCCTGCTCCGTGATGCACGAGG |
| | | CCCTGCACAATCACTACACCCAGAAGTCCCTGTCTCTGAG |
| | | CCCCGGCAAA |
| 218 | TPP-49058 | GAGGTGCAGTTGGTCCAATCAGGAGCAGAAGTCAAAAAA |
| | LRRC15 | CCCGGTGCTTCCGTTAAAGTATCTTGCAAAGCTAGCGGGT |
| | Heavy Chain | ACAAGTTCTCTTCCTATTGGATCGAGTGGGTTAAGCAGGC |
| | Hole (TCR) | ACCTGGACAGGGTCTTGAATGGATAGGCGAAATCCTTCCC |
| | | GGTAGCGACACCACAAACTATGCTCAAAAGTTCCAAGATA |
| | | GAGTTACTTTCACTTCAGATACTTCCATATCTACTGCATAT |
| | | ATGGAACTGTCCCGTTTGAGGAGTGATGACACAGCAGTGT |
| | | ACTACTGTGCCCGTGACCGTGGAAACTACCGTGCATGGTT |
| | | CGGCTATTGGGGTCAGGGTACACTCGTAACAGTGAGCTCA |
| | | GCAAGCACCAAGGGACCCTCTGTGTTCCCTCTGGCTCCTT |
| | | CCAGCAAGTCTACCTCTGGCGGAACAGCTGCTCTGGGCTG |
| | | CCTGGTCAAGGACTACTTTCCTGAGCCTGTGACCGTGTCCT |
| | | GGAACTCTGGCGCTCTGACATCTGGCGTGCACACCTTTCC |
| | | AGCTGTGCTGCAGTCCTCCGGCCTGTACTCTCTGTCCTCTG |
| | | TCGTGACCGTGCCTTCCAGCTCTCTGGGAACCCAGACCTA |
| | | CATCTGCAATGTGAACCACAAGCCTTCCAACACCAAGGTG |
| | | GACAAGAGAGTGGAACCAAAAAGCTGTGACAAGACTGGT |
| | | GGCTCAGACGTACAATTGGTTGAAAGTGGTGGGGGCGTA |
| | | GTCCAGCCCGGAGGCAGCCTCCGACTTTCCTGCGTCGCAT |
| | | CTGGATACGTCCATAAAATCAACTTTTACGGCTGGTACAG |
| | | ACAAGCACCAGGGAAAGAGCGGGAAAAAGTTGCCCACAT |
| | | ATCCATTGGCGACCAGACAGATTATGCCGACAGCGCCAAG |
| | | GGTCGATTTACTATAAGTCGGGACGAATCAAAGAATACTG |
| | | TGTATCTCCAGATGAACAGTCTTCGACCTGAAGACACCGC |
| | | CGCTTATTATTGTAGGGCTTTGTCCAGGATCTGGCCATACG |
| | | ATTACTGGGGGCAAGGGACACTCGTAACCGTTAGTTCTGG |
| | | TGGCGGAGGATCTACCCACACCTGTCCTCCATGCCCTGCT |
| | | CCACCCGTGGCCGGCCCTTCCGTGTTCCTGTTTCCTCCAAA |
| | | GCCTAAGGACACCCTGATGATCTCTCGGACCCCTGAAGTG |
| | | ACCTGCGTGGTGGTGGATGTGAAGCACGAGGATCCCGAA |
| | | GTGAAGTTCAATTGGTACGTGGACGGCGTGGAAGTGCACA |
| | | ACGCCAAGACCAAGCCTAGAGAGGAACAGTACAACTCCA |
| | | CCTACAGAGTGGTGTCCGTGCTGACCGTGCTGCACCAGGA |
| | | TTGGCTGAACGGCAAAGAGTACAAGTGCAAGGTGTCCAA |
| | | CAAGGCCCTGCCTGCCCCCATCGAAAAGACCATCTCTAAG |
| | | GCTAAGGGCCAGCCTCGCGAGCCTCAAGTCTGTACACTGC |
| | | CTCCTAGCCGGGAAGAGATGACCAAGAACCAGGTGTCCCT |
| | | GTCTTGCGCCGTGAAGGGCTTCTACCCTTCCGATATCGCC |
| | | GTGGAATGGGAGTCCAATGGCCAGCCTGAGAACAACTAC |
| | | AAGACAACCCCTCCTGTGCTGGACTCCGACGGCTCATTCT |
| | | TCCTGGTGTCCAAGCTGACAGTGGACAAGTCCAGATGGCA |
| | | GCAGGGCAACGTGTTCTCCTGCTCTGTGATGCACGAGGCC |
| | | CTGCACAACCGGTTCACCCAGAAATCCCTGTCTCTGTCCC |
| | | CTGGCAAG |
| 219 | TPP-49058 | GACATCCAGATGACCCAGAGTCCCTCTAGTCTCTCAGCTT |
| | LRRC15 | CCGTGGGGGACCGCGTCACTATAACTTGCCGTGCCTCACA |
| | Light Chain | AGACATATCAAACTACCTCAACTGGTATCAACAAAAGCCT |
| | | GGAAAAGCTCCTAAGTTTTTGATTTATTACACTTCCAGGCT |
| | | GGAGTCCGGTGTCCCATCCAGGTTCAGCGGCAGTGGAAGC |
| | | GGAACCGACTATACACTGACTATTTCTAGCTTGCAACCCG |
| | | AGGACTTTGCCACCTATTATTGCCAGCAAGGTAACGCCCT |
| | | GCCCTGGACCTTCGGTGGCGGAACCAAGGTGGAAATCAA |
| | | GCGGACAGTGGCCGCACCGTCCGTGTTCATCTTCCCACCT |
| | | TCCGACGAGCAGCTGAAGTCTGGCACAGCCTCTGTCGTGT |
| | | GCCTGCTGAACAACTTCTACCCTCGGGAAGCCAAGGTGCA |
| | | GTGGAAGGTGGACAATGCCCTGCAGTCCGGCAACTCCCAA |
| | | GAGTCTGTGACCGAGCAGGACTCCAAGGACAGCACCTAC |
| | | AGCCTGTCCTCCACACTGACCCTGTCCAAGGCCGACTACG |
| | | AGAAGCACAAGGTGTACGCCTGCGAAGTGACCCATCAGG |
| | | GCCTGTCTAGCCCTGTGACCAAGTCTTTCAACCGGGGCGA |
| | | GTGC |
| 220 | C_{H1} with | ASTKGPSVFPLAPSSKSTSGGTADLGCLVKDYFPEPVTVSW |
| | A141D | NSGALTSGVHTFPAVLQSSGLYSLSSVVTVPSSSLGTQTYIC |
| | | NVNHKPSNTKVDKRVEPKSC |
| 221 | IgG1 CH2 | APELLGGPSVFLFPPKPKDTLMISRTPEVTCVVVDVSHEDPEV |
| | WT(IMGT) | KFNWYVDGVEVHNAKTKPREEQYNSTYRVVSVLTVLHQD |
| | alternate | WLNGKEYKCKVSNKALPAPIEKTISKAK |
| 222 | IgG1 CH2 Fc- | APPVAGPSVFLFPPKPKDTLMISRTPEVTCVVVDVKHEDPEV |
| | null (IMGT) | KFNWYVDGVEVHNAKTKPREEQYNSTYRVVSVLTVLHQD |
| | alternate | WLNGKEYKCKVSNKALPAPIEKTISKAK |

### Example 2: In Vitro Testing of LM1486 Binding Proteins

The activity of the novel LM1486 (CD20) binding proteins disclosed herein were demonstrated.

### In Vitro Cytolysis

### Method

An assay was conducted to measure cytolysis of LM1486 compared to known CD3 engagers. The assay was run for 4 days with an OCI-Ly18 CD20+ DLBCL line and PBMCs at an E:T ratio of 5:1. Target B cell survival was assessed by flow cytometry.

### Results

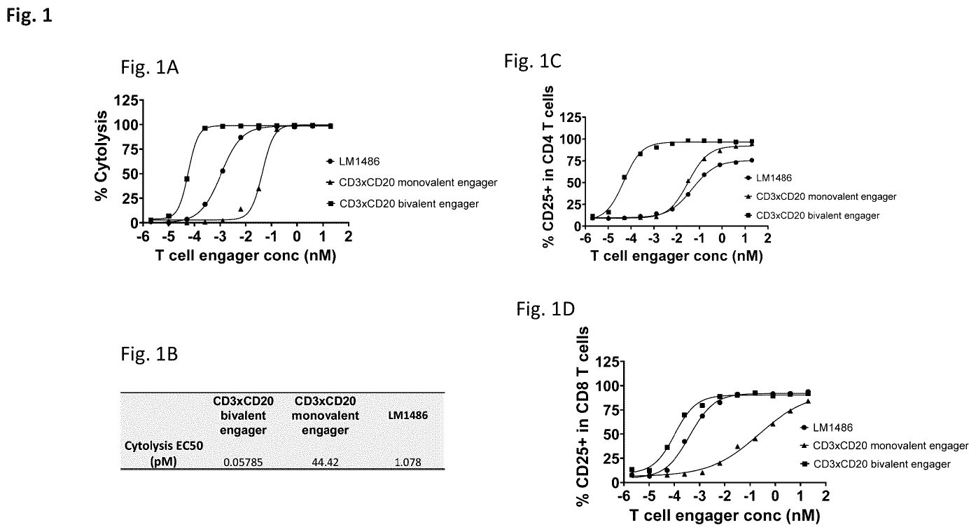

FIGS. 1A and 1B show potent in vitro cytolysis by LM1486 compared to conventional CD3 engagers. FIG. 1A shows the in vitro cytolytic activity of LM1486 and CD3 engagers with monovalent or bivalent engagement to CD20. FIG. 1B shows the corresponding EC₅₀s in the cytolysis in vitro assay.

Comparison of CD4 T Cell and CD8 T Cell Activation with Known CD3 Engager

### Method

At the end of the cytolysis assay, T cell activation status was evaluated by flow cytometry.

### Results

A CD8-biased T cell activation profile was induced by LM1486 compared to conventional CD3 engagers. FIG. 1C shows corresponding CD4 T cell activation profiles as % of cells expressing CD25 on the cell surface. FIG. 1D shows corresponding CD8 T cell activation profiles as % of cells expressing CD25.

Comparison of Cytokine Release Profiles with known CD3 Engagers

### Method

The cytokine release profiles were measured in the supernatant of an in vitro cytolysis experiment using a multiplexing assay.

### Results

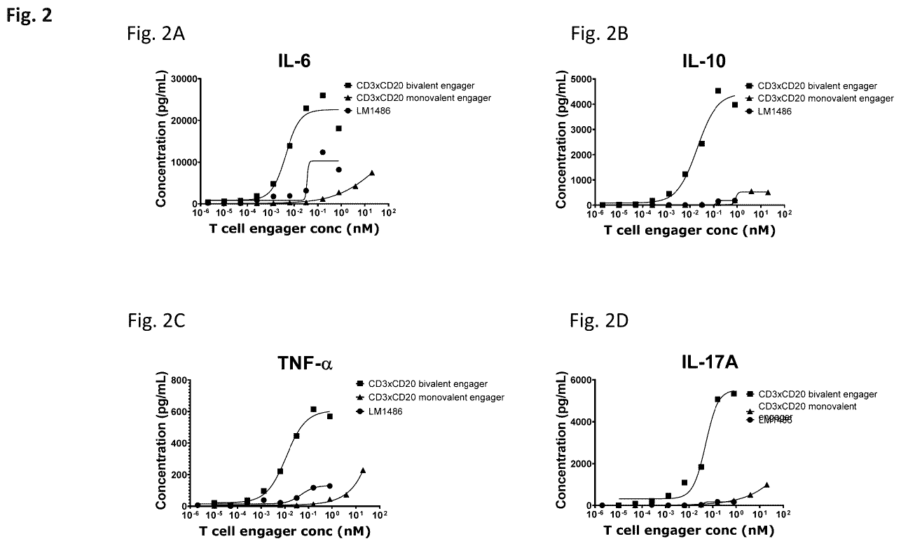

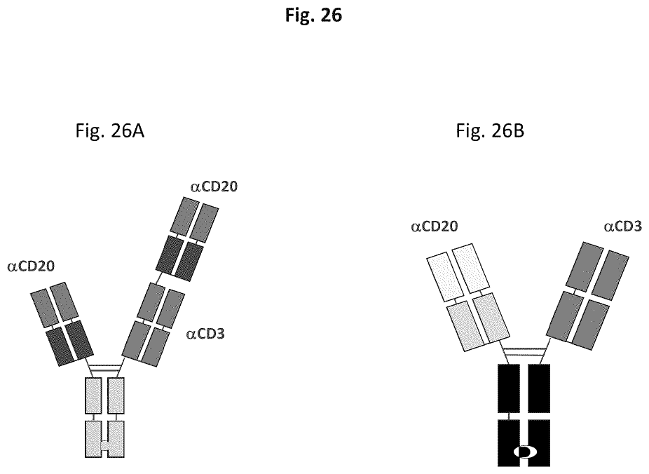

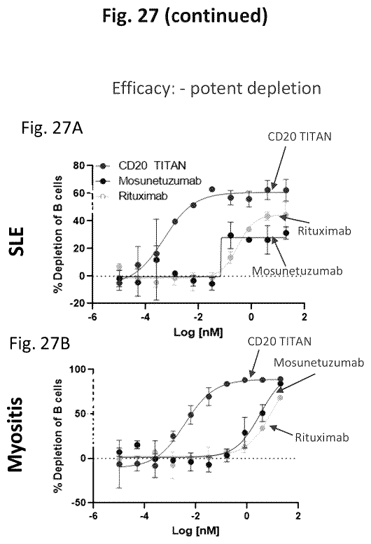

FIGS. 2A-2D show in vitro cytokine release profiles of LM1486 compared to conventional CD3 engagers: IL-6 (FIG. 2A), IL-10 (FIG. 2B), TNF-α (FIG. 2C), and IL-17A (FIG. 2D). The results show that LM1486 induced much lower levels of cytokines when compared with the CD3×CD20 bivalent engager (FIG. 26A) and levels similar to the CD3×CD20 monovalent engager.

### In Vitro Cytolysis in Different CD20+ B Cell Lines

### Method

The in vitro cytolytic activity of LM1486 was measured with different CD20+ B cell lines incubated with PBMCs at an E:T ratio of 5:1. The assay was run for 3 to 4 days and target B cell survival was assessed by flow cytometry.

### Results

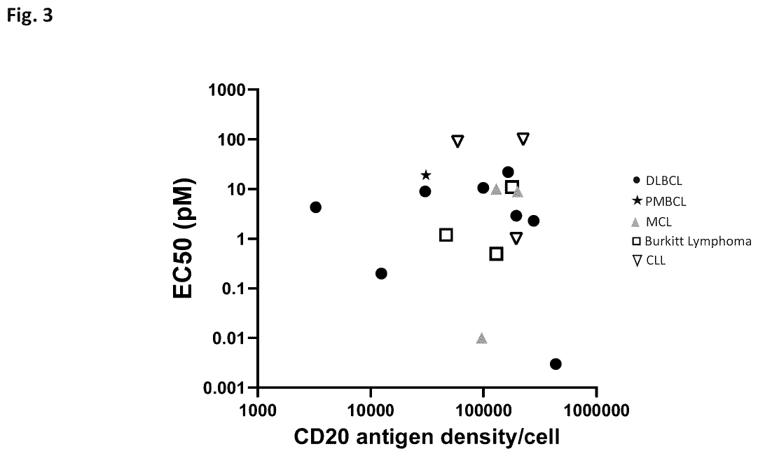

FIG. 3 shows potent in vitro cytolytic activity of LM1486 across an array of B cell lines expressing various levels of CD20 antigen density and also shows the absence of correlation between CD20 density and EC₅₀s.

Comparison of Cytolytic Activity of LM1486 with CD8 and TCR Null Controls

### Method

LM1486 was compared with control binding proteins TENG0501 (TCR null) and TENG0502 (CD8 null). The assay was run with an OCI-Ly18 CD20+ DLBCL line and PBMCs at an E:T ratio of 5:1, and target B cell survival and T cell activation profiles were assessed by flow cytometry.

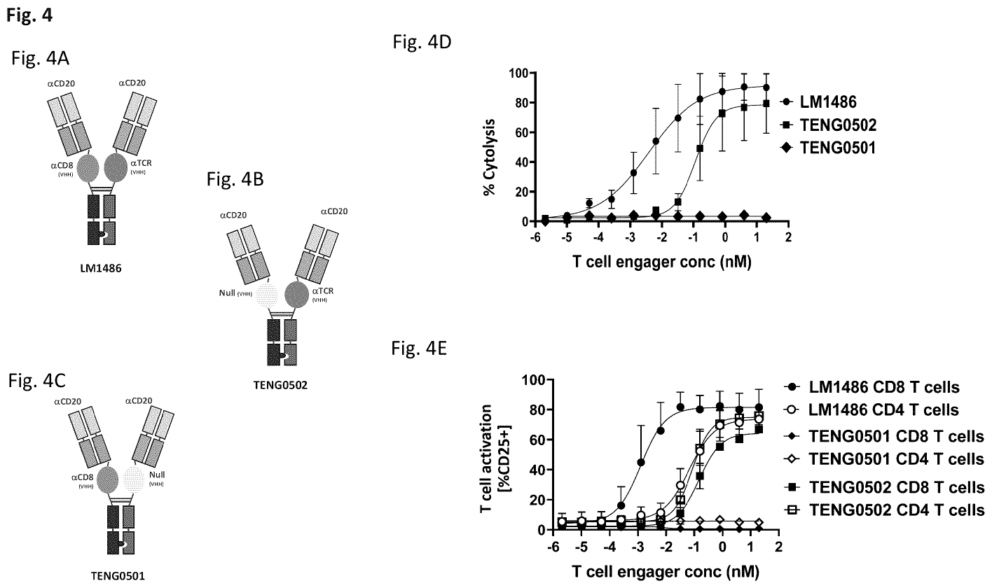

FIGS. 4A-4C illustrate the structures of LM1486 (FIG. 4A) and the structures of modified binding proteins with a null binding domain replacing the anti-CD8 domain (FIG. 4B) or a null binding domain replacing the anti-TCR binding domain (FIG. 4C).

### Results

FIG. 4D shows the in vitro cytolytic activity of LM1486 and control binding proteins TENG0501 and TENG0502. FIG. 4E shows corresponding T cell activation profiles as % CD4 and CD8 T cells expressing CD25 assessed by flow cytometry. The data demonstrates that the LM1486 TCR binding domain is essential to cytolytic activity and that CD8 arm enhances potency and confers CD8-biased T cell activation.

Comparison of Cytolytic Activity of LM1486 with Single Null Binding Domain Controls

### Method

LM1486 was compared with control binding proteins TENG0500 (null binding protein replacing anti-CD20 domain on anti-CD8 arm) and TENG0499 (null binding protein replacing anti-CD20 domain on anti-TCR arm). The assay was run with OCI-Ly18 CD20+ DLBCL line and PBMCs at an E:T ratio of 5:1, and target B cell killing and T cell activation profiles were assessed by flow cytometry.

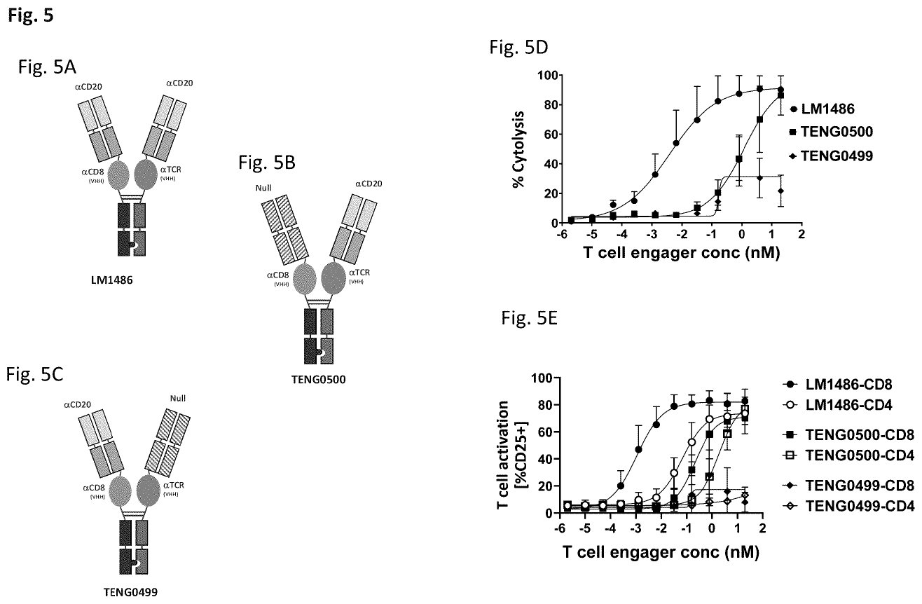

FIGS. 5A-5C illustrate the structure of LM1486 (FIG. 5A) and the structure of modified binding proteins with a null binding domain replacing the anti-CD20 domain on the anti-CD8 arm (FIG. 5B) or with a null binding domain replacing the anti-CD20 binding domain on the anti-TCR arm (FIG. 5C).

### Results

FIG. 5D shows the in vitro cytolytic activity of LM1486 and control binding proteins TENG0499 and TENG0500. FIG. 5E shows corresponding T cell activation profiles as % CD4 and CD8 T cells expressing CD25. These data demonstrate that CD20 binding on the TCR arm is required to achieve cytolysis.

Comparison of Cytolytic Activity of LM1486 with LM1486-2

LM1486 differs from LM1486-2 by the presence of an RF mutation in the TCR heavy chain of LM1486 (see SEQ ID NO: 110), H318R and Y319F.

### Method

The assay was run for 3 days with OCI-Ly18 CD20+ DLBCL line and PBMCs at an E:T ratio of 5:1. B cell survival and T cell activation profiles were assessed by flow cytometry.

### Results

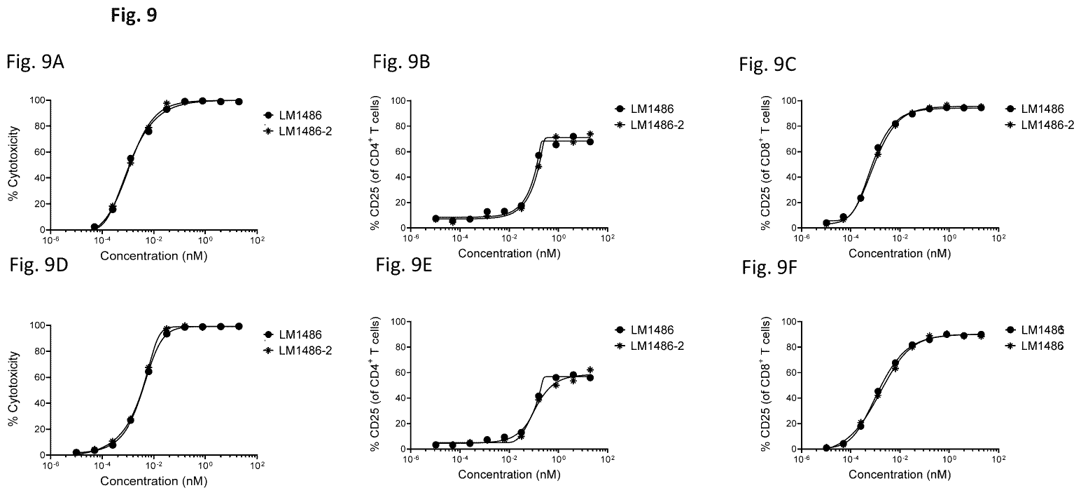

FIG. 9 shows overlapping in vitro cytolysis profiles for LM1486 and LM1486-2. FIGS. 9A and 9D show cytotoxicity profiles in a range of T cell engager concentrations for 2 different PBMC donors. FIGS. 9B, 9C, and FIG. 9E, 9F show associated CD4 and CD8 T cell activation profiles as % of CD25 surface expression for 2 donors, respectively.

### Example 3: In Vivo Efficacy of LM1486 in B Cell Model

### Assessing Anti-Tumor Efficacy In Vivo in a Disseminated B Cell Model

### Method

At day 6, animals were humanized with 5e6 PBMCs. Animals were then treated i.p. twice a week with PBS, or LM1486 at 0.8, 0.08, or 0.008 mg/kg. Tumor burden was monitored twice weekly by bioluminescence imaging and total flux (p/s) was recorded.

### Results

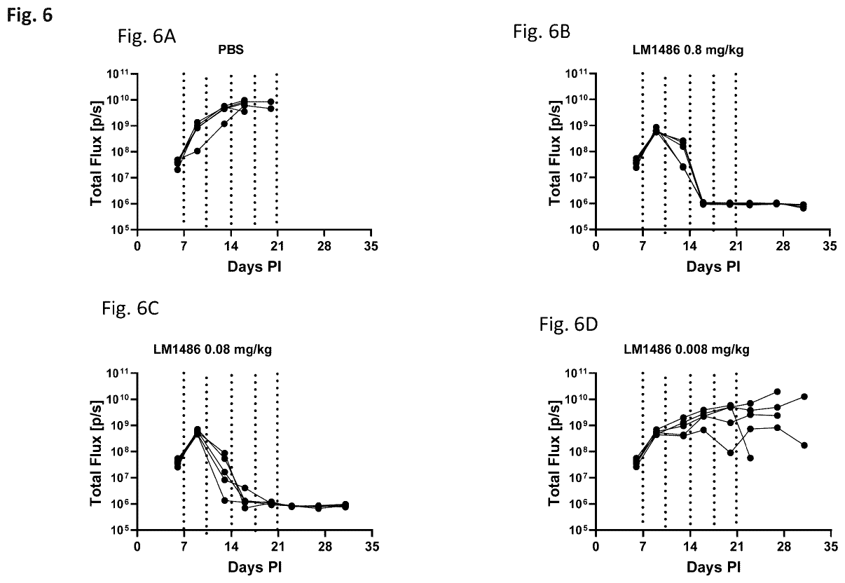

The results demonstrate that the LM1486 binding protein was efficacious in vivo. FIG. 6 shows tumor burden in a disseminated model of the PBS treated groups (FIG. 6A), groups treated LM1486 at 0.8 mg/kg (FIG. 6B), 0.08 mg/kg (FIG. 6C), and 0.008 mg/kg (FIG. 6D). These data demonstrate that LM1486 confers potent anti-tumor efficacy in vivo with as low as 0.008 mg/kg.

### Comparing Tumor Growth Inhibition, Survival Benefit, and Superior Safety Over Known CD3×CD20 Known Engagers

### Method

CD34+ engrafted NSG mice (n=8-9/group) were implanted s.c. on the flank with 5e6 OCI-Ly18 cells. Animals were then treated i.p. with 1 mg/kg of T cell engagers (CD3×CD20 monovalent (FIG. 26B) or bivalent engager (FIG. 26A) or LM1486 or PBS) at day 1, followed by weekly engager dosing until day 21. Tumor growth was monitored twice weekly until study end. Animals were euthanized when tumor reached 2000 mm³. Percentage survival is reported for all treatment groups up to study end (day 42), weight loss was monitored over time as a surrogate assessment of safety.

### Results

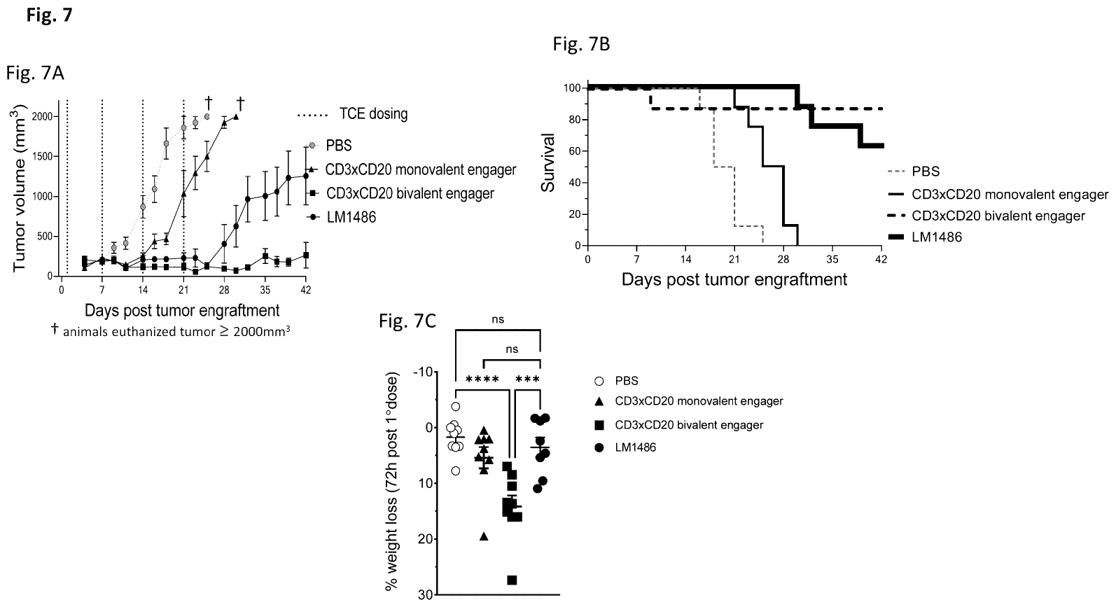

FIG. 7 shows LM1486 potent tumor growth inhibition, survival benefit, and superior safety over known CD3 engagers in a fully humanized in vivo model with subcutaneous B cell tumor. FIGS. 7A-7C shows LM1486 potent tumor growth inhibition, survival benefit, and superior safety over conventional CD3 engagers in a fully humanized in vivo model with subcutaneous B cell tumor. FIG. 7C shows the percentage body weigh loss recorded 72h post first dose of T cell engager.

### Anti-Tumor Efficacy of LM1486 in a Disseminated DLBCL In Vivo Model Compared to Known CD3×CD20 Engagers

### Method

NSG mice were injected i.v. with Toledo B cell line (DLBCL origin) expressing Luciferase (Toledo-Luc) at day 0. At day 7, mice were humanized with 10e6 PBMCs. At day 21, a single dose of T cell engager was injected at i.v at 1 mg/kg for LM1486 and equimolar concentrations for the other molecules. At day 24, tumor burden was evaluated using bioluminescence imaging (BLI). Total flux (p/s) reflecting tumor burden are displayed for each treatment group. 3h post engager treatment, animals were bled, and levels of circulating cytokines were evaluated using a multiplexing assay.

### Results

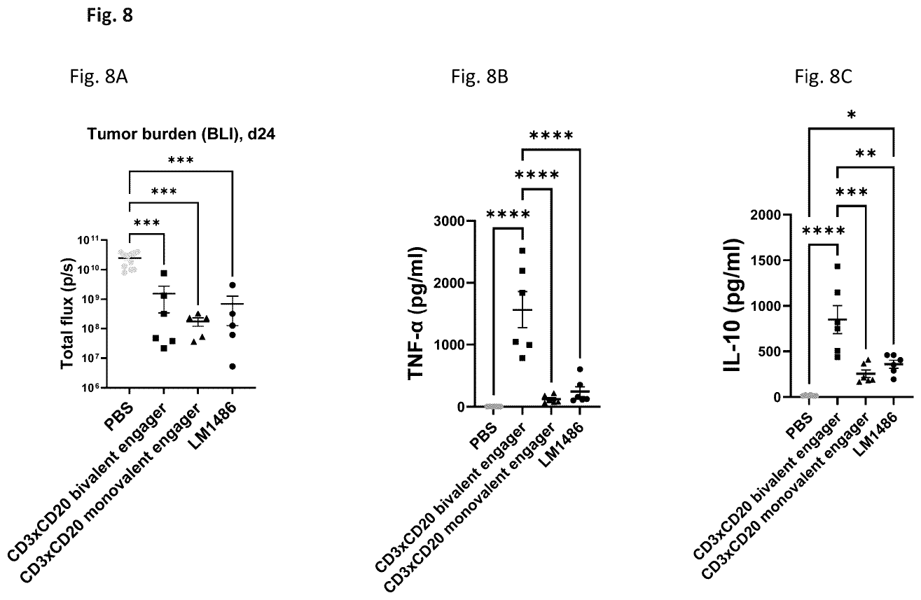

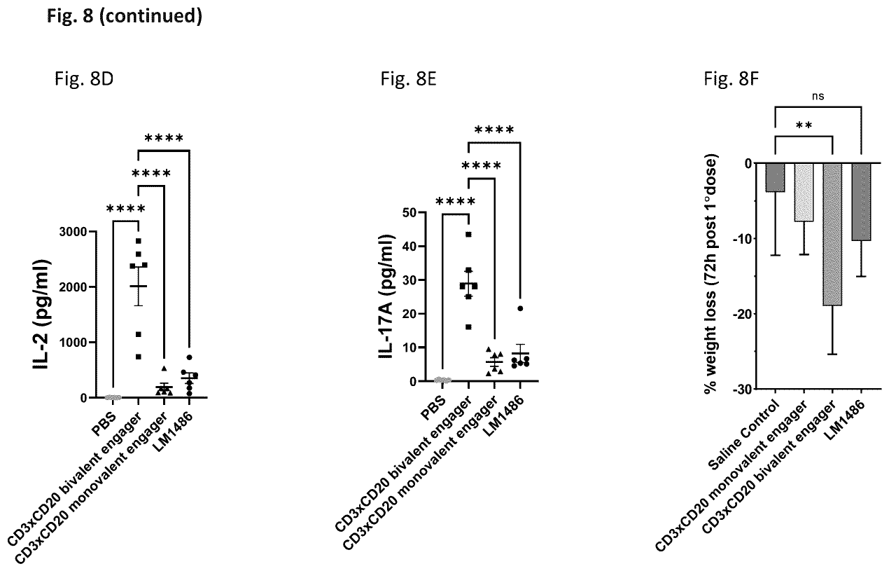

FIGS. 8B-E show reduced cytokine release: TNF-α (pg/mL) (FIG. 8B), IL-10 (pg/mL) (FIG. 8C), IL-2 (pg/mL) (FIG. 8D) and IL-17A (pg/mL) (FIG. 8E) compared to a known CD3×CD20 bivalent engager (FIG. 26A). Further, FIG. 8F shows reduced weight loss compared to the same known CD3×CD20 bivalent engager. LM1486, in a disseminated DLBCL in vivo model, has a superior safety profile compared to a known CD3×CD20 bivalent engager (FIG. 26A) (reduced systemic cytokine and reduced weight loss).

### Example 4: In Vitro Testing of LRC150016 Binding Proteins

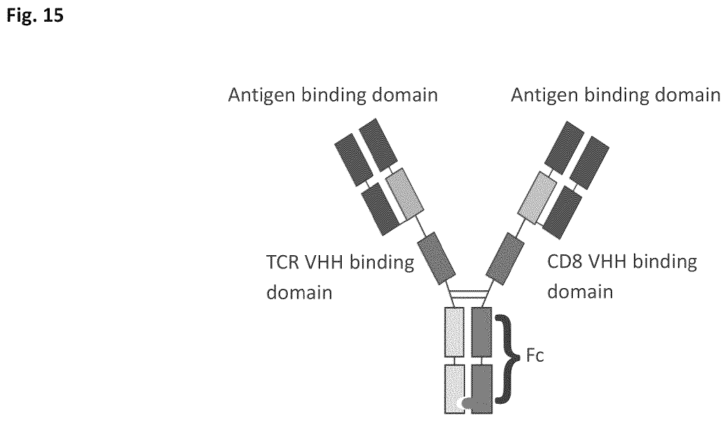

A novel T-cell engager, LRC150016 (LRRC15), was generated, featuring two LRRC15-binding domains, a CD8 binding domain and a TCR binding domain (FIG. 15).

### Results

### Binding Protein Structure and Cell Binding Activity.

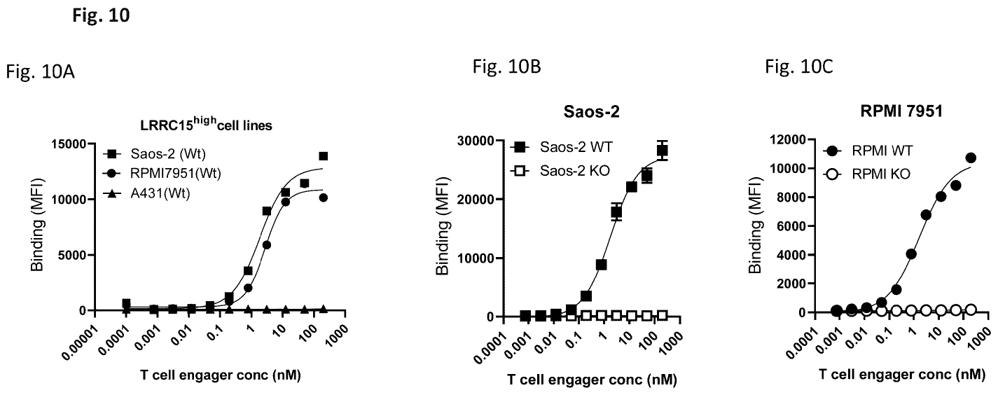

LRC150016 was assessed for binding onto LRRC15^{Pos} and LRRC15^{neg} cell lines. LRC150016 engager shows specific binding to LRRC15 expressing cell lines. The assay was performed by incubating LRC150016 with LRRC15^{pos} cell lines (FIG. 10A) Saos-2 WT (FIG. 10B) and RPMI WT (FIG. 10C)), LRRC15^{neg} cell lines (A431, Saos-2 KO and RPMI-7951 KO), for 1h at 4° C. LRC150016 was detected using anti-human Fc Alexa Fluor 647 conjugated secondary antibody (MFI). LRC150016 is able to bind to LRRC15^{pos} tumour cell lines Saos-2 and RPMI7951, but not to the LRRC15^{neg} cell line A431 (FIG. 10A). When LRRC15 is knocked out from Saos2 WT and from RMPI-7951 WT cells, the binding of LRC150016 is lost, demonstrating that the observed binding is specifically due to LRRC15 protein binding (FIG. 10B and FIG. 10C).

### LRC150016 Mediates T-Cell Effector Functions in an Antigen (LRRC15) Dependent Manner.

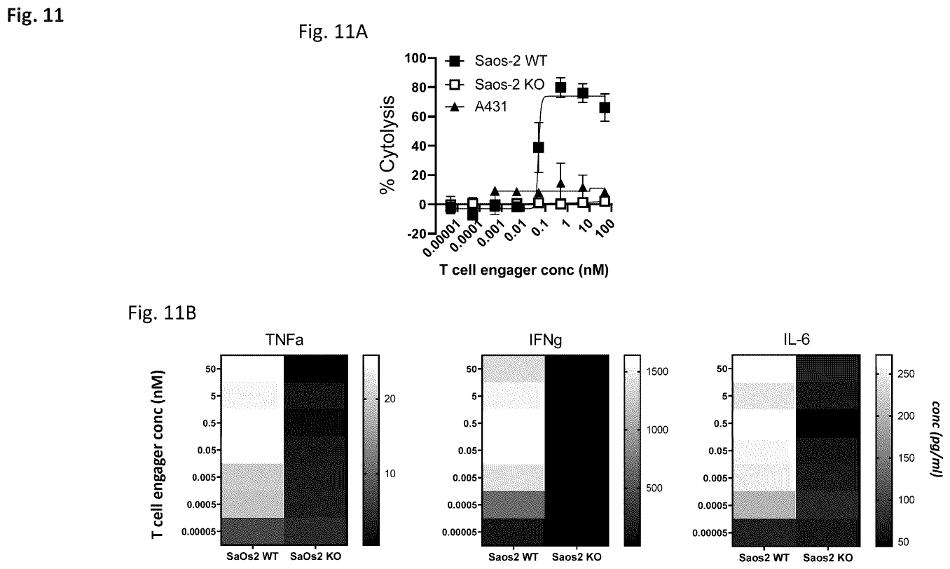

LRC150016 was assessed for in-vitro T-cell effector functions on Saos-2 WT(LRRC15⁺) and Saos-2 KO(LRRC15⁻) target cell lines. Purified human T cells were co-cultured with target cell lines in the presence of LRC150016 at an E:T ratio of 4:1 for 3 days. % of target cytolysis (cytotoxicity) was measured by CELL-TITRE-GLO. Cytokines (TNFα, IFNγ and IL-6) were measured in culture medium collected post 3 days of co-culture by a multiplexing cytokine assay. FIG. 11A depicts that LRC150016 induces a dose-dependent strong cytolysis of Saos-2 WT (LRRC15⁺), but not Saos-2 KO (LRRC15⁻) target cells. FIG. 11B shows cytokines associated with the induction of T-cell effector function mediated by LRC150016 upon co-culture of T-cells with Saos-2 WT cells, but not with LRRC15-KO Saos-2 cells. FIG. 11A-11B shows LRC150016 engager mediates T-cell effector functions in an antigen (LRRC15) dependent manner.

### LRC150016 Specifically Induces Activation of CD8 T-Cells, Over CD4 T-Cells, Against LRRC15⁺ Saos-2 Cells.

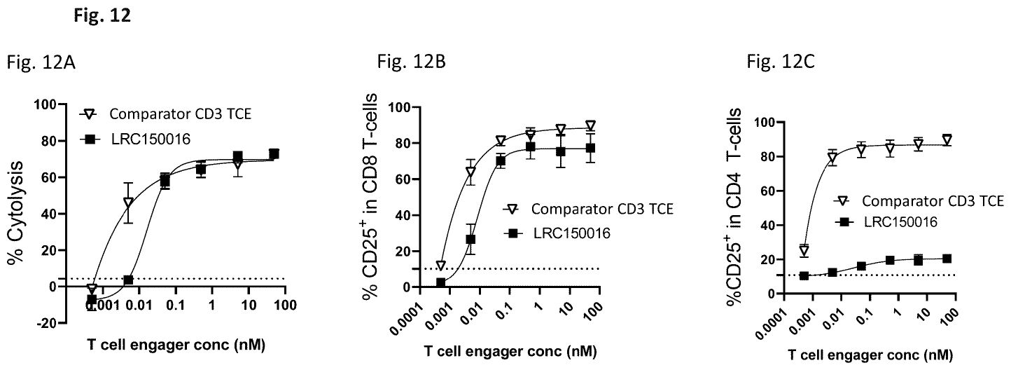

To compare LRC150016 with a Comparator bispecific LRRC15/CD3 T-cell engager, whole healthy human PBMCs (effectors) were co-cultured with Saos-2 WT (LRRC15+) target cells in the presence of molecules for a period of 4 days at an E:T ratio 4:1 (FIG. 12A). Post 4 days co-culture, target cell cytolysis, activation of T-cells and secreted cytokines were measured by CELL-TITRE-GLO, CD25 surface expression assessment by flow cytometry and a multiplex cytokine assay respectively. Activation of CD8 T-cells in this co-culture, measured by upregulation of CD25 is similar for both molecules (FIG. 12B), but the CD3-targeted bispecific also strongly induces unwanted activation of CD4 T-cells, also shown by CD25 upregulation (FIG. 12C). FIG. 12 demonstrates potent in vitro cytolysis and CD8-biased T-cell activation profile induced by LRC150016, compared to Comparator CD3 TCE.

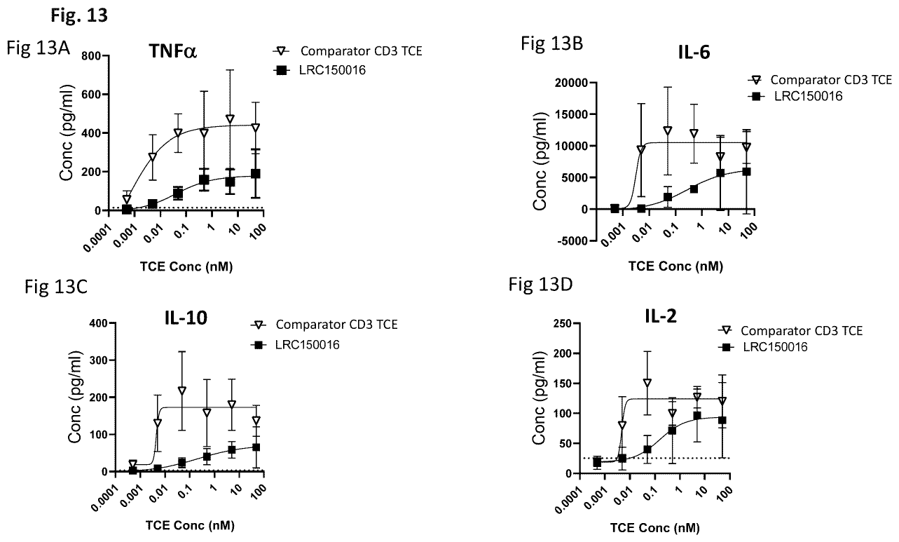

Despite similar target cell cytolysis activity, LRC150016 induced lower levels of cytokines, compared to the CD3-targeting Comparator TCE (FIGS. 13A-D). FIGS. 13A-13D show the in vitro cytokine profile of LRC150016, compared to the Comparator CD3 TCE. Cytokine profiles were measured in the supernatant of whole PBMC in vitro cytolysis experiments using a multiplexing cytokine assay. TNFα (FIG. 13A), IL-6 (FIG. 13B), IL-10 (FIG. 13C), and IL-2 (FIG. 13D).

### LRC150016 Mediates T-Cell Cytolytic Activity Against LRRC15⁺ Cells Across a Range of LRRC15 Expression Levels.

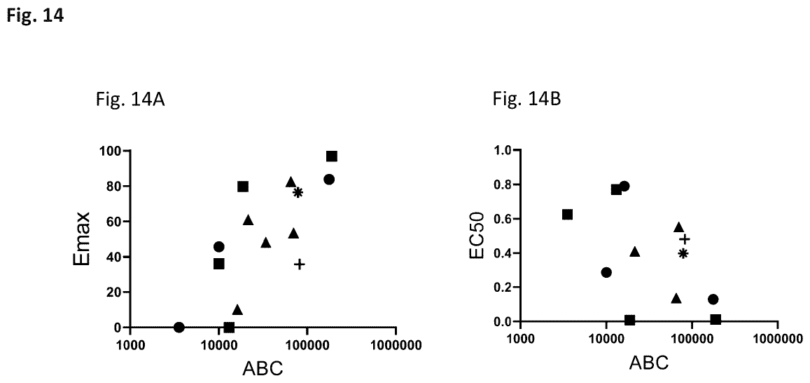

To understand the correlation between LRRC15 copy number (density) on target cells and LRC150016 mediated target cell cytolysis, purified human T cells were co-cultured with LRRC15^{pos} target cell lines, from various indications, in the presence of LRC150016 for a period of 6-days at an E:T ratio of 4:1. Target cell cytolysis was measured by CELL-TITRE-GLO. Eₘₐₓ and EC₅₀ values were derived from these experiments and projected against LRRC15 copy number of corresponding target cells. In co-cultures of tumour cells with isolated CD3 T-cells, LRC150016 induces tumour cell cytolysis of cells with more than 10-fold variation in LRRC15 antigen copy number (FIG. 14). FIG. 14A and FIG. 14B demonstrates that LRC150016 shows specific in vitro cytolytic activity across an array of cell lines expressing various levels of LRRC15.

### Example 5: In Vitro and In Vivo Testing of LM1653 (GPC3) Binding Proteins

### Binding Kinetics Assay

### Method

Binding kinetics were measured by biolayer interferometry on an Octet384 instrument. Streptavidin (SA) biosensors were loaded with biotinylated protein antigens (ACRO Biosystems, Newark, DE) in PBS pH 7.2, 1 mg/mL BSA, 0.05% (v/v) TWEEN (Kinetic buffer). The loaded biosensors were washed in the same buffer before carrying out association and dissociation measurements with various antibodies for the indicated times. Kinetic parameters (Kₒₙ and K_{off}) and affinities (K_{D}) were calculated from a non-linear fit of the data using the OCTET software v.12.2.1.24.

### Cell Binding Assay

### Method

Cell bindings were measured by flow cytometry. Human hepatoma cells HepG2 and Hep3B, human and cyno PBMC were incubated with serial diluted GPC3 TITAN molecule on ice for 30 minutes, washed with FACS buffer and stained with goat anti-human-IgG-APC (Jackson ImmunoResearch) on ice for 30 minutes followed by flow cytometry analysis.

### Results

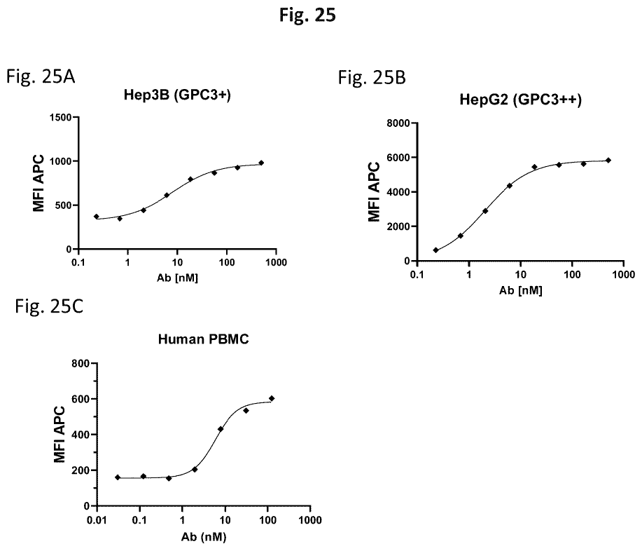

FIG. 25 shows LM1653 binds human hepatoma and effector cells. FIG. 25 shows that GPC3 TITAN LM1653 binds human hepatoma cells Hep3B at EC₅₀: 8 nM (FIG. 25A) and HepG2 at EC₅₀: 2 nM (FIG. 25B). GPC3 TITAN binds to human PBMC at EC₅₀: 6 nM (FIG. 25C). Cell bindings were measured by flow cytometry. Human hepatoma cells HepG2 and Hep3B, human PBMC were incubated with serial diluted GPC3 TITAN molecule on ice for 30 minutes, washed with FACS buffer and stained with goat anti-human-IgG-APC (Jackson ImmunoResearch) on ice for 30 minutes followed by flow cytometry analysis.

TABLE 2
Kinetic analysis of anti-GPC3 Fab on GPC3 protein

| GPC3 | Kₐ (1/Ms) | K_{dis} (1/s) | K_{D} (M) |
|---|---|---|---|
| human | 2.26E05 | 4.22E−04 | 1.87E−09 |

TABLE 3
Kinetic analysis of anti-CD8 VHH on CD8α protein

| CD8α | Kₐ (1/Ms) | K_{dis} (1/s) | K_{D} (M) |
|---|---|---|---|
| Human | 5.17E05 | 2.86E−02 | 5.53E−08 |

Table 2 shows kinetic analysis of anti-GPC3 fab on soluble recombinant human GPC3 protein interaction. Table 3 shows kinetic analysis of anti-CD8 VHH on soluble recombinant human CD8a protein interaction via Biolayer Interferomerty on an OCTET RED384.

Binding kinetics were measured by biolayer interferometry on an OCTET 384 instrument. Streptavidin (SA) biosensors were loaded with biotinylated protein antigens (ACRO Biosystems, Newark, DE) in PBS pH 7.2, 1 mg/mL BSA, 0.05% (v/v) TWEEN (Kinetic buffer). The loaded biosensors were washed in the same buffer before carrying out association and dissociation measurements with various antibodies for the indicated times. Kinetic parameters (Kₒₙ and K_{off}) and affinities (K_{D}) were calculated from a non-linear fit of the data using the OCTET software v.12.2.1.24.

### In Vitro Cytolysis Induced by LM1653 Compared to a Conventional GPC3×CD3 Engager, Against Hepatocellular Carcinoma (HCC) Cell Lines

### Method

GPC3 expression was detected with a commercially available anti-human GPC3 antibody and quantified using the BD Quantibrite kit. Cytotoxicity assay against the indicated HCC cell line was run for 4 days using PBMCs at an E:T ratio of 10:1, using the XCELLIGENCE system.

### Results

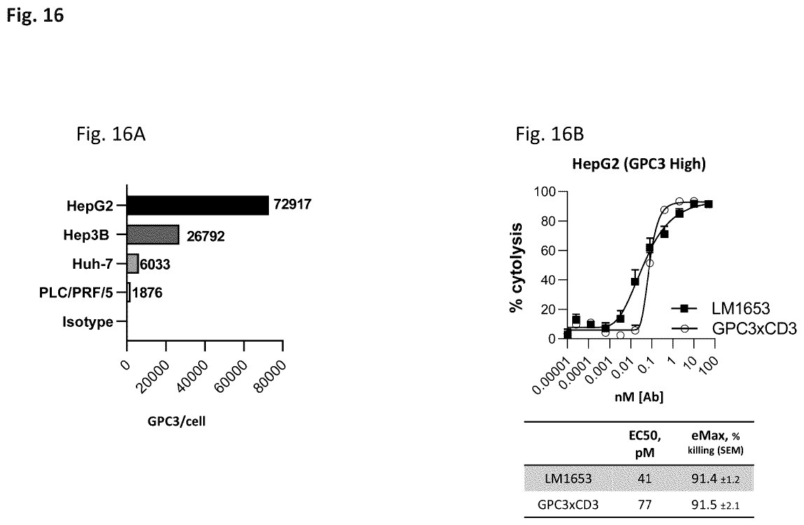

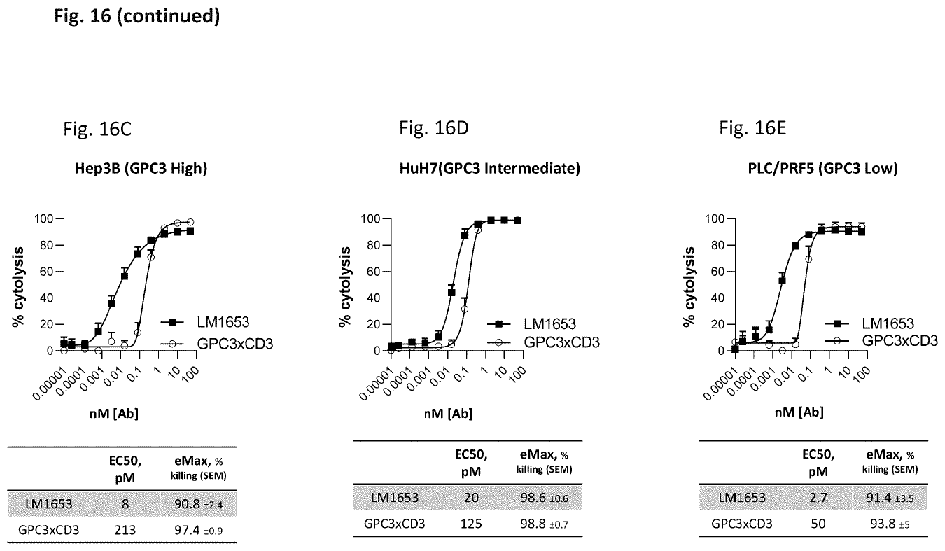

FIG. 16 shows in vitro cytolysis induced by LM1653 compared to a conventional GPC3×CD3 engager, against hepatocellular carcinoma (HCC) cell lines expressing different levels of GPC3. FIG. 16A shows surface expression of GPC3 across 4 HCC cell lines. GPC3 expression was detected with a commercially available anti-human GPC3 antibody and quantified using the BD Quantibrite kit. FIG. 16B-E shows the in vitro cytolytic activity of LM1653 and of a monovalent GPC3×CD3 engager against the HCC cell lines HepG2 (FIG. 16B), Hep3B (FIG. 16C), Huh-7 (FIG. 16D), and PLC/PRF/5 (FIG. 16E). Graphs show mean±SEM (n=3-6 PBMC donors). The tables in the Figures show the corresponding EC₅₀ and killing Eₘₐₓ for each cell line.

### T-Cell Activation Profiles

### Method

T cell activation profiles were determined by flow cytometry. Activation profile was defined as % of parental cells expressing CD25.

### Results

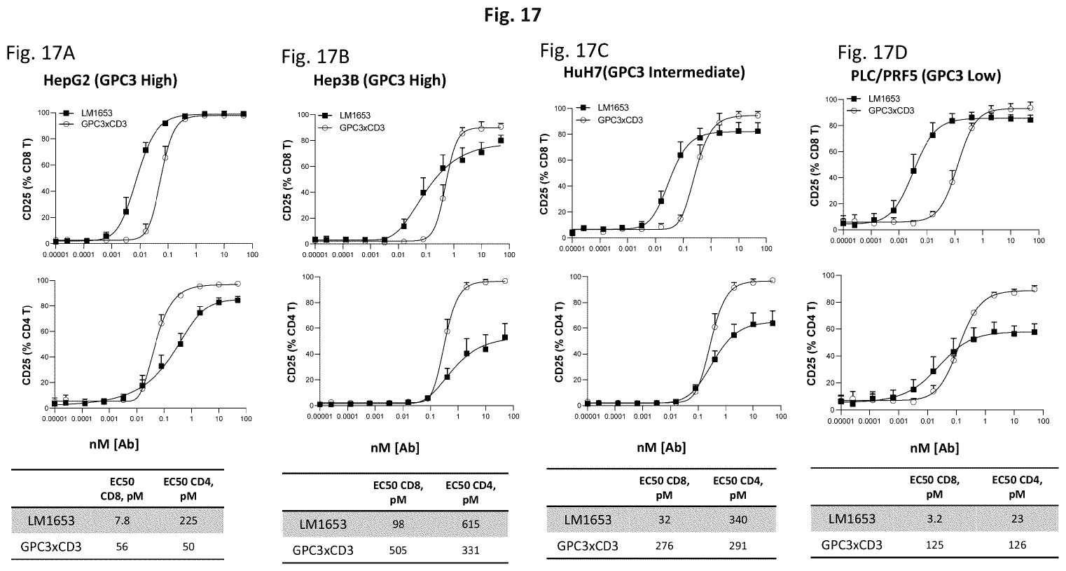

FIG. 17 shows CD8-biased T cell activation profile induced by LM1653 compared to a conventional GPC3×CD3 engager, against HCC cell line expressing different levels of GPC3. FIG. 17 shows CD8+ T cell (upper panels) and CD4+ T cell (bottom panels) activation profiles induced by LM1653 and by a monovalent GPC3×CD3 engager in cytotoxicity assay against HCC cell lines HepG2 (FIG. 17A), Hep3B (FIG. 17B), Huh-7 (FIG. 17C), and PLC/PRF/5 (FIG. 17D). Graphs show mean±SEM (n=3-6 PBMC donors). The tables show the EC₅₀ for CD8 and CD4 T cell activation for each cell line.

Comparison of Cytokine Release Profiles with a Known CD3 Engager

### Method

Cytokine released in supernatant were measured with a multiplex assay (Luminex).

### Results

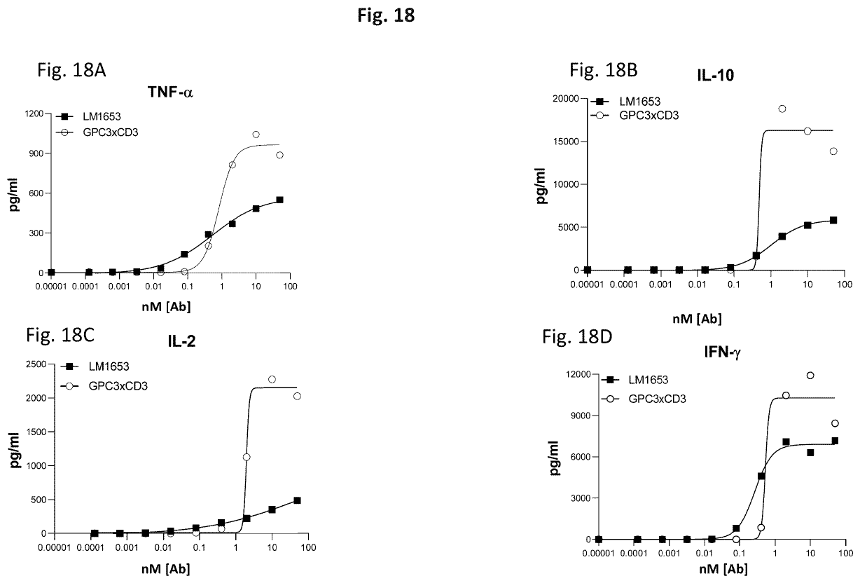

FIG. 18 shows in vitro cytokine release profiles of LM1653 compared to a conventional GPC3×CD3 engager. FIG. 18 shows levels of secreted cytokines, TNF-α (FIG. 18A), IL-10 (FIG. 18B), IL-2 (FIG. 18C), and IFN-7 (FIG. 18D), in an in vitro cytolysis assay, using HCC Hep3B cell line as target cells at E:T ratio of 10:1. Results from one representative PBMC donor of 3 are shown.

### T Cell Activation and Cytokine Release Profile Induced by LM1653 Compared to a Conventional GPC3×CD3 Engager

### Method

Donor PBMCs (n=3) were incubated with LM1653 and a conventional GPC3×CD3 engager for 48 hours. CD8 and CD4 T cell activation profiles (defined as % of parental cells expressing CD25) were determined by flow cytometry. Cytokines released in supernatants at 48 hours were measured using a multiplex assay (LUMINEX).

### Results

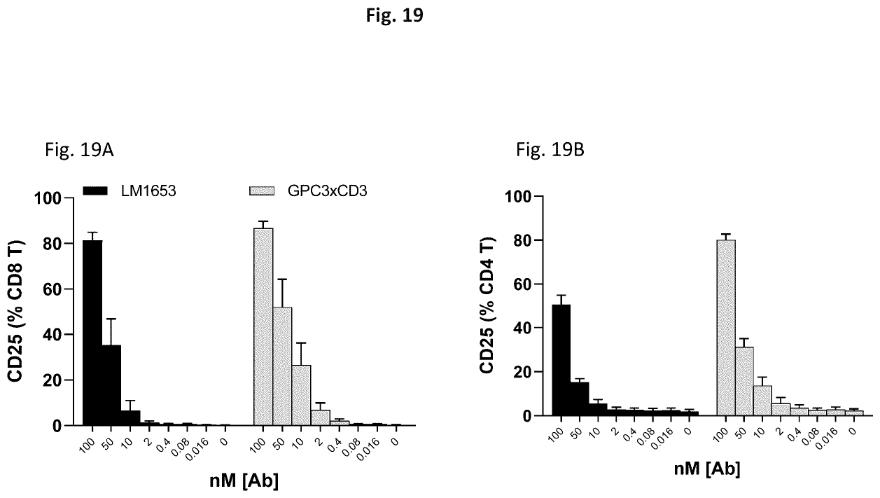

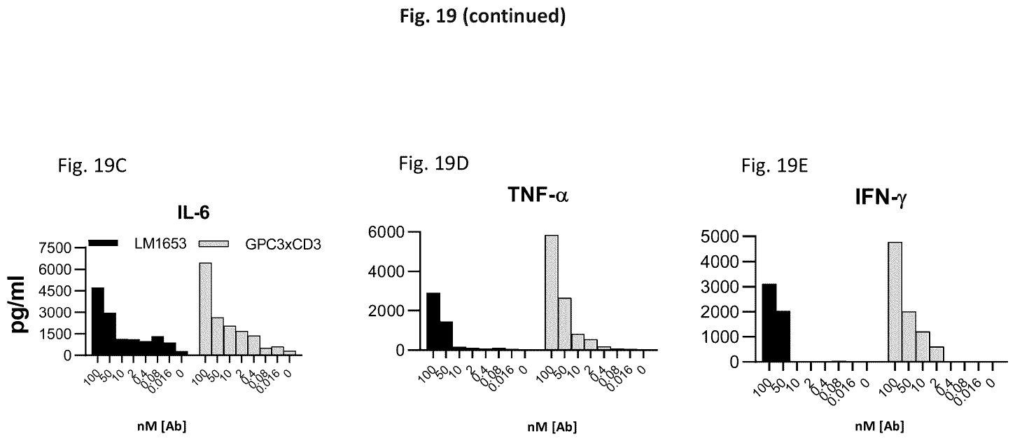

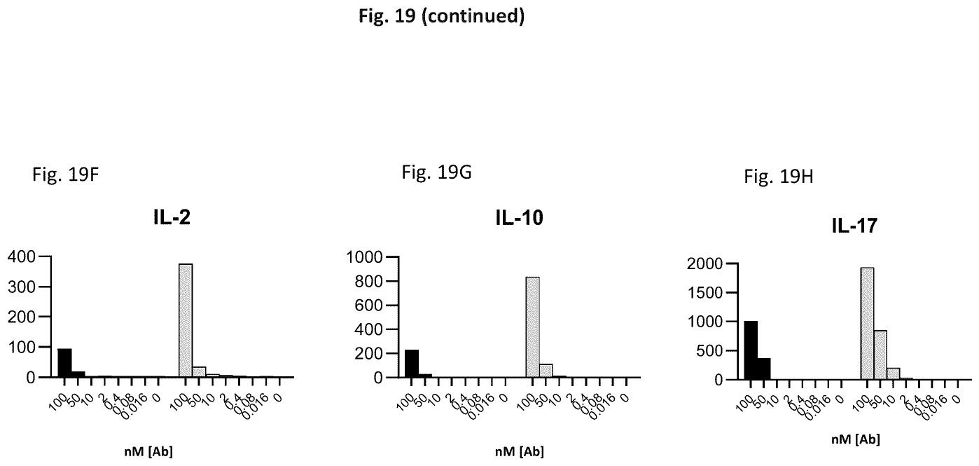

FIG. 19 shows non-specific T cell activation and cytokine release profile induced by LM1653 compared to a conventional GPC3×CD3 engager. FIG. 19 shows non-specific activity of LM1653 and of a conventional GPC3×CD3 engager in a plate-bound assay. Donor PBMCs (n=3) were incubated with LM1653 and a conventional GPC3×CD3 engager for 48 hours. CD8 and CD4 T cell activation profiles (defined as % of parental cells expressing CD25) are depicted in FIG. 19A and FIG. 19B, respectively. Histograms represent mean±SEM; *, p<0.05, 2-way ANOVA. Released IL-6 (FIG. 19C), TNF-α (FIG. 19D), IFN-7 (FIG. 19E), IL-2 (FIG. 19F), IL-10 (FIG. 19G), and IL-17 (FIG. 19H) were measured in the supernatant at 48 hours, using a multiplex assay (LUMINEX). Results from one representative donor PBMC are shown.

### In Vitro Cytolysis and Cytokine Release Profile Induced by LM1653 and by a Conventional GPC3×CD3 Engager

### Method

The assay was run for 4 days using either purified CD4 or CD8 T cells at an E:T ratio of 5:1, using the XCELLIGENCE systems. Target cells were HCC GPC3⁺ Hep3B. Cytokine released in supernatants were measured with a multiplex assay (LUMINEX).

### Results

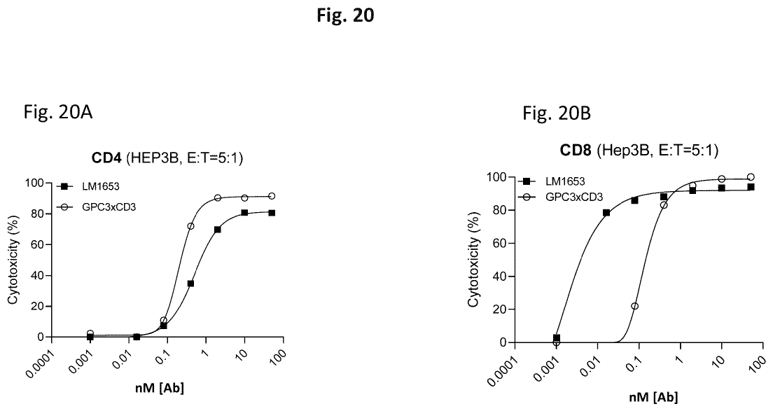

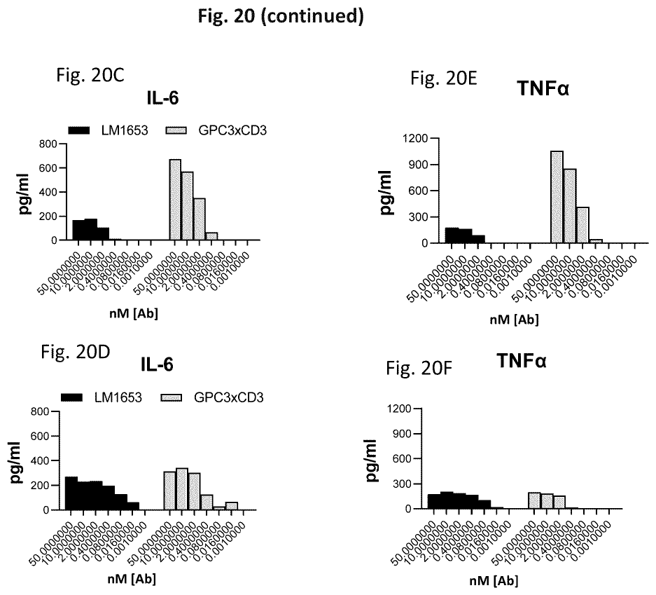

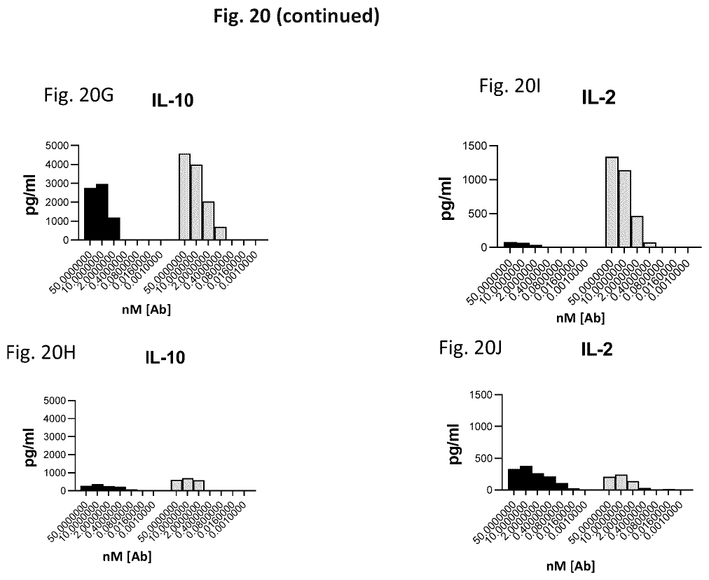

FIG. 20 shows in vitro cytolysis and cytokine release profile induced by LM1653 and by a conventional GPC3×CD3 engager, using purified CD4 and CD8 T cells. FIG. 20 shows the in vitro cytolytic activity (FIG. 20A-B) and cytokine secretion (FIG. 20C-J) of LM1653 and of a monovalent GPC3×CD3 engager using purified CD4 T cells (upper panels) and purified CD8 T cells (bottom panels), in a XCELLIGENCE assay.

In Vitro Cytolysis Induced by LM1653 Against GPC3⁺ Hep3B

### Method

Dissociated tumor cells from HCC patients were used as source of effector cells in this assay, using the XCELLIGENCE systems. Tumor-infiltrating T cells were added to the culture at E:T ratio of 2:1 and 3:1, respectively. Target cells were HCC GPC3+ Hep3B.

### Results

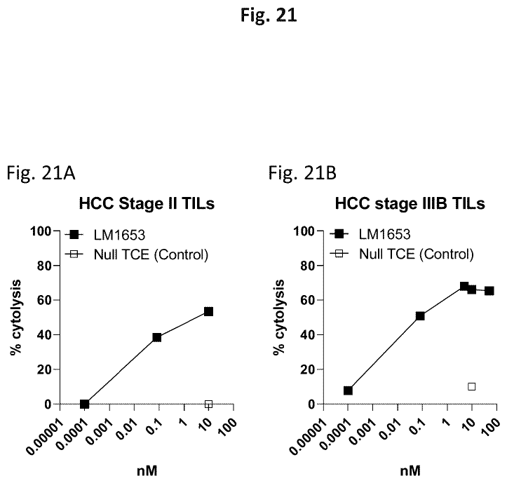

FIG. 21 shows in vitro cytolysis induced by LM1653 against GPC3⁺ Hep3B, using HCC patient-derived tumor infiltrating lymphocytes. FIG. 21 shows LM1653-dependent killing of GPC3⁺ Hep3B cell line by patients-derived tumor infiltrating lymphocytes obtained from HCC stage II (FIG. 21A) and stage IIIB (FIG. 21B). Tumor-infiltrating T cells were added to the culture at E:T ratio of 2:1 and 3:1, respectively. Cytotoxicity activity is shown for LM1653 and a null control compound unable to bind GPC3.

### In Vitro Cytolysis Induced by LM1653 Against GPC3+ Lung and Ovarian Cancer Cells

### Method

Cytotoxicity assays against the indicated ovarian and lung cell lines were run for 4 days using PBMCs at an E:T ratio of 10:1, using the XCELLIGENCE system.

### Results

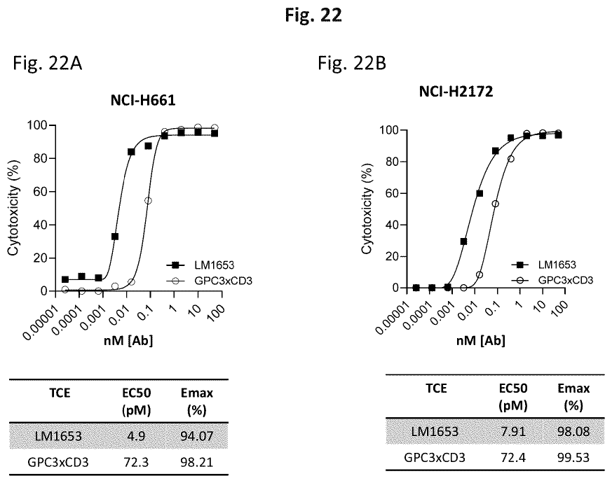

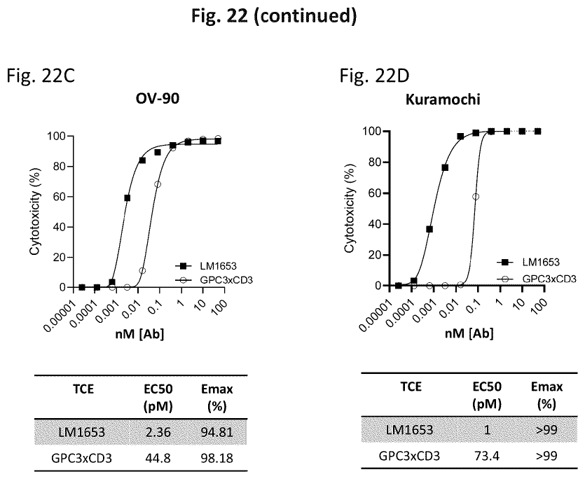

FIG. 22 shows in vitro cytolysis induced by LM1653 compared to a conventional GPC3×CD3 engager, against GPC3+ lung and ovarian cancer cell. FIG. 22 shows the in vitro cytolytic activity of LM1653 and of a monovalent GPC3×CD3 engager against non small cell lung cancer lines NCI-H661 (FIG. 22A) and NCI-H2172 (FIG. 22B) and ovarian cancer cell lines OV-90 (FIG. 22C) and Kuramochi (FIG. 22D). The tables in the figures show the corresponding EC₅₀ and killing Eₘₐₓ for each cell line

### In Vitro Cytolysis and T Cell Activation Profile Induced by LM1653 and LM1653-2.

### Method

Cytotoxicity assays against the indicated HCC cell line were run for 4 days using and cytolysis activity was measured at day 4, using PBMC at the ratio of 10:1 in the XCELLIGENCE system (n=1 PBMC donor). T cell activation profile was determined in cytotoxicity assays by flow cytometry.

### Results

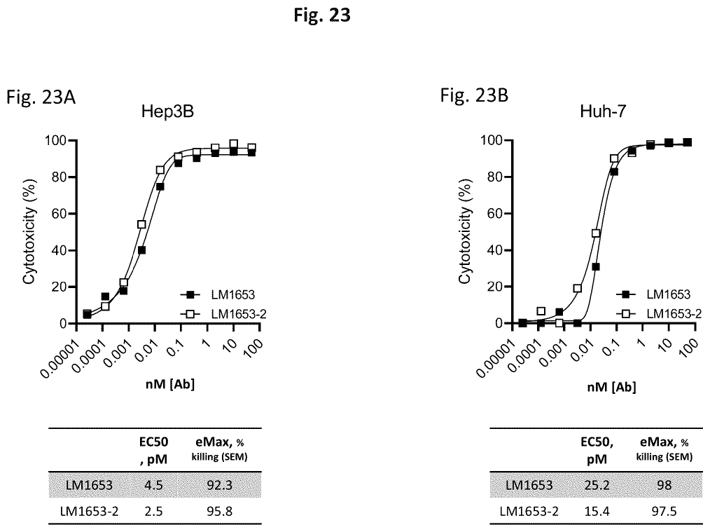

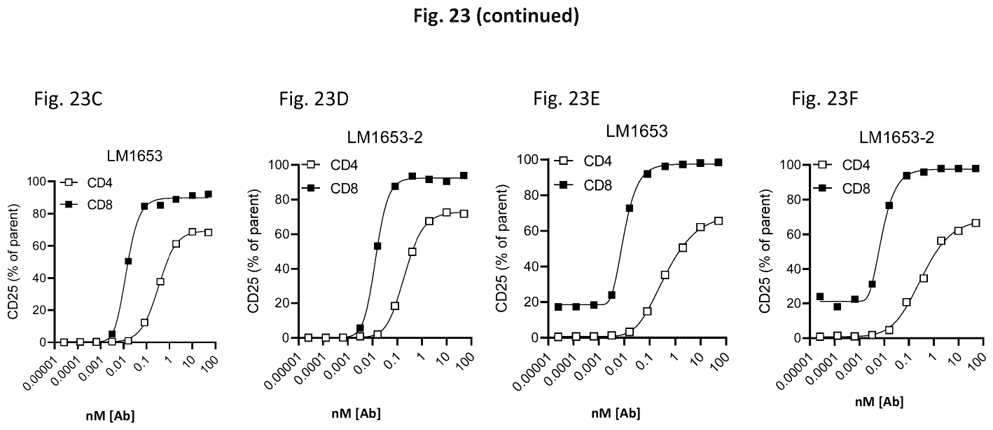

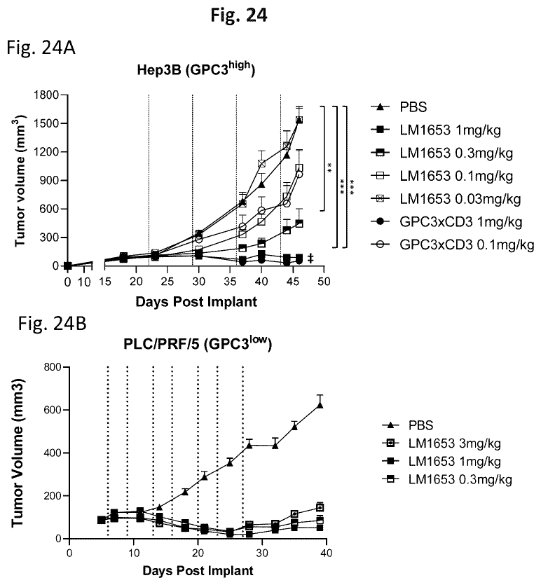

FIG. 23 shows comparable in vitro cytolysis and T cell activation profile induced by LM1653 and LM1653-2 (same amino acid sequence as LM1653, but produced in a different lot). FIG. 23A-B The tables show the corresponding EC₅₀ and killing Eₘₐₓ for GPC3+ cell line Hep3B (FIG. 23A) and Huh-7 (FIG. 23B). T cell activation profile was determined in cytotoxicity assay, using the Hep3B (FIG. 23C-D) and Huh-7 cell line (FIG. 23E-F). The percentage of CD8 (filled symbol) and CD4 (empty symbol) T cells expressing CD25 is reported.

In Vivo Anti-Tumor Activity of LM1653 Against GPC3^{high} and GPC3^{low} HCC Xenograft Models.

### Method

### GPC3^{high}

At day 0, NSG mice (n=10/group) were implanted SC with 5e06 Hep3B cells, expressing high levels of GPC3. On day 21, animals were humanized with 10e06 panT cells expanded in vitro. Animals were treated starting on day 22 IP once a week with PBS, LM1653 (1, 0.3, 0.1 and 0.03 mg/kg) and conventional GPC3×CD3 engager (1 and 0.1 mg/kg), for a total of 4 injections. Tumor burden was measured overtime and reported as mean±SEM. $, 40% tumor-free; **, p<0.01 and ***, p<0.001, 2-way Anova (FIG. 24A).

### GPC3^{low}

At day 0, NSG mice (n=10/group) were implanted SC with 5e06 PLC/PRF/5 cells, expressing low levels of GPC3. On day 5, animals were humanized with 10e06 panT cells expanded in vitro. Animals were treated starting on day 6 IP twice a week with PBS and LM1653 (3, 1, and 0.3 mg/kg), for a total of 7 injections. Tumor burden was measured overtime and reported as mean±SEM. **, p<0.0001, 2-way Anova (FIG. 24B).

### Results

FIG. 24 shows that LM1653 confers anti-tumor efficacy in vivo against GPC3^{high} and GPC3^{low} HCC xenograft models. Dotted lines represent TCE administrations. In vivo anti-tumour activity and associated cytokine secretion of LM1653 in Hep3B tumour bearing mice

### Method

NSG mice (n=11-12 for each treatment group) were irradiated with 200 cGy at Day −3 and implanted IP with 7×10⁶ Hep3B-Luciferase cells at Day 0. At Day 8, mice were engrafted IV with 1×10⁷ human PBMCs from one healthy donor. At Day 22, mice were injected IP with either vehicle (PBS), LM1653-2 or GPC3×CD3 at 1 mg/kg. Tumor burden was monitored twice a week by bioluminescence imaging (BLI) until Day 24, 2 days post treatment.

### Results

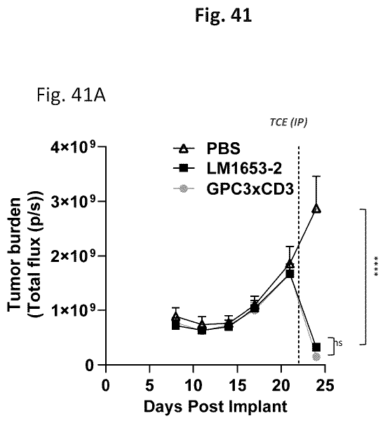

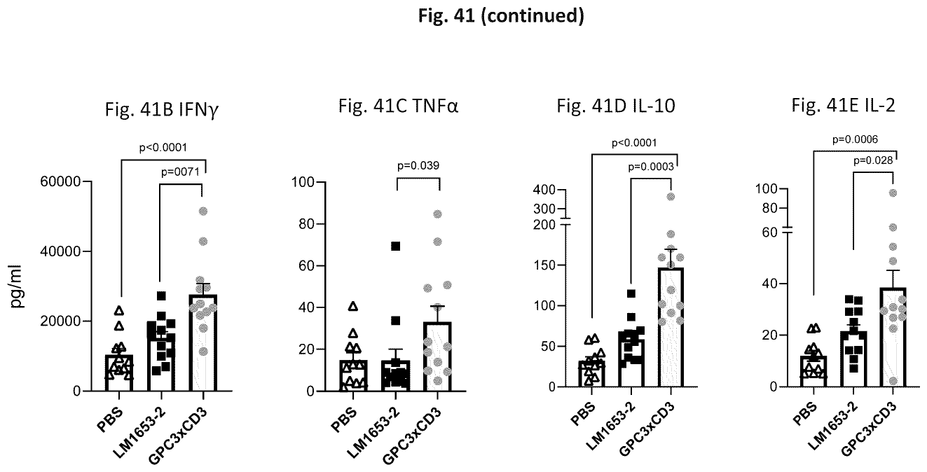

FIG. 41 shows that LM1653 induced significant tumor growth inhibition in Hep3B tumor bearing mice without significant associated cytokine secretion, in comparison to a conventional GPC3×CD3 engager.

FIG. 41A shows the tumor growth inhibition induced by both LM1653-2 and a conventional GPC3×CD3 engager. The mean of tumor burden+/−SEM across the course of the experiment in each treatment group is shown. Dashed line indicate day of treatment. The statistical significance of the differences in tumor burden overtime was determined using 2-way ANOVA with Tukey's multiple comparisons test (****p<0.0001; ns=not significant).

In FIG. 41B-E, serum cytokine concentrations were evaluated at 6 hours post TCE administration by multiplex assays. Individual values as well as mean±SEM for each treatment group is plotted for IFN-7 (FIG. 41B), TNF-α (FIG. 41C), IL-10 (FIG. 41D), and IL-2 (FIG. 41E). The statistical significance of the differences was determined using One-way ANOVA with Tukey's multiple comparisons test.

### In Vivo Tumour Growth Inhibition Activity and Associated Cytokine Secretion of LM1653-3 in NCI-H661 Lung Tumor Bearing Mice

### Methods

NSG mice (n=5-7 for each treatment group) were irradiated with 200 cGy at Day −3 and implanted IV with 2.5×10⁶ NCI-H661-Luciferase cells at Day 0. At Day 12, mice were engrafted IV with 1×10⁷ human PBMCs from one healthy donor. Starting at Day 27, mice were injected IV with either vehicle (PBS) or LM1653-3 at 1 and 0.3 mg/kg 2QW, for a total of 4 doses. OKT3 was used as positive control for T cell activation and cytokine secretion. LM1653-3 has the same amino acid sequence as LM1653 but was produced in a different lot.

### Results

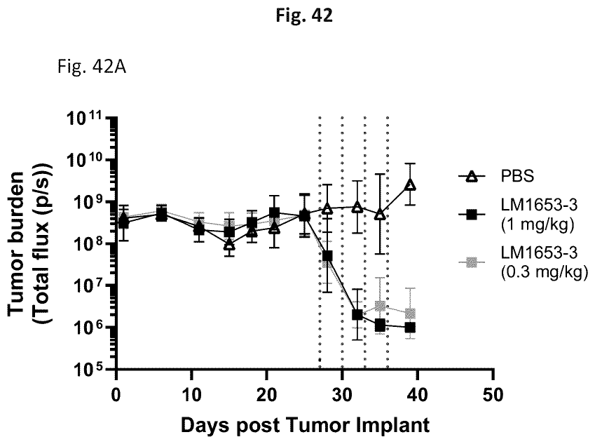

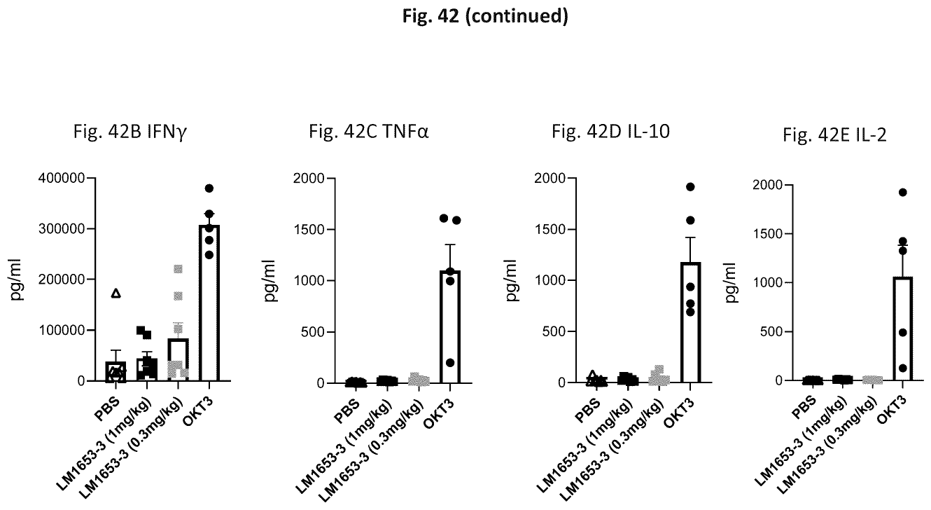

FIG. 42A shows that tumor growth inhibition was induced at both doses of LM1653-3.

FIG. 42 shows that LM1653-3 induced significant tumor growth inhibition in NCI-H661 lung tumor bearing mice without significant associated cytokine secretion, in comparison to vehicle-treated animals. Tumor burden was monitored twice a week by bioluminescence imaging (BLI). The geometric mean of tumor burden+/−SD across the course of the experiment in each treatment group is shown. Dashed line indicate days of treatment. The statistical significance of the differences in tumor burden overtime was determined using 2-way ANOVA with Tukey's multiple comparisons test (*, p<0.05; **p<0.01; ****p<0.0001). LM1653-3 exhibits the same in vitro bioactivity profile of LM1653 and LM1653-2.

In FIG. 42B-E, serum cytokine concentrations were evaluated at 3 hours post 1^{st} TCE or OKT3 administration by multiplex assays. Individual values as well as mean±SEM for each treatment group is plotted for IFN-7 (FIG. 42B), TNF-α (FIG. 42C), IL-10 (FIG. 42D), and IL-2 (FIG. 42E).

### Example 6: Efficacy of LM1653 (GPC3 TITAN) in Cynomolgous Monkey Model

### Method

Kinetic measurements to soluble forms of GPC3 and CD8ab were performed using a BIACORE instrument. K_{D} were calculated as the ratio of k_{off}/kₒₙ from a non-linear fit of the data. Values are presented as mean±standard deviation (SD) of three experiments. Owing to assay limitations, kinetic values for TCR binding were unable to be determined accurately.

PBMC from 3 different healthy human or 4 cynomolgus monkey donors were incubated with HCC GPC3+ HepG2 at an effector to target ratio of 10:1. LM1653 was added in a 5-fold serial dilution starting from 50 nM for a total of 9 dilution doses. The percentage of cytolysis was assessed using XCELLIGENCE impedance-based RTCA MP analyzer at 48 hours post PBMC addition.

Cynomolgus monkey (n=3 per treatment group and per sex) were administered LM1653 weekly by intravenous (bolus) or subcutaneous injection for 1-month (5 doses), at doses of 1 mg/kg/week IV, 6 mg/kg/week IV or 6 mg/kg/week SC followed by a 2-week recovery period. Animals that received vehicle only as both IV & SC were used as controls.

### Results

Table 4 shows the binding kinetics of LM1653 to recombinant human and cynomolgus monkey GPC3 and CD8 Proteins.

TABLE 4

| Antigen | Kₒₙ (M⁻¹s⁻¹) | K_{off} (s⁻¹) | K_{D} (nM) |
|---|---|---|---|
| Human GPC3 | 3.41 ± 0.29 E+05 | 2.26 ± 0.04 E−04 | 0.67 ± 0.05 |
| Cynomolgus | 3.26 ± 0.04 E+05 | 2.67 ± 0.20 E−04 | 0.82 ± 0.06 |
| monkey | | | |
| GPC3 | | | |
| Human CD8ab | 3.40 ± 0.23 E+05 | 2.80 ± 0.18 E−02 | 82.7 ± 11.0 |
| Cynomolgus | 5.27 ± 0.92 E+05 | 3.29 ± 0.59 E−02 | 62.4 ± 4.1 |
| monkey | | | |
| CD8ab | | | |

Table 5 shows LM1653-Induced T cell dependent cellular cytotoxicity (TDCC) in HCC HepG2 Cell Line, using human and cynomolgus Monkey PBMCs. Table 5 shows TDCC EC₅₀ and Eₘₐₓ median with interquartile range (IQR) for both human and cynomolgus monkey PBMCs.

TABLE 5

| | T cell Dependent Cellular Cytotoxicity | | | |
|---|---|---|---|---|
| | Median EC₅₀ (pM) | | | |
| Species | (IQR = Q3 − Q1) | | Eₘₐₓ (%) | |
| Human (n = 3) | 8.28 | (23.01) | 89.72 | (14.27) |
| Cynomolgus monkey (n = 4) | 31.55 | (85.54) | 67.36 | (24.60) |

| | | |
|---|---|---|
| Fold differences (c vs h) | 3.8 | 0.76 |

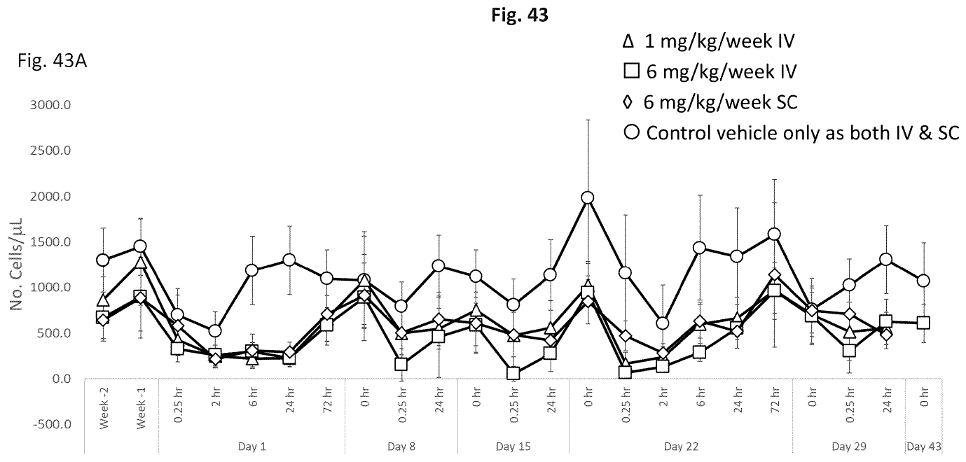

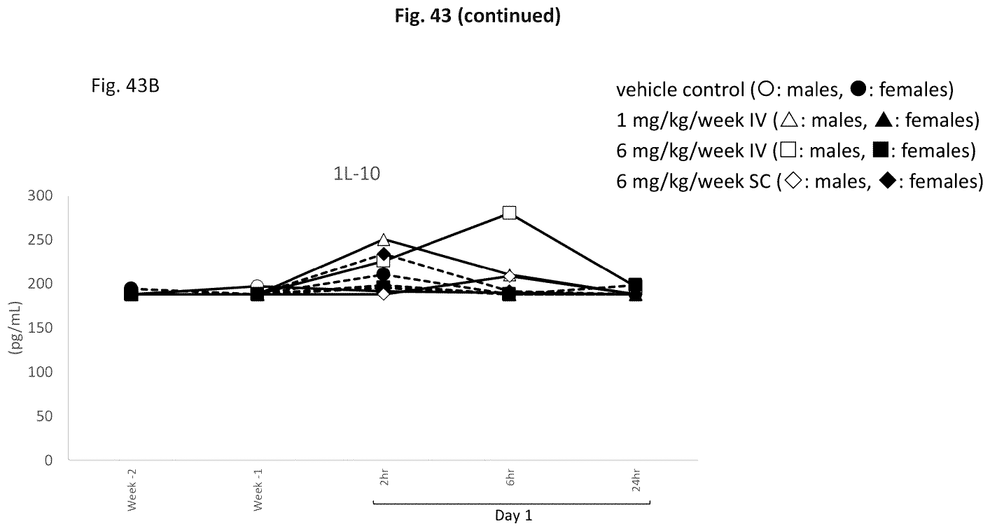

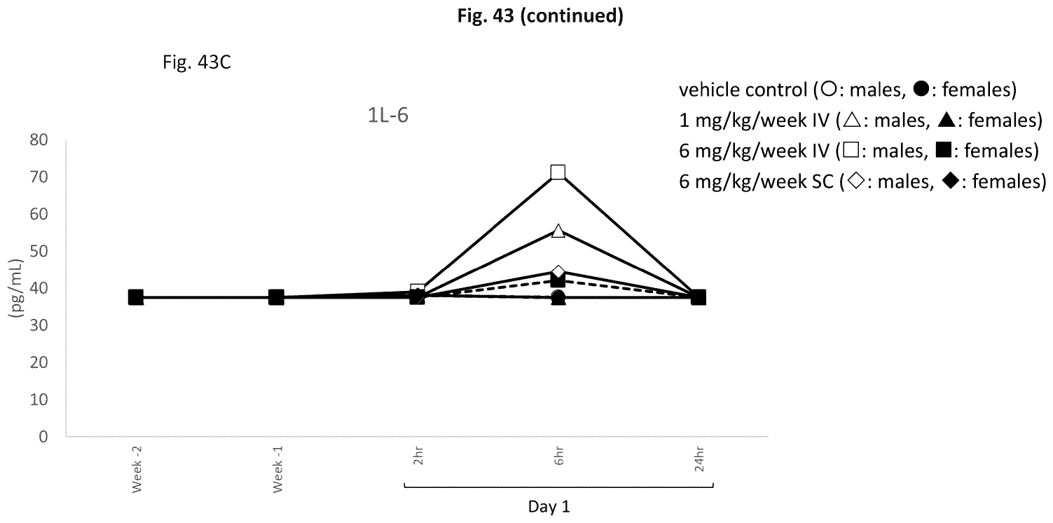

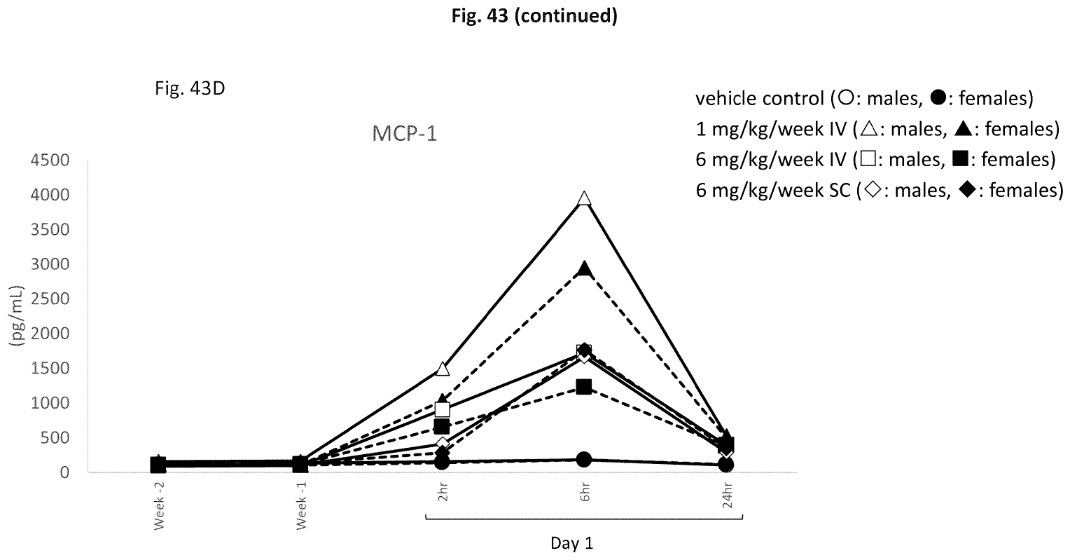

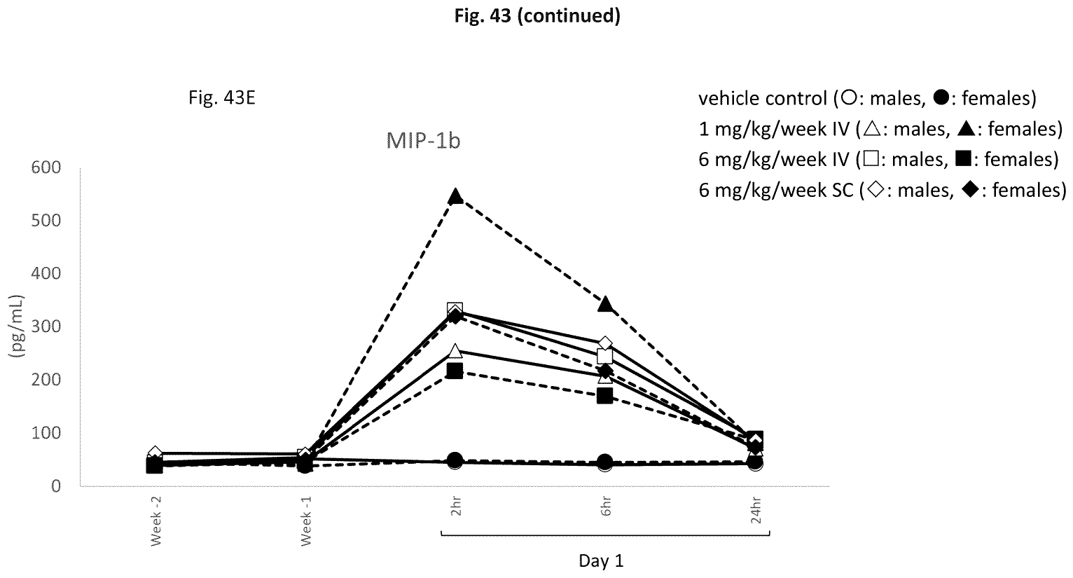

FIG. 43 shows the effect of LM1653 administration in cynomolgus monkey. FIG. 43A shows the absolute number of CD8 T cells in blood over the course of treatment. Administration of LM1653 was associated with a transient decline in the number of CD8 T cells in the blood, as a result of T cell margination. Margination is considered an expected effect of LM1653 activity, as consequence of drug interaction with target T cells.

FIGS. 43B-E show serum cytokine and chemokine levels following the first dose administration. LM1653 was associated with a transient increase in the chemokines MCP-1 and MIP-1b, that are associated with CD8 T cell migration and function. Only marginal and transient increases were observed for CRS-associated cytokines IL-10 and IL-6, in agreement with the preclinical data generated in vitro and in mice. LM1653 activity is accompanied by only minimal increases in circulating cytokine level, potentially lowering the risk of CRS-associated toxicity in patients. Solid and dotted lines represent values obtained for male and female, respectively, and vehicle control.

### Example 7: Humanisation of Anti-TCR VHH Framework Residues

Anti-TCR VHH amino acid sequence of SEQ ID NO: 42 is of camelid nature. Humanization of the amino acid residues to minimize immunogenicity was performed, where non-vernier framework residues were altered to match closest human germline sequence IGHV3-23*1. During humanization, the nanobody hallmark residues Y42, and K52 in framework 2 were not altered. The lead humanized VHH panel were generated where the hallmark residues E49 and R50 were either humanized or not humanized. A total of approximately 40 humanized variants were assessed with a 90% binding retention threshold set. Prior to introduction in trispecific antibody format, humanized clones were tested for binding retention by generation as Fc fusions followed by binding kinetics by cell binding. Mutations from the top performing variants in Fc fusion were combined to generate 6 humanized anti TCR VHH containing a range of 5 to 10 humanized residues. These were integrated into the final trispecific format. The final lead panel was assessed for cytotoxicity and T cell activation. All variants matched the criteria in final format. The following TCR V_{HH}s were selected: hu2.1 (SEQ ID NO: 43), hu2.2 (SEQ ID NO: 44), hu2.3 (SEQ ID NO: 45), hu2.4 (SEQ ID NO: 46), hu2.5 (SEQ ID NO: 47) and hu2.6 (SEQ ID NO: 48).

### Example 8: Efficacy of LM1486 (CD20 TITAN) in Inflammatory Disease and Autoimmune Disorder Models

### Methods

### Human Peripheral Blood Mononuclear Cells (PBMCs)

Primary human PBMCs were isolated from the whole blood obtained from SLE or myositis patients. PBMCs were counted and 100K/well PBMCs were treated with varying concentrations of the CD20 TITAN molecule in a 96-well plate. PBMCs were cultured for three days at 37° C. in a CO₂ incubator under sterile conditions. The depletion of B cells was measured at the end of three days by flow cytometry analysis and IC₅₀ values were calculated using Prism GraphPad software.

### Humanized Mouse Model

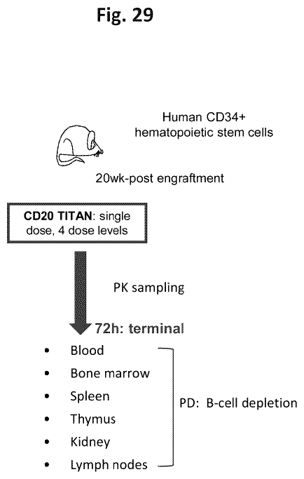

FIG. 29 depicts the method steps. NOD-scid IL2Rgamma^{null} (NSG) mice were obtained from Jackson lab at about 22 weeks post-engraftment, randomized and treated with either vehicle (PBS), NIP228 control at 1 mg/kg or either 0.03 mg/kg, 0.1 mg/kg, 0.3 mg/kg, or 1 mg/kg of LM1486 via intraperitoneal (i.p.) injection. 72 hours post-treatment, blood and tissues were collected and processed for further analysis.

The spleens were meshed through cell strainers with a syringe plunger with 5 mL of neutralize buffer (RPMI1640 (GIBCO) containing 5% FBS (GIBCO) and 2 mM EDTA (INVITROGEN) and centrifuged at 460 g for 5 minutes at 4° C. Cell pellets were resuspended with 1 mL of ACK lysis buffer (GIBCO) for 1 minute, then 2 mL of neutralize buffer was added before centrifugation at 460 g for 5 minutes at 4° C., the pellets were then resuspended with 2 mL of neutralize buffer.

The thymuses were meshed through cell strainers with a syringe plunger with 5 mL of neutralize buffer and centrifuged at 460 g for 5 minutes at 4° C. Cell Pellets were resuspended in 2 mL of neutralize buffer.

Bone marrow cells were flushed out from the femur and tibia from a hind leg using a 25G needle and passed through a cell strainer to remove any bone spicules, muscle or cell clumps, and red cells were lysed with ACK lysis buffer (GIBCO); after centrifugation the pellet was resuspended in cold neutralize buffer.

Blood samples were collected in EDTA pre-treated tube (BD) and centrifuged at 500 g for 10 minutes at 4° C. to separate the plasma from cells. The cell pellets were resuspended in 5 mL of ACK lysis buffer (GIBCO) for 90 seconds at room temperature to lyse the red cells. Washed with 5 mL of neutralize buffer and resuspended in 1 mL cold neutralize buffer.

Capsules and extra connected tissues were removed from all kidney samples by forceps and were gently crushed with a syringe plunger. Dissociation was obtained by using Miltenyi gentleMACS™ dissociation protocol.

In summary, samples were collected into a Miltenyi gentleMACS C tube with 2 mL Multi tissue dissociation medium and incubated on program “37 Multi E” for 30 minutes at 37° C. The cell suspensions were neutralized by adding 2 mL of neutralize buffer and filtered through 40 um cell strainers. After centrifugation, the pellets were resuspended in 40% percoll (GE HEALTHCARE) and centrifugated at 580 g for 20 minutes at room temperature. The cells on bottom were collected and washed with neutralize buffer, then resuspended into 1 mL of cold neutralize buffer. For flow cytometry analysis: 2 million cells were incubated with Live/Dead NIR 780 (THERMOFISHER) at 1:2000 dilution in 200 μl of RPMI1640 containing 2 g Fc blocker (BIOLEGEND) for 5 minutes at 4° C. Cells were washed with FACS buffer by centrifugation at 460 g for 5 minutes at 4° C. Pellets were resuspended in 50 μl of antibody mix for staining with anti-human CD3 BUV 395 (BD, 1:100dilution), anti-human CD19 BUV737 (BD, 1:200 dilution), anti-human CD8a BV711 (BIOLEGEND, 1:400 dilution), anti-human CD2 BB515 (BD, 1:1200 dilution), anti-human CD25 PE (BIOLEGEND, 1:50 dilution), anti-human CD4 PE-Cy7 (BIOLEGEND, 1:400 dilution), anti-human CD45 APC (BIOLEGEND, 1:200), anti-mouse CD45 BV510 (BIOLEGEND, 1:400 dilution) containing 2.5 μl Superbright staining buffer and incubating for 30 minutes on ice. Cells were washed twice with FACS buffer, and then fixed by adding 100 μl of 1× fixation buffer (EBIOSCIENCE) at room temperature for 20 minutes. After centrifugation, cells were resuspended in 200 μl FACS buffer. Flow cytometry was performed using Symphony A3 (BD BIOSCIENCE). Analysis was conducted with FLOJO software.

Lymph nodes were fixed for 48 hours in 4% PFA, dehydrated and embedded into paraffin blocks. Four micrometer thick tissue sections were cut and stained using an immunofluorescence panel with OPAL dyes (AKOYA BIOSCIENCES) for detection: anti-CD19 (EPR5906, Abcam; OPAL 520), anti-CD25 (EPR6452, Abcam; OPAL 570), anti-CD8 (D8A8Y, Cell Signaling Technology; OPAL 620), and anti-CD4 (EPR6855, Abcam; OPAL 480). Digital whole-slide images were generated using PhenoImager HT (AKOYA BIOSCIENCES) and cell classification and tissue area quantification was performed using VISIOPHARM v.2023.09.5.15777.

### Results

Calculation of IC₅₀ values for B cell depletion and maximum depletion of B cells in SLE and myositis patients derived PBMCs are depicted in Table 6.

TABLE 6
B cell depletion and max depletion of B cells in SLE and Myositis

| | B cell depletion IC₅₀ | % avg. Max B cell |
|---|---|---|
| Patient ID | (pM) | depletion |
| Myo 109785 | 107.5 | 96 |
| Myo 107370 | 2.8 | 94 |
| Myo 120323 | 7.1 | 62 |
| Myo 109852 | 1.1 | 86 |
| Myo 112243 | 3.8 | 89 |
| Myo 123136 | 13.3 | 68 |
| Myo 138502 | 38.1 | 84 |
| SLE 29144 | 8.5 | 81 |
| SLE 89313 | 1.2 | 88 |
| SLE 20643 | 11.5 | 60 |
| SLE 23199 | 1.4 | 27 |
| SLE 91744 | 19.1 | 41 |
| SLE 23150 | 0.5 | 63 |
| SLE 29942 | 0.3 | 41 |
| SLE 29941 | 1.8 | 75 |

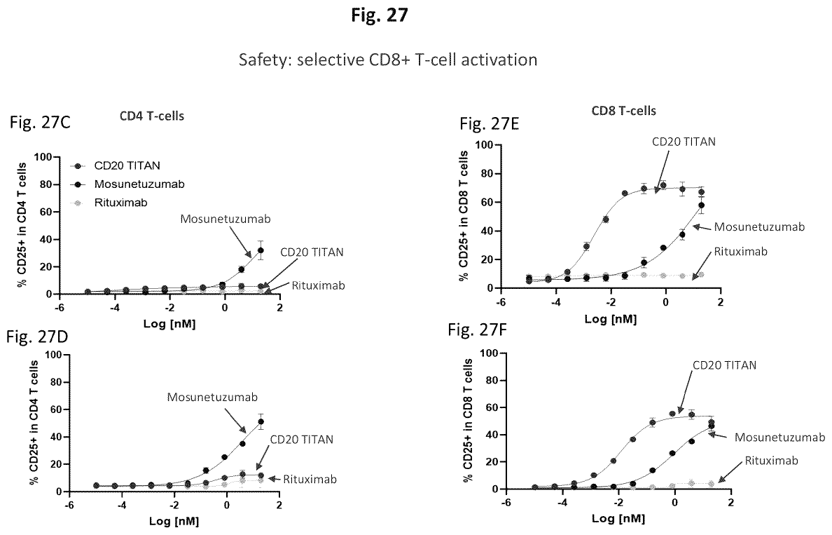

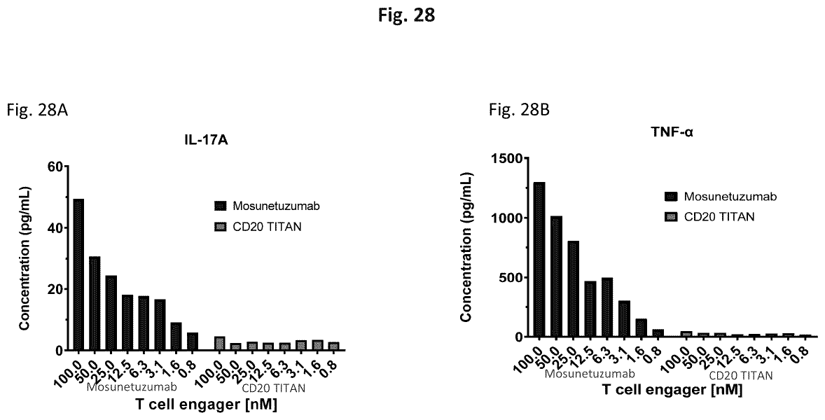

FIGS. 27 and 28 demonstrate the efficacy and safety of LM1486 (CD20 TITAN). LM1486 showed superior and potent B cell depletion (FIGS. 27A and B) in cells from patients with SLE and myositis, while also demonstrating excellent safety with selective CD8+ T-cell activation (FIGS. 27E and F) and CD4 T-cell sparing activity (FIGS. 27C and D). FIGS. 28A and B demonstrate reduced cytokine release of IL-17A (FIG. 28A) and TNFα (FIG. 28B) in an assay of non-specific T-cell activation.

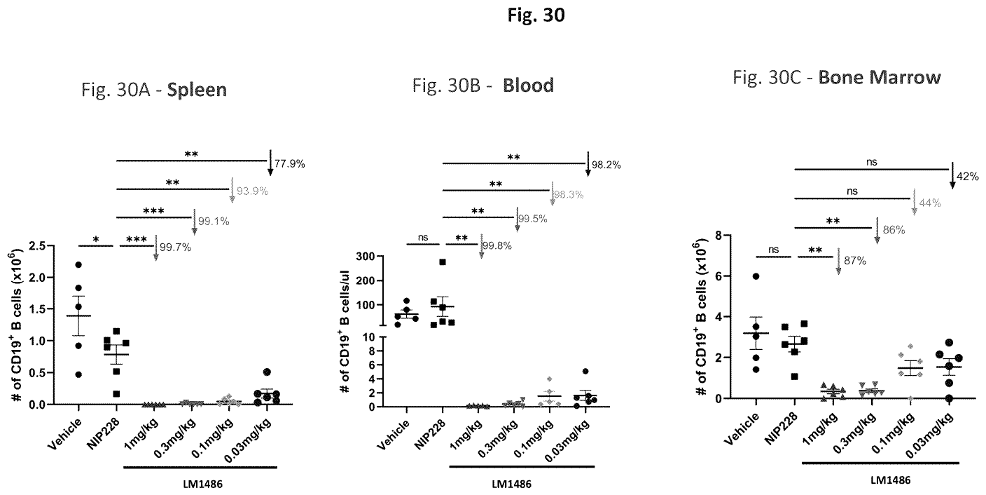

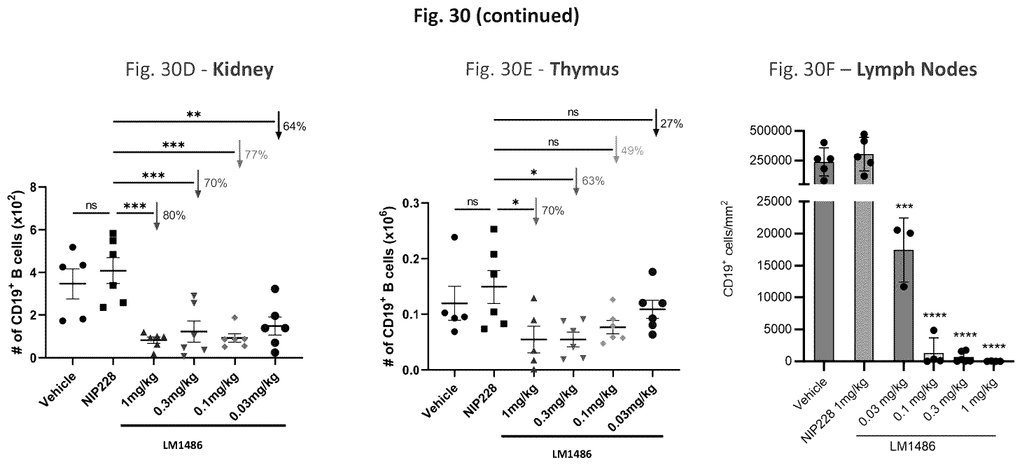

LM1486 (CD20 TITAN) achieved deep depletion of B cells in blood and tissue compartments in a humanized mouse model, 72 hours after 1 dose treatment in human-CD34+ cells engrafted NSG mice by flow cytometry. FIGS. 30A-F depict depletion of B cells in spleen (FIG. 30A), blood (FIG. 30B), bone marrow (FIG. 30C), kidney (FIG. 30D), thymus (FIG. 30E), and lymph nodes (FIG. 30F) in a humanized mouse model after a single dose LM1486 (CD20 TITAN). Depletion was more pronounced in peripheral blood and spleen.

### Example 9: Efficacy of LM1486 (CD20 TITAN) in Cynomolgous Monkey Model

### Method

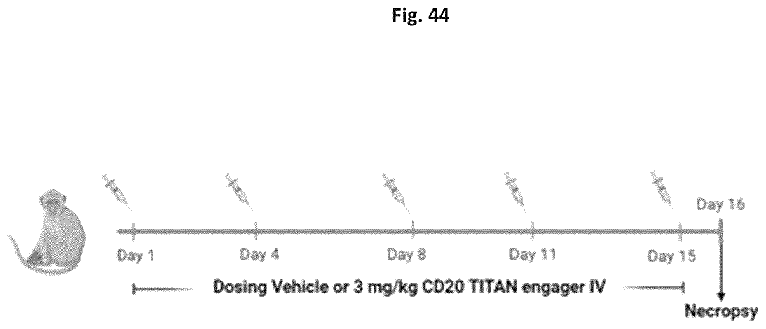

Cynomolgus monkeys were dosed twice weekly with either vehicle (n=10) or CD20 TITAN at 3 mg/kg (n=10). The CD20 TITAN group were dosed for a total of 5 doses and necropsy performed on Day 16 (FIG. 44). Mean±SEM B-cell counts (CD19+) were evaluated by flow cytometry and presented for peripheral blood (time course from Day −14 to Day 16) and tissues (Day 16). Absolute circulating CD8+ and CD4+ T cells post first dose and activated CD8/CD4 (CD25+) fold difference over baseline were assessed during the first week of treatment. Finally, group mean plasma cytokine profiles (IL-6, TNFα and IFNγ) were also assessed as a time course from Day −14 to Day 16.

### Results

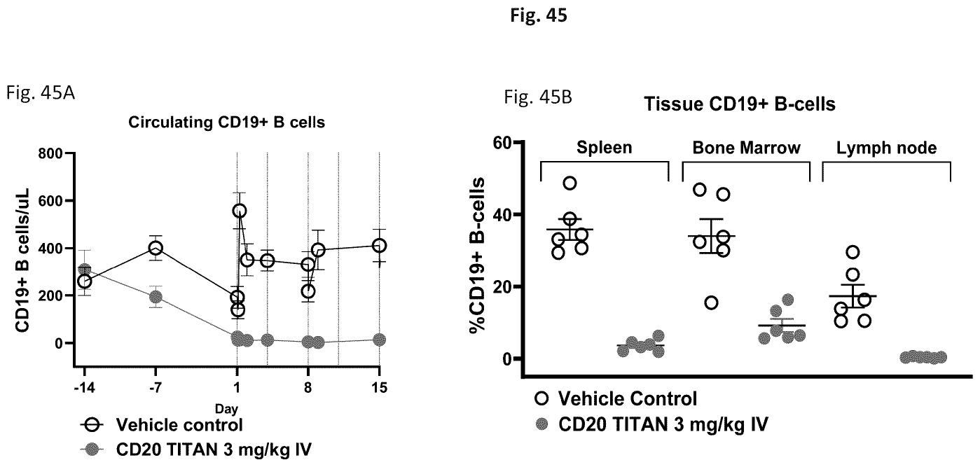

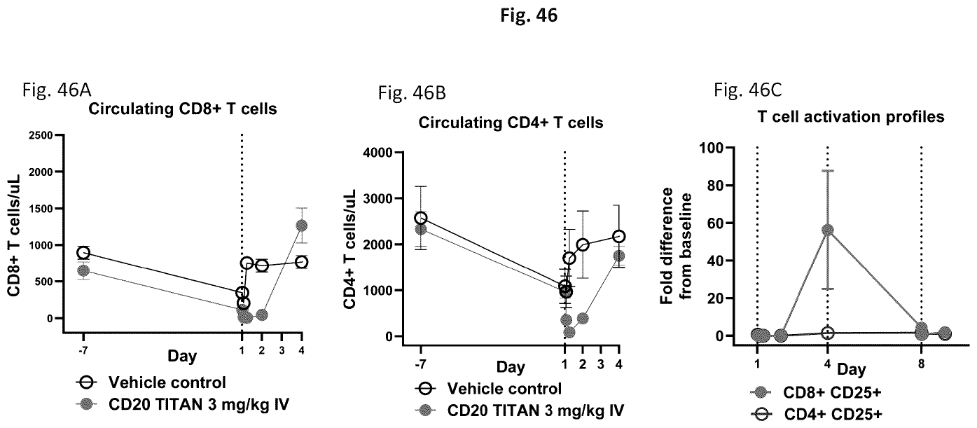

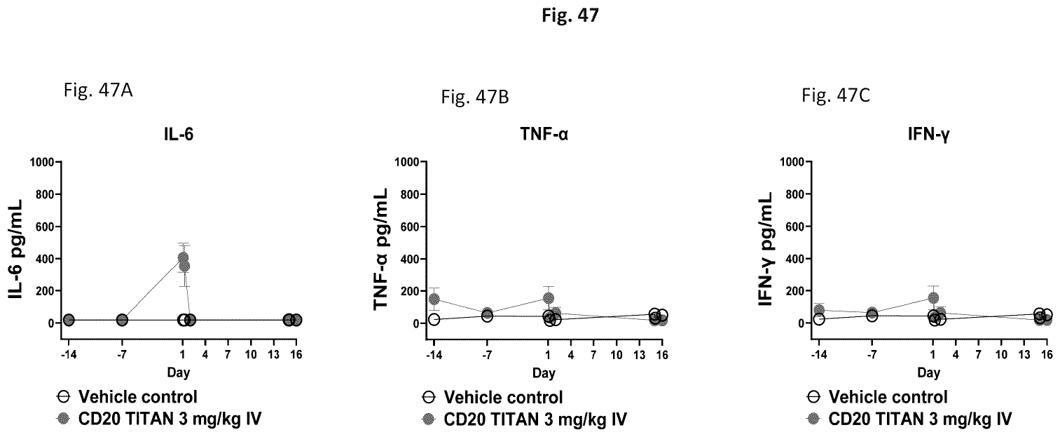

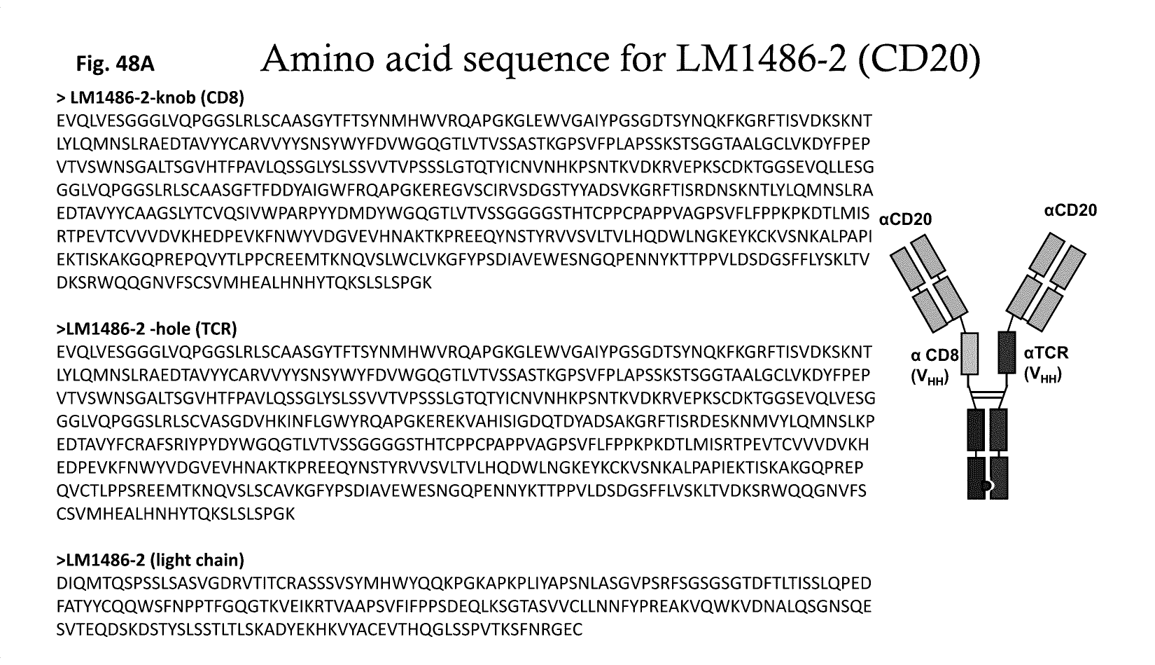

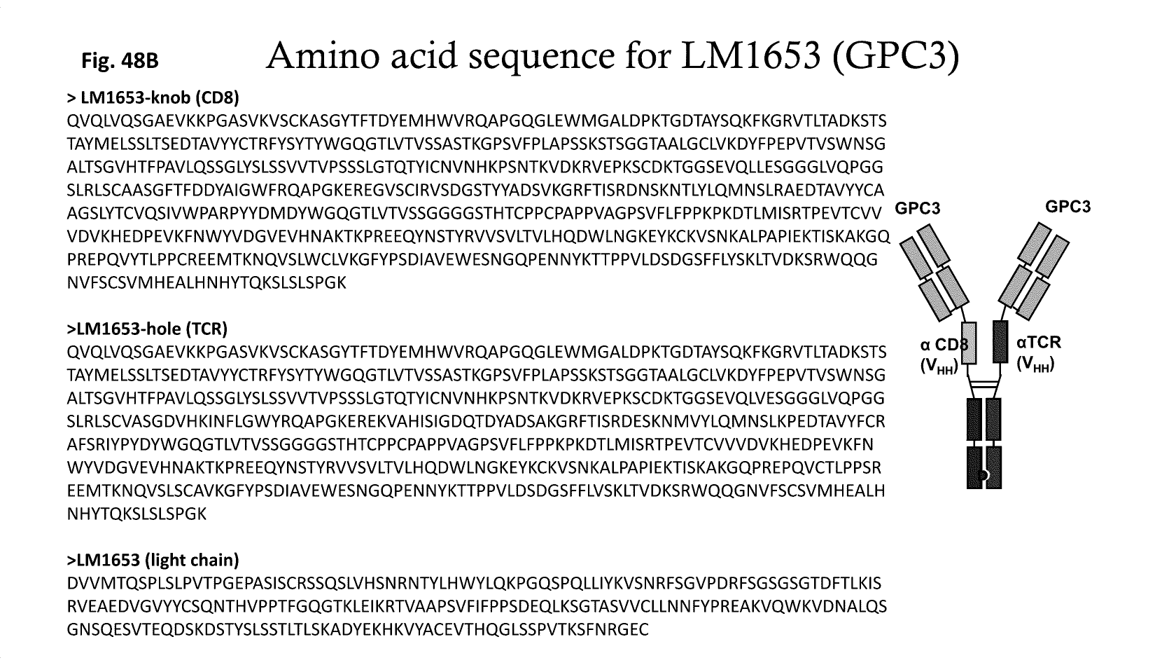

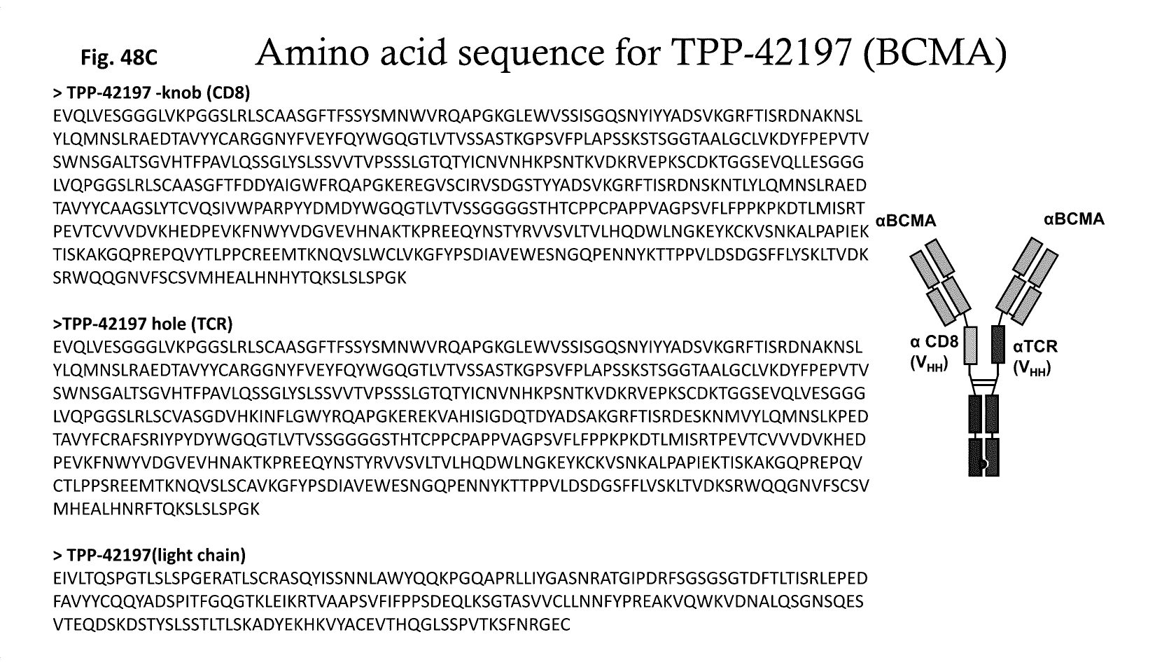

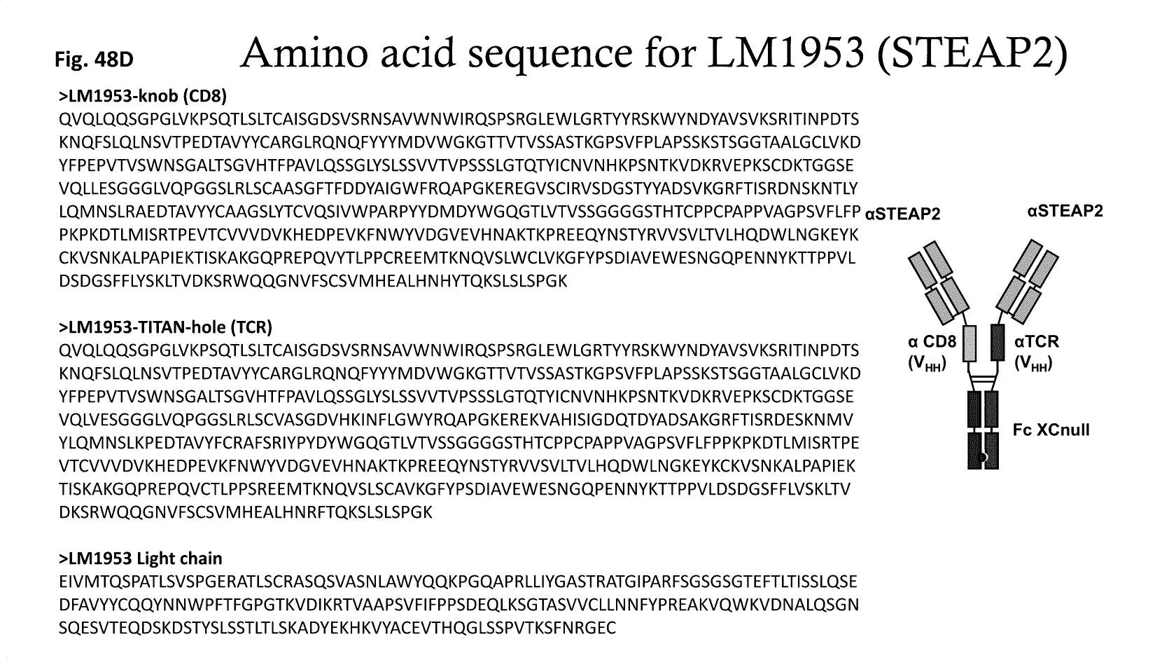

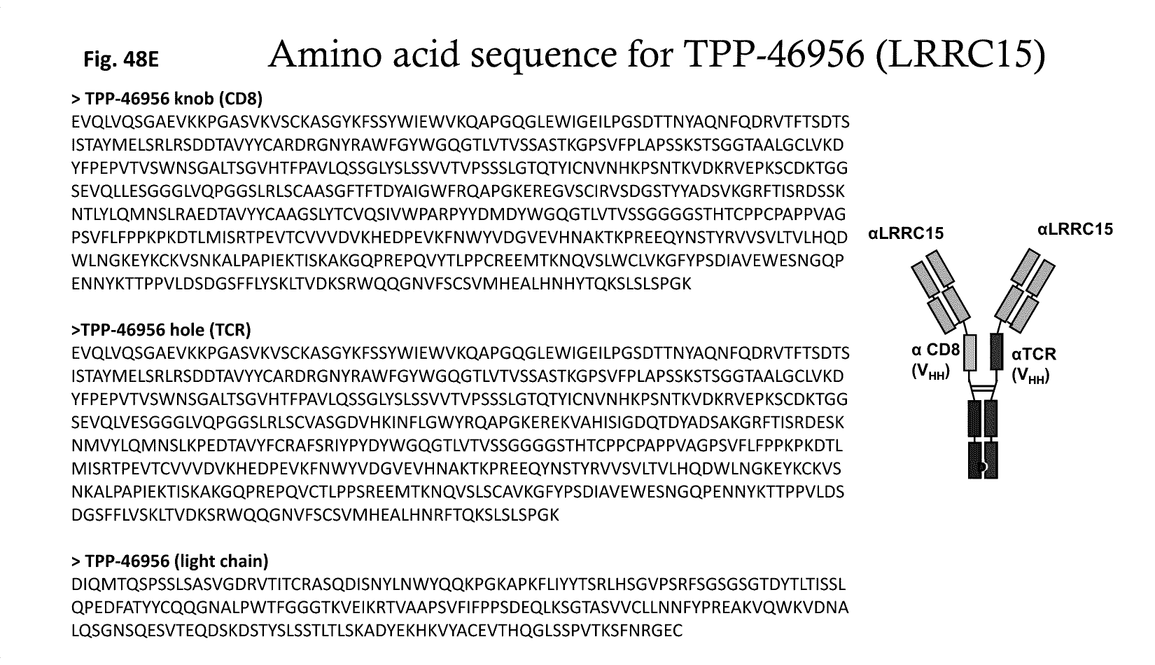

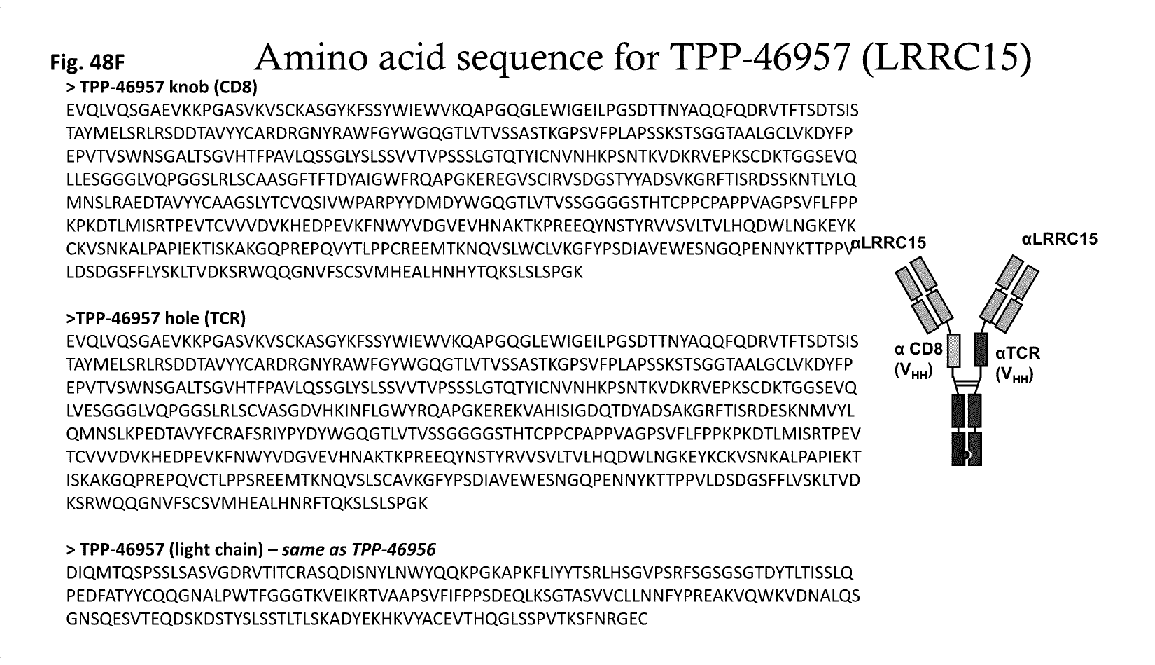

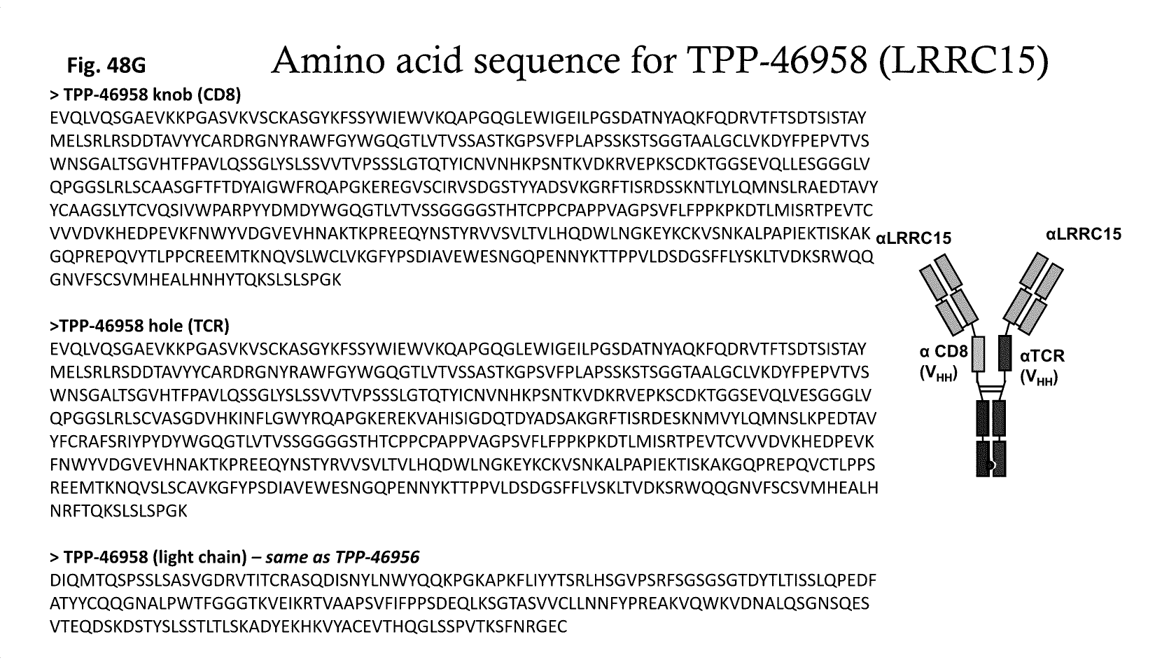

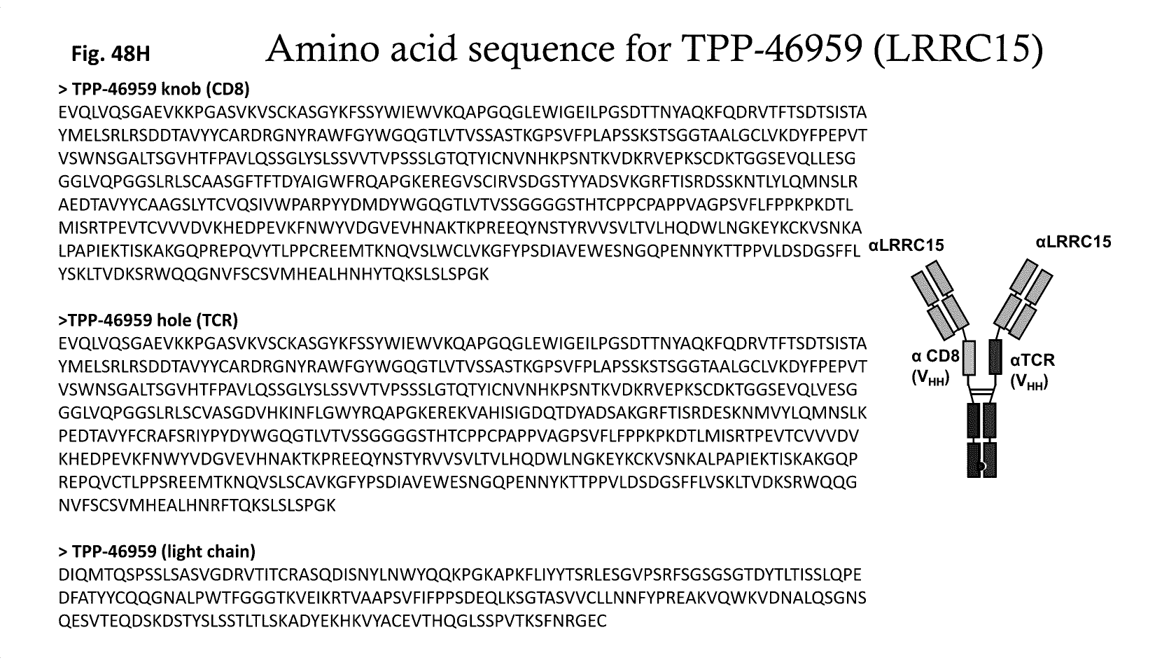

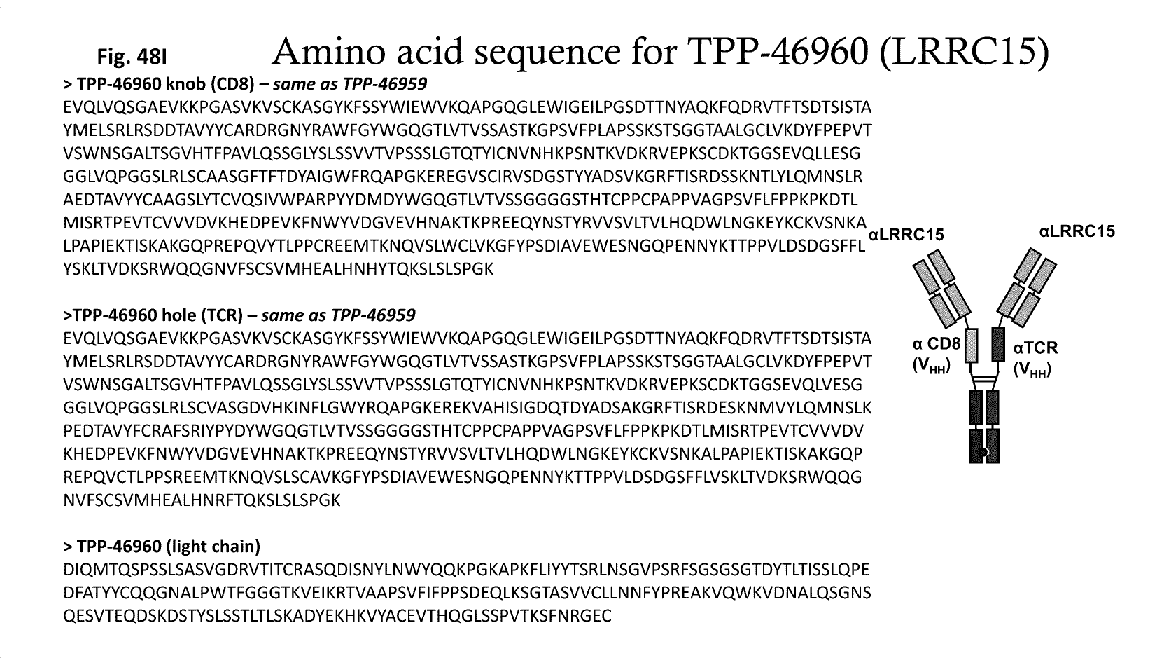

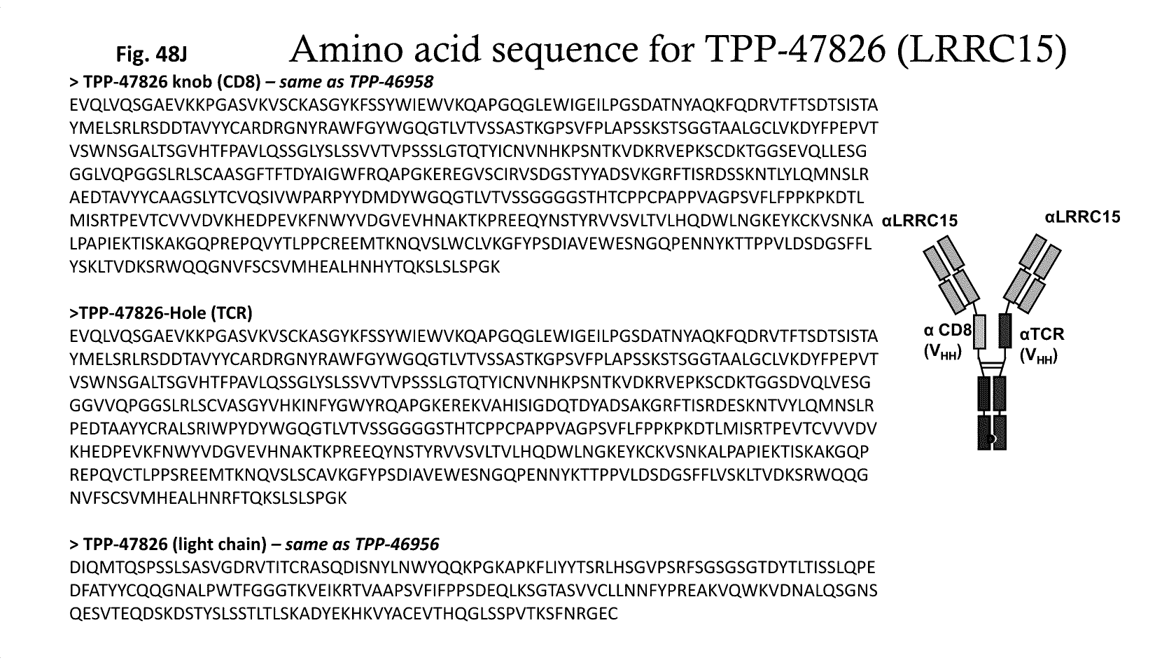

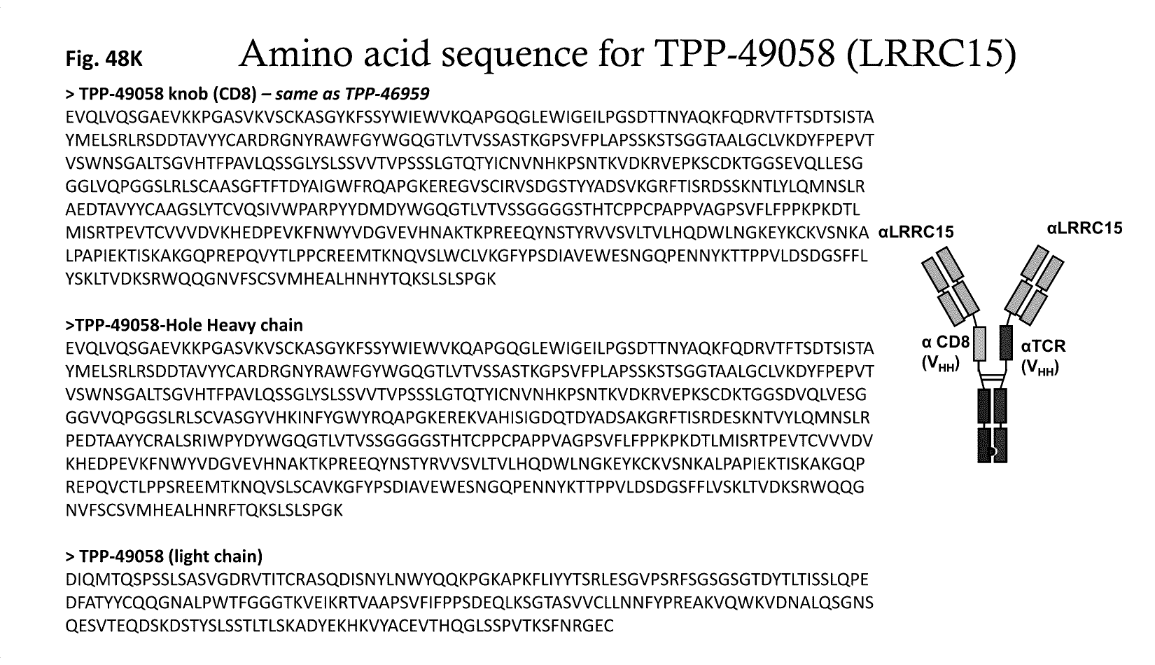

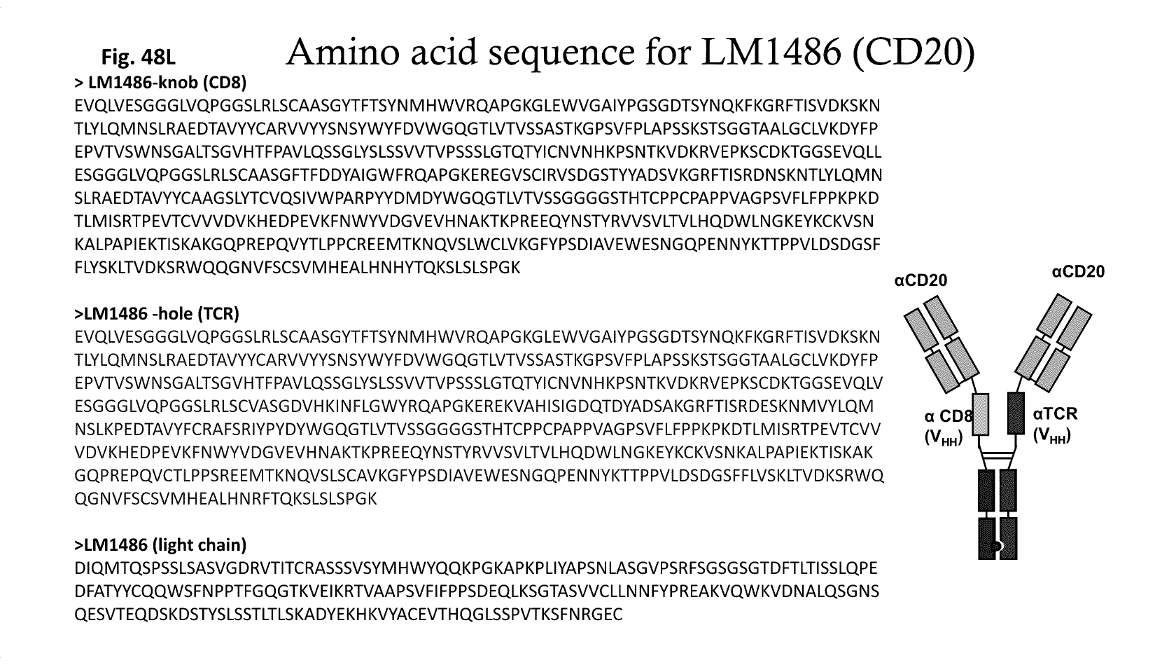

In a cynomolgus monkey model, LM1486 (CD20 TITAN) exhibits potent B cell depletion activity in cynomolgus monkey with low systemic cytokine release and associated toxicity at 3 mg/kg IV. FIG. 45 shows the depletion of circulating CD19+ B cells (FIG. 45A) and tissue specific CD19+ B cells in spleen, bone marrow and lymph nodes (FIG. 45B). FIG. 46 shows shows T cell margination and preferential CD8 activation. Absolute circulating CD8+(FIG. 46A) and CD4+ T (FIG. 46B) cells post first dose and activated CD8/CD4 (CD25+) (FIG. 46C) show a fold difference over baseline during the first week of treatment. FIG. 47 shows the group mean plasma cytokine profiles for IL-6 (FIG. 47A), TNFα (FIG. 47B), and IFNγ (FIG. 47C) as a time course from Day −14 to Day 16.

### Example 10: In Vitro and In Vivo Testing of LM1953 (STEAP2 TITAN)

The activity of the LM1953 (STEAP2, see FIG. 43D) binding protein disclosed herein was demonstrated.

### In Vitro Cytolysis, T Cell Activation and Cytokine Release Profiles

### Method

An assay was conducted to measure cytotoxic activity of LM1953. The assay was run for 3 days with the STEAP2+ prostate cancer lines 22Rv1 and C4-2 and PBMCs from 8 donors at an E:T ratio of 10:1 in an XCELLIGENCE assay. At the end of the cytolysis assay, T cell activation status was evaluated by flow cytometry and the cytokine release profiles were measured in the supernatant using a multiplexing assay.

### Results

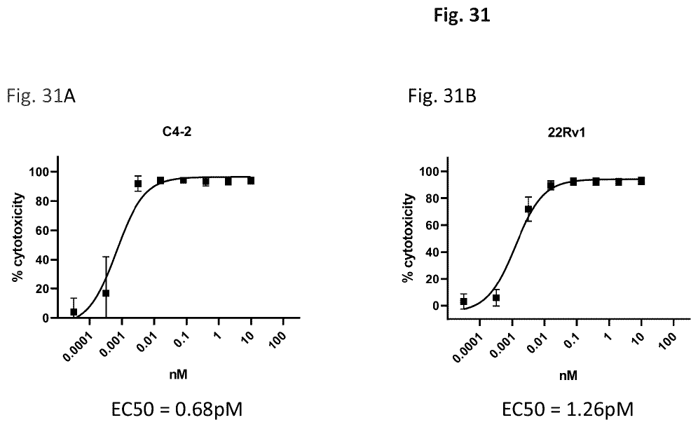

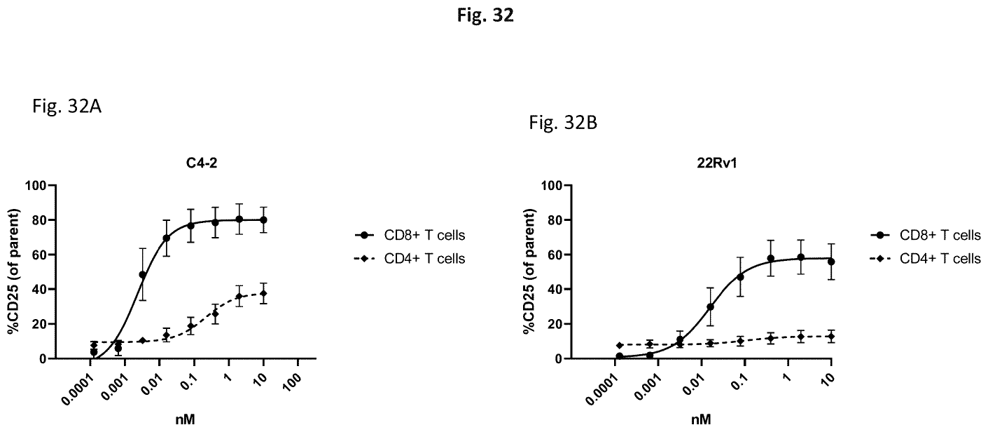

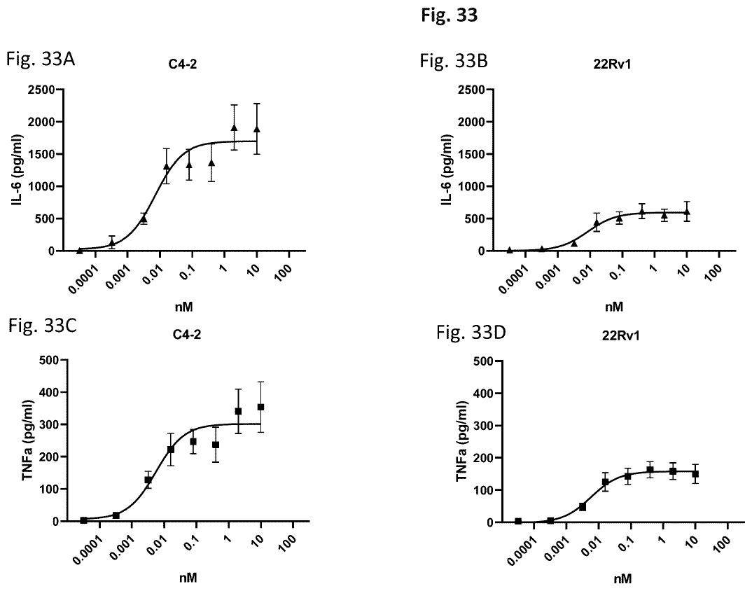

FIGS. 31A and B show potent in vitro cytolysis by LM1953. FIG. 31A shows the in vitro cytolytic activity of LM1953 on C4-2 cells and the corresponding EC₅₀. FIG. 31B shows the in vitro cytolytic activity of LM1953 on 22Rv1 cells and the corresponding EC₅₀. A CD8-biased T cell activation profile was induced by LM1953. FIG. 32A shows corresponding CD4 and CD8 T cell activation profiles as % of cells expressing CD25 on the cell surface in the C4-2 cytolysis assay and FIG. 32B in the 22Rv1 cytolysis assay. FIG. 33A-D show in vitro cytokine release profiles of LM1953 on C4-2 cells for IL-6 (FIG. 33A) and TNF-α (FIG. 33B), and on 22Rv1 cells for IL-6 (FIG. 33C) and TNF-α (FIG. 33D).

### Anti-Tumor Efficacy of LM1953 in a Humanized Prostate Cancer Xenograft Cell Line Model

### Method

Male NSG MHC I/II DKO mice were humanized i.v. with 10e6 PBMCs from 3 different donors (n=4/group) on day −7 then 5e6 22Rv1 tumor cells were injected s.c. on the flank on day 0. On days 2, 6, 10, 14, 17 and 21, 0.41 mg/kg of LM1953 was injected i.p. and tumor size was evaluated using calipers.

### Results

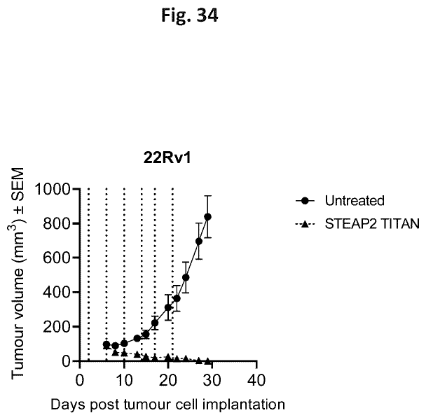

FIG. 34 shows that LM1953 confers anti-tumor efficacy in vivo against STEAP2+ prostate cancer xenograft model 22Rv1.

### Example 11: LRC150016 (LRRC15) Fab Variants with Improved Developability Profiles

Sequence variants of the LRC150016 V_{H}/V_{L} were designed to improve the developability and humanisation of the LRRC15 Fab. These variants were generated as human IgG1 format antibodies by standard methods and tested for retention of binding to human, mouse and cyno LRRC15 recombinant protein. Notably, it was found that variants with more human germline residues within H-CDR2 (SEQ ID NOs: 136, 147, 152 and 157) retained binding to LRRC15.

Selected LRRC15-binding Fab variants (see Table 7 below) were then formatted as T-cell engagers in the CD8/TCR format (see FIG. 15), with 2×LRRC15-binding Fab arms, 1×TCR binding VHH and 1×CD8 binding VHH. The CD8-binding sequence was replaced with a variant modified to remove two sequence liabilities identified in the original variant. One of these changes is in CDR1 by IGMT definition. These T-cell engagers were tested for their ability to induce CD8-mediated cytolysis of LRRC15-expressing tumour cell lines in co-culture with human PBMCs.

TABLE 7

| | LRRC15 Fab | LRRC15 Fab | | |
|---|---|---|---|---|
| Candidate # | VH | VL | TCR VHH | CD8 VHH |
| LRC150016 | SEQ ID NO: | SEQ ID NO: | SEQ ID NO: | SEQ ID NO: |
| | 31 | 32 | 42 | 52 |
| TPP-46956 | SEQ ID NO: | SEQ ID NO: | SEQ ID NO: | SEQ ID NO: |
| | 141 | 142 | 42 | 168 |
| TPP-46957 | SEQ ID NO: | SEQ ID NO: | SEQ ID NO: | SEQ ID NO: |
| | 148 | 142 | 42 | 168 |
| TPP-46958 | SEQ ID NO: | SEQ ID NO: | SEQ ID NO: | SEQ ID NO: |
| | 153 | 142 | 42 | 168 |
| TPP-46959 | SEQ ID NO: | SEQ ID NO: | SEQ ID NO: | SEQ ID NO: |
| | 159 | 160 | 42 | 168 |
| TPP-46960 | SEQ ID NO: | SEQ ID NO: | SEQ ID NO: | SEQ ID NO: |
| | 159 | 166 | 42 | 168 |
| TPP-47826 | SEQ ID NO: | SEQ ID NO: | SEQ ID NO: | SEQ ID NO: |
| | 153 | 142 | 172 | 168 |
| TPP-49058 | SEQ ID NO: | SEQ ID NO: | SEQ ID NO: | SEQ ID NO: |
| | 159 | 160 | 172 | 168 |

The affinities of the T-cell engagers to human, mouse and cyno LRRC15 recombinant proteins were measured by Surface Plasmon Resonance using a Single-Cycle Kinetics (SCK) method on a Biacore 8K+ system (CYTIVA).

The T-cell engagers were also tested for unwanted binding to cells transiently transfected with human EphB6. TPP-46959 was identified as having reduced binding to EphB6-expressing cells. This molecule differs from TPP-46960 at the L-CDR2 (SEQ ID NO: 158) where residue 55 is E55 in TPP-46959, compared to H55 in huM25 and N55 in TPP-46960.

LRRC15 (huM25) Fab Sequence Variants Affinity Determination—Single Cycle Kinetics Measurements.

The T-cell engagers were captured on a PROTEIN G chip and exposed to a series of increasing concentrations of human, mouse or cyno LRRC15 recombinant extracellular domain proteins (Acro Biosystems PROTEIN G). The fully corrected binding data were globally fitted to a 1:1 binding model to determine the K_{D} using the Biacore™ Insight Evaluation Software (CYTIVA). Coloured sensorgram trace represents the raw data and black line the global 1:1 fit to that data. Values for kinetics parameters is shown in Table 8 are averages for 3 or 4 replicate sets of measurements.

TABLE 8

| T-cell | LRRC15 | kₐ (M⁻¹ s⁻¹) | | k_{d} (s⁻¹) | | K_{D} (nM) | |
|---|---|---|---|---|---|---|---|
| engager | Species | Average | SD | Average | SD | Average | SD |
| TPP-46956 | human | 3.05E+05 | 2.61E+03 | 6.84E−04 | 1.10E−05 | 2.24 | 0.04 |
| | cyno | 1.70E+05 | 1.24E+03 | 8.18E−04 | 2.39E−05 | 4.80 | 0.1 |
| | mouse | 2.05E+05 | 5.16E+02 | 2.06E−04 | 7.55E−06 | 1.01 | 0.04 |
| TPP-46957 | human | 2.64E+05 | 4.73E+03 | 6.00E−04 | 5.56E−06 | 2.27 | 0.06 |
| | cyno | 1.65E+05 | 3.23E+03 | 7.50E−04 | 4.02E−06 | 4.55 | 0.1 |
| | mouse | 1.98E+05 | 8.16E+02 | 1.88E−04 | 9.95E−06 | 0.953 | 0.05 |
| TPP-46958 | human | 2.83E+05 | 4.25E+03 | 8.77E−04 | 3.54E−05 | 3.10 | 0.08 |
| | cyno | 1.70E+05 | 2.45E+03 | 1.09E−03 | 7.34E−05 | 6.40 | 0.5 |
| | mouse | 1.96E+05 | 4.04E+03 | 3.43E−04 | 5.59E−05 | 1.75 | 0.3 |
| TPP-46959 | human | 3.51E+05 | 1.99E+04 | 4.68E−04 | 5.14E−05 | 1.34 | 0.2 |
| | cyno | 1.89E+05 | 2.40E+03 | 5.68E−04 | 6.13E−05 | 3.00 | 0.3 |
| | mouse | 2.38E+05 | 3.70E+03 | 1.44E−04 | 6.40E−06 | 0.608 | 0.04 |
| TPP-46960 | human | 2.97E+05 | 1.04E+04 | 2.91E−04 | 1.65E−05 | 0.980 | 0.07 |
| | cyno | 1.70E+05 | 5.75E+03 | 3.79E−04 | 2.94E−05 | 2.24 | 0.2 |
| | mouse | 2.00E+05 | 3.32E+03 | 5.64E−05 | 2.56E−05 | 0.281 | 0.13 |

LRRC15 (huM25) Fab Sequence Variants Functional Assessment.

Whole PBMCs (3 donors) were cultured with LRRC15^{pos} Saos-2 Wt target cell line for 3 days at an E:T ratio of 10 to 1. LRC150016 and its LRRC15 Fab sequence variants mediated T cell activation, as measured by % CD25 surface expression. Tabular data (Table 9 below) refers TCE molecule EC₅₀ (pM) and Eₘₐₓ (0%) values for CD8 T-cells activation.

TABLE 9

| TCE | EC₅₀ (pM) | Eₘₐₓ (%) |
|---|---|---|
| LRC150016 | 3.3 | 52.31 |
| TPP-46956 | 3.9 | 53.32 |
| TPP-46957 | 3.7 | 49.69 |
| TPP-46958 | 3.2 | 55.17 |
| TPP-46859 | 2.0 | 54.63 |
| TPP-46960 | 2.8 | 55.06 |

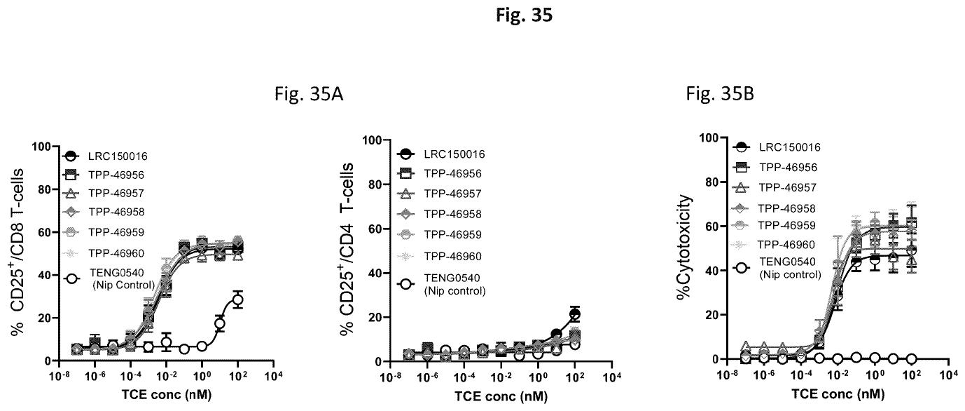

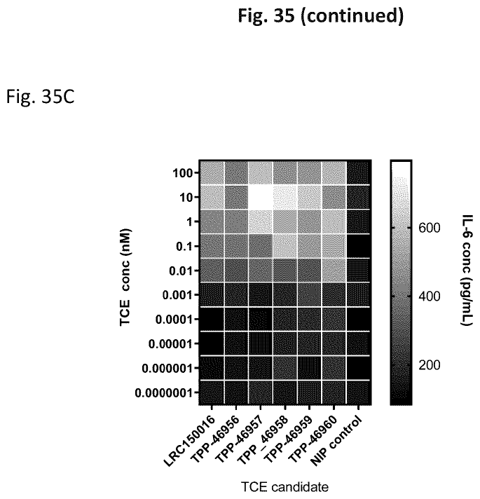

Results are shown in FIG. 35A, values are mean (±SEM) frequency of T-cell activation.

Dose-response curves demonstrated that both LRC150016 and the LRRC15 Fab sequence variant TCEs mediate T-cell activation in a dose dependent manner, as measured by the CD25 surface expression, following 3 days of whole PBMCs co-culture with LRRC15^{pos} Saos-2 Wt Target cells. Similar to LRC150016, all five LRRC15 Fab sequence variant TCEs selectively activate CD8+ T-cells, while showing no activation of CD4+ T cells. Values shown here are the mean (±SEM) frequency of CD25⁺ cells among CD8 and CD4 T-cells. n=3 donors from a representative experiment.

### In-Vitro Cytolytic Activity of LRC150016 and its LRRC15 Fab Sequence Variants Against LRRC15^{pos} Saos-2 WT Target Cell Line

Target cytolysis was assessed using CELL-TITER-GLO. Tabular data refers to TCE molecule EC₅₀ (pM) and Eₘₐₓ (%) values for target cell cytolysis, see Table 10.

Results are shown in FIG. 35B. Dose-response curves show that compared to LRC150016, all five LRRC15 Fab sequence variant TCEs mediate similar or slightly higher levels of target (Saos-2 Wt) cell cytolysis, as measured by the cytolysis Eₘₐₓ values, after 3 days of Saos-2 target cell co-culture with whole PBMC effector cells. Target cell cytolysis was assessed using CELL-TITER-GLO. Nip 228 control TCE did not mediate any target cell cytotoxicity. Values shown here are the mean (±SEM) frequency of target cell cytolysis (cytotoxicity). n=3 donors from a representative experiment.

TABLE 10

| TCE | EC₅₀ (pM) | Eₘₐₓ (%) |
|---|---|---|
| LRC150016 | 6.7 | 46.84 |
| TPP-46956 | 8.7 | 59.65 |
| TPP-46957 | 5.3 | 49.82 |
| TPP-46958 | 6.1 | 57.72 |
| TPP-46859 | 4.4 | 60.02 |
| TPP-46960 | 4.3 | 60.57 |

### Evaluation of Cytokine (IL-6) Profile in the Supernatant of Whole PBMCs Co-Culture Assay.

Whole PBMCs were cultured with LRRC15^{pos} Saos-2 Wt target cells either in the presence of LRC150016 or one of five LRRC15 fab sequence variant TCEs for a period of 3 days at an ET ratio of 10 to 1. IL-6, a surrogate for cytokine release syndrome (CRS), was measured in the culture supernatant after 3-days of co-culture using standard ELISA. Values shown here are the mean (±SEM) frequency of IL-6 (pg/mL) in the cell culture supernatant. Results are shown in FIG. 35C, the LRRC15 variants had a similar effect on IL-6 levels as LRC150016.

### EphB6 Off-Target Binding Assessment

Ad293 cells were transiently transfected with a plasmid expressing human EphB6. Binding of the LRRC15 T-cell engagers was evaluated by flow cytometry.

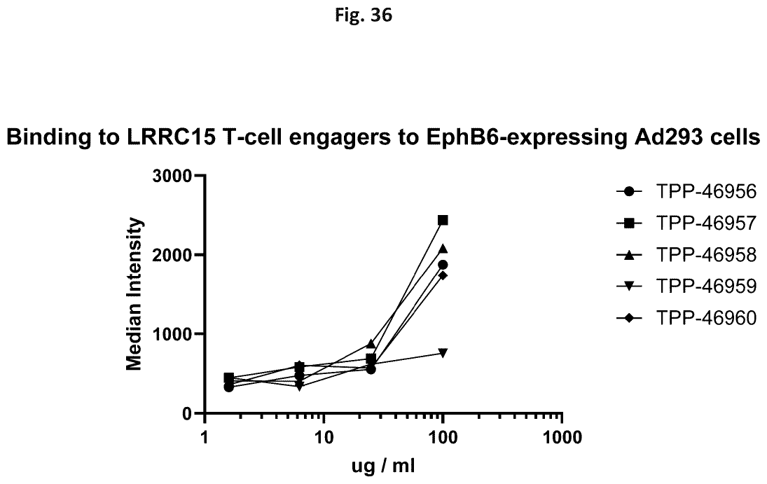

Results are shown in FIG. 36, TPP-46959 showed reduced off-target binding to EphB6 expressing AD293 cells, compared to other sequence variants.

### Example 12: TPP-42197 (BCMA TITAN) Depletion of the Plasmablast/Plasma Cells in Primary Assays

### Method

Healthy donor PBMCs were thawed, counted and naïve B cells were separated by magnetic beads. The cells were cultured with 1 g/ml CpG and 100U/ml IL-2 for 5 days in RPMI media enriched with ATCC to induce B cell differentiation into plasmablasts. Following 5 days of differentiation, autologous PBMCs were thawed and co-cultured with differentiated plasmablasts at a ratio of 1:5 target to effector cells. To maintain this ration, the % of CD3+ T cells per donor was assessed by flow cytometry and the number of PBMCs to be cultured, was determined so it will keep this ratio between plasmablasts and T cells (50,000 plasmablasts with 250,000 CD3+ T cells). The cells were stimulated with increasing concentrations (0.0000003 nM to 20 nM) of either TPP-42197 or the comparator, “Comparator T” (CD3×BCMA TCE) for 48h. At the end of the experiment, the supernatant was collected and the levels of cytokine secretion from the cells was assessed by MSD ELISA. The level of plasmablast cytolysis as well as the level of T cell activation for both CD8+ cytotoxic T cells and CD4+ helper T cells were measured by flow cytometry.

### Results

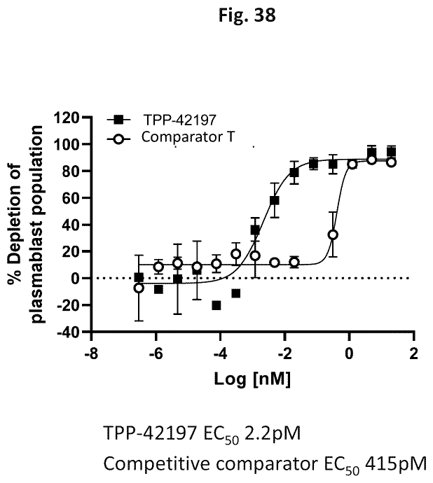

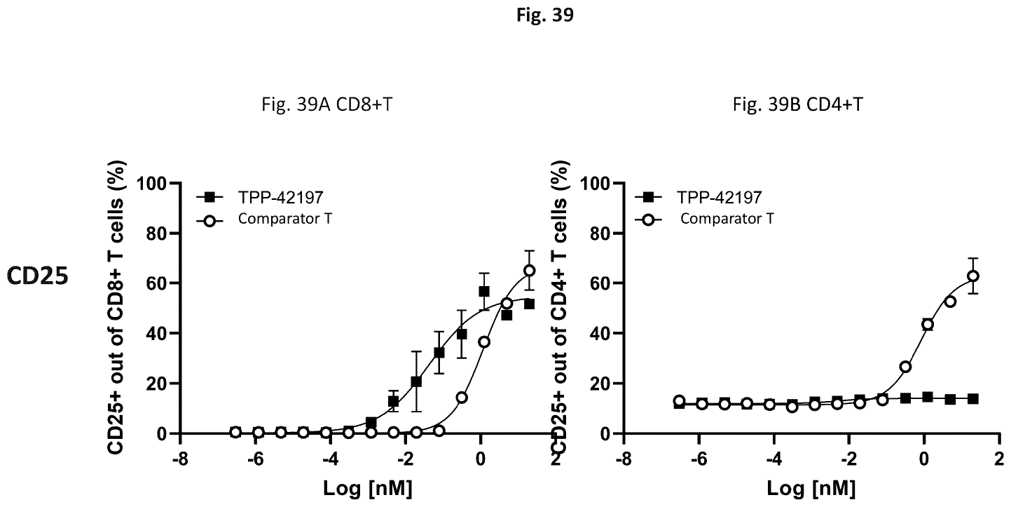

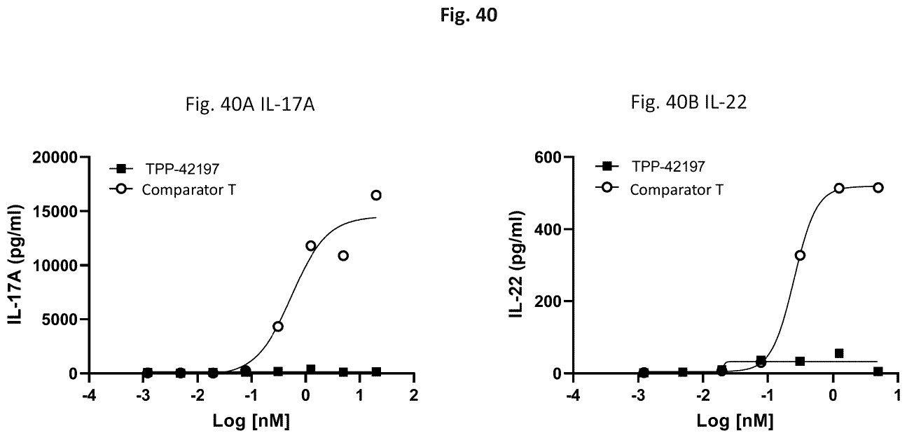

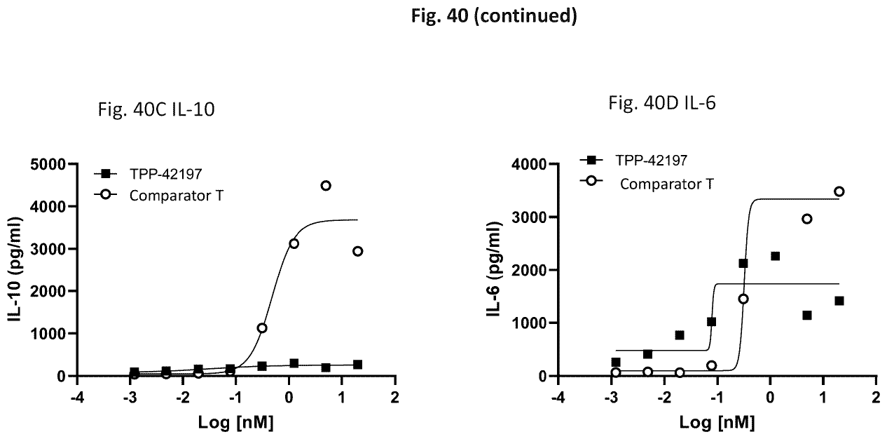

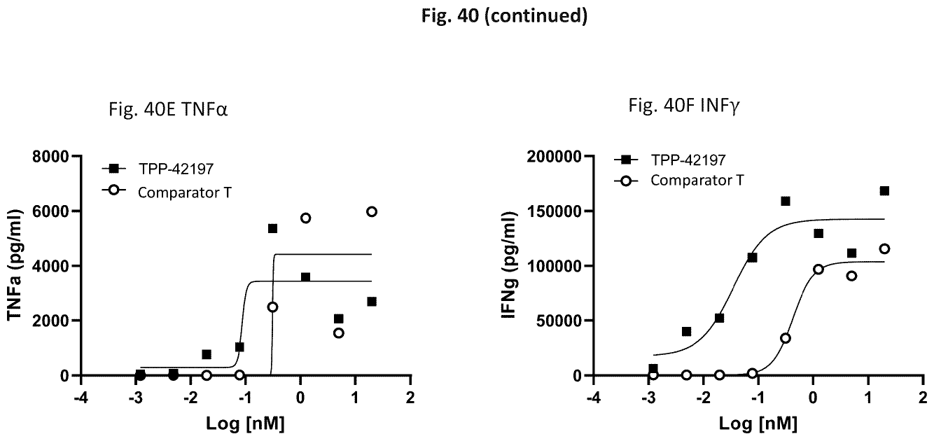

TPP-42197 was found to have a better potency than Comparator T and a better EC₅₀ (2.2 pM versus 415 pM respectively), see FIG. 38. In addition, TPP-42197 induced a selective activation of CD8+ T cells and no activation of CD4+ T cells, the latter being major contributors of cytokine secretion (FIG. 39A-B). In contrast, Comparator T induced activation of both CD8+ and CD4+ T cells. The cytokine profile of TPP-42197 was shown to be superior to that of Comparator T with lower cytokine secretion in general and particularly in those involved in cytokine release syndrome (CRS) events (see FIGS. 40A-F)

### TCP-42197Fully Depletes Engrafted Human BCMA+ Cells in Bone Marrow and Spleen

To assess the ability of TCP-42197 to deplete primary B cells expressing BCMA in vivo, a pharmacodynamic (PD) study was performed in the xenogeneic model of graft versus host disease (XenoGvHD). To prepare for this XenoGvHD PD study, healthy human donor peripheral blood mononuclear cells (PBMCs) were isolated from fresh Leukopak (StemExpress™) and stored frozen (liquid nitrogen tank) until the engraftment.

On Day −1, 8-10 week old female NOD scid gamma mice (NSG; Jackson Laboratories) were pre-conditioned with sub-lethal whole-body irradiation (1 Gy). On Day 0, 15 million total PBMC from a healthy human donor was injected intravenously. On day 9, mice received either phosphate buffered saline (PBS), 1 mg/kg TCP-42197, 10 mg/kg TCP-42197 or 10 mg/kg isotype control, intraperitoneally (n=5-9 mice/group). On Day 12, mice were euthanized, and tissues were collected for FACS analysis to determine whether these TCEs depleted the BCMA+ cells present in this in vivo model.

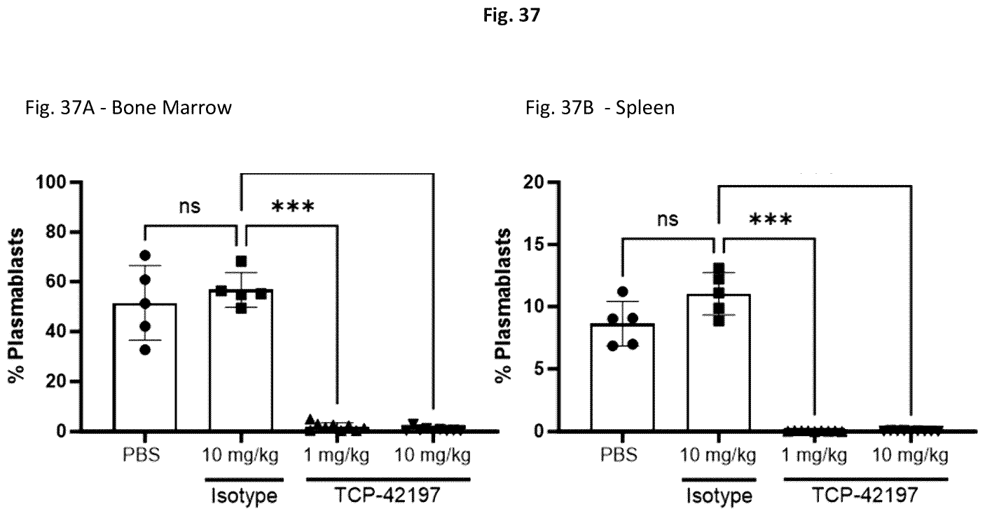

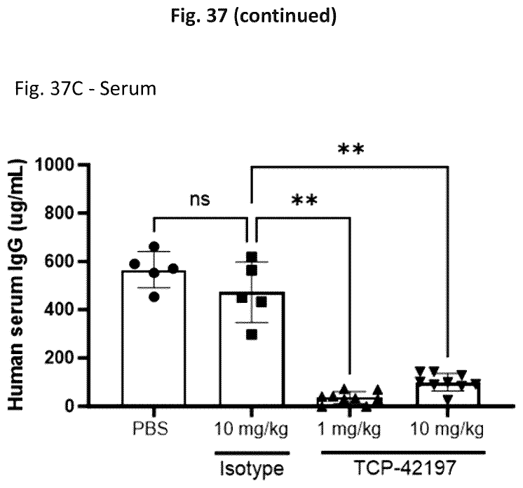

Plasmablasts, defined by flow cytometric analysis as human CD45⁺CD27⁺CD38⁺CD3⁻CD56⁻CD14⁻CD16⁻, were depleted in mice three days following a single treatment of TCP-42197 in both the bone marrow and in the spleen (FIGS. 37A and 37B). Serum human IgG was significantly lower in the TCP-42197 treatment groups compared to the isotype control treated group (FIG. 37C), reflecting the absence of BCMA+ antibody secreting cells in the TCP-42197 treated mice.

We claim:

1. A binding protein comprising four polypeptide chains that form two tumor-associated antigen (TAA) binding sites, a T cell receptor binding site, and a T cell co-stimulatory molecule binding site, wherein the first and second polypeptide chains have a structure represented by the formula:
V_{L}-C_{L}
a third polypeptide chain has a structure represented by the formula:
V_{H1}-C_{H1}-V_{HHa}-F_{Ca}
and a fourth polypeptide chain has a structure represented by the formula:
V_{H1}-C_{H1}-V_{HHb}-F_{Cb}
wherein the first polypeptide and the third polypeptide form the first of the two TAA binding sites, and the second polypeptide and the fourth polypeptide form the second of the two TAA binding sites, and
wherein:
V_{L} is an immunoglobulin light chain variable domain and V_{H1} is an immunoglobulin heavy chain variable domain that together form a TAA binding domain that specifically binds a tumor-associated antigen;
C_{L} is an immunoglobulin light chain constant domain;
C_{H1} is an immunoglobulin CH1 heavy chain constant domain;
V_{HHa} is a single chain variable domain that specifically binds a T cell receptor;
V_{HHb} is a single chain variable domain that specifically binds T cell co-stimulatory molecule;
F_{Ca} is C_{H2a} and C_{H3a} immunoglobulin heavy chain constant domains; and
F_{Cb} is C_{H2b} and C_{H3b} immunoglobulin heavy chain constant domains.

2. The binding protein of claim 1, further comprising L₁, a linker positioned between C_{H1} and V_{HHa} on the third polypeptide chain, and L₂, a linker positioned between V_{HHa} and the F_{Ca} on the third polypeptide chain, wherein L₁ and L₂ are each independently a linker or are absent.

3. The binding protein of either claim 1 or claim 2, further comprising L₃, a linker positioned between C_{H1} and V_{HHb} on the fourth polypeptide chain, and L₄, a linker positioned between V_{HHb} and F_{Cb} on the fourth polypeptide chain, wherein L₃ and L₄ are each independently a linker or are absent.

4. The binding protein of any one of claims 1-3 further comprising H₁, an immunoglobulin hinge region positioned between C_{H1} and V_{HHa} on the third polypeptide chain, and H₂, an immunoglobulin hinge region positioned between V_{HHa} and the F_{Ca} on the third polypeptide chain, wherein H₁ and H₂ are each independently an immunoglobulin hinge region or are absent.

5. The binding protein of claim 4, wherein H₁ comprises SEQ ID NO: 53, SEQ ID NO: 54, SEQ ID NO: 90 or is absent, and wherein H₂comprises SEQ ID NO: 53, SEQ ID NO: 55, SEQ ID NO: 90 or is absent.

6. The binding protein of any one of claims 1-5, further comprising H₃, an immunoglobulin hinge region positioned between C_{H1} and V_{HHb} on the fourth polypeptide chain, and H₄, an immunoglobulin hinge region positioned between V_{HHb} and the F_{Cb} on the fourth polypeptide chain, wherein H₃ and H₄ are each independently an immunoglobulin hinge region or are absent.

7. The binding protein of claim 6, wherein H₃ comprises SEQ ID NO: 53, DK (SEQ ID NO: 54), SEQ ID NO: 90 or is absent, and wherein H₄ comprises SEQ ID NO: 53, SEQ ID NO: 55, SEQ ID NO: 91 or is absent.

8. The binding protein of any one of claims 1-7, wherein the first and second polypeptide chains have a structure represented by the formula:
V_{L}-C_{L}
a third polypeptide chain has a structure represented by the formula:
V_{H1}-C_{H1}-H₁-L₁-V_{HHa}-H₂-L₂-F_{Ca} or
V_{H1}-C_{H1}-H₁-L₁-V_{HHa} L₂-H₂-F_{Ca}
and a fourth polypeptide chain has a structure represented by the formula:
V_{H1}-C_{H1}-H₃-L₃-V_{HHb}-H₄-L₄-F_{Cb} or
V_{H1}-C_{H1}-H₃-L₃-V_{HHb}-L₄-H₄-F_{Cb}.

9. The binding protein of any one of claims 1-8, wherein F_{Ca} and/or F_{Cb} is from an IgG antibody.

10. The binding protein of claim 9, wherein the F_{Ca} and/or the Fe is from an IgG1 antibody.

11. The binding protein of any one of claims 1-10, wherein the binding protein activates T cells only when bound to a tumor associated antigen at one or both of the tumor-associated antigen binding sites.

12. The binding protein of any one of claims 2-11, wherein L₁, L₂, L₃, and/or L₄ comprise one or more repeats of the amino acid sequence of SEQ ID NO: 37 and/or SEQ ID NO: 38.

13. The binding protein of any one of claims 1-12, wherein the V_{HHa} comprises a heavy chain CDR1 (HCDR1), a heavy chain CDR2 (HCDR2) and a heavy chain CDR3 (HCDR3), comprising the amino acid sequences of SEQ ID NO: 39, SEQ ID NO: 40 and SEQ ID NO: 41, respectively.

14. The binding protein of any one of claims 1-12, wherein the V_{HHa} comprises a heavy chain CDR1 (HCDR1), a heavy chain CDR2 (HCDR2) and a heavy chain CDR3 (HCDR3) comprising the amino acid sequence of SEQ ID NO: 62, SEQ ID NO: 40 and SEQ ID NO: 41, respectively.

15. The binding protein of any one of claims 1-14, wherein the V_{HHa} Comprises the amino acid sequence at least 90%, at least 91% at least 92%, at least 93%, at least 94%, at least 95%, at least 96%, at least 97%, at least 98%, or at least 99% identical to any one of SEQ ID NOs: 42-48, or any one of SEQ ID NOs: 42-48.

16. The binding protein of any one of claims 1-12, wherein the V_{HHa} comprises a heavy chain CDR1 (HCDR1), a heavy chain CDR2 (HCDR2) and a heavy chain CDR3 (HCDR3), comprising the amino acid sequences of SEQ ID NO: 173, SEQ ID NO: 174, and SEQ ID NO: 175 respectively.

17. The binding protein of any one of claims 1-12 or 16, wherein the V_{HHa} Comprises the amino acid sequence at least 90%, at least 91% at least 92%, at least 93%, at least 94%, at least 95%, at least 96%, at least 97%, at least 98%, or at least 99% identical to SEQ ID NO: 172, or SEQ ID NO: 172.

18. The binding protein of any one of claims 1-17, wherein the T cell costimulatory molecule is CD8.

19. The binding protein of any one of claims 1-18, wherein the V_{HHb} comprises a heavy chain CDR1 (HCDR1), a heavy chain CDR2 (HCDR2) and a heavy chain CDR3 (HCDR3) comprising the amino acid sequences of SEQ ID NO: 49, SEQ ID NO: 50 and SEQ ID NO: 51, respectively.

20. The binding protein of any one of claims 1-19, wherein the V_{HHb} Comprises an amino acid sequence at least 90%, at least 91% at least 92%, at least 93%, at least 94%, at least 95%, at least 96%, at least 97%, at least 98%, or at least 99% identical to SEQ ID NO: 52, or SEQ ID NO: 52.

21. The binding protein of any one of claims 1-18, wherein the V_{HHb} comprises a heavy chain CDR1 (HCDR1), a heavy chain CDR2 (HCDR2) and a heavy chain CDR3 (HCDR3) comprising the amino acid sequences of SEQ ID NO: 169, SEQ ID NO: 170 and SEQ ID NO: 171, respectively.

22. The binding protein of any one of claims 1-21, wherein the V_{HHb} Comprises an amino acid sequence at least 90%, at least 91% at least 92%, at least 93%, at least 94%, at least 95%, at least 96%, at least 97%, at least 98%, or at least 99% identical to SEQ ID NO: 168, or SEQ ID NO: 168.

23. The binding protein of any one of claims 1-22, wherein C_{H3a} and C_{H3b} in each of F_{Ca} and F_{Cb} comprise modifications to facilitate heterodimerization of F_{Ca} and F_{Cb}.

24. The binding protein of claim 23, wherein the modifications are substitutions to generate a knob in one of F_{Ca} and F_{Cb} and a hole in the other of F_{Ca} and F_{Cb}, wherein the knob is a substitution to tryptophan at position 366, and wherein the hole is a substitution of one or more of the following:
(i) a substitution to valine at position 407;
(ii) a substitution to serine at position 366; and
(iii) a substitution to alanine at position 368
and wherein the numbering is according to the Eu index.

25. The binding protein of claim 24, wherein the F_{Ca} or the F_{Cb} containing the knob further comprises a cysteine at position 354 and/or the F_{Ca} or the F_{Cb} containing the hole comprises a cysteine at position 349, and wherein the numbering is according to the Eu index.

26. The binding protein of any one of claims 1-25 wherein the C_{H2a} and C_{H2b} immunoglobulin heavy chain constant domains each comprise the following substitutions: E233P/L₂₃₄V/L₂₃₅A/G236del/S267K, and wherein the numbering is according to the Eu index.

27. The binding protein of any one of claims 1-26, wherein either the C_{H3a} immunoglobulin heavy chain constant domain or C_{H3b} immunoglobulin heavy chain constant domain comprises a H435R and Y436F substitution, and wherein the numbering is according to the Eu index.

28. The binding protein of any one of claims 1-27, wherein F_{Ca} comprises the amino acid sequence of SEQ ID NO: 56 or SEQ ID NO: 57.

29. The binding protein of any one of claims 1-28, wherein F_{Cb} comprises the amino acid sequence of SEQ ID NO: 58.

30. The binding protein of any one of claims 1-29, wherein the third polypeptide chain comprises either SEQ ID NO: 59 or SEQ ID NO: 60 and the fourth polypeptide chain comprises SEQ ID NO: 61.

31. The binding protein of any one of claims 1-30, wherein the C_{L} of the first polypeptide chain and/or the C_{L} of the second polypeptide chain comprises a constant light chain lambda region (CLλ); and the binding protein comprises at least one lambda charge pair wherein the lambda charge pair is either between the CLλ of the first polypeptide chain and a corresponding C_{H1} that is the C_{H1} of the third polypeptide chain, or the lambda charge pair is between the CLλ of the second polypeptide and a corresponding C_{H1} that is the C_{H1} of the fourth polypeptide chain, and where the lambda charge pair is located at one or more of the following pairs of positions:
(i) position 117 in the CLλ and position 141 in the corresponding C_{H1};
(ii) position 117 in the CLλ and position 185 in the corresponding C_{H1};
(iii) position 119 in the CLλ and position 128 in the corresponding C_{H1};
(iv) position 134 in the CLλ and position 128 in the corresponding C_{H1};
(v) position 134 in the CLλ and position 145 in the corresponding C_{H1};
(vi) position 134 in the CLλ and position 183 in the corresponding C_{H1};
(vii) position 136 in the CLλ and position 185 in the corresponding C_{H1};
(viii) position 178 in the CLλ and position 173 in the corresponding C_{H1}; and
(ix) position 117 in the CLλ and position 187 in the corresponding C_{H1};
wherein the lambda charge pair comprises a positively charged amino acid residue selected from arginine, lysine and histidine located at one of the positions in the lambda charge pair and a negatively charged amino acid residue selected from aspartic acid, glutamic acid, serine and threonine located at the other position in the lambda charge pair; and
wherein the numbering is according to the Eu index.

32. The binding protein of claim 31, wherein the lambda charge pair is selected from the following list:
(a) arginine at position 117 of the CLλ and aspartic acid at position 141 in the corresponding C_{H1};
(b) arginine at position 117 of the CLλ and glutamic acid at position 141 in the corresponding C_{H1};
(c) arginine at position 117 of the CLλ and serine at position 141 in the corresponding C_{H1};
(d) arginine at position 117 of the CLλ and threonine at position 141 in the corresponding C_{H1};
(e) lysine at position 117 of the CLλ and aspartic acid at position 141 in the corresponding C_{H1};
(f) lysine at position 117 of the CLλ and glutamic acid at position 141 in the corresponding C_{H1};
(g) lysine at position 117 of the CLλ and serine at position 141 in the corresponding C_{H1}; and
(h) lysine at position 117 of the CLλ and threonine at position 141 in the corresponding C_{H1}.

33. The binding protein of either claim 31 or claim 32, wherein the CLλ of the first polypeptide chain and/or CLλ of the second polypeptide chain each comprises an amino acid sequence having at least 90%, 91%, 92%, 93%, 94%, 95%, 96%, 97%, 98%, or 99% identity to SEQ ID NOs: 98-99.

34. The binding protein of any one of claims 1-33, wherein the C_{L} of the first polypeptide chain and/or the C_{L} of the second polypeptide chain comprises a constant light chain kappa region (CLκ); and a kappa charge pair wherein the kappa charge pair is either between the CLκ of the first polypeptide chain and a corresponding C_{H1} that is the C_{H1} of the third polypeptide chain, or the kappa charge pair is between the CLκ of the second polypeptide and a corresponding C_{H1} that is the C_{H1} of the fourth polypeptide chain; wherein the kappa charge pair is located at position 117 in the CLκ and position 141 in the corresponding C_{H1} and comprises a positively charged amino acid residue selected from arginine, lysine, and histidine located at one of the positions in the kappa charge pair, and a negatively charged amino acid residue selected from aspartic acid, glutamic acid, serine and threonine located at the other position in the kappa charge pair.

35. The binding protein of claim 34, wherein the negatively charged amino acid residue in the kappa charge pair is located at position 133 of the CLκ, and the positively charged amino acid residue is located at position 183 of the corresponding C_{H1}, optionally wherein the negatively charged amino acid residue at position 133 of the CLκ is a glutamic acid, and optionally wherein the positively charged amino acid residue at position 183 of the corresponding C_{H1} is a lysine.

36. The binding protein of either claim 34 or claim 35, wherein the CLκ of the first polypeptide chain or the second polypeptide chain comprises an amino acid sequence having at least 90%, 91%, 92%, 93%, 94%, 95%, 96%, 97%, 98%, or 99% identity to SEQ ID NO: 100.

37. The binding protein of any one of claims 34-36 wherein the corresponding C_{H1} in each of the one or more lambda charge pairs comprises an amino acid sequence having at least 90%, 91%, 92%, 93%, 94%, 95%, 96%, 97%, 98%, or 99% to SEQ ID NO: 95-97, and/or the corresponding C_{H1} in the kappa charge pair comprises an amino acid sequence having at least 90%, 91%, 92%, 93%, 94%, 95%, 96%, 97%, 98%, or 99% to SEQ ID NO: 100.

38. The binding protein of any one of claims 31-37, wherein the CLλ comprises an amino acid sequence according to SEQ ID NO: 105, and the corresponding C_{H1} Comprises an amino acid sequence according to SEQ ID NO: 104 or SEQ ID NO: 220.

39. The binding protein of any one of claims 1-38, wherein either:
(i) a disulfide link between the C_{L} of the first polypeptide chain and the C_{H1} of the third polypeptide chain is formed between a pair of cysteines engineered into the C_{L} of the first polypeptide chain and the C_{H1} of the third polypeptide chain, and a disulfide link between the C_{L} of the second polypeptide chain and the C_{H1} of the fourth polypeptide chain is formed between a pair of native cysteines; or
(ii) a disulfide link between the C_{L} of the second polypeptide chain and the C_{H1} of the fourth polypeptide chain is formed between a pair of cysteines engineered into the C_{L} of the second polypeptide chain and the C_{H1} of the fourth polypeptide chain, and a disulfide link between the C_{L} of the first polypeptide chain and the C_{H1} of the third polypeptide chain is formed between a pair of native cysteines.

40. The binding protein of claim 39, wherein the pair of cysteines are engineered into either:
(i) the C_{L} of the first polypeptide chain and the C_{H1} of the third polypeptide chain, wherein the C_{L} of the first polypeptide chain is a CLλ and wherein the pair of engineered cysteines are located at position 122 of the CLλ of the first polypeptide chain and position 126 of the C_{H1} of the third polypeptide chain, and wherein the C_{L}, of the first polypeptide chain comprises a non-cysteine residue at position 212 and the C_{H1} of the third polypeptide chain comprises a non-cysteine residue at position 220, optionally wherein the non-cysteine residues are valines; or
(ii) the C_{L} of the second polypeptide chain and the C_{H1} of the fourth polypeptide chain, wherein the C_{L} of the first polypeptide chain is a CLλ and wherein the pair of engineered cysteines are located at position 122 of the CLλ of the second polypeptide chain and position 126 of the C_{H1} of the fourth polypeptide chain, and wherein the CLλ of the second polypeptide chain comprises a non-cysteine residue at position 212 and the C_{H1} of the fourth polypeptide chain comprises a non-cysteine residue at position 220, optionally wherein the non-cysteine residues are valines.

41. The binding protein of claim 39, wherein the pair of cysteines are engineered into either:
(i) the C_{L} of the first polypeptide chain and the C_{H1} of the third polypeptide chain, wherein the C_{L} of the first polypeptide chain is a CLκ and wherein the pair of engineered cysteines are located at position 121 of the CLκ of the first polypeptide chain and position 126 of the C_{H1} of the third polypeptide chain, and wherein the CLκ of the first polypeptide chain comprises a non-cysteine residue at position 214 and the C_{H1} of the third polypeptide chain comprises a non-cysteine residue at position 220, optionally wherein the non-cysteine residues are valines; or
(ii) the C_{L} of the second polypeptide chain and the C_{H1} of the fourth polypeptide chain, wherein the C_{L} of the first polypeptide chain is a CLκ and wherein the pair of engineered cysteines are located at position 121 of the CLκ of the second polypeptide chain and position 126 of the C_{H1} of the fourth polypeptide chain, and wherein the CLκ of the second polypeptide chain comprises a non-cysteine residue at position 214 and the C_{H1} of the fourth polypeptide chain comprises a non-cysteine residue at position 220, optionally wherein the non-cysteine residues are valines.

42. The binding protein of any one of claims 39-41, wherein the CLλ comprises an amino acid sequence according to SEQ ID NO: 107, and the corresponding C_{H1} comprises an amino acid sequence according to SEQ ID NO: 106.

43. The binding protein of any one of claims 1-42, wherein the tumor-associated antigen (TAA) is CD20, Glypican-3 (GPC3), or leucine rich repeat containing 15 (LRRC15).

44. The binding protein of any one of claims 1-42, wherein the tumor-associated antigen (TAA) is B-cell maturation antigen (BCMA) or six-transmembrane epithelial antigen of prostate-2 (STEAP2).

45. The binding protein of any one of claims 1-43, wherein the TAA binding domain binds to CD20 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2) and a light chain CDR3 (LCDR3), comprising the amino acid sequences of SEQ ID NO: 1, SEQ ID NO: 2, SEQ ID NO: 3, SEQ ID NO: 4, SEQ ID NO: 5 and SEQ ID NO: 6, respectively.

46. The binding protein of any one of claims 1-43, wherein the TAA binding domain binds to CD20 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2) and a light chain CDR3 (LCDR3), comprising the amino acid sequences of SEQ ID NO: 182, SEQ ID NO: 2, SEQ ID NO: 3, SEQ ID NO: 4, SEQ ID NO: 5 and SEQ ID NO: 6, respectively.

47. The binding protein of claim 45 or claim 46, wherein the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 7 and SEQ ID NO: 8, respectively; or comprises a V_{H} chain according to SEQ ID NO: 7 and a V_{L} according to SEQ ID NO: 8.

48. The binding protein of any one of claims 1-43 wherein the TAA binding domain binds to GPC3 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 13, SEQ ID NO: 14, SEQ ID NO: 15, SEQ ID NO: 16, SEQ ID NO: 17, and SEQ ID NO: 18, respectively.

49. The binding protein of claim 48, wherein the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 19 and SEQ ID NO: 20, respectively; or comprises a V_{H} chain according to SEQ ID NO: 19 and a V_{L} according to SEQ ID NO: 20.

50. The binding protein of any one of claims 1-43, wherein the TAA binding domain binds to LRRC15 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 25, SEQ ID NO: 26, SEQ ID NO: 27, SEQ ID NO: 28, SEQ ID NO: 29, and SEQ ID NO: 30 respectively.

51. The binding protein of claim 50, wherein the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 31 and SEQ ID NO: 32, respectively; or comprises a V_{H} chain according to SEQ ID NO: 31 and a V_{L} according to SEQ ID NO: 32.

52. The binding protein of any one of claims 1-43, wherein the TAA binding domain binds to LRRC15 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 136, SEQ ID NO: 137, SEQ ID NO: 138, SEQ ID NO: 139, and SEQ ID NO: 140 respectively.

53. The binding protein of claim 52, wherein the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 141 and SEQ ID NO: 142, respectively; or comprises a V_{H} chain according to SEQ ID NO: 141 and a V_{L} according to SEQ ID NO: 142.

54. The binding protein of any one of claims 1-43, wherein the TAA binding domain binds to LRRC15 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 147, SEQ ID NO: 137, SEQ ID NO: 138, SEQ ID NO: 139, and SEQ ID NO: 140 respectively.

55. The binding protein of claim 54, wherein the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 148 and SEQ ID NO: 142, respectively; or comprises a V_{H} chain according to SEQ ID NO: 148 and a V_{L} according to SEQ ID NO: 142.

56. The binding protein of any one of claims 1-43, wherein the TAA binding domain binds to LRRC15 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 152, SEQ ID NO: 137, SEQ ID NO: 138, SEQ ID NO: 139, and SEQ ID NO: 140 respectively.

57. The binding protein of claim 56, wherein the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 153 and SEQ ID NO: 142, respectively; or comprises a V_{H} chain according to SEQ ID NO: 153 and a V_{L} according to SEQ ID NO: 142.

58. The binding protein of any one of claims 1-43, wherein the TAA binding domain binds to LRRC15 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 157, SEQ ID NO: 137, SEQ ID NO: 138, SEQ ID NO: 158, and SEQ ID NO: 140 respectively.

59. The binding protein of claim 58, wherein the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 159 and SEQ ID NO: 160, respectively; or comprises a V_{H} chain according to SEQ ID NO: 159 and a V_{L} according to SEQ ID NO: 160.

60. The binding protein of any one of claims 1-43, wherein the TAA binding domain binds to LRRC15 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 157, SEQ ID NO: 137, SEQ ID NO: 138, SEQ ID NO: 165, and SEQ ID NO: 140 respectively.

61. The binding protein of claim 60, wherein the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 159 and SEQ ID NO: 166, respectively; or comprises a V_{H} chain according to SEQ ID NO: 159 and a V_{L} according to SEQ ID NO: 166.

62. The binding protein of any one of claims 1-42 or 44, wherein the TAA binding domain binds to BCMA and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 111, SEQ ID NO: 112, SEQ ID NO: 113, SEQ ID NO: 114, SEQ ID NO: 115, and SEQ ID NO: 116, respectively.

63. The binding protein of claim 62, wherein the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 117 and SEQ ID NO: 118, respectively; or comprises a V_{H} chain according to SEQ ID NO: 117 and a V_{L} according to SEQ ID NO: 118.

64. The binding protein of any one of claims 1-42 or 44, wherein the TAA binding domain binds to STEAP2 and comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 123, SEQ ID NO: 124, SEQ ID NO: 125, SEQ ID NO: 126, SEQ ID NO: 127, and SEQ ID NO: 128, respectively.

65. The binding protein of claim 64, wherein the TAA binding domain comprises a V_{H1} domain and a V_{L} domain that is at least 90%, at least 91% at least 92%, at least 93%, at least 94%, at least 95%, at least 96%, at least 97%, at least 98%, or at least 99% identical to SEQ ID NO: 129 and SEQ ID NO: 130, respectively; or comprises a V_{H} chain according to SEQ ID NO: 129 and a V_{L} according to SEQ ID NO: 130.

66. The binding protein of any one of claims 1-43 or 45-65 comprising the amino acid of sequences of SEQ ID NO: 9, SEQ ID NO: 11, and SEQ ID NO: 12.

67. The binding protein of any one of claims 1-43 or 45-65 comprising the amino acid of sequences of SEQ ID NO: 21, SEQ ID NO: 23, and SEQ ID NO: 24.

68. The binding protein of any one of claims 1-43 or 45-65 comprising the amino acid of sequences of SEQ ID NO: 33, SEQ ID NO: 35, and SEQ ID NO: 36.

69. The binding protein of any one of claims 1-42 or 44-65 comprising the amino acid of sequences of SEQ ID NO: 119, SEQ ID NO: 121, and SEQ ID NO: 122.

70. The binding protein of any one of claims 1-42 or 44-65 comprising the amino acid of sequences of SEQ ID NO: 131, SEQ ID NO: 134, and SEQ ID NO: 133.

71. The binding protein of any one of claims 1-43 or 45-65 comprising the amino acid of sequences of SEQ ID NO: 143, SEQ ID NO: 145, and SEQ ID NO: 146.

72. The binding protein of any one of claims 1-43 or 45-65 comprising the amino acid of sequences of SEQ ID NO: 143, SEQ ID NO: 150, and SEQ ID NO: 151.

73. The binding protein of any one of claims 1-43 or 45-65 comprising the amino acid of sequences of SEQ ID NO: 143, SEQ ID NO: 155, and SEQ ID NO: 156.

74. The binding protein of any one of claims 1-43 or 45-65 comprising the amino acid of sequences of SEQ ID NO: 161, SEQ ID NO: 163, and SEQ ID NO: 164.

75. The binding protein of any one of claims 1-43 or 45-65 comprising the amino acid of sequences of SEQ ID NO: 167, SEQ ID NO: 163, and SEQ ID NO: 164.

76. The binding protein of any one of claims 1-43 or 45-65 comprising the amino acid of sequences of SEQ ID NO: 161, SEQ ID NO: 177, and SEQ ID NO: 164.

77. The binding protein of any one of claims 1-43 or 45-65 comprising the amino acid of sequences of SEQ ID NO: 143, SEQ ID NO: 176, and SEQ ID NO: 156.

78. The binding protein of claim 66, wherein the first and second polypeptide chains comprise the amino acid sequence of SEQ ID NO: 9, the third polypeptide chain comprises the amino acid sequence of SEQ ID NO: 11, and the fourth polypeptide chain comprises the amino acid sequence of SEQ ID NO: 12.

79. The binding protein of claim 67, wherein the first and second polypeptide chains comprise the amino acid sequence of SEQ ID NO: 21, the third polypeptide chain comprises the amino acid sequence of SEQ ID NO: 23, and the fourth polypeptide chain comprises the amino acid sequence of SEQ ID NO: 24.

80. The binding protein of claim 68, wherein the first and second polypeptide chains comprise the amino acid sequence of SEQ ID NO: 33, the third polypeptide chain comprises the amino acid sequence of SEQ ID NO: 35, and the fourth polypeptide chain comprises the amino acid sequence of SEQ ID NO: 36.

81. The binding protein of claim 69, wherein the first and second polypeptide chains comprise the amino acid sequence of SEQ ID NO: 119, the third polypeptide chain comprises the amino acid sequence of SEQ ID NO: 121, and the fourth polypeptide chain comprises the amino acid sequence of SEQ ID NO: 122.

82. The binding protein of claim 70, wherein the first and second polypeptide chains comprise the amino acid sequence of SEQ ID NO: 131, the third polypeptide chain comprises the amino acid sequence of SEQ ID NO: 134, and the fourth polypeptide chain comprises the amino acid sequence of SEQ ID NO: 133.

83. The binding protein of claim 71, wherein the first and second polypeptide chains comprise the amino acid sequence of SEQ ID NO: 143, the third polypeptide chain comprises the amino acid sequence of SEQ ID NO: 145, and the fourth polypeptide chain comprises the amino acid sequence of SEQ ID NO: 146.

84. The binding protein of claim 72, wherein the first and second polypeptide chains comprise the amino acid sequence of SEQ ID NO: 143, the third polypeptide chain comprises the amino acid sequence of SEQ ID NO: 150, and the fourth polypeptide chain comprises the amino acid sequence of SEQ ID NO: 151.

85. The binding protein of claim 73, wherein the first and second polypeptide chains comprise the amino acid sequence of SEQ ID NO: 143, the third polypeptide chain comprises the amino acid sequence of SEQ ID NO: 155, and the fourth polypeptide chain comprises the amino acid sequence of SEQ ID NO: 156.

86. The binding protein of claim 74, wherein the first and second polypeptide chains comprise the amino acid sequence of SEQ ID NO: 161, the third polypeptide chain comprises the amino acid sequence of SEQ ID NO: 163, and the fourth polypeptide chain comprises the amino acid sequence of SEQ ID NO: 164.

87. The binding protein of claim 75, wherein the first and second polypeptide chains comprise the amino acid sequence of SEQ ID NO: 167, the third polypeptide chain comprises the amino acid sequence of SEQ ID NO: 163, and the fourth polypeptide chain comprises the amino acid sequence of SEQ ID NO: 164.

88. The binding protein of claim 76, wherein the first and second polypeptide chains comprise the amino acid sequence of SEQ ID NO: 161, the third polypeptide chain comprises the amino acid sequence of SEQ ID NO: 177, and the fourth polypeptide chain comprises the amino acid sequence of SEQ ID NO: 164.

89. The binding protein of claim 77, wherein the first and second polypeptide chains comprise the amino acid sequence of SEQ ID NO: 143, the third polypeptide chain comprises the amino acid sequence of SEQ ID NO: 176, and the fourth polypeptide chain comprises the amino acid sequence of SEQ ID NO: 156.

90. A pharmaceutical composition comprising the binding protein of any one of claims 1-89 and a pharmaceutically acceptable carrier.

91. An isolated nucleic acid molecule encoding the binding protein of any one of claims 1-89.

92. The isolated nucleic acid molecule according to claim 91 comprising the nucleotide sequence according to any one of SEQ ID NOs: 184-186.

93. The isolated nucleic acid molecule according to claim 91 comprising the nucleotide sequence according to any one of SEQ ID NOs: 187-189.

94. The isolated nucleic acid molecule according to claim 91 comprising the nucleotide sequence according to any one of SEQ ID NOs: 190-192.

95. The isolated nucleic acid molecule according to claim 91 comprising the nucleotide sequence according to any one of SEQ ID NOs: 193-195.

96. The isolated nucleic acid molecule according to claim 91 comprising the nucleotide sequence according to any one of SEQ ID NOs: 196-198.

97. The isolated nucleic acid molecule according to claim 91 comprising the nucleotide sequence according to any one of SEQ ID NOs: 199-201.

98. The isolated nucleic acid molecule according to claim 91 comprising the nucleotide sequence according to any one of SEQ ID NOs: 202-204.

99. The isolated nucleic acid molecule according to claim 91 comprising the nucleotide sequence according to any one of SEQ ID NOs: 205-207.

100. The isolated nucleic acid molecule according to claim 91 comprising the nucleotide sequence according to any one of SEQ ID NOs: 208-210.

101. The isolated nucleic acid molecule according to claim 91 comprising the nucleotide sequence according to any one of SEQ ID NOs: 211-213.

102. The isolated nucleic acid molecule according to claim 91 comprising the nucleotide sequence according to any one of SEQ ID NOs: 214-216.

103. The isolated nucleic acid molecule according to claim 91 comprising the nucleotide sequence according to any one of SEQ ID NOs: 217-219.

104. A vector comprising the isolated nucleic acid molecule of any one of claims 91-103.

105. A method of treating cancer in a subject in need thereof, comprising administering a therapeutically effective amount of the binding protein of any one of claims 1-89 or the pharmaceutical composition of claim 90 to the subject.

106. A method of treating an inflammatory disease and/or autoimmune disorder in a subject in need thereof, comprising administering a therapeutically effective amount of the binding protein of any one of claims 1-89 or the pharmaceutical composition of claim 90 to the subject.

107. The method of claim 105, wherein the cancer is a B-cell malignancy, liver cancer, hepatocellular carcinoma (HCC), lung cancer, non-small cell lung cancer (NSCLC), squamous non-small cell lung cancer (sqNSCLC), ovarian cancer, clear cell ovarian cancer, carcinoma, Merkel cell carcinoma, gastric cancer, hepatoblastoma, nephroblastoma, melanoma, sarcoma, renal cell carcinoma, head and neck squamous cell carcinoma, urothelial cell carcinoma, osteosarcoma, glioblastoma, thyroid cancer, multiple myeloma, prostate cancer or Ewing's sarcoma.

108. The method of claim 106, wherein the inflammatory disease and/or autoimmune disorder is scleroderma, systemic lupus erythematosus (SLE), myositis, rheumatoid arthritis (RA), Sjogren's syndrome, or anti-neutrophil cytoplasmic autoantibody (ANCA) vasculitis.

109. The method of claim 105, wherein the tumor-associated antigen of the binding protein is CD20 and the cancer is a B-cell malignancy.

110. The method of claim 109, wherein the B-cell malignancy is non-Hodgkin's lymphoma (NHL), chronic lymphocytic leukemia (CLL), mantle cell lymphoma (MCL), diffuse large B-cell lymphoma (DLBCL), follicular lymphoma (FL), Burkett lymphoma, primary mediastinal large B cell lymphoma (PMBCL) or small lymphocytic lymphoma (SLL).

111. The method of claim 105, wherein the tumor-associated antigen of the binding protein is Glypican-3 (GPC3), and the cancer is liver cancer, hepatocellular carcinoma (HCC), non-small cell lung cancer (NSCLC), squamous non-small cell lung cancer (sqNSCLC), ovarian cancer, clear cell ovarian cancer, carcinoma, Merkel cell carcinoma, gastric cancer, hepatoblastoma, or nephroblastoma.

112. The method of claim 105, wherein the tumor-associated antigen of the binding protein is leucine rich repeat containing 15 (LRRC15), and the cancer is melanoma, sarcoma, renal cell carcinoma, head and neck squamous cell carcinoma, urothelial cell carcinoma, osteosarcoma, glioblastoma, lung cancer, non-small cell lung cancer, or thyroid cancer.

113. The method of claim 105, wherein the tumor-associated antigen of the binding protein is B cell maturation antigen (BCMA), and the cancer is multiple myeloma.

114. The method of claim 106, wherein the tumor-associated antigen of the binding protein is B cell maturation antigen (BCMA), and the autoimmune disorder is scleroderma, systemic lupus erythematosus (SLE), myositis, rheumatoid arthritis (RA) or Sjogren's syndrome.

115. The method of claim 105, wherein the tumor-associated antigen of the binding protein is STEAP2, and the cancer is prostate cancer or Ewing's sarcoma.

116. A method of treating systemic lupus erythematosus (SLE) in a subject in need thereof, comprising administering a therapeutically effective amount of the binding protein of any one of claims 1-89 or the pharmaceutical composition of claim 90 to the subject.

117. A method of treating myositis in a subject in need thereof, comprising administering a therapeutically effective amount of the binding protein of any one of claims 1-89 or the pharmaceutical composition of claim 90 to the subject.

118. A method of treating scleroderma in a subject in need thereof, comprising administering a therapeutically effective amount of the binding protein of any one of claims 1-89 or the pharmaceutical composition of claim 90 to the subject.

119. A method of treating rheumatoid arthritis (RA) in a subject in need thereof, comprising administering a therapeutically effective amount of the binding protein of any one of claims 1-89 or the pharmaceutical composition of claim 90 to the subject.

120. A method of treating Sjogren's syndrome in a subject in need thereof, comprising administering a therapeutically effective amount of the binding protein of any one of claims 1-89 or the pharmaceutical composition of claim 90 to the subject.

121. A method of treating anti-neutrophil cytoplasmic autoantibody (ANCA) vasculitis in a subject in need thereof, comprising administering a therapeutically effective amount of the binding protein of any one of claims 1-89 or the pharmaceutical composition of claim 90 to the subject.

122. A method of treating a B cell malignancy in a subject in need thereof, comprising administering a therapeutically effective amount of the binding protein of any one of claims 1-89 or the pharmaceutical composition of claim 90 to the subject.

123. A method of treating hepatocellular carcinoma in a subject in need thereof, comprising administering a therapeutically effective amount of the binding protein of any one of claims 1-89 or the pharmaceutical composition of claim 90 to the subject.

124. A method of treating osteosarcoma in a subject in need thereof, comprising administering a therapeutically effective amount of the binding protein of any one of claims 1-89 or the pharmaceutical composition of claim 90 to the subject.

125. A method of treating multiple myeloma in a subject in need thereof, comprising administering a therapeutically effective amount of the binding protein of any one of claims 1-89 or the pharmaceutical composition of claim 90 to the subject.

126. The method of any one of claims 105 to 125, wherein the binding protein preferentially activates a subset of T cells in the subject.

127. The method of claim 126, wherein the subset of T cells are CD8+ T cells.

128. The method of claim 127, wherein the CD8+ T cells are preferentially activated as compared to CD4+ T cells.

129. The method of any one of claims 126-128, wherein the activation of T cells is determined by measuring the percentage of surface CD25+ T cells.

130. The method of claim 129, wherein the percentage of surface CD25+ T cells that are CD8+ T cells is higher than the percentage of surface CD25+ T cells that are CD4+ T cells.

131. The method of any one of claims 105-130, wherein the binding protein is administered by subcutaneous administration.

132. A binding protein of any one of claims 1-89, or the pharmaceutical composition of claim 90, for use as a medicament.

133. A binding protein of any one of claims 1-89, or the pharmaceutical composition of claim 90, for use in the treatment of cancer.

134. A binding protein of any one of claims 1-89, or the pharmaceutical composition of claim 90, for use in the treatment of an inflammatory disease and/or autoimmune disorder.

135. The binding protein or pharmaceutical composition for use of claim 133, wherein the tumor-associated antigen is CD20, and the cancer is a B-cell malignancy.

136. The binding protein or pharmaceutical composition for use of claim 135, wherein the B-cell malignancy is non-Hodgkin's lymphoma (NHL), chronic lymphocytic leukemia (CLL), mantle cell lymphoma (MCL), diffuse large B-cell lymphoma (DLBCL), follicular lymphoma (FL), Burkett lymphoma, primary mediastinal large B cell lymphoma (PMBCL) or small lymphocytic lymphoma (SLL).

137. The binding protein or pharmaceutical composition for use of claim 133, wherein the tumor-associated antigen is Glypican-3 (GPC3), and the cancer is liver cancer or hepatocellular carcinoma (HCC), non-small cell lung cancer (NSCLC), squamous non-small cell lung cancer (sqNSCLC), ovarian cancer, clear cell ovarian cancer, carcinoma, Merkel cell carcinoma, gastric cancer, hepatoblastoma, or nephroblastoma.

138. The binding protein or pharmaceutical composition for use of claim 133, wherein the tumor-associated antigen is leucine rich repeat containing 15 (LRRC15) and the cancer is melanoma, sarcoma, renal cell carcinoma, head and neck squamous cell carcinoma, urothelial cell carcinoma, osteosarcoma, glioblastoma, lung cancer, non-small cell lung cancer, or thyroid cancer.

139. The binding protein or pharmaceutical composition for use of claim 133, wherein the tumor-associated antigen is BCMA, and the cancer is multiple myeloma.

140. The binding protein or pharmaceutical composition for use of claim 133, wherein the tumor-associated antigen is STEAP2, and the cancer is prostate cancer or Ewing's sarcoma.

141. The binding protein or pharmaceutical composition for use of claim 134, wherein the inflammatory disease and/or autoimmune disorder is scleroderma, systemic lupus erythematosus (SLE), myositis, rheumatoid arthritis (RA), Sjogren's syndrome, or ANCA vasculitis.

142. A binding protein of any one of claims 1-89, or the pharmaceutical composition of claim 90, for use in the treatment of systemic lupus erythematosus (SLE).

143. A binding protein of any one of claims 1-89, or the pharmaceutical composition of claim 90, for use in the treatment of myositis.

144. A binding protein of any one of claims 1-89, or the pharmaceutical composition of claim 90, for use in the treatment of scleroderma.

145. A binding protein of any one of claims 1-89, or the pharmaceutical composition of claim 90, for use in the treatment of rheumatoid arthritis (RA).

146. A binding protein of any one of claims 1-89, or the pharmaceutical composition of claim 90, for use in the treatment of Sjogren's syndrome.

147. A binding protein of any one of claims 1-89, or the pharmaceutical composition of claim 90, for use in the treatment of ANCA vasculitis.

148. A binding protein of any one of claims 1-89, or the pharmaceutical composition of claim 90, for use in the treatment of a B-cell malignancy.

149. A binding protein of any one of claims 1-89, or the pharmaceutical composition of claim 90, for use in the treatment of hepatocellular carcinoma.

150. A binding protein of any one of claims 1-89, or the pharmaceutical composition of claim 90, for use in the treatment of osteosarcoma.

151. A binding protein of any one of claims 1-89, or the pharmaceutical composition of claim 90, for use in the treatment of multiple myeloma.

152. Use of a binding protein of any one of claims 1-89, or the pharmaceutical composition of claim 90, for the manufacture of a medicament for treating cancer.

153. Use of a binding protein of any one of claims 1-89, or the pharmaceutical composition of claim 90, for the manufacture of a medicament for treating an inflammatory and/or autoimmune disorder.

154. The use of claim 152, wherein the cancer is a B-cell malignancy, liver cancer, or HCC.

155. The use of claim 154, wherein the B-cell malignancy is non-Hodgkin's lymphoma (NHL), chronic lymphocytic leukemia (CLL), mantle cell lymphoma (MCL), diffuse large B-cell lymphoma (DLBCL), follicular lymphoma (FL), Burkett lymphoma, primary mediastinal large B cell lymphoma (PMBCL) or small lymphocytic lymphoma (SLL).

156. The use of claim 152, wherein the cancer is melanoma, sarcoma, renal cell carcinoma, head and neck squamous cell carcinoma, urothelial cell carcinoma, osteosarcoma, glioblastoma, lung cancer, non-small cell lung cancer, or thyroid cancer.

157. The use of claim 153, wherein the inflammatory and/or autoimmune disorder is scleroderma, systemic lupus erythematosus (SLE), myositis, rheumatoid arthritis (RA), Sjogren's syndrome, or ANCA vasculitis.

158. Use of a binding protein of any one of claims 1-89, or the pharmaceutical composition of claim 90, for the manufacture of a medicament for the treatment of systemic lupus erythematosus (SLE).

159. Use of a binding protein of any one of claims 1-89, or the pharmaceutical composition of claim 90, for the manufacture of a medicament for the treatment of myositis.

160. Use of a binding protein of any one of claims 1-89, or the pharmaceutical composition of claim 90, for the manufacture of a medicament for the treatment of scleroderma.

161. Use of a binding protein of any one of claims 1-89, or the pharmaceutical composition of claim 90, for the manufacture of a medicament for the treatment of rheumatoid arthritis (RA).

162. Use of a binding protein of any one of claims 1-89, or the pharmaceutical composition of claim 90, for the manufacture of a medicament for the treatment of Sjogren's syndrome.

163. Use of a binding protein of any one of claims 1-89, or the pharmaceutical composition of claim 90, for the manufacture of a medicament for the treatment of ANCA vasculitis.

164. Use of a binding protein of any one of claims 1-89, or the pharmaceutical composition of claim 90, for the manufacture of a medicament for the treatment of a B-cell malignancy.

165. Use of a binding protein of any one of claims 1-89, or the pharmaceutical composition of claim 90, for the manufacture of a medicament for the treatment of hepatocellular carcinoma.

166. Use of a binding protein of any one of claims 1-89, or the pharmaceutical composition of claim 90, for the manufacture of a medicament for the treatment of osteosarcoma.

167. Use of a binding protein of any one of claims 1-89, or the pharmaceutical composition of claim 90, for the manufacture of a medicament for the treatment of multiple myeloma.

168. A binding protein comprising a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 136, SEQ ID NO: 137, SEQ ID NO: 138, SEQ ID NO: 139, and SEQ ID NO: 140 respectively.

169. The binding protein of claim 168, comprising a V_{H} chain according to SEQ ID NO: 141 and a V_{L} according to SEQ ID NO: 142.

170. A binding protein comprising a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 147, SEQ ID NO: 137, SEQ ID NO: 138, SEQ ID NO: 139, and SEQ ID NO: 140 respectively.

171. The binding protein of claim 170, comprising a V_{H} chain according to SEQ ID NO: 148 and a V_{L} according to SEQ ID NO: 142.

172. A binding protein comprising a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 152, SEQ ID NO: 137, SEQ ID NO: 138, SEQ ID NO: 139, and SEQ ID NO: 140 respectively.

173. The binding protein of claim 172, comprising a V_{H} chain according to SEQ ID NO: 153 and a V_{L} according to SEQ ID NO: 142.

174. A binding protein comprising a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 157, SEQ ID NO: 137, SEQ ID NO: 138, SEQ ID NO: 158, and SEQ ID NO: 140 respectively.

175. The binding protein of claim 174, comprising a V_{H} chain according to SEQ ID NO: 159 and a V_{L} according to SEQ ID NO: 160.

176. A binding protein comprising a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequences of SEQ ID NO: 135, SEQ ID NO: 157, SEQ ID NO: 137, SEQ ID NO: 138, SEQ ID NO: 165, and SEQ ID NO: 140 respectively.

177. The binding protein of claim 176, comprising a V_{H} chain according to SEQ ID NO: 159 and a V_{L} according to SEQ ID NO: 166.

178. A binding protein comprising a heavy chain CDR1 (HCDR1), a heavy chain CDR2 (HCDR2) and a heavy chain CDR3 (HCDR3) comprising the amino acid sequences of SEQ ID NO: 169, SEQ ID NO: 170 and SEQ ID NO: 171 respectively.

179. The binding protein of claim 178, comprising a VHH chain according to SEQ ID NO: 168.

180. A binding protein comprising a heavy chain CDR1 (HCDR1), a heavy chain CDR2 (HCDR2) and a heavy chain CDR3 (HCDR3) comprising the amino acid sequences of SEQ ID NO: 62, SEQ ID NO: 40 and SEQ ID NO: 41 respectively.

181. The binding protein of claim 180, comprising a VHH chain according to SEQ ID NO: 43 or SEQ ID NO: 45.

182. A binding protein, comprising a VHH chain according to SEQ ID NO: 44, SEQ ID NO: 46, SEQ ID NO: 47 or SEQ ID NO: 48.
